SEQUENCE LISTING (58 sequences)

SEQ ID NO: 1 - protein, 159 aa, Mycobacterium tuberculosis
MKRGLTVAVAGAAILVAGLSGCSSNKSTTGSGETTTAAGTTASPGAASGPKVVIDGKDQNVTGSVVCTTAAGNVNIAIGGAATGIAAVLTDGNPPEVKSVGLGNVNGVTLGYTSGTGQGNASATKDGSHYKITGTATGVDMANPMSPVNKSFEIEVTCS

SEQ ID NO: 2 - protein, 374 aa, Mycobacterium tuberculosis
MKIRLHTLLAVLTAAPLLLAAAGCGSKPPSGSPETGAGAGTVATTPASSPVTLAETGSTLLYPLFNLWGPAFHERYPNVTITAQGTGSGAGIAQAAAGTVNIGASDAYLSEGDMAAHKGLMNIALAISAQQVNYNLPGVSEHLKLNGKVLAAMYQGTIKTWDDPQIAALNPGVNLPGTAVVPLHRSDGSGDTFLFTQYLSKQDPEGWGKSPGFGTTVDFPAVPGALGENGNGGMVTGCAETPGCVAYIGISFLDQASQRGLGEAQLGNSSGNFLLPDAQSIQAAAAGFASKTPANQAISMIDGPAPDGYPIINYEYAIVNNRQKDAATAQTLQAFLHWAITDGNKASFLDQVHFQPLPPAVVKLSDALIATISS

SEQ ID NO: 3 - protein, 26 aa, Homo sapiens
MRVMAPRTLILLLSGALALTETWAGS

SEQ ID NO: 4 - protein, 27 aa, Herpes simplex virus 1
MGGAAARLGAVILFVVIVGLHGVRSKY

SEQ ID NO: 5 - protein, 27 aa, Herpes simplex virus 1
MGGAAARLGAVILFVVIVGLHGVRGKY

SEQ ID NO: 6 - protein, 25 aa, Herpes simplex virus 2
MGRLTSGVGTAALLVVAVGLRVVCA

SEQ ID NO: 7 - protein, 28 aa, Herpes simplex virus 2
MGRLTSGVGTAALLVVAVGLRVVCAKYA

SEQ ID NO: 8 - protein, 19 aa, SARS CoV-2
MFVFLVLLPLVSSQCVNLT

SEQ ID NO: 9 - protein, 21 aa, Homo sapiens
MDWIWRILFLVGAATGAHSQM

SEQ ID NO: 10 - protein, 20 aa, Homo sapiens
METPAQLLFLLLLWLPDTTG

SEQ ID NO: 11 - protein, 18 aa, Synthetic construct
MDWTWILFLVAAATRVHS

SEQ ID NO: 12 - protein, 24 aa, Japanese encephalitis virus
MLGSNSGQRVVFTILLLLVAPAYS

SEQ ID NO: 13 - protein, 17 aa, Vesicular stomatitis virus
MKCLLYLAFLFIGVNCA

SEQ ID NO: 14 - protein, 19 aa, Homo sapiens
MELGLSWIFLLAILKGVQC

SEQ ID NO: 15 - protein, 19 aa, Homo sapiens
MELGLRWVFLVAILEGVQC

SEQ ID NO: 16 - protein, 19 aa, Homo sapiens
MKHLWFFLLLVAAPRWVLS

SEQ ID NO: 17 - protein, 19 aa, Homo sapiens
MDWTWRILFLVAAATGAHS

SEQ ID NO: 18 - protein, 19 aa, Homo sapiens
MDWTWRFLFVVAAATGVQS

SEQ ID NO: 19 - protein, 19 aa, Homo sapiens
MEFGLSWLFLVAILKGVQC

SEQ ID NO: 20 - protein, 19 aa, Homo sapiens
MEFGLSWVFLVALFRGVQC

SEQ ID NO: 21 - protein, 26 aa, Homo sapiens
MDLLHKNMKHLWFFLLLVAAPRWVLS

SEQ ID NO: 22 - protein, 22 aa, Homo sapiens
MDMRVPAQLLGLLLLWLSGARC

SEQ ID NO: 23 - protein, 22 aa, Homo sapiens
MKYLLPTAAAGLLLLAAQPAMA

SEQ ID NO: 24 - RNA, 81 nt, Herpes simplex virus 1
  source 1..81: mol_type = transcribed RNA
atggggggggctgccgccaggttgggggccgtgattttgtttgtcgtcatagtgggcctccatggggtccgcagcaaatat

SEQ ID NO: 25 - RNA, 81 nt, Synthetic construct
  source 1..81: mol_type = other RNA
atgggaggagccgccgccagactgggagccgtgatcctgttcgtggtgatcgtgggactgcatggagtgagaagcaagtac

SEQ ID NO: 26 - RNA, 57 nt, Synthetic construct
  source 1..57: mol_type = other RNA
atgtttgtgtttcttgtgctgctgcctcttgtgtcttctcagtgtgtgaatttgaca

SEQ ID NO: 27 - RNA, 63 nt, Synthetic construct
  source 1..63: mol_type = other RNA
atggattggatttggagaatcctgttcctcgtgggagccgctacaggagcccactcccagatg

SEQ ID NO: 28 - RNA, 57 nt, Homo sapiens
  source 1..57: mol_type = transcribed RNA
atggagttgggactgagctggattttccttttggctattttaaaaggtgtccagtgt

SEQ ID NO: 29 - RNA, 57 nt, Homo sapiens
  source 1..57: mol_type = transcribed RNA
atggaactggggctccgctgggttttccttgttgctattttagaaggtgtccagtgt

SEQ ID NO: 30 - RNA, 57 nt, Homo sapiens
  source 1..57: mol_type = transcribed RNA
atgaaacacctgtggttcttcctcctgctggtggcagctcccagatgggtcctgtcc

SEQ ID NO: 31 - RNA, 57 nt, Homo sapiens
  source 1..57: mol_type = transcribed RNA
atggactggacctggaggatcctcttcttggtggcagcagcaacaggtgcccactcg

SEQ ID NO: 32 - RNA, 57 nt, Homo sapiens
  source 1..57: mol_type = transcribed RNA
atggactggacctggaggttcctctttgtggtggcagcagctacaggtgtccagtcc

SEQ ID NO: 33 - RNA, 57 nt, Homo sapiens
  source 1..57: mol_type = transcribed RNA
atggagtttgggctgagctggctttttcttgtggcgattctaaaaggtgtccagtgt

SEQ ID NO: 34 - RNA, 57 nt, Homo sapiens
  source 1..57: mol_type = transcribed RNA
atggagtttgggctgagctgggttttcctcgttgctctttttagaggtgtccagtgt

SEQ ID NO: 35 - RNA, 78 nt, Synthetic construct
  source 1..78: mol_type = other RNA
atggacctcctgcacaagaacatgaaacacctgtggttcttcctcctcctggtggcagctcccagatgggtgctgtcc

SEQ ID NO: 36 - RNA, 66 nt, Homo sapiens
  source 1..66: mol_type = transcribed RNA
atggacatgagggtccctgctcagctcctggggctcctgctgctctggctctcaggtgccagatgt

SEQ ID NO: 37 - RNA, 66 nt, Synthetic construct
  source 1..66: mol_type = other RNA
atgaaatacctattgcctacggcagccgctggattgttattactcgcggcccagccggccatggcc

SEQ ID NO: 38 - protein, 55 aa, Synthetic construct
IVGIVAGLAVLAVVVIGAVVATVMCRRKSSGGKGGSYSQAASSDSAQGSDVSLTA

SEQ ID NO: 39 - RNA, 47 nt, synthetic construct
  source 1..47: mol_type = other RNA
aactagtattcttctggtccccacagactcagagagaacccgccacc

SEQ ID NO: 40 - RNA, 278 nt, synthetic construct
  source 1..278: mol_type = other RNA
ctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacacc

SEQ ID NO: 41 - RNA, 110 nt, synthetic construct
  source 1..110: mol_type = other RNA
aaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 42 - protein, 8 aa, Synthetic construct
DYKDDDDK

SEQ ID NO: 43 - protein, 159 aa, synthetic construct
MKRGLTVAVAGAAILVAGLSGCSSQKSTTGSGETTTAAGTTASPGAASGPKVVIDGKDQQVTGSVVCTTAAGNVNIAIGGAATGIAAVLTDGNPPEVKSVGLGNVNGVTLGYTSGTGQGQASATKDGSHYKITGTATGVDMANPMSPVNKSFEIEVTCS

SEQ ID NO: 44 - protein, 374 aa, synthetic construct
MKIRLHTLLAVLTAAPLLLAAAGCGSKPPSGSPETGAGAGTVATTPASSPVTLAETGSTLLYPLFNLWGPAFHERYPQVTITAQGTGSGAGIAQAAAGTVNIGASDAYLSEGDMAAHKGLMNIALAISAQQVNYNLPGVSEHLKLNGKVLAAMYQGTIKTWDDPQIAALNPGVNLPGTAVVPLHRSDGSGDTFLFTQYLSKQDPEGWGKSPGFGTTVDFPAVPGALGENGNGGMVTGCAETPGCVAYIGISFLDQASQRGLGEAQLGQSSGNFLLPDAQSIQAAAAGFASKTPANQAISMIDGPAPDGYPIINYEYAIVNNRQKDAATAQTLQAFLHWAITDGNKASFLDQVHFQPLPPAVVKLSDALIATISS

SEQ ID NO: 45 - protein, 134 aa, synthetic construct
KSTTGSGETTTAAGTTASPGAASGPKVVIDGKDQNVTGSVVCTTAAGNVNIAIGGAATGIAAVLTDGNPPEVKSVGLGNVNGVTLGYTSGTGQGNASATKDGSHYKITGTATGVDMANPMSPVNKSFEIEVTCS

SEQ ID NO: 46 - protein, 348 aa, synthetic construct
KPPSGSPETGAGAGTVATTPASSPVTLAETGSTLLYPLFNLWGPAFHERYPNVTITAQGTGSGAGIAQAAAGTVNIGASDAYLSEGDMAAHKGLMNIALAISAQQVNYNLPGVSEHLKLNGKVLAAMYQGTIKTWDDPQIAALNPGVNLPGTAVVPLHRSDGSGDTFLFTQYLSKQDPEGWGKSPGFGTTVDFPAVPGALGENGNGGMVTGCAETPGCVAYIGISFLDQASQRGLGEAQLGNSSGNFLLPDAQSIQAAAAGFASKTPANQAISMIDGPAPDGYPIINYEYAIVNNRQKDAATAQTLQAFLHWAITDGNKASFLDQVHFQPLPPAVVKLSDALIATISS

SEQ ID NO: 47 - RNA, 477 nt, synthetic construct
  source 1..477: mol_type = other RNA
gtgaaaaggggcctgacagttgctgttgccggcgctgctattctggtggctggactgtctggctgcagcagcaacaagagcacaacaggcagcggcgagacaacaacagccgccggaacaacagcttctcctggcgctgcttctggccccaaggttgtgatcgatggcaaggaccagaacgtgaccggcagcgtcgtgtgtacaaccgccgctggcaatgtgaatatcgccattggcggagccgccacaggcattgctgctgtgctgacagatggcaaccctcctgaagtgaagtctgtcggcctgggcaatgtcaacggcgtgacactgggctacacaagcggaacaggacagggcaatgccagcgccacaaaggatggcagccactacaagatcaccggcacagctacaggcgtggacatggccaatcctatgagccccgtgaacaagtccttcgagatcgaagtgacctgcagc

SEQ ID NO: 48 - RNA, 585 nt, synthetic construct
  source 1..585: mol_type = other RNA
atgagagtgatggcccccagaaccctgatcctgctgctgtctggcgccctggccctgacagagacatgggccggaagcgactacaaagacgatgacgacaaggtgaaaaggggcctgacagttgctgttgccggcgctgctattctggtggctggactgtctggctgcagcagcaacaagagcacaacaggcagcggcgagacaacaacagccgccggaacaacagcttctcctggcgctgcttctggccccaaggttgtgatcgatggcaaggaccagaacgtgaccggcagcgtcgtgtgtacaaccgccgctggcaatgtgaatatcgccattggcggagccgccacaggcattgctgctgtgctgacagatggcaaccctcctgaagtgaagtctgtcggcctgggcaatgtcaacggcgtgacactgggctacacaagcggaacaggacagggcaatgccagcgccacaaaggatggcagccactacaagatcaccggcacagctacaggcgtggacatggccaatcctatgagccccgtgaacaagtccttcgagatcgaagtgacctgcagctgatga

SEQ ID NO: 49 - RNA, 477 nt, synthetic construct
  source 1..477: mol_type = other RNA
atgaaaaggggcctgacagttgctgttgccggcgctgctattctggtggctggactgtctggctgcagcagccagaaaagcacaacaggcagcggcgagacaacaacagccgccggaacaacagcttctcctggcgctgcttctggccccaaggttgtgatcgatggcaaggaccagcaggtgaccggcagcgtcgtgtgtacaaccgccgctggcaatgtgaatatcgccattggcggagccgccacaggcattgctgctgtgctgacagatggcaaccctcctgaagtgaagtctgtcggcctgggcaatgtcaacggcgtgacactgggctacacaagcggaacaggacagggccaggccagcgccacaaaggatggcagccactacaagatcaccggcacagctacaggcgtggacatggccaatcctatgagccccgtgaacaagtccttcgagatcgaagtgacctgcagc

SEQ ID NO: 50 - RNA, 402 nt, synthetic construct
  source 1..402: mol_type = other RNA
aagtccacaacaggcagcggcgagacaacaacagccgccggaacaacagcttctcctggcgctgcttctggccccaaggttgtgatcgatggcaaggaccagaacgtgaccggcagcgtcgtgtgtacaaccgccgctggcaatgtgaatatcgccattggcggagccgccacaggcattgctgctgtgctgacagatggcaaccctcctgaagtgaagtctgtcggcctgggcaatgtcaacggcgtgacactgggctacacaagcggaacaggacagggcaatgccagcgccacaaaggatggcagccactacaagatcaccggcacagctacaggcgtggacatggccaatcctatgagccccgtgaacaagtccttcgagatcgaagtgacctgcagc

SEQ ID NO: 51 - RNA, 510 nt, synthetic construct
  source 1..510: mol_type = other RNA
atgagagtgatggcccccagaaccctgatcctgctgctgtctggcgccctggccctgacagagacatgggccggaagcgactacaaagacgatgacgacaagaagtccacaacaggcagcggcgagacaacaacagccgccggaacaacagcttctcctggcgctgcttctggccccaaggttgtgatcgatggcaaggaccagaacgtgaccggcagcgtcgtgtgtacaaccgccgctggcaatgtgaatatcgccattggcggagccgccacaggcattgctgctgtgctgacagatggcaaccctcctgaagtgaagtctgtcggcctgggcaatgtcaacggcgtgacactgggctacacaagcggaacaggacagggcaatgccagcgccacaaaggatggcagccactacaagatcaccggcacagctacaggcgtggacatggccaatcctatgagccccgtgaacaagtccttcgagatcgaagtgacctgcagctgatga

SEQ ID NO: 52 - RNA, 480 nt, Mycobacterium tuberculosis
  source 1..480: mol_type = other RNA
gtgaagcgtggactgacggtcgcggtagccggagccgccattctggtcgcaggtctttccggatgttcaagcaacaagtcgactacaggaagcggtgagaccacgaccgcggcaggcacgacggcaagccccggcgccgcctccgggccgaaggtcgtcatcgacggtaaggaccagaacgtcaccggctccgtggtgtgcacaaccgcggccggcaatgtcaacatcgcgatcggcggggcggcgaccggcattgccgccgtgctcaccgacggcaaccctccggaggtgaagtccgttgggctcggtaacgtcaacggcgtcacgctgggatacacgtcgggcaccggacagggtaacgcctcggcaaccaaggacggcagccactacaagatcactgggaccgctaccggggtcgacatggccaacccgatgtcaccggtgaacaagtcgttcgaaatcgaggtgacctgttcctaa

SEQ ID NO: 53 - RNA, 1122 nt, synthetic construct
  source 1..1122: mol_type = other RNA
gtgaagatcagactgcacacactgctggccgtgctgacagctgctcctctgcttcttgctgccgccggatgtggatctaagcctccttctggctctcctgaaacaggcgctggtgctggcacagtggctacaacaccagcttctagccctgtgacactggccgagacaggctctacactgctgtaccctctgttcaacctgtggggccctgccttccacgagagataccccaacgtgaccatcacagcccaaggcacaggatctggcgccggaattgctcaagctgccgctggcaccgtgaatatcggagcctctgatgcctacctgagcgagggcgatatggccgctcacaagggcctgatgaatattgccctggccatcagcgcccagcaagtgaactataacctgcctggcgtgtccgagcacctgaagctgaatggaaaagtgctggccgccatgtaccagggcaccatcaagacctgggacgaccctcagatcgccgctctgaatccgggtgttaatctgcctggaaccgccgtggtgcctctgcacagatctgatggaagcggcgacaccttcctgttcacccagtacctgagcaagcaggaccctgaaggctggggcaagtctcctggctttggcaccaccgtggatttccctgctgttcctggtgctctgggcgagaatggcaatggcggcatggttaccggatgcgccgaaacacctggctgcgtggcctatatcggcatcagctttctggaccaggccagccagagaggactgggagaagctcagctgggcaacagcagcggcaatttcctgctgcctgacgctcagtctatccaggctgcagctgccggctttgcctctaagacaccagccaatcaggccatctccatgatcgacggccctgctcctgatggctaccccatcatcaactacgagtacgccatcgtgaacaaccggcagaaggatgccgccacagctcagacactgcaggcctttctgcactgggccatcaccgatggaaacaaggccagcttcctggatcaggtgcacttccagccactgcctccagccgtggtcaaactgtctgatgccctgatcgccaccatcagcagc

SEQ ID NO: 54 - RNA, 1230 nt, synthetic construct
  source 1..1230: mol_type = other RNA
atgagagtgatggcccccagaaccctgatcctgctgctgtctggcgccctggccctgacagagacatgggccggaagcgactacaaagacgatgacgacaaggtgaagatcagactgcacacactgctggccgtgctgacagctgctcctctgcttcttgctgccgccggatgtggatctaagcctccttctggctctcctgaaacaggcgctggtgctggcacagtggctacaacaccagcttctagccctgtgacactggccgagacaggctctacactgctgtaccctctgttcaacctgtggggccctgccttccacgagagataccccaacgtgaccatcacagcccaaggcacaggatctggcgccggaattgctcaagctgccgctggcaccgtgaatatcggagcctctgatgcctacctgagcgagggcgatatggccgctcacaagggcctgatgaatattgccctggccatcagcgcccagcaagtgaactataacctgcctggcgtgtccgagcacctgaagctgaatggaaaagtgctggccgccatgtaccagggcaccatcaagacctgggacgaccctcagatcgccgctctgaatccgggtgttaatctgcctggaaccgccgtggtgcctctgcacagatctgatggaagcggcgacaccttcctgttcacccagtacctgagcaagcaggaccctgaaggctggggcaagtctcctggctttggcaccaccgtggatttccctgctgttcctggtgctctgggcgagaatggcaatggcggcatggttaccggatgcgccgaaacacctggctgcgtggcctatatcggcatcagctttctggaccaggccagccagagaggactgggagaagctcagctgggcaacagcagcggcaatttcctgctgcctgacgctcagtctatccaggctgcagctgccggctttgcctctaagacaccagccaatcaggccatctccatgatcgacggccctgctcctgatggctaccccatcatcaactacgagtacgccatcgtgaacaaccggcagaaggatgccgccacagctcagacactgcaggcctttctgcactgggccatcaccgatggaaacaaggccagcttcctggatcaggtgcacttccagccactgcctccagccgtggtcaaactgtctgatgccctgatcgccaccatcagcagctgatga

SEQ ID NO: 55 - RNA, 1122 nt, synthetic construct
  source 1..1122: mol_type = other RNA
atgaagatcagactgcacacactgctggccgtgctgacagctgctcctctgcttcttgctgccgccggatgtggatctaagcctccttctggctctcctgaaacaggcgctggtgctggcacagtggctacaacaccagcttctagccctgtgacactggccgagacaggctctacactgctgtaccctctgttcaacctgtggggccctgccttccacgagagatacccccaggtgaccatcacagcccaaggcacaggatctggcgccggaattgctcaagctgccgctggcaccgtgaatatcggagcctctgatgcctacctgagcgagggcgatatggccgctcacaagggcctgatgaatattgccctggccatcagcgcccagcaagtgaactataacctgcctggcgtgtccgagcacctgaagctgaatggaaaagtgctggccgccatgtaccagggcaccatcaagacctgggacgaccctcagatcgccgctctgaatccgggtgttaatctgcctggaaccgccgtggtgcctctgcacagatctgatggaagcggcgacaccttcctgttcacccagtacctgagcaagcaggaccctgaaggctggggcaagtctcctggctttggcaccaccgtggatttccctgctgttcctggtgctctgggcgagaatggcaatggcggcatggttaccggatgcgccgaaacacctggctgcgtggcctatatcggcatcagctttctggaccaggccagccagagaggactgggagaagctcagctgggccagagcagcggcaatttcctgctgcctgacgctcagtctatccaggctgcagctgccggctttgcctctaagacaccagccaatcaggccatctccatgatcgacggccctgctcctgatggctaccccatcatcaactacgagtacgccatcgtgaacaaccggcagaaggatgccgccacagctcagacactgcaggcctttctgcactgggccatcaccgatggaaacaaggccagcttcctggatcaggtgcacttccagccactgcctccagccgtggtcaaactgtctgatgccctgatcgccaccatcagcagc

SEQ ID NO: 56 - RNA, 1044 nt, synthetic construct
  source 1..1044: mol_type = other RNA
aagcctcctagcggctctcctgaaacaggtgctggtgctggcacagtggccaccacaccagcatcttctccagtgacactggccgagacaggctctaccctgctgtaccctctgttcaacctgtggggccctgccttccacgagagataccccaacgtgaccatcacagcccaaggcacaggatctggcgccggaattgctcaagctgccgccggaacagtgaatatcggagcctctgatgcctacctgagcgagggcgatatggccgctcacaagggcctgatgaatattgccctggccatcagcgcccagcaagtgaactataacctgcctggcgtgtccgagcacctgaagctgaatggaaaagtgctggccgccatgtaccagggcaccatcaagacctgggacgaccctcagatcgccgctctgaatccgggtgttaatctgcctggaaccgccgtggtgcctctgcacagatctgatggaagcggcgacaccttcctgttcacccagtacctgagcaagcaggaccctgaaggctggggcaagtctcctggctttggcaccaccgtggatttccctgctgttcctggtgctctgggcgagaatggcaatggcggcatggttaccggatgcgccgaaacacctggctgcgtggcctatatcggcatcagctttctggaccaggccagccagagaggactgggagaagctcagctgggcaacagcagcggcaatttcctgctgcctgacgctcagtctatccaggctgctgctgccggctttgcctctaagacaccagccaatcaggccatctccatgatcgacggccctgctcctgatggctaccccatcatcaactacgagtacgccatcgtgaacaaccggcagaaggatgccgccacagctcagacactgcaggcctttctgcactgggccatcaccgatggaaacaaggccagcttcctggatcaggtgcacttccagccactgcctccagccgtggtcaaactgtctgatgccctgatcgccaccatcagcagc

SEQ ID NO: 57 - RNA, 1152 nt, synthetic construct
  source 1..1152: mol_type = other RNA
atgagagtgatggcccccagaaccctgatcctgctgctgtctggcgccctggccctgacagagacatgggccggaagcgactacaaagacgatgacgacaagaagcctcctagcggctctcctgaaacaggtgctggtgctggcacagtggccaccacaccagcatcttctccagtgacactggccgagacaggctctaccctgctgtaccctctgttcaacctgtggggccctgccttccacgagagataccccaacgtgaccatcacagcccaaggcacaggatctggcgccggaattgctcaagctgccgccggaacagtgaatatcggagcctctgatgcctacctgagcgagggcgatatggccgctcacaagggcctgatgaatattgccctggccatcagcgcccagcaagtgaactataacctgcctggcgtgtccgagcacctgaagctgaatggaaaagtgctggccgccatgtaccagggcaccatcaagacctgggacgaccctcagatcgccgctctgaatccgggtgttaatctgcctggaaccgccgtggtgcctctgcacagatctgatggaagcggcgacaccttcctgttcacccagtacctgagcaagcaggaccctgaaggctggggcaagtctcctggctttggcaccaccgtggatttccctgctgttcctggtgctctgggcgagaatggcaatggcggcatggttaccggatgcgccgaaacacctggctgcgtggcctatatcggcatcagctttctggaccaggccagccagagaggactgggagaagctcagctgggcaacagcagcggcaatttcctgctgcctgacgctcagtctatccaggctgctgctgccggctttgcctctaagacaccagccaatcaggccatctccatgatcgacggccctgctcctgatggctaccccatcatcaactacgagtacgccatcgtgaacaaccggcagaaggatgccgccacagctcagacactgcaggcctttctgcactgggccatcaccgatggaaacaaggccagcttcctggatcaggtgcacttccagccactgcctccagccgtggtcaaactgtctgatgccctgatcgccaccatcagcagctgatga

SEQ ID NO: 58 - RNA, 1125 nt, Mycobacterium tuberculosis
  source 1..1125: mol_type = other RNA
gtgaaaattcgtttgcatacgctgttggccgtgttgaccgctgcgccgctgctgctagcagcggcgggctgtggctcgaaaccaccgagcggttcgcctgaaacgggcgccggcgccggtactgtcgcgactacccccgcgtcgtcgccggtgacgttggcggagaccggtagcacgctgctctacccgctgttcaacctgtggggtccggcctttcacgagaggtatccgaacgtcacgatcaccgctcagggcaccggttctggtgccgggatcgcgcaggccgccgccgggacggtcaacattggggcctccgacgcctatctgtcggaaggtgatatggccgcgcacaaggggctgatgaacatcgcgctagccatctccgctcagcaggtcaactacaacctgcccggagtgagcgagcacctcaagctgaacggaaaagtcctggcggccatgtaccagggcaccatcaaaacctgggacgacccgcagatcgctgcgctcaaccccggcgtgaacctgcccggcaccgcggtagttccgctgcaccgctccgacgggtccggtgacaccttcttgttcacccagtacctgtccaagcaagatcccgagggctggggcaagtcgcccggcttcggcaccaccgtcgacttcccggcggtgccgggtgcgctgggtgagaacggcaacggcggcatggtgaccggttgcgccgagacaccgggctgcgtggcctatatcggcatcagcttcctcgaccaggccagtcaacggggactcggcgaggcccaactaggcaatagctctggcaatttcttgttgcccgacgcgcaaagcattcaggccgcggcggctggcttcgcatcgaaaaccccggcgaaccaggcgatttcgatgatcgacgggcccgccccggacggctacccgatcatcaactacgagtacgccatcgtcaacaaccggcaaaaggacgccgccaccgcgcagaccttgcaggcatttctgcactgggcgatcaccgacggcaacaaggcctcgttcctcgaccaggttcatttccagccgctgccgcccgcggtggtgaagttgtctgacgcgttgatcgcgacgatttccagctag

(19) United States
(12) Patent Application Publication
(10) Pub. No.: US 2026/0284168 A1
(43) Pub. Date: Sep. 24, 2026

(54) RNA FOR PREVENTING OR TREATING TUBERCULOSIS

(71) Applicants: BIONTECH SE, Mainz (DE); NATIONAL UNIVERSITY OF SINGAPORE, Singapore (SG); THE REGENTS OF THE UNIVERSITY OF CALIFORNIA, Oakland, CA (US)

(72) Inventors: Louis Simon ATES, Mainz (DE); Charles Lefco DULBERGER, Cambridge, MA (US); Julia UEBELE, Mainz (DE); Babak JAVID, Oakland, CA (US); Paul Anthony MACARY, Singapore (SG); Sandro HOFFMANN, Mainz (DE)

(21) Appl. No.: 19/474,867

(22) PCT Filed: Apr. 12, 2024
(86) PCT No.: PCT/US2024/024498

§ 371 Date: Oct. 13, 2025

Related U.S. Application Data
(60) Provisional application No. 63/496,142, filed on Apr. 14, 2023.
(60) Provisional application No. 63/568,992, filed on Mar. 22, 2024.

(51) Int. Cl. A61K 39/04; A61K 9/00; A61K 9/1272; A61K 9/51; A61K 39/00; A61P 31/06; C07K 14/005; C07K 14/35

(52) U.S. Cl. CPC A61K 39/04; A61K 9/0019; A61K 9/1272; A61K 9/5123; A61P 31/06; C07K 14/005; C07K 14/35; A61K 2039/53; A61K 2039/54; A61K 2039/545; A61K 2039/55522; A61K 2039/55555; A61K 2039/575; C07K 2319/02; C12N 2710/16622; C12N 2830/50

(57) ABSTRACT

The disclosure provides agents and methods for preventing or treating tuberculosis using RNA. The RNA encoding antigens of Mycobacterium tuberculosis, immunogenic variants or fragments thereof is formulated and administered in a way that the antigens, variants or fragments are produced by cells of a subject.

### CROSS REFERENCE TO RELATED APPLICATIONS

The present application claims priority to U.S. Provisional Patent Application Nos. 63/496,142, filed Apr. 14, 2023, and 63/568,992, filed Mar. 22, 2024, each of which is hereby incorporated by reference in its entirety.

### TECHNICAL FIELD

The disclosure provides agents and methods for preventing or treating tuberculosis using RNA. The RNA encoding antigens of Mycobacterium tuberculosis, immunogenic variants or fragments thereof is formulated and administered in a way that the antigens, variants or fragments are produced by cells of a subject, in particular after intramuscular or intravenous administration of the RNA.

### BACKGROUND

The use of RNA to deliver foreign genetic information into target cells offers an attractive alternative to DNA. The advantages of RNA include transient expression and non-transforming character. RNA does not require nucleus infiltration for expression and moreover cannot integrate into the host genome, thereby eliminating the risk of oncogenesis.

The COVID-19 pandemic has showcased the utility and advantages of RNA technology for vaccination, as out of all COVID-19 vaccines under development, the first two to have received emergency use authorization by the FDA were RNA-based. The biotechnology response to the COVID-19 pandemic has highlighted the speed and flexibility of mRNA vaccines, and reveals mRNA therapeutics to be a powerful tool to address epidemic outbreaks caused by newly emerging viruses. The relative simplicity of the development process and flexibility of the manufacturing platform can markedly accelerate clinical development. As such, mRNA-based vaccine technology has attracted a lot of attention during the COVID-19 pandemic.

The first authorized vaccine was developed by BioNTech in collaboration with Pfizer. The RNA of this vaccine, BNT162b2, encodes full length spike protein modified by two proline mutations to stabilize the prefusion conformation. The RNA incorporates 1-methyl-pseudouridine, which dampens innate immune sensing and increases mRNA translation in vivo and is formulated in lipid nanoparticles (LNP). BNT162b2 is administered to adults intramuscularly (IM) in two 30 μg doses given 21 days apart.

Tuberculosis (TB) is caused by the bacterial pathogen Mycobacterium tuberculosis (Mtb) and, in rarer cases, by other pathogens from the Mycobacteriaceae family and is the leading cause of death from a single infectious agent. Mtb is a gram-positive, rod-shaped bacterium from the Mycobacteriaceae family. The more than 4,000 genes encoded within an approximately 4 million base pair genome render Mtb a complex pathogenic organism. This is further emphasized by the atypical composition of its cell wall, which has a high lipid content.

Despite the observed trend for reduction in TB cases and TB-related deaths for the last 20 years, 1.42 million people died from TB alone in 2019. In addition to the active form of TB, difficulties arise from latent TB infection (LTBI), when the infected patient doesn't present clinical symptoms. The estimated 2 billion latently infected individuals worldwide pose a huge and unpredictable reservoir of Mtb (WORLD HEALTH ORGANIZATION. Global tuberculosis report 2019. Geneva, WORLD HEALTH ORGANIZATION; 2019. ISBN: 978-92-4-156571-4). The high prevalence of HIV-1 infections further increases the risk for TB disease acquisition, activation of a latent TB infection, and death from HIV-TB co-infection. In 2009, 0.2 million deaths were related to HIV-TB comorbidity. The complexity of the Mtb cell wall makes the bacterium resistant to environmental impact and to therapy with certain antibiotics. The latter further complicates anti-TB treatment especially in low- and middle-income countries (WORLD HEALTH ORGANIZATION. Global tuberculosis report 2020. Geneva, WORLD HEALTH ORGANIZATION; 2020. ISBN: 978-92-4-001313-1).

An attenuated strain of Mycobacterium bovis, bacillus Calmette-Guérin (BCG), is the only licensed TB vaccine, introduced in 1921. The use of the live vaccine BCG is not recommended for immunocompromised individuals and the protective efficacy against pulmonary TB conferred by immunization with BCG is highly variable, ranging from 50-80%. Moreover, passaging of BCG over the decades further attenuated the currently used BCG strains, reducing its protective efficacy (Brosch R, et al. Proc. Natl. Acad. Sci. U.S.A., 2007; 104 (13): 5596-5601). Thus, there is an unmet medical need for a safer and more effective vaccine to prevent TB, especially for a vaccine that can be administered to immunocompromised individuals.

The pipeline of clinical trials for TB vaccine candidates comprises use of live, live-attenuated, and inactivated mycobacteria, and of Mtb antigens as recombinant protein (subunit vaccine) (TuBerculosis Vaccine Initiative (TBVI). Available from: https://www.tbvi.eu/what-we-do/pipeline-of-vaccines/). The drawbacks from these vaccine platforms are i. their low safety, due to replication-competent live vaccines still being infectious, ii. low immunogenicity of inactivated vaccines, and iii, the need for addition of adjuvants to subunit vaccines to enhance immunogenicity. To date, most vaccine candidates have failed to demonstrate better protection from TB or from the development of TB compared to placebo in clinical trials.

For all these reasons, novel agents for preventing or treating tuberculosis are required.

### SUMMARY

The present disclosure provides compositions which are useful as TB vaccines. The compositions provided herein comprise RNA for delivering Mtb antigens to a subject. The findings described herein demonstrate that RNA described herein, e.g., non-modified uridine containing mRNA (uRNA) or nucleoside modified mRNA (modRNA), expressing antigens of Mycobacterium tuberculosis, immunogenic variants or fragments thereof, is useful for preventing or treating tuberculosis. The RNA encoding antigens of Mycobacterium tuberculosis, immunogenic variants or fragments thereof is formulated and administered in a way that the antigens, variants or fragments can be produced and preferably secreted by patient cells to prevent or combat tuberculosis.

Mtb displays differential gene expression patterns during its active and dormant (non-dividing) phases (Andersen P, et al. Cold Spring Harb Perspect Med, 2014; 4 (6): a018523). To prevent development of TB, there should be immunity against antigens specific for each of the various stages of Mtb infection. The TB vaccine candidate developed here comprising the RNA components described above is designed to induce protective immune responses against antigens specific for different stages of Mtb infection.

Unlike the attenuated vaccine BCG, this TB vaccine candidate does not carry the risks associated with infection and may therefore be given to people who cannot be administered live organism (such as pregnant women and immunocompromised persons).

In one aspect, the disclosure provides an RNA molecule comprising a sequence encoding at least one full-length antigen or antigen fragment representing at least one Mycobacterium tuberculosis antigen or immunogenic variant thereof, wherein the Mycobacterium tuberculosis antigen is selected from the group comprising LpqH and PstS1.

In some embodiments, if a Mycobacterium tuberculosis antigen or immunogenic variant thereof is represented by antigen fragment(s), the number of antigen fragments may be 1, 2, 3, 4, 5 or more fragments. In some embodiments, said fragments may be overlapping or non-overlapping. In some embodiments, said fragments may be separated by polypeptide linkers.

In some embodiments of the RNA molecule, the sequence of one or more of the at least one full-length antigens or antigen fragments is altered by removal of a predicted bacterial signal peptide.

In some embodiments, the sequence of one or more of the at least one full-length antigens or antigen fragments is altered by addition of a non-native human, bacterial or viral signal peptide to its N-terminus.

In some embodiments, the non-native signal peptide is selected from the group comprising a HSV-1 glycoprotein D signal peptide, a HSV-2 glycoprotein D signal peptide, a human Ig heavy chain signal peptide, a HuIgGk signal peptide, an IgE heavy chain epsilon-1 signal peptide, a Japanese encephalitis PRM signal sequence or a VSVg protein signal sequence.

In some embodiments, the non-native signal peptide is a viral signal peptide, preferably, wherein the non-native signal peptide is a HSV-1 glycoprotein D signal peptide.

In some embodiments, the amino acid sequence of one or more of the at least one full-length antigens or antigen fragments is altered by replacing at least one transmembrane domain with a disrupted transmembrane domain. In some embodiments, the amino acid sequence of one or more of the at least one full-length antigens or antigen fragments is altered by addition of a non-native trafficking domain and/or non-native transmembrane domain to its C-terminus.

In some embodiments, the non-native trafficking domain is an MHC class I trafficking domain and/or wherein the non-native transmembrane domain is a human, bacterial or viral transmembrane domain.

In some embodiments:
a) the LpqH antigen comprises the amino acid sequence of any one of SEQ ID NOS: 1, 43 or 45 and an immunogenic variant thereof comprises an amino acid sequence having at least 99%, 98%, 97%, 96%, 95%, 90%, 85%, or 80% identity to the amino acid sequence of SEQ ID NO: 1, 43 or 45;
b) the PstS1 antigen comprises the amino acid sequence of any one of SEQ ID NOs: 2, 44 or 46 and an immunogenic variant thereof comprises an amino acid sequence having at least 99%, 98%, 97%, 96%, 95%, 90%, 85%, or 80% identity to the amino acid sequence of SEQ ID NO: 2, 44 or 46;
c) the non-native signal peptide comprises an amino acid sequence selected from the group of SEQ ID NOs: 3 to 23, amino acid sequences having at least 96%, 92%, 88%, 84% or 80% identity to SEQ ID NOs: 3 to 23, amino acid sequences encoded by a nucleotide sequence selected from the group of SEQ ID NOs: 24 to 37 and amino acid sequences having at least 96%, 92%, 88%, 84% or 80% identity to amino acid sequences encoded by a nucleotide sequence selected from the group of SEQ ID NOs: 24 to 37; and/or
d) the non-native trafficking signal comprises the amino acid sequence SEQ ID NO: 38 or an amino acid sequence having at least 98%, 96%, 94%, 92%, 90%, 85% or 80% identity to the amino acid sequence of SEQ ID NO: 38.

In some embodiments, the RNA molecule comprises a 5′ cap. In some embodiments, the 5′ cap comprises a cap1 structure. In some embodiments, the 5′-cap comprises m₂^{7,3′-O}Gppp (m₁^{2′-O})ApG.

In some embodiments, the RNA molecule comprises a 5′-UTR. In some embodiments, the 5′-UTR comprises a modified human alpha-globin 5′-UTR. In some embodiments, the 5′-UTR comprises the nucleotide sequence of SEQ ID NO: 39, or a nucleotide sequence having at least 98%, 96%, 94%, or 80% identity to the nucleotide sequence of SEQ ID NO: 39.

In some embodiments, the RNA comprises a 3′-UTR. In some embodiments, the 3′-UTR comprises a first sequence from the amino terminal enhancer of split (AES) messenger RNA and a second sequence from the mitochondrial encoded 12S ribosomal RNA. In some embodiments, the 3′-UTR comprises the nucleotide sequence of SEQ ID NO: 40, or a nucleotide sequence having at least 99%, 98%, 97%, 96%, 95%, 90%, 85%, or 80% identity to the nucleotide sequence of SEQ ID NO: 40.

In some embodiments, the RNA molecule comprises a polyA sequence. In some embodiments, the polyA sequence is an interrupted sequence of A nucleotides. In some embodiments, the polyA sequence comprises 30 adenine nucleotides followed by 70 adenine nucleotides, wherein the 30 adenine nucleotides and 70 adenine nucleotides are separated by a nucleotide linker sequence of 10 nucleotides. In some embodiments, the polyA sequence is or comprises the nucleotide sequence of SEQ ID NO: 41, or a nucleotide sequence having at least 99%, 98%, 97%, 96%, 95%, 90%, 85%, or 80% identity to the nucleotide sequence of SEQ ID NO: 41.

In some embodiments, the RNA molecule comprises a 5′-cap, a 5′-UTR, a 3′-UTR, and a polyA sequence.

In some embodiments, the RNA molecule comprises modified nucleotides, nucleosides or nucleobases. In some embodiments, the RNA molecule comprises modified uridines. In some embodiments, the RNA molecule comprises modified uridines in place of all uridines. In some embodiments, the modified uridines are N1-methyl-pseudouridine.

In some embodiments, the coding sequence of the RNA molecule is codon-optimized and/or is characterized in that its G/C content is increased compared to the parental sequence.

In one aspect, the disclosure provides a protein encoded by the RNA molecule disclosed herein.

In one aspect, the disclosure provides a DNA molecule encoding the RNA molecule disclosed herein.

In one aspect, the disclosure provides a pharmaceutical composition comprising one or more RNA molecules disclosed herein.

In some embodiments of the pharmaceutical composition, the one or more RNA molecule is formulated in a lipid formulation, such as in lipid nanoparticles or liposomes.

In some embodiments, the lipid formulation comprises each of:
a) a cationically ionizable lipid;
b) a steroid;
c) a neutral lipid; and
d) a polymer-conjugated lipid.

In some embodiments, the cationically ionizable lipid is present in a concentration ranging from about 40 to about 60 mol percent of the total lipids. In some embodiments, the steroid is present in a concentration ranging from about 30 to about 50 mol percent of the total lipids. In some embodiments, the neutral lipid is present in a concentration ranging from about 5 to about 15 mol percent of the total lipids. In some embodiments, the polymer-conjugated lipid is present in a concentration ranging from about 1 to about 10 mol percent of the total lipids. In some embodiments, the cationically ionizable lipid is within a range of about 40 to about 60 mole percent, the steroid is within a range of about 30 to about 50 mole percent, the neutral lipid is within a range of about 5 to about 15 mole percent, and the polymer-conjugated lipid is within a range of about 1 to about 10 mole percent.

In some embodiments, the cationically ionizable lipid comprises ((4-hydroxybutyl) azanediyl)bis(hexane-6,1-diyl)bis(2-hexyldecanoate). In some embodiments, the steroid comprises cholesterol. In some embodiments, the neutral lipid comprises a phospholipid. In some embodiments, the phospholipid comprises distearoylphosphatidylcholine (DSPC). In some embodiments, the polymer-conjugated lipid comprises a polyethylene glycol (PEG)-lipid. In some embodiments, the PEG-lipid comprises 2-[(polyethylene glycol)-2000]-N,N-ditetradecylacetamide.

In some embodiments, the lipid formulation comprises:
(a) ((4-hydroxybutyl) azanediyl)bis(hexane-6,1-diyl)bis(2-hexyldecanoate);
(b) cholesterol;
(c) distearoylphosphatidylcholine (DSPC); and
(d) 2-[(polyethylene glycol)-2000]-N,N-ditetradecylacetamide.

In some embodiments, ((4-hydroxybutyl) azanediyl)bis(hexane-6,1-diyl)bis(2-hexyldecanoate) is within a range of about 40 to about 60 mole percent, cholesterol is within a range of about 30 to about 50 mole percent, distearoylphosphatidylcholine (DSPC) is within a range of about 5 to about 15 mole percent, and 2-[(polyethylene glycol)-2000]-N,N-ditetradecylacetamide is within a range of about 1 to about 10 mole percent.

In some embodiments, the pharmaceutical composition further comprises one or more pharmaceutically acceptable carriers, diluents, excipients and/or adjuvants. In some embodiments, the adjuvants comprise an RNA encoding one or more immunomodulating molecules, such as cytokines. In some embodiments, the adjuvants comprise one or more immunity inducing or immune-modulating moieties. In some embodiments, the one or more immunity inducing or immune-modulating moieties comprise a peptidoglycan moiety.

In some embodiments, the one or more RNA molecules are in a liquid formulation.

In some embodiments, the one or more RNA molecules are in a frozen formulation.

In some embodiments, the one or more RNA molecules are in a lyophilized formulation.

In some embodiments, the one or more RNA molecules are formulated for injection. In some embodiments, the one or more RNA molecules are formulated for intramuscular administration.

In some embodiments, the pharmaceutical composition is formulated for administration in human.

In one aspect, the disclosure provides a kit comprising one or more pharmaceutical composition disclosed herein.

In some embodiments, the kit comprises two or more pharmaceutical compositions which comprise the same or different RNA molecules disclosed herein.

In some embodiments, the kit further comprises instructions for use of the one or more pharmaceutical composition for treating or preventing tuberculosis.

In one aspect, the disclosure provides the RNA molecule, protein, DNA molecule, pharmaceutical composition or kit disclosed herein for use as a medicament.

In some embodiments of the RNA molecule, protein, DNA molecule, pharmaceutical composition or kit for use, the use comprises a therapeutic or prophylactic treatment of a disease or disorder in a subject.

In some embodiments, the use comprises the use as a vaccine against a disease or disorder in a subject.

In some embodiments, the subject is a human infected with the disease or disorder or in danger of contracting the disease or disorder.

In one aspect, the disclosure provides the RNA molecule, protein, DNA molecule, pharmaceutical composition or kit disclosed herein for use in treating or preventing tuberculosis in a subject.

In some embodiments of the RNA molecule, protein, DNA molecule, pharmaceutical composition or kit for use, the subject is a human infected with tuberculosis or in danger of contracting tuberculosis.

In some embodiments, the use is as a vaccine for preventing tuberculosis.

In one aspect, the disclosure provides the use of the RNA molecule, the protein, the DNA molecule, the pharmaceutical composition or the kit disclosed herein for the manufacture of a medicament for treating or preventing tuberculosis.

In one aspect, the disclosure a method of vaccinating a subject comprising administering the RNA molecule, the protein, the DNA, the pharmaceutical composition or the kit disclosed herein to the subject.

In some embodiments of the method, the vaccination is for preventing tuberculosis.

In some embodiments, administration is intramuscular administration.

In some embodiments, the method comprises administering to the subject at least one dose of the RNA molecule, protein, DNA molecule, pharmaceutical composition or kit.

In some embodiments, the method comprises administering to the subject at least two doses of the RNA molecule, protein, DNA molecule, pharmaceutical composition or kit.

In some embodiments, an amount of the RNA molecule of at least 10 μg per dose is administered.

In some embodiments, the subject is a human.

In some embodiments of the RNA molecule, chimeric protein, pharmaceutical composition or kit for use, the use or the method disclosed herein, the tuberculosis is caused by an infection with a Mycobacterium.

In some embodiments, the Mycobacterium is selected from the group of Mycobacterium tuberculosis, Mycobacterium bovis, Mycobacterium caprae, Mycobacterium orygis, Mycobacterium africanum, Mycobacterium microti, Mycobacterium canetti and Mycobacterium pinnipedii.

In some embodiments, the Mycobacterium is Mycobacterium tuberculosis.

### BRIEF DESCRIPTION OF THE FIGURES

FIG. 1: In vitro expression of full-length antigen candidates

Each of the indicated antigens was modified with a MHC-I derived signal peptide (husec) and an N-terminal Flag-tag. In proteins with a predicted human signal peptide, a version with (WT) and without (ΔSP) was tested. In proteins with a predicted transmembrane domain, this domain was disrupted by amino acid substitutions (dL_N). The coding sequences were codon optimized for human expression and encoded on modified RNA. Depicted are Western blots stained with anti-flag (top) and anti-tubulin antibody (bottom). The molecular weight (kDa) is depicted on the left of each blot.

FIG. 2: FACS in vitro expression of flag-tagged PstS1 and LpqH construct

Robust protein expression was observed in HEK293T cells transfected with mRNA constructs encoding PstS1 and LpqH. HEK293T cells were transfected with flag-tagged B) wt or C) 41-26 as well as with the D) wt or E) A1-25 and the protein-expression was quantified by flow cytometry via a flag-tag specific antibody.

FIG. 3: In vivo expression of full-length antigen candidates

Immunogenicity of full-length antigens in C57BL/6 mice. Two weeks after the boost, mice were sacrificed and splenocytes were isolated and stimulated with the appropriate library of overlapping peptides or indicated controls. Cytokine production was measured by fluorospot. A) Cytokine production measured as spot forming units of total splenocytes. Each dot represents a single animal tested in technical triplicate, error bars depict the standard deviation. B) Bifunctional (i.e. producing at least two cytokines), or trifunctional responses in MACS-separated CD4+ cells (Left) or CD8+ (right cells) measured by fluorospot. Each bar represents the average SFU-value measured in technical triplicate after pooling 4 animals per group.

FIG. 4: Mice immunized with PstS1-A1-26 or LpqH-full-length constructs express antibodies specific for recombinant PstS1 and LpqH proteins.

Immunization of mice with PstS1-41-26 or LpqH-full-length constructs resulted in the production of antibodies specific for PstS1 and LpqH proteins. Enzyme-linked immunosorbent assays (ELISA) were performed to evaluate the induction of antibodies against recombinant A) PstS1 and B) LpqH proteins by PstS1-41-26 or LpqH-full-length constructs.

FIG. 5: Antibodies induced by mRNA vaccine candidates recognize native PstS1 and LpqH present in Mtb lysate.

Antibodies induced by mRNA vaccine candidates recognize native PstS1 and LpqH present in H37Rv lysate. Enzyme-linked immunosorbent assays (ELISA) were performed to evaluate the induction of antibodies against H37Rv lysate by PstS1-41-26 or LpqH-full-length constructs. A range of concentrations of coated antigen was used in the assays.

FIG. 6: mRNA encoded PstS1 and LpqH is translated by HEK293T cells and recognized by monoclonal antibodies.

Panels A-C illustrate the gating strategy for single, live HEK293T cells. Transfection of HEK293T cells with 0.5 μg of mRNA encoding either pstS1 (E) or LpqH (F) resulted in a significant increase in the expression levels of the respective protein as measured by the monoclonal human anti-PstS1 antibody P4-163 (E), or a murine monoclonal anti-LpqH antibody (F), as compared to the untransfected control (D).

FIG. 7: PstS1- and LpqH-specific monoclonal antibodies (mAbs) complexed with M. tuberculosis-HN878 (Mtb-HN878) elicit IL-1B due to NLRP3 inflammasome activation.

Infection of activated and PMA differentiated THP-1 (A) and primary human monocyte-derived macrophages (B) with Mtb-Ab immune complexes caused IL-1B release for Mtb-specific IgG1 mAbs (PstS1-specific p4-36 and p4-163), but not isotype controls (mGO.53 and IgG1) respectively. LPS+Nigericin as positive control for NLRP3 stimulation. (C) Release of IL-1B was not restricted only to PstS1-specific mAbs, since the LpqH-specific mAb 009 also led to IL-1B release from BMDM. (D) Mtb: mAb immune complexes failed to cause IL-1B release in THP-1 macrophages in which Caspase-1 was deleted, or in primary murine bone-marrow-derived macrophages (BMDM) in which NLRP3 was deleted (E). (F) Pharmacologic inhibition of Caspase-1 with VX765 or NLRP3 with MCC950 also inhibited IL-1ß release. Ns p>0.05, * p<0.05, ** p<0.01, *** p<0.001 by one-way ANOVA. 3-5 biological replicates per experiment and results representative of 2-5 independent experiments.

FIG. 8: NLRP3 inhibition in mice inhibits PstS1-specific mediated early immunity against Mtb-HN878. Mice were injected once intraperitoneally with anti-PstS1 antibody p4-36 or isotype control one day prior to aerosol infection with Mtb-HN878. Mice were treated daily with either the specific NLRP3 inhibitor MCC950 or vehicle, both intraperitoneally. Mice were euthanized 2 weeks post infection, and lung bacterial burden measured. ns p>0.05, * p<0.05 ** p<0.01 by one-way ANOVA and post-hoc correction.

FIG. 9: Monoclonal antibodies against LpqH and PstS1 bind to the surface of live Mtb.

A) Flow cytometry gating strategy for H37Ra mycobacteria. The arrows indicate beads with a size of 2 and 1 μm, which were included in the control sample to ensure gating on events of the correct size and reproducibility of the data. Representative flow cytometry plots of H37Ra mycobacteria stained with the B) anti-human IgG secondary antibody, C) monoclonal anti-PstS1 antibody D), anti-mouse IgG secondary antibody, E) monoclonal anti-LpqH antibody and F) monoclonal antibody specific for a non-related antigen. Histograms summarizing the surface staining of the monoclonal G) anti-PstS1 and H) anti-LpqH antibodies. All samples were stained overnight with DMN-Tre, a green-fluorescent probe.

FIG. 10: Antibodies induced by LpqH and PstS1 encoding mRNA bind to the surface of live Mtb.

Representative flow cytometry plots of H37Ra mycobacteria stained with polyclonal serum of C57BL/6 mice immunized with two doses of A) NaCl, B) a non-related mRNA-encoded antigen, C) a LpqH encoding mRNA or D) a PstS1 encoding mRNA. E) Titration curves of sera diluted between 1:1500 and 1:12000 in 1:2 steps. The frequencies of DMN-Tre and surface IgG double positive cells are plotted for each group across all dilutions. F) Histogram summarizing the surface staining of the polyclonal serum of mice immunized with either a PstS1 or LpqH encoding mRNA, a non-related antigen or NaCl at a dilution of 1:6000.

### DETAILED DESCRIPTION

Although the present disclosure is further described in more detail below, it is to be understood that this disclosure is not limited to the particular methodologies, protocols and reagents described herein as these may vary. It is also to be understood that the terminology used herein is for the purpose of describing particular embodiments only, and is not intended to limit the scope of the present disclosure which will be limited only by the appended claims. Unless defined otherwise, all technical and scientific terms used herein have the same meanings as commonly understood by one of ordinary skill in the art.

In the following, the elements of the present disclosure will be described in more detail. These elements are listed with specific embodiments, however, it should be understood that they may be combined in any manner and in any number to create additional embodiments. The variously described examples and preferred embodiments should not be construed to limit the present disclosure to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

For example, the present disclosure describes combinations of sequence molecules which may have different levels of sequence identity to a specified sequence, e.g., (i) sequence molecule A comprising the sequence of SEQ ID NO: a, or a sequence having at least 99%, 98%, 97%, 96%, 95%, 90%, 85%, or 80% identity to the sequence of SEQ ID NO: a, (ii) sequence molecule B comprising the sequence of SEQ ID NO: b, or a sequence having at least 99%, 98%, 97%, 96%, 95%, 90%, 85%, or 80% identity to the sequence of SEQ ID NO: b etc. It should be understood that the sequence molecules may be combined in any of the identity levels specified. In some embodiments, the sequence molecules are combined such that the identity levels are identical; e.g., (i) sequence molecule A comprising the sequence of SEQ ID NO: a, (ii) sequence molecule B comprising the sequence of SEQ ID NO: b etc., or (i) sequence molecule A comprising a sequence having at least 90% identity to the sequence of SEQ ID NO: a, (ii) sequence molecule B comprising a sequence having at least 90% identity to the sequence of SEQ ID NO: b etc. In some embodiments, the identity levels are independently selected and are partially or entirely different from each other, i.e., the sequence molecules are combined such that the identity levels are not identical; e.g., (i) sequence molecule A comprising the sequence of SEQ ID NO: a, (ii) sequence molecule B comprising a sequence having at least 90% identity to the sequence of SEQ ID NO: b etc., or (i) sequence molecule A comprising a sequence having at least 90% identity to the sequence of SEQ ID NO: a, (ii) sequence molecule B comprising a sequence having at least 85% identity to the sequence of SEQ ID NO: b etc.

The practice of the present disclosure will employ, unless otherwise indicated, conventional chemistry, biochemistry, cell biology, immunology, and recombinant DNA techniques which are explained in the literature in the field.

Throughout this specification and the claims which follow, unless the context requires otherwise, the word “comprise”, and variations such as “comprises” and “comprising”, will be understood to imply the inclusion of a stated feature, element, member, integer or step or group of features, elements, members, integers or steps but not the exclusion of any other feature, element, member, integer or step or group of features, elements, members, integers or steps. The term “consisting essentially of” limits the scope of a claim or disclosure to the specified features, elements, members, integers, or steps and those that do not materially affect the basic and novel characteristic(s) of the claim or disclosure. The term “consisting of” limits the scope of a claim or disclosure to the specified features, elements, members, integers, or steps. The term “comprising” encompasses the term “consisting essentially of” which, in turn, encompasses the term “consisting of”. Thus, at each occurrence in the present application, the term “comprising” may be replaced with the term “consisting essentially of” or “consisting of”. Likewise, at each occurrence in the present application, the term “consisting essentially of” may be replaced with the term “consisting of”.

The terms “a”, “an” and “the” and similar references used in the context of describing the present disclosure (especially in the context of the claims) are to be construed to cover both the singular and the plural, unless otherwise indicated herein or clearly contradicted by the context.

All methods described herein can be performed in any suitable order unless otherwise indicated herein or otherwise clearly contradicted by the context.

The use of any and all examples, or exemplary language (e.g., “such as”), provided herein is intended merely to better illustrate the present disclosure and does not pose a limitation on the scope of the present disclosure otherwise claimed. No language in the specification should be construed as indicating any non-claimed element essential to the practice of the present disclosure.

The term “optional” or “optionally” as used herein means that the subsequently described event, circumstance or condition may or may not occur, and that the description includes instances where said event, circumstance, or condition occurs and instances in which it does not occur.

Where used herein, “and/or” is to be taken as specific disclosure of each of the two specified features or components with or without the other. For example, “X and/or Y” is to be taken as specific disclosure of each of (i) X, (ii) Y, and (iii) X and Y, just as if each is set out individually herein.

In the context of the present disclosure, the term “about” denotes an interval of accuracy that the person of ordinary skill will understand to still ensure the technical effect of the feature in question. The term typically indicates deviation from the indicated numerical value by ±10%, ±5%, ±4%, ±3%, ±2%, ±1%, ±0.9%, ±0.8%, ±0.7%, ±0.6%, ±0.5%, ±0.4%, ±0.3%, ±0.2%, ±0.1%, ±0.05%, and for example ±0.01%. In some embodiments, “about” indicates deviation from the indicated numerical value by ±10%. In some embodiments, “about” indicates deviation from the indicated numerical value by ±5%. In some embodiments, “about” indicates deviation from the indicated numerical value by ±4%. In some embodiments, “about” indicates deviation from the indicated numerical value by ±3%. In some embodiments, “about” indicates deviation from the indicated numerical value by ±2%. In some embodiments, “about” indicates deviation from the indicated numerical value by ±1%. In some embodiments, “about” indicates deviation from the indicated numerical value by ±0.9%. In some embodiments, “about” indicates deviation from the indicated numerical value by ±0.8%. In some embodiments, “about” indicates deviation from the indicated numerical value by ±0.7%. In some embodiments, “about” indicates deviation from the indicated numerical value by ±0.6%. In some embodiments, “about” indicates deviation from the indicated numerical value by ±0.5%. In some embodiments, “about” indicates deviation from the indicated numerical value by ±0.4%. In some embodiments, “about” indicates deviation from the indicated numerical value by ±0.3%. In some embodiments, “about” indicates deviation from the indicated numerical value by ±0.2%. In some embodiments, “about” indicates deviation from the indicated numerical value by ±0.1%. In some embodiments, “about” indicates deviation from the indicated numerical value by ±0.05%. In some embodiments, “about” indicates deviation from the indicated numerical value by ±0.01%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect.

Recitation of ranges of values herein is merely intended to serve as a shorthand method of referring individually to each separate value falling within the range. Unless otherwise indicated herein, each individual value is incorporated into the specification as if it were individually recited herein.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

It should be noted for unambiguousness that whenever a sequence is referred to as being the sequence between the nucleotide at position x and the nucleotide at position y, the resulting sequence includes both the nucleotide at position x and the nucleotide at position y. Similarly, whenever a sequence is referred to as being the sequence between the amino acid at position x and the amino acid at position y, the resulting sequence includes both the amino acid at position x and the amino acid at position y. Moreover, while the sequences described herein, in particular in the sequence listing, refer to DNA molecules, it is clear that when it is stated in the description or the claims that an RNA comprises a nucleotide sequence as described herein, in particular in the sequence listing, the nucleotide sequence referred to is actually identical to the base-sequence of the DNA molecule described herein, in particular in the sequence listing, e.g., represented in a SEQ ID NO referred to, except that thymine is replaced by uracil.

In the following, definitions and embodiments will be provided which apply to all aspects of the present disclosure. Terms which are defined in the following have the meanings as defined, unless otherwise indicated. Any undefined terms have their art recognized meanings.

Mycobacterium tuberculosis (Mtb) is a non-motile, slowly growing and rod shaped (2-4 μm in length and 0.2-0.5 μm in width) bacterium. Mtb is gram-positive, obligate aerobe, requires a host for growth and reproduction, and does not form spores.

The term “tuberculosis” or “TB” is used to describe the infection caused by the infective agent “Mycobacterium tuberculosis” or “Mtb”. Tuberculosis is a potentially fatal contagious disease that can affect almost any part of the body but is most frequently an infection of the lungs. While the majority of tuberculosis infections is caused by Mycobacterium tuberculosis, there are other Mycobacterium species that can cause tuberculosis as well. These species include Mycobacterium bovis, Mycobacterium caprae, Mycobacterium orygis, Mycobacterium africanum, Mycobacterium microti, Mycobacterium canetti and Mycobacterium pinnipedii. Mycobacterium tuberculosis and some other mycobacteria are transmitted by airborne droplet nuclei produced when an individual with active disease coughs, speaks, or sneezes. When inhaled, the droplet nuclei reach the alveoli of the lung. In susceptible individuals the organisms may then multiply and spread through lymphatics to the lymph nodes, and through the bloodstream to other sites such as the lung apices, bone marrow, kidneys, and meninges. Infections with other Mycobacterium species, such as Mycobacterium bovis or Mycobacterium caprae are also associated with the consumption of un-pasteurized milk from infected animals. The development of acquired immunity in 2 to 10 weeks results in a halt to bacterial multiplication. Lesions heal and the individual remains asymptomatic. Mycobacteria can remain dormant (latent TB) in the body after infection for years, concealed in the phagocytosed cells, and never develop into the disease. Such an individual is said to have tuberculous infection without disease, and will show a positive tuberculin test. The clinical status of latent TB is traditionally associated with the transition of Mtb to a dormant state in response to non-optimal growth conditions in vivo due to activation of the host immune response. Dormancy is a specific physiological state characterized by significant cessation of metabolic activity and growth, whereas resuscitation from dormancy is a process of restoring cell activity followed by bacterial multiplication, which in case of Mtb can lead to disease progression. The risk of developing active disease with clinical symptoms diminishes with time and may never occur, but is a lifelong risk. Approximately 5% of individuals with tuberculous infection progress to active disease.

Terms such as “reduce” or “inhibit” as used herein means the ability to cause an overall decrease, for example, of about 5% or greater, about 10% or greater, about 15% or greater, about 20% or greater, about 25% or greater, about 30% or greater, about 40% or greater, about 50% or greater, or about 75% or greater, in the level. The term “inhibit” or similar phrases includes a complete or essentially complete inhibition, i.e. a reduction to zero or essentially to zero.

Terms such as “enhance” as used herein means the ability to cause an overall increase, or enhancement, for example, by at least about 5% or greater, about 10% or greater, about 15% or greater, about 20% or greater, about 25% or greater, about 30% or greater, about 40% or greater, about 50% or greater, about 75% or greater, or about 100% or greater in the level.

“Physiological pH” as used herein refers to a pH of about 7.4. In some embodiments, physiological pH is from 7.3 to 7.5. In some embodiments, physiological pH is from 7.35 to 7.45. In some embodiments, physiological pH is 7.3, 7.35, 7.4, 7.45, or 7.5.

As used in the present disclosure, “% w/v” refers to weight by volume percent, which is a unit of concentration measuring the amount of solute in grams (g) expressed as a percent of the total volume of solution in milliliters (mL).

As used in the present disclosure, “% by weight” refers to weight percent, which is a unit of concentration measuring the amount of a substance in grams (g) expressed as a percent of the total weight of the total composition in grams (g).

As used in the present disclosure, “mol %” is defined as the ratio of the number of moles of one component to the total number of moles of all components, multiplied by 100.

As used in the present disclosure, “mol % of the total lipid” is defined as the ratio of the number of moles of one lipid component to the total number of moles of all lipids, multiplied by 100. In this context, in some embodiments, the term “total lipid” includes lipids and lipid-like material.

The term “ionic strength” refers to the mathematical relationship between the number of different kinds of ionic species in a particular solution and their respective charges. Thus, ionic strength I is represented mathematically by the formula:

$$I = \frac{1}{2} \cdot {\sum }_{i} {z}_{i}^{2} \cdot {c}_{i}$$

in which c is the molar concentration of a particular ionic species and z the absolute value of its charge. The sum 2 is taken over all the different kinds of ions (i) in solution.

According to the disclosure, the term “ionic strength” in some embodiments relates to the presence of monovalent ions. Regarding the presence of divalent ions, in particular divalent cations, their concentration or effective concentration (presence of free ions) due to the presence of chelating agents is, in some embodiments, sufficiently low so as to prevent degradation of the nucleic acid. In some embodiments, the concentration or effective concentration of divalent ions is below the catalytic level for hydrolysis of the phosphodiester bonds between nucleotides such as RNA nucleotides. In some embodiments, the concentration of free divalent ions is 20 μM or less. In some embodiments, there are no or essentially no free divalent ions.

“Osmolality” refers to the concentration of a particular solute expressed as the number of osmoles of solute per kilogram of solvent.

The term “lyophilizing” or “lyophilization” refers to the freeze-drying of a substance by freezing it and then reducing the surrounding pressure (e.g., below 15 Pa, such as below 10 Pa, below 5 Pa, or 1 Pa or less) to allow the frozen medium in the substance to sublimate directly from the solid phase to the gas phase. Thus, the terms “lyophilizing” and “freeze-drying” are used herein interchangeably.

The term “spray-drying” refers to spray-drying a substance by mixing (heated) gas with a fluid that is atomized (sprayed) within a vessel (spray dryer), where the solvent from the formed droplets evaporates, leading to a dry powder.

The term “reconstitute” relates to adding a solvent such as water to a dried product to return it to a liquid state such as its original liquid state.

The term “recombinant” in the context of the present disclosure means “made through genetic engineering”. In some embodiments, a “recombinant object” in the context of the present disclosure is not occurring naturally.

The term “naturally occurring” as used herein refers to the fact that an object can be found in nature. For example, a peptide or nucleic acid that is present in an organism (including viruses) and can be isolated from a source in nature and which has not been intentionally modified by man in the laboratory is naturally occurring. The term “found in nature” means “present in nature” and includes known objects as well as objects that have not yet been discovered and/or isolated from nature, but that may be discovered and/or isolated in the future from a natural source. As used herein, the terms “room temperature” and “ambient temperature” are used interchangeably herein and refer to temperatures from at least about 15° C., e.g., from about 15° C. to about 35° C., from about 15° C. to about 30° C., from about 15° C. to about 25° C., or from about 17° C. to about 22° C. Such temperatures will include 15° C., 16° C., 17° C., 18° C., 19° C., 20° C., 21° C. and 22° C.

The term “EDTA” refers to ethylenediaminetetraacetic acid disodium salt. All concentrations are given with respect to the EDTA disodium salt.

The term “cryoprotectant” relates to a substance that is added to a formulation in order to protect the active ingredients during the freezing stages.

The term “lyoprotectant” relates to a substance that is added to a formulation in order to protect the active ingredients during the drying stages.

According to the present disclosure, the term “peptide” refers to substances which comprise about two or more, about 3 or more, about 4 or more, about 6 or more, about 8 or more, about 10 or more, about 13 or more, about 16 or more, about 20 or more, and up to about 50, about 100 or about 150, consecutive amino acids linked to one another via peptide bonds. The term “polypeptide” refers to large peptides, in particular peptides having at least about 151 amino acids. “Peptides” and “polypeptides” are both protein molecules, although the terms “protein” and “polypeptide” are used herein usually as synonyms.

The term “biological activity” means the response of a biological system to a molecule. Such biological systems may be, for example, a cell or an organism. In some embodiments, such response is therapeutically or pharmaceutically useful.

The term “portion” refers to a fraction. With respect to a particular structure such as an amino acid sequence or protein the term “portion” thereof may designate a continuous or a discontinuous fraction of said structure.

The terms “part” and “fragment” are used interchangeably herein and refer to a continuous element. For example, a part of a structure such as an amino acid sequence or protein refers to a continuous element of said structure. When used in context of a composition, the term “part” means a portion of the composition. For example, a part of a composition may be any portion from 0.1% to 99.9% (such as 0.1%, 0.5%, 1%, 5%, 10%, 50%, 90%, or 99%) of said composition.

“Fragment”, with reference to an amino acid sequence (peptide or polypeptide), relates to a part of an amino acid sequence, i.e. a sequence which represents the amino acid sequence shortened at the N-terminus and/or C-terminus. A fragment shortened at the C-terminus (N-terminal fragment) is obtainable, e.g., by translation of a truncated open reading frame that lacks the 3′-end of the open reading frame. A fragment shortened at the N-terminus (C-terminal fragment) is obtainable, e.g., by translation of a truncated open reading frame that lacks the 5′-end of the open reading frame, as long as the truncated open reading frame comprises a start codon that serves to initiate translation. A fragment of an amino acid sequence comprises, e.g., at least 50%, at least 60%, at least 70%, at least 80%, at least 90% of the amino acid residues from an amino acid sequence. A fragment of an amino acid sequence comprises, e.g., at least 5, at least 6, at least 7, in particular at least 8, at least 10, at least 12, at least 15, at least 20, at least 30, at least 50, or at least 100 consecutive amino acids from an amino acid sequence. A fragment of an amino acid sequence comprises, e.g., a sequence of up to 8, in particular up to 10, up to 12, up to 15, up to 20, up to 30, up to 50, up to 80, up to 100, up to 150 or up to 200 consecutive amino acids of the amino acid sequence. The phrase “full-length antigen or antigen fragment(s) representing a Mycobacterium tuberculosis antigen or immunogenic variant thereof” as used herein refers to the Mycobacterium tuberculosis antigen or an immunogenic variant of the Mycobacterium tuberculosis antigen, or one or more fragments of the Mycobacterium tuberculosis antigen or an immunogenic variant of the Mycobacterium tuberculosis antigen, wherein the fragments may or may not be overlapping. An immunogenic variant of a Mycobacterium tuberculosis antigen or one or more fragments of a Mycobacterium tuberculosis antigen or an immunogenic variant of a Mycobacterium tuberculosis antigen are capable of inducing an immune response against the Mycobacterium tuberculosis antigen when delivered to a subject, e.g. in the form of a protein or an RNA transcribed by a cell of the subject. In some embodiments, a fragment of a Mycobacterium tuberculosis antigen or an immunogenic variant of a Mycobacterium tuberculosis antigen comprises at least one epitope, e.g., at least one T cell epitope, of a Mycobacterium tuberculosis antigen or an immunologically equivalent variant of said at least one epitope. In some embodiments, a fragment of a Mycobacterium tuberculosis antigen or an immunogenic variant of a Mycobacterium tuberculosis antigen comprises a fragment of, e.g., at least 5, at least 6, at least 7, in particular at least 8, at least 10, at least 12, at least 15, at least 20, at least 30, at least 50, or at least 100 consecutive amino acids of said Mycobacterium tuberculosis antigen or immunogenic variant of a Mycobacterium tuberculosis antigen.

The phrase “encoding at least one full-length antigen or antigen fragment representing at least one Mycobacterium tuberculosis antigen or immunogenic variant thereof” with respect to RNA encompasses monocistronic and polycistronic RNAs.

If only one Mycobacterium tuberculosis antigen or immunogenic variant thereof is represented, the RNA may encode the full length Mycobacterium tuberculosis antigen or immunogenic variant thereof and/or may encode one or more fragments of the Mycobacterium tuberculosis antigen or immunogenic variant thereof. If the RNA encodes a full length Mycobacterium tuberculosis antigen or immunogenic variant thereof and at least one fragment of the Mycobacterium tuberculosis antigen or immunogenic variant thereof, the full length Mycobacterium tuberculosis antigen or immunogenic variant thereof and one or more of the at least one fragment of the Mycobacterium tuberculosis antigen or immunogenic variant thereof may be encoded by different open reading frames located on the same or on different RNA molecules. If the RNA encodes more than one fragment of the Mycobacterium tuberculosis antigen or immunogenic variant thereof, one or more of the more than one fragment of the Mycobacterium tuberculosis antigen or immunogenic variant thereof may be encoded by different open reading frames located on the same or on different RNA molecules.

If more than one Mycobacterium tuberculosis antigen or immunogenic variant thereof is represented, the RNA may either encode the full-length antigen of one or more Mycobacterium tuberculosis antigen or immunogenic variant thereof, may encode one or more fragments of one or more Mycobacterium tuberculosis antigen or immunogenic variant thereof, or a combination thereof. In some embodiments, the RNA encodes the full-length antigen of each of the more than one Mycobacterium tuberculosis antigen or immunogenic variant thereof. In some embodiments, the RNA encodes one or more fragments of each of the more than one Mycobacterium tuberculosis antigen or immunogenic variant thereof. In some embodiments, the RNA encodes the full-length antigen of some of the more than one Mycobacterium tuberculosis antigen or immunogenic variant thereof and encodes one or more fragments of some of the more than one Mycobacterium tuberculosis antigen or immunogenic variant thereof, wherein the RNA may encode the full-length antigen as well as one or more fragments of the same Mycobacterium tuberculosis antigen or immunogenic variant thereof. The full-length antigens and/or fragments discussed above may be encoded by the same or different open reading frames located on the same or on different RNA molecules.

The term “chimeric protein” is used herein as a synonym for “fusion protein” and means a protein comprising two or more subunits, such as a full-length antigen, antigen fragment and/or other functional amino acid sequence. Preferably, the fusion protein is a translational fusion between the two or more subunits. The translational fusion may be generated by genetically engineering the coding nucleotide sequence for one subunit in a reading frame with the coding nucleotide sequence of a further subunit. Subunits may be interspersed by a polypeptide linker.

“Variant,” as used herein and with reference to an amino acid sequence (peptide or polypeptide), is meant an amino acid sequence that differs from a parent amino acid sequence by virtue of at least one amino acid (e.g., a different amino acid, or a modification of the same amino acid). The parent amino acid sequence may be a naturally occurring or wild type (WT) amino acid sequence, or may be a modified version of a wild type amino acid sequence. In some embodiments, the variant amino acid sequence has at least one amino acid difference as compared to the parent amino acid sequence, e.g., from 1 to about 20 amino acid differences, such as from 1 to about 10 or from 1 to about 5 amino acid differences compared to the parent.

By “wild type” or “WT” or “native” herein is meant an amino acid sequence that is found in nature, including allelic variations and/or naturally occurring mutations. A wild type amino acid sequence, peptide or polypeptide has an amino acid sequence that has not been intentionally modified by man.

The term “non-native” as used herein in conjunction with amino acid sequences is meant to refer to amino acid sequences not found in nature, i.e., that have been intentionally modified by man-either in sequence or in sequence context. In one embodiment, a non-native signal peptide sequence fused or operatively linked to a Mycobacterium tuberculosis antigen denotes that said signal peptide in nature does not occur fused or operatively linked to said Mycobacterium tuberculosis antigen, either because said signal peptide can naturally be found fused or operatively linked only to other Mycobacterium tuberculosis antigens or only in other organisms, such as mammals, e.g. human, other bacteria besides Mycobacterium tuberculosis or viruses. Embodiments for such exogenous signal peptides are provided herein. In another embodiment, a non-native signal peptide has been mutated in a purposeful manner (e.g., by random mutagenesis and targeted selection or by guided mutagenesis techniques, including, e.g., sequence synthesis) in order to obtain certain functional properties or to eliminate certain functional properties, resulting in a signal peptide structurally and functionally distinct from a signal peptide found in nature fused or operatively linked to the Mycobacterium tuberculosis antigen in question.

“Variant,” as used herein and with reference to an amino acid sequence (peptide or polypeptide), is meant an amino acid sequence that differs from a parent amino acid sequence by virtue of at least one amino acid (e.g., a different amino acid, or a modification of the same amino acid). The parent amino acid sequence may be a naturally occurring or wild type (WT) amino acid sequence, or may be a modified version of a wild type amino acid sequence. In some embodiments, the variant amino acid sequence has at least one amino acid difference as compared to the parent amino acid sequence, e.g., from 1 to about 20 amino acid differences, such as from 1 to about 10 or from 1 to about 5 amino acid differences compared to the parent.

For the purposes of the present disclosure, “variants” of an amino acid sequence (peptide or polypeptide) may comprise amino acid insertion variants, amino acid addition variants, amino acid deletion variants and/or amino acid substitution variants. The term “variant” includes all mutants, splice variants, post-translationally modified variants, conformations, isoforms, allelic variants, species variants, and species homologs, in particular those which are naturally occurring. The term “variant” includes, in particular, fragments of an amino acid sequence.

Amino acid insertion variants comprise insertions of single or two or more amino acids in a particular amino acid sequence. In the case of amino acid sequence variants having an insertion, one or more amino acid residues are inserted into a particular site in an amino acid sequence, although random insertion with appropriate screening of the resulting product is also possible. Amino acid addition variants comprise amino- and/or carboxy-terminal fusions of one or more amino acids, such as 1, 2, 3, 5, 10, 20, 30, 50, or more amino acids. Amino acid deletion variants are characterized by the removal of one or more amino acids from the sequence, such as by removal of 1, 2, 3, 5, 10, 20, 30, 50, or more amino acids. The deletions may be in any position of the protein. Amino acid deletion variants that comprise the deletion at the N-terminal and/or C-terminal end of the protein are also called N-terminal and/or C-terminal truncation variants. Amino acid substitution variants are characterized by at least one residue in the sequence being removed and another residue being inserted in its place. Preference is given to the modifications being in positions in the amino acid sequence which are not conserved between homologous peptides or polypeptides and/or to replacing amino acids with other ones having similar properties. In some embodiments, amino acid changes in peptide and polypeptide variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids. In some embodiments, conservative amino acid substitutions include substitutions within the following groups:
glycine, alanine;
valine, isoleucine, leucine;
aspartic acid, glutamic acid;
asparagine, glutamine;
serine, threonine;
lysine, arginine; and
phenylalanine, tyrosine.

In some embodiments, the degree of similarity, such as identity between a given amino acid sequence and an amino acid sequence which is a variant of said given amino acid sequence, will be at least about 60%, 70%, 80%, 81%, 82%, 83%, 84%, 85%, 86%, 87%, 88%, 89%, 90%, 91%, 92%, 93%, 94%, 95%, 96%, 97%, 98%, or 99%. In some embodiments, the degree of similarity or identity is given for an amino acid region which is at least about 10%, at least about 20%, at least about 30%, at least about 40%, at least about 50%, at least about 60%, at least about 70%, at least about 80%, at least about 90% or about 100% of the entire length of the reference amino acid sequence. For example, if the reference amino acid sequence consists of 200 amino acids, the degree of similarity or identity is given, e.g., for at least about 20, at least about 40, at least about 60, at least about 80, at least about 100, at least about 120, at least about 140, at least about 160, at least about 180, or about 200 amino acids, in some embodiments, continuous amino acids. In some embodiments, the degree of similarity or identity is given for the entire length of the reference amino acid sequence. The alignment for determining sequence similarity, such as sequence identity, can be done with art known tools, such as using the best sequence alignment, for example, using Align, using standard settings, preferably EMBOSS::needle, Matrix: Blosum62, Gap Open 10.0, Gap Extend 0.5.

“Sequence similarity” indicates the percentage of amino acids that either are identical or that represent conservative amino acid substitutions. “Sequence identity” between two amino acid sequences indicates the percentage of amino acids that are identical between the sequences. “Sequence identity” between two nucleic acid sequences indicates the percentage of nucleotides that are identical between the sequences.

The terms “% identical” and “% identity” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing the sequences, after optimal alignment, with respect to a segment or “window of comparison”, in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Neddleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N and TFASTA in Wisconsin Genetics Software Package, Genetics Computer Group, 575 Science Drive, Madison, Wis.). In some embodiments, percent identity of two sequences is determined using the BLASTN or BLASTP algorithm, as available on the United States National Center for Biotechnology Information (NCBI) website (e.g., at blast.ncbi.nlm.nih.gov/Blast.cgi?PAGE_TYPE=BlastSearch&BLAST_SPEC=blast2seq&LINK_LOC=align2seq). In some embodiments, the algorithm parameters used for BLASTN algorithm on the NCBI website include: (i) Expect Threshold set to 10; (ii) Word Size set to 28; (iii) Max matches in a query range set to 0; (iv) Match/Mismatch Scores set to 1,-2; (v) Gap Costs set to Linear; and (vi) the filter for low complexity regions being used. In some embodiments, the algorithm parameters used for BLASTP algorithm on the NCBI website include: (i) Expect Threshold set to 10; (ii) Word Size set to 3; (iii) Max matches in a query range set to 0; (iv) Matrix set to BLOSUM62; (v) Gap Costs set to Existence: 11 Extension: 1; and (vi) conditional compositional score matrix adjustment.

Percentage identity is obtained by determining the number of identical positions at which the sequences to be compared correspond, dividing this number by the number of positions compared (e.g., the number of positions in the reference sequence) and multiplying this result by 100.

In some embodiments, the degree of similarity or identity is given for a region which is at least about 50%, at least about 60%, at least about 70%, at least about 80%, at least about 90% or about 100% of the entire length of the reference sequence. For example, if the reference nucleic acid sequence consists of 200 nucleotides, the degree of identity is given for at least about 100, at least about 120, at least about 140, at least about 160, at least about 180, or about 200 nucleotides, in some embodiments continuous nucleotides. In some embodiments, the degree of similarity or identity is given for the entire length of the reference sequence.

Homologous amino acid sequences exhibit according to the disclosure at least 40%, in particular at least 50%, at least 60%, at least 70%, at least 80%, at least 90% and, e.g., at least 95%, at least 98 or at least 99% identity of the amino acid residues.

The amino acid sequence variants described herein may readily be prepared by the skilled person, for example, by recombinant DNA manipulation. The manipulation of DNA sequences for preparing peptides or polypeptides having substitutions, additions, insertions or deletions, is described in detail in Molecular Cloning: A Laboratory Manual, 4th Edition, M. R. Green and J. Sambrook eds., Cold Spring Harbor Laboratory Press, Cold Spring Harbor 2012, for example. Furthermore, the peptides, polypeptides and amino acid variants described herein may be readily prepared with the aid of known peptide synthesis techniques such as, for example, by solid phase synthesis and similar methods.

In some embodiments, a fragment or variant of an amino acid sequence (peptide or polypeptide) is a “functional fragment” or “functional variant”. The term “functional fragment” or “functional variant” of an amino acid sequence relates to any fragment or variant exhibiting one or more functional properties identical or similar to those of the amino acid sequence from which it is derived, i.e., it is functionally equivalent. With respect to antigens or antigenic sequences, one particular function is one or more immunogenic activities displayed by the amino acid sequence from which the fragment or variant is derived. The term “functional fragment” or “functional variant”, as used herein, in particular refers to a variant molecule or sequence that comprises an amino acid sequence that is altered by one or more amino acids compared to the amino acid sequence of the parent molecule or sequence and that is still capable of fulfilling one or more of the functions of the parent molecule or sequence, e.g., inducing an immune response. In some embodiments, the modifications in the amino acid sequence of the parent molecule or sequence do not significantly affect or alter the characteristics of the molecule or sequence. In different embodiments, the function of the functional fragment or functional variant may be reduced but still significantly present, e.g., function of the functional fragment or functional variant may be at least 50%, at least 60%, at least 70%, at least 80%, or at least 90% of the parent molecule or sequence. However, in other embodiments, function of the functional fragment or functional variant may be enhanced compared to the parent molecule or sequence.

An amino acid sequence (peptide or polypeptide) “derived from” a designated amino acid sequence (peptide or polypeptide) refers to the origin of the first amino acid sequence. In some embodiments, the amino acid sequence which is derived from a particular amino acid sequence has an amino acid sequence that is identical, essentially identical or homologous to that particular sequence or a fragment thereof. Amino acid sequences derived from a particular amino acid sequence may be variants of that particular sequence or a fragment thereof. For example, it will be understood by one of ordinary skill in the art that the antigens suitable for use herein may be altered such that they vary in sequence from the naturally occurring or native sequences from which they were derived, while retaining the desirable activity of the native sequences.

In some embodiments, “isolated” means removed (e.g., purified) from the natural state or from an artificial composition, such as a composition from a production process. For example, a nucleic acid, peptide or polypeptide naturally present in a living animal is not “isolated”, but the same nucleic acid, peptide or polypeptide partially or completely separated from the coexisting materials of its natural state is “isolated”. An isolated nucleic acid, peptide or polypeptide can exist in substantially purified form, or can exist in a non-native environment such as, for example, a host cell.

The term “transfection” relates to the introduction of nucleic acids, in particular RNA, into a cell. For purposes of the present disclosure, the term “transfection” also includes the introduction of a nucleic acid into a cell or the uptake of a nucleic acid by such cell, wherein the cell may be present in a subject, e.g., a patient, or the cell may be in vitro, e.g., outside of a patient. Thus, according to the present disclosure, a cell for transfection of a nucleic acid described herein can be present in vitro or in vivo, e.g. the cell can form part of an organ, a tissue and/or the body of a patient. According to the disclosure, transfection can be transient or stable. For some applications of transfection, it is sufficient if the transfected genetic material is only transiently expressed. RNA can be transfected into cells to transiently express its coded protein. Since the nucleic acid introduced in the transfection process is usually not integrated into the nuclear genome, the foreign nucleic acid will be diluted through mitosis or degraded. Cells allowing episomal amplification of nucleic acids greatly reduce the rate of dilution. If it is desired that the transfected nucleic acid actually remains in the genome of the cell and its daughter cells, a stable transfection must occur. Such stable transfection can be achieved by using virus-based systems or transposon-based systems for transfection, for example. Generally, nucleic acid encoding antigen is transiently transfected into cells. RNA can be transfected into cells to transiently express its coded protein.

The disclosure includes analogs of a peptide or polypeptide. According to the present disclosure, an analog of a peptide or polypeptide is a modified form of said peptide or polypeptide from which it has been derived and has at least one functional property of said peptide or polypeptide. E.g., a pharmacological active analog of a peptide or polypeptide has at least one of the pharmacological activities of the peptide or polypeptide from which the analog has been derived. Such modifications include any chemical modification and comprise single or multiple substitutions, deletions and/or additions of any molecules associated with the peptide or polypeptide, such as carbohydrates, lipids and/or peptides or polypeptides. In some embodiments, “analogs” of peptides or polypeptides include those modified forms resulting from glycosylation, acetylation, phosphorylation, amidation, palmitoylation, myristoylation, isoprenylation, lipidation, alkylation, derivatization, introduction of protective/blocking groups, proteolytic cleavage or binding to an antibody or to another cellular ligand. The term “analog” also extends to all functional chemical equivalents of said peptides and polypeptides.

As used herein, the terms “linked”, “fused”, or “fusion” are used interchangeably. These terms refer to the joining together of two or more elements or components or domains.

As used herein “endogenous” refers to any material from or produced inside an organism, cell, tissue or system. As used herein, the term “exogenous” refers to any material introduced from or produced outside an organism, cell, tissue or system.

According to various embodiments of the present disclosure, a nucleic acid such as RNA encoding a peptide or polypeptide is taken up by or introduced, i.e. transfected or transduced, into a cell which cell may be present in vitro or in a subject, resulting in expression of said peptide or polypeptide. The cell may, e.g., express the encoded peptide or polypeptide intracellularly (e.g. in the cytoplasm and/or in the nucleus), may secrete the encoded peptide or polypeptide, and/or may express it on the surface. In some embodiments, the cell secretes the encoded peptide or polypeptide.

According to the present disclosure, terms such as “nucleic acid expressing” and “nucleic acid encoding” or similar terms are used interchangeably herein and with respect to a particular peptide or polypeptide mean that the nucleic acid, if present in the appropriate environment, e.g. within a cell, can be expressed to produce said peptide or polypeptide.

In particular, the term “encoding” refers to the inherent property of specific sequences of nucleotides in a polynucleotide, such as a gene, a cDNA, or an RNA (in particular, mRNA), to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

In this respect, an “open reading frame” or “ORF” is a continuous stretch of codons beginning with a start codon and ending with a stop codon.

The term “expression” as used herein includes the transcription and/or translation of a particular nucleotide sequence. In the context of the present disclosure, the term “transcription” relates to a process, wherein the genetic code in a DNA sequence is transcribed into RNA (especially mRNA). Subsequently, the RNA may be translated into peptide or polypeptide.

With respect to RNA, the term “expression” or “translation” relates to the process in the ribosomes of a cell by which a strand of mRNA directs the assembly of a sequence of amino acids to make a peptide or polypeptide.

A medical preparation, in particular kit, described herein may comprise instructional material or instructions. As used herein, “instructional material” or “instructions” includes a publication, a recording, a diagram, or any other medium of expression which can be used to communicate the usefulness of the compositions and methods of the present disclosure. The instructional material of the kit of the present disclosure may, for example, be affixed to a container which contains the compositions/formulations of the present disclosure or be shipped together with a container which contains the compositions/formulations. Alternatively, the instructional material may be shipped separately from the container with the intention that the instructional material and the compositions be used cooperatively by the recipient.

The term “set”, e.g., as used herein in the context of “set of full-length antigens and antigen fragments”, means more than 1, e.g., 2 or more, 3 or more, 4 or more, 5 or more, 6 or more, 7 or more, or 8 or more.

The term “at least one” as used herein in the context of “at least one RNA molecule” means 1 or more, e.g., 2 or more, 3 or more, 4 or more, 5 or more, 6 or more, 7 or more, or 8 or more. In some embodiments, the term “at least one” refers to 1, 2, 3, 4, 5, 6, 7, or 8. In some embodiments, “at least one RNA molecule” refers to a set of RNA molecules, e.g., 2, 3, 4 or more RNA molecules, wherein each RNA molecule encodes an amino acid sequence comprising at least one full-length Mtb antigen or antigen fragment, immunogenic variants or fragments thereof, e.g., an amino acid sequence comprising two different Mtb antigens, immunogenic variants or fragments thereof. In some embodiments, such at least one RNA molecule or set of RNA molecules comprises the RNA molecules in a mixtures, which mixture may be obtainable by transcribing in a common reaction a mixture of DNA templates encoding said RNA molecules. Prodrugs of a particular compound described herein are those compounds that upon administration to an individual undergo chemical conversion under physiological conditions to provide the particular compound. Additionally, prodrugs can be converted to the particular compound by chemical or biochemical methods in an ex vivo environment. For example, prodrugs can be slowly converted to the particular compound when, for example, placed in a transdermal patch reservoir with a suitable enzyme or chemical reagent. Exemplary prodrugs are esters (using an alcohol or a carboxy group contained in the particular compound) or amides (using an amino or a carboxy group contained in the particular compound) which are hydrolyzable in vivo. Specifically, any amino group which is contained in the particular compound and which bears at least one hydrogen atom can be converted into a prodrug form. Typical N-prodrug forms include carbamates, Mannich bases, enamines, and enaminones.

In the present specification, a structural formula of a compound may represent a certain isomer of said compound. It is to be understood, however, that the present disclosure includes all isomers such as geometrical isomers, optical isomers based on an asymmetrical carbon, stereoisomers, tautomers and the like which occur structurally and isomer mixtures and is not limited to the description of the formula. Furthermore, in the present specification, a structural formula of a compound may represent a specific salt and/or solvate of said compound. It is to be understood, however, that the present disclosure includes all salts (e.g., pharmaceutically acceptable salts) and solvates (e.g., hydrates) and is not limited to the description of the specific salt and/or solvate.

“Isomers” are compounds having the same molecular formula but differ in structure (“structural isomers”) or in the geometrical (spatial) positioning of the functional groups and/or atoms (“stereoisomers”). “Enantiomers” are a pair of stereoisomers which are non-superimposable mirror-images of each other. A “racemic mixture” or “racemate” contains a pair of enantiomers in equal amounts and is denoted by the prefix (+). “Diastereomers” are stereoisomers which are non-superimposable and which are not mirror-images of each other. “Tautomers” are structural isomers of the same chemical substance that spontaneously and reversibly interconvert into each other, even when pure, due to the migration of individual atoms or groups of atoms; i.e., the tautomers are in a dynamic chemical equilibrium with each other. An example of tautomers are the isomers of the keto-enol-tautomerism. “Conformers” are stereoisomers that can be interconverted just by rotations about formally single bonds, and include—in particular—those leading to different 3-dimensional forms of (hetero) cyclic rings, such as chair, half-chair, boat, and twist-boat forms of cyclohexane.

The term “solvate” as used herein refers to an addition complex of a dissolved material in a solvent (such as an organic solvent (e.g., an aliphatic alcohol (such as methanol, ethanol, n-propanol, isopropanol), acetone, acetonitrile, ether, and the like), water or a mixture of two or more of these liquids), wherein the addition complex exists in the form of a crystal or mixed crystal. The amount of solvent contained in the addition complex may be stoichiometric or non-stoichiometric. A “hydrate” is a solvate wherein the solvent is water.

In isotopically labeled compounds one or more atoms are replaced by a corresponding atom having the same number of protons but differing in the number of neutrons. For example, a hydrogen atom may be replaced by a deuterium or tritium atom. Exemplary isotopes which can be used in the present disclosure include deuterium, tritium, ¹¹C, ¹³C, ¹⁴C, ¹⁵N, ¹⁸F, ³²P, ³²S, ³⁵S, ³⁶Cl, and ¹²⁵I.

The term “average diameter” refers to the mean hydrodynamic diameter of particles as measured by dynamic light scattering (DLS) with data analysis using the so-called cumulant algorithm, which provides as results the so-called Z_{average} with the dimension of a length, and the polydispersity index (PDI), which is dimensionless (Koppel, D., J. Chem. Phys. 57, 1972, pp 4814-4820, ISO 13321). Here “average diameter”, “diameter” or “size” for particles is used synonymously with this value of the Z_{average}.

In some embodiments, the “polydispersity index” is calculated based on dynamic light scattering measurements by the so-called cumulant analysis as mentioned in the definition of the “average diameter”. Under certain prerequisites, it can be taken as a measure of the size distribution of an ensemble of nanoparticles.

The “radius of gyration” (abbreviated herein as R_{g}) of a particle about an axis of rotation is the radial distance of a point from the axis of rotation at which, if the whole mass of the particle is assumed to be concentrated, its moment of inertia about the given axis would be the same as with its actual distribution of mass. Mathematically, R_{g} is the root mean square distance of the particle's components from either its center of mass or a given axis. For example, for a macromolecule composed of n mass elements, of masses mᵢ(i=1, 2, 3, . . . , n), located at fixed distances sᵢ from the center of mass, R_{g} is the square-root of the mass average of sᵢ² over all mass elements and can be calculated as follows:

$${R}_{g} = {( {\sum }_{i = 1}^{n} {m}_{i} \cdot {s}_{i}^{2} / {\sum }_{i = 1}^{n} {m}_{i} )}^{1 / 2}$$

The radius of gyration can be determined or calculated experimentally, e.g., by using light scattering. In particular, for small scattering vectors q the structure function S is defined as follows:

$$S ( \vec{q} ) \approx N \cdot ( 1 - \frac{{q}^{2} \cdot {R}_{g}^{2}}{3} )$$

wherein N is the number of components (Guinier's law).

The “hydrodynamic radius” (which is sometimes called “Stokes radius” or “Stokes-Einstein radius”) of a particle is the radius of a hypothetical hard sphere that diffuses at the same rate as said particle. The hydrodynamic radius is related to the mobility of the particle, taking into account not only size but also solvent effects. For example, a smaller charged particle with stronger hydration may have a greater hydrodynamic radius than a larger charged particle with weaker hydration. This is because the smaller particle drags a greater number of water molecules with it as it moves through the solution. Since the actual dimensions of the particle in a solvent are not directly measurable, the hydrodynamic radius may be defined by the Stokes-Einstein equation:

$${R}_{h} = \frac{{k}_{g} \cdot T}{6 \cdot π \cdot η \cdot D}$$

wherein k_{B} is the Boltzmann constant; T is the temperature; η is the viscosity of the solvent; and D is the diffusion coefficient. The diffusion coefficient can be determined experimentally, e.g., by using dynamic light scattering (DLS). Thus, one procedure to determine the hydrodynamic radius of a particle or a population of particles (such as the hydrodynamic radius of particles contained in a sample or control composition as disclosed herein or the hydrodynamic radius of a particle peak obtained from subjecting such a sample or control composition to field-flow fractionation) is to measure the DLS signal of said particle or population of particles (such as DLS signal of particles contained in a sample or control composition as disclosed herein or the DLS signal of a particle peak obtained from subjecting such a sample or control composition to field-flow fractionation).

The expression “light scattering” as used herein refers to the physical process where light is forced to deviate from a straight trajectory by one or more paths due to localized non-uniformities in the medium through which the light passes.

The term “UV” means ultraviolet and designates a band of the electromagnetic spectrum with a wavelength from 10 nm to 400 nm, i.e., shorter than that of visible light but longer than X-rays.

The expression “multi-angle light scattering” or “MALS” as used herein relates to a technique for measuring the light scattered by a sample into a plurality of angles. “Multi-angle” means in this respect that scattered light can be detected at different discrete angles as measured, for example, by a single detector moved over a range including the specific angles selected or an array of detectors fixed at specific angular locations. In certain embodiments, the light source used in MALS is a laser source (MALLS: multi-angle laser light scattering). Based on the MALS signal of a composition comprising particles and by using an appropriate formalism (e.g., Zimm plot, Berry plot, or Debye plot), it is possible to determine the radius of gyration (R_{g}) and, thus, the size of said particles. Preferably, the Zimm plot is a graphical presentation using the following equation:

$$\frac{{R}_{θ}}{K * c} = {M}_{w} P ( θ ) - 2 {A}_{2} {cM}_{w}^{2} {P}^{2} ( θ )$$

wherein c is the mass concentration of the particles in the solvent (g/mL); A₂ is the second virial coefficient (mol·mL/g²); P(θ) is a form factor relating to the dependence of scattered light intensity on angle; R_{θ} is the excess Rayleigh ratio (cm⁻¹); and K* is an optical constant that is equal to 4π²ηₒ (dn/dc)²λ₀⁻⁴N_{A}⁻¹, where ηₒ is the refractive index of the solvent at the incident radiation (vacuum) wavelength, λ₀ is the incident radiation (vacuum) wavelength (nm), N_{A} is Avogadro's number (mol⁻¹), and dn/dc is the differential refractive index increment (mL/g) (cf., e.g., Buchholz et al. (Electrophoresis 22 (2001), 4118-4128); B. H. Zimm (J. Chem. Phys. 13 (1945), 141; P. Debye (J. Appl. Phys. 15 (1944): 338; and W. Burchard (Anal. Chem. 75 (2003), 4279-4291). Preferably, the Berry plot is calculated using the following term or the reciprocal thereof:

$$\sqrt{\frac{{R}_{θ}}{K * c}}$$

wherein c, R_{θ} and K* are as defined above. Preferably, the Debye plot is calculated using the following term or the reciprocal thereof:

$$\frac{K * c}{{R}_{θ}}$$

wherein c, R_{θ} and K* are as defined above.

The expression “dynamic light scattering” or “DLS” as used herein refers to a technique to determine the size and size distribution profile of particles, in particular with respect to the hydrodynamic radius of the particles. A monochromatic light source, usually a laser, is shot through a polarizer and into a sample. The scattered light then goes through a second polarizer where it is detected and the resulting image is projected onto a screen. The particles in the solution are being hit with the light and diffract the light in all directions. The diffracted light from the particles can either interfere constructively (light regions) or destructively (dark regions). This process is repeated at short time intervals and the resulting set of speckle patterns are analyzed by an autocorrelator that compares the intensity of light at each spot over time.

The expression “static light scattering” or “SLS” as used herein refers to a technique to determine the size and size distribution profile of particles, in particular with respect to the radius of gyration of the particles, and/or the molar mass of particles. A high-intensity monochromatic light, usually a laser, is launched in a solution containing the particles. One or many detectors are used to measure the scattering intensity at one or many angles. The angular dependence is needed to obtain accurate measurements of both molar mass and size for all macromolecules of radius. Hence simultaneous measurements at several angles relative to the direction of incident light, known as multi-angle light scattering (MALS) or multi-angle laser light scattering (MALLS), is generally regarded as the standard implementation of static light scattering.

### Nucleic Acids

The term “nucleic acid” comprises deoxyribonucleic acid (DNA), ribonucleic acid (RNA), combinations thereof, and modified forms thereof. The term comprises genomic DNA, cDNA, mRNA, recombinantly produced and chemically synthesized molecules. In some embodiments, a nucleic acid is DNA. In some embodiments, a nucleic acid is RNA. In some embodiments, a nucleic acid is a mixture of DNA and RNA. A nucleic acid may be present as a single-stranded or double-stranded and linear or covalently circularly closed molecule. A nucleic acid can be isolated. The term “isolated nucleic acid” means, according to the present disclosure, that the nucleic acid (i) was amplified in vitro, for example via polymerase chain reaction (PCR) for DNA or in vitro transcription (using, e.g., an RNA polymerase) for RNA, (ii) was produced recombinantly by cloning, (iii) was purified, for example, by cleavage and separation by gel electrophoresis, or (iv) was synthesized, for example, by chemical synthesis.

The term “nucleoside” (abbreviated herein as “N”) relates to compounds which can be thought of as nucleotides without a phosphate group. While a nucleoside is a nucleobase linked to a sugar (e.g., ribose or deoxyribose), a nucleotide is composed of a nucleoside and one or more phosphate groups. Examples of nucleosides include cytidine, uridine, pseudouridine, adenosine, and guanosine.

The five standard nucleosides which usually make up naturally occurring nucleic acids are uridine, adenosine, thymidine, cytidine and guanosine. The five nucleosides are commonly abbreviated to their one letter codes U, A, T, C and G, respectively. However, thymidine is more commonly written as “dT” (“d” represents “deoxy”) as it contains a 2′-deoxyribofuranose moiety rather than the ribofuranose ring found in uridine. This is because thymidine is found in deoxyribonucleic acid (DNA) and not ribonucleic acid (RNA). Conversely, uridine is found in RNA and not DNA. The remaining three nucleosides may be found in both RNA and DNA. In RNA, they would be represented as A, C and G, whereas in DNA they would be represented as dA, dC and dG.

A modified purine (A or G) or pyrimidine (C, T, or U) base moiety is, in some embodiments, modified by one or more alkyl groups, e.g., one or more C₁₋₄ alkyl groups, e.g., one or more methyl groups. Particular examples of modified purine or pyrimidine base moieties include N⁷-alkyl-guanine, N⁶-alkyl-adenine, 5-alkyl-cytosine, 5-alkyl-uracil, and N (1)-alkyl-uracil, such as N⁷-C₁₋₄ alkyl-guanine, N⁶-C₁₋₄ alkyl-adenine, 5-C₁₋₄ alkyl-cytosine, 5-C₁₋₄ alkyl-uracil, and N(1)-C₁₋₄ alkyl-uracil, preferably N⁷-methyl-guanine, N⁶-methyl-adenine, 5-methyl-cytosine, 5-methyl-uracil, and N(1)-methyl-uracil.

### DNA

Herein, the term “DNA” relates to a nucleic acid molecule which is entirely or at least substantially composed of deoxyribonucleotide residues. In preferred embodiments, the DNA contains all or a majority of deoxyribonucleotide residues. As used herein, “deoxyribonucleotide” refers to a nucleotide which lacks a hydroxyl group at the 2′-position of a β-D-ribofuranosyl group. DNA encompasses without limitation, double stranded DNA, single stranded DNA, isolated DNA such as partially purified DNA, essentially pure DNA, synthetic DNA, recombinantly produced DNA, as well as modified DNA that differs from naturally occurring DNA by the addition, deletion, substitution and/or alteration of one or more nucleotides. Such alterations may refer to addition of non-nucleotide material to internal DNA nucleotides or to the end(s) of DNA. It is also contemplated herein that nucleotides in DNA may be non-standard nucleotides, such as chemically synthesized nucleotides or ribonucleotides. For the present disclosure, these altered DNAs are considered analogs of naturally-occurring DNA. A molecule contains “a majority of deoxyribonucleotide residues” if the content of deoxyribonucleotide residues in the molecule is more than 50% (such as at least 55%, at least 60%, at least 65%, at least 70%, at least 75%, at least 80%, at least 85%, at least 90%, at least 95%, at least 96%, at least 97%, at least 98%, at least 99%), based on the total number of nucleotide residues in the molecule. The total number of nucleotide residues in a molecule is the sum of all nucleotide residues (irrespective of whether the nucleotide residues are standard (i.e., naturally occurring) nucleotide residues or analogs thereof).

DNA may be recombinant DNA and may be obtained by cloning of a nucleic acid, in particular cDNA. The cDNA may be obtained by reverse transcription of RNA.

### RNA

The term “RNA” relates to a nucleic acid molecule which includes ribonucleotide residues. In preferred embodiments, the RNA contains all or a majority of ribonucleotide residues. As used herein, “ribonucleotide” refers to a nucleotide with a hydroxyl group at the 2′-position of a β-D-ribofuranosyl group. RNA encompasses without limitation, double stranded RNA, single stranded RNA, isolated RNA such as partially purified RNA, essentially pure RNA, synthetic RNA, recombinantly produced RNA, as well as modified RNA that differs from naturally occurring RNA by the addition, deletion, substitution and/or alteration of one or more nucleotides. Such alterations may refer to addition of non-nucleotide material to internal RNA nucleotides or to the end(s) of RNA. It is also contemplated herein that nucleotides in RNA may be non-standard nucleotides, such as chemically synthesized nucleotides or deoxynucleotides. For the present disclosure, these altered/modified nucleotides can be referred to as analogs of naturally occurring nucleotides, and the corresponding RNAs containing such altered/modified nucleotides (i.e., altered/modified RNAs) can be referred to as analogs of naturally occurring RNAs. A molecule contains “a majority of ribonucleotide residues” if the content of ribonucleotide residues in the molecule is more than 50% (such as at least 55%, at least 60%, at least 65%, at least 70%, at least 75%, at least 80%, at least 85%, at least 90%, at least 95%, at least 96%, at least 97%, at least 98%, at least 99%), based on the total number of nucleotide residues in the molecule. The total number of nucleotide residues in a molecule is the sum of all nucleotide residues (irrespective of whether the nucleotide residues are standard (i.e., naturally occurring) nucleotide residues or analogs thereof).

“RNA” includes mRNA, tRNA, ribosomal RNA (rRNA), small nuclear RNA (snRNA), self-amplifying RNA (saRNA), trans-amplifying RNA (taRNA), single-stranded RNA (ssRNA), dsRNA, inhibitory RNA (such as antisense ssRNA, small interfering RNA (siRNA), or microRNA (miRNA)), activating RNA (such as small activating RNA) and immunostimulatory RNA (isRNA). In some embodiments, “RNA” refers to mRNA.

The term “in vitro transcription” or “IVT” as used herein means that the transcription (i.e., the generation of RNA) is conducted in a cell-free manner. I.e., IVT does not use living/cultured cells but rather the transcription machinery extracted from cells (e.g., cell lysates or the isolated components thereof, including an RNA polymerase (preferably T7, T3 or SP6 polymerase)).

According to the present disclosure, the term “RNA” includes “mRNA”. According to the present disclosure, the term “mRNA” means “messenger-RNA” and includes a “transcript” which may be generated by using a DNA template. Generally, mRNA encodes a peptide or polypeptide.

mRNA is single-stranded but may contain self-complementary sequences that allow parts of the mRNA to fold and pair with itself to form double helices.

According to the present disclosure, “dsRNA” means double-stranded RNA and is RNA with two partially or completely complementary strands.

In preferred embodiments of the present disclosure, the mRNA relates to an RNA transcript which encodes a peptide or polypeptide.

In some embodiments, the mRNA which preferably encodes a peptide or polypeptide has a length of at least 45 nucleotides (such as at least 60, at least 90, at least 100, at least 200, at least 300, at least 400, at least 500, at least 600, at least 700, at least 800, at least 900, at least 1,000, at least 1,500, at least 2,000, at least 2,500, at least 3,000, at least 3,500, at least 4,000, at least 4,500, at least 5,000, at least 6,000, at least 7,000, at least 8,000, at least 9,000 nucleotides), preferably up to 15,000, such as up to 14,000, up to 13,000, up to 12,000 nucleotides, up to 11,000 nucleotides or up to 10,000 nucleotides.

As established in the art, mRNA generally contains a 5′ untranslated region (5′-UTR), a peptide/polypeptide coding region and a 3′ untranslated region (3′-UTR). In some embodiments, the mRNA is produced by in vitro transcription or chemical synthesis. In some embodiments, the mRNA is produced by in vitro transcription using a DNA template. The in vitro transcription methodology is known to the skilled person; cf., e.g., Molecular Cloning: A Laboratory Manual, 4th Edition, M. R. Green and J. Sambrook eds., Cold Spring Harbor Laboratory Press, Cold Spring Harbor 2012. Furthermore, a variety of in vitro transcription kits is commercially available, e.g., from Thermo Fisher Scientific (such as TranscriptAid™ T7 kit, MEGAscript® T7 kit, MAXIscript®), New England BioLabs Inc. (such as HiScribe™ T7 kit, HiScribe™ T7 ARCA mRNA kit), Promega (such as RiboMAX™, HeLaScribe®, Riboprobe® systems), Jena Bioscience (such as SP6 or T7 transcription kits), and Epicentre (such as AmpliScribe™). For providing modified mRNA, correspondingly modified nucleotides, such as modified naturally occurring nucleotides, non-naturally occurring nucleotides and/or modified non-naturally occurring nucleotides, can be incorporated during synthesis (preferably in vitro transcription), or modifications can be effected in and/or added to the mRNA after transcription.

In some embodiments, RNA is in vitro transcribed RNA (IVT-RNA) and may be obtained by in vitro transcription of an appropriate DNA template. The promoter for controlling transcription can be any promoter for any RNA polymerase. Particular examples of RNA polymerases are the T7, T3, and SP6 RNA polymerases. Preferably, the in vitro transcription is controlled by a T7 or SP6 promoter. A DNA template for in vitro transcription may be obtained by cloning of a nucleic acid, in particular cDNA, and introducing it into an appropriate vector for in vitro transcription. The cDNA may be obtained by reverse transcription of RNA.

In some embodiments of the present disclosure, the RNA is “replicon RNA” or simply a “replicon”, in particular “self-replicating RNA” or “self-amplifying RNA”. In certain embodiments, the replicon or self-replicating RNA is derived from or comprises elements derived from an ssRNA virus, in particular a positive-stranded ssRNA virus such as an alphavirus. Alphaviruses are typical representatives of positive-stranded RNA viruses. Alphaviruses replicate in the cytoplasm of infected cells (for review of the alphaviral life cycle see José et al., Future Microbiol., 2009, vol. 4, pp. 837-856). The total genome length of many alphaviruses typically ranges between 11,000 and 12,000 nucleotides, and the genomic RNA typically has a 5′-cap, and a 3′ poly(A) tail. The genome of alphaviruses encodes non-structural proteins (involved in transcription, modification and replication of viral RNA and in protein modification) and structural proteins (forming the virus particle). There are typically two open reading frames (ORFs) in the genome. The four non-structural proteins (nsP1-nsP4) are typically encoded together by a first ORF beginning near the 5′ terminus of the genome, while alphavirus structural proteins are encoded together by a second ORF which is found downstream of the first ORF and extends near the 3′ terminus of the genome. Typically, the first ORF is larger than the second ORF, the ratio being roughly 2:1. In cells infected by an alphavirus, only the nucleic acid sequence encoding non-structural proteins is translated from the genomic RNA, while the genetic information encoding structural proteins is translatable from a subgenomic transcript, which is an RNA molecule that resembles eukaryotic messenger RNA (mRNA; Gould et al., 2010, Antiviral Res., vol. 87 pp. 111-124). Following infection, i.e. at early stages of the viral life cycle, the (+) stranded genomic RNA directly acts like a messenger RNA for the translation of the open reading frame encoding the non-structural poly-protein (nsP1234).

Alphavirus-derived vectors have been proposed for delivery of foreign genetic information into target cells or target organisms. In simple approaches, the open reading frame encoding alphaviral structural proteins is replaced by an open reading frame encoding a protein of interest. Alphavirus-based trans-replication (trans-amplification) systems rely on alphavirus nucleotide sequence elements on two separate nucleic acid molecules: one nucleic acid molecule encodes a viral replicase, and the other nucleic acid molecule is capable of being replicated by said replicase in trans (hence the designation trans-replication system). Trans-replication requires the presence of both these nucleic acid molecules in a given host cell. The nucleic acid molecule capable of being replicated by the replicase in trans must comprise certain alphaviral sequence elements to allow recognition and RNA synthesis by the alphaviral replicase.

In some embodiments of the present disclosure, the RNA (in particular, mRNA) described herein (e.g., contained in the compositions/formulations of the present disclosure and/or used in the methods of the present disclosure) contains one or more modifications, e.g., in order to increase its stability and/or increase translation efficiency and/or decrease immunogenicity and/or decrease cytotoxicity. For example, in order to increase expression of the RNA (in particular, mRNA), it may be modified within the coding region, i.e., the sequence encoding the expressed peptide or polypeptide, preferably without altering the sequence of the expressed peptide or polypeptide. Such modifications are described, for example, in WO 2007/036366 and PCT/EP2019/056502, and include the following: a 5′-cap structure; an extension or truncation of the naturally occurring poly(A) tail; an alteration of the 5′- and/or 3′-untranslated regions (UTR) such as introduction of a UTR which is not related to the coding region of said RNA; the replacement of one or more naturally occurring nucleotides with synthetic nucleotides; and codon optimization (e.g., to alter, preferably increase, the GC content of the RNA). A combination of the above described modifications, i.e., incorporation of a 5′-cap structure, incorporation of a poly-A sequence, unmasking of a poly-A sequence, alteration of the 5′- and/or 3′-UTR (such as incorporation of one or more 3′-UTRs), replacing one or more naturally occurring nucleotides with synthetic nucleotides (e.g., 5-methylcytidine for cytidine and/or pseudouridine (W) or N(1)-methylpseudouridine (m1) or 5-methyluridine (m5U) for uridine), and codon optimization, has a synergistic influence on the stability of RNA (preferably mRNA) and increase in translation efficiency. Thus, in some embodiments, the RNA (in particular, mRNA) described in the present disclosure contains a combination of at least two, at least three, at least four or all five of the above-mentioned modifications, i.e., (i) incorporation of a 5′-cap structure, (ii) incorporation of a poly-A sequence, unmasking of a poly-A sequence; (iii) alteration of the 5′- and/or 3′-UTR (such as incorporation of one or more 3′-UTRs); (iv) replacing one or more naturally occurring nucleotides with synthetic nucleotides (e.g., 5-methylcytidine for cytidine and/or pseudouridine (ψ) or N (1)-methylpseudouridine (m1ψ) or 5-methyluridine (m5U) for uridine), and (v) codon optimization.

### 5′-Cap

In some embodiments, the RNA (in particular, mRNA) described herein comprises a 5′-cap structure. In some embodiments, the RNA does not have uncapped 5′-triphosphates. In some embodiments, the RNA (in particular, mRNA) may comprise a conventional 5′-cap and/or a 5′-cap analog. The term “conventional 5′-cap” refers to a cap structure found on the 5′-end of an RNA molecule and generally comprises a guanosine 5′-triphosphate (Gppp) which is connected via its triphosphate moiety to the 5′-end of the next nucleotide of the RNA (i.e., the guanosine is connected via a 5′ to 5′ triphosphate linkage to the rest of the RNA). The guanosine may be methylated at position N⁷ (resulting in the cap structure m⁷Gppp). The term “5′-cap analog” includes a 5′-cap which is based on a conventional 5′-cap but which has been modified at either the 2′- or 3′-position of the m⁷guanosine structure in order to avoid an integration of the 5′-cap analog in the reverse orientation (such 5′-cap analogs are also called anti-reverse cap analogs (ARCAs)). Particularly preferred 5′-cap analogs are those having one or more substitutions at the bridging and non-bridging oxygen in the phosphate bridge, such as phosphorothioate modified 5′-cap analogs at the β-phosphate (such as m₂^{7,2′O}G(5′)ppSp(5′) G (referred to as beta-S-ARCA or β-S-ARCA)), as described in PCT/EP2019/056502. Providing an RNA (in particular, mRNA) with a 5′-cap structure as described herein may be achieved by in vitro transcription of a DNA template in presence of a corresponding 5′-cap compound, wherein said 5′-cap structure is co-transcriptionally incorporated into the generated RNA (in particular, mRNA) strand, or the RNA (in particular, mRNA) may be generated, for example, by in vitro transcription, and the 5′-cap structure may be attached to the RNA post-transcriptionally using capping enzymes, for example, capping enzymes of vaccinia virus.

In some embodiments, the RNA (in particular, mRNA) comprises a 5′-cap structure selected from the group consisting of m₂^{7,2′O}G(5)ppSp(5′)G (in particular its D1 diastereomer), m₂^{7,3′O}G (5′)ppp(5′) G, and m₂^{7,3-O}Gppp (m₁^{2′-O})ApG. In some embodiments, RNA comprises m₂^{7,2′O}G(5′)ppSp(5′) G (in particular its D1 diastereomer) as 5′-cap structure. In some embodiments, RNA comprises m₂^{7,3′-O}Gppp (m₁^{2′-O})ApG as 5′-cap structure.

In some embodiments, the RNA (in particular, mRNA) comprises a cap0, cap1, or cap2, preferably cap1 or cap2. According to the present disclosure, the term “cap0” means the structure “m⁷GpppN”, wherein N is any nucleoside bearing an OH moiety at position 2′. According to the present disclosure, the term “cap1” means the structure “m⁷GpppNm”, wherein Nm is any nucleoside bearing an OCH₃ moiety at position 2′. According to the present disclosure, the term “cap2” means the structure “m⁷GpppNmNm”, wherein each Nm is independently any nucleoside bearing an OCH₃ moiety at position 2′.

The 5′-cap analog beta-S-ARCA (β-S-ARCA) has the following structure:

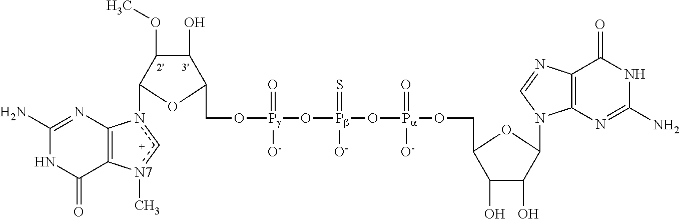

The “D1 diastereomer of beta-S-ARCA” or “beta-S-ARCA (D1)” is the diastereomer of beta-S-ARCA which elutes first on an HPLC column compared to the D2 diastereomer of beta-S-ARCA (beta-S-ARCA (D2)) and thus exhibits a shorter retention time. The HPLC preferably is an analytical HPLC. In some embodiments, a Supelcosil LC-18-T RP column, preferably of the format: 5 μm, 4.6×250 mm is used for separation, whereby a flow rate of 1.3 ml/min can be applied. In some embodiments, a gradient of methanol in ammonium acetate, for example, a 0-25% linear gradient of methanol in 0.05 M ammonium acetate, pH=5.9, within 15 min is used. UV-detection (VWD) can be performed at 260 nm and fluorescence detection (FLD) can be performed with excitation at 280 nm and detection at 337 nm.

The 5′-cap analog m₂^{7,3′-O}Gppp (m₁^{2′-O})ApG (also referred to as m₂^{7,3′O}G(5′)ppp(5′)m^{2′-O}ApG) which is a building block of a cap1 has the following structure:

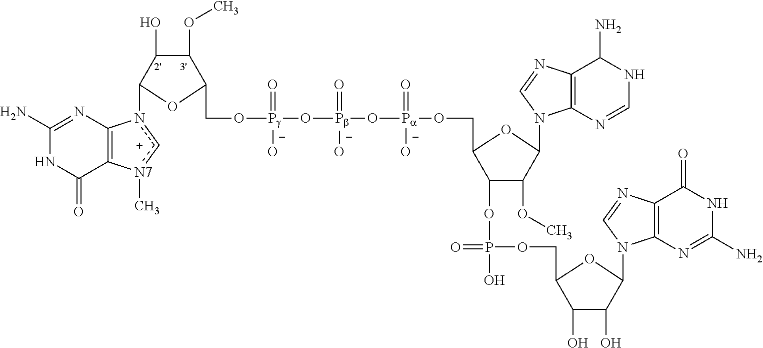

An exemplary cap0 mRNA comprising β-S-ARCA and mRNA has the following structure:

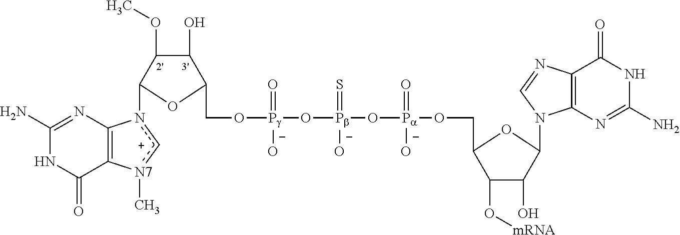

An exemplary cap0 mRNA comprising m₂^{7,3′O}G(5′)ppp(5′)G and mRNA has the following structure:

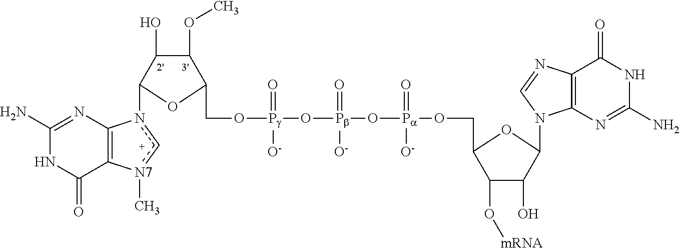

An exemplary cap1 mRNA comprising m₂^{7,3′-O}Gppp (m₁^{2′-O})ApG and mRNA has the following structure:

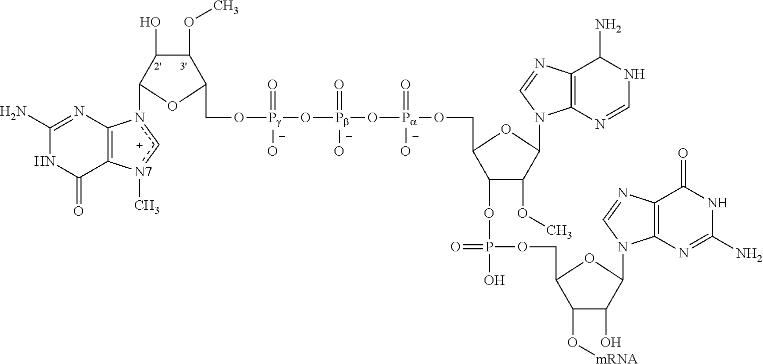

### Poly-A Tail

As used herein, the term “poly-A tail” or “poly-A sequence” refers to an uninterrupted or interrupted sequence of adenylate residues which is typically located at the 3′-end of an RNA (in particular, mRNA) molecule. Poly-A tails or poly-A sequences are known to those of skill in the art and may follow the 3′-UTR in the RNAs (in particular, mRNAs) described herein. An uninterrupted poly-A tail is characterized by consecutive adenylate residues. In nature, an uninterrupted poly-A tail is typical. RNAs (in particular, mRNAs) disclosed herein can have a poly-A tail attached to the free 3′-end of the RNA by a template-independent RNA polymerase after transcription or a poly-A tail encoded by DNA and transcribed by a template-dependent RNA polymerase.

It has been demonstrated that a poly-A tail of about 120 A nucleotides has a beneficial influence on the levels of RNA in transfected eukaryotic cells, as well as on the levels of protein that is translated from an open reading frame that is present upstream (5′) of the poly-A tail (Holtkamp et al., 2006, Blood, vol. 108, pp. 4009-4017).

The poly-A tail may be of any length. In some embodiments, a poly-A tail comprises, essentially consists of, or consists of at least 20, at least 30, at least 40, at least 80, or at least 100 and up to 500, up to 400, up to 300, up to 200, or up to 150 A nucleotides, and, in particular, about 120 A nucleotides. In this context, “essentially consists of” means that most nucleotides in the poly-A tail, typically at least 75%, at least 80%, at least 85%, at least 90%, at least 95%, at least 96%, at least 97%, at least 98%, or at least 99% by number of nucleotides in the poly-A tail are A nucleotides, but permits that remaining nucleotides are nucleotides other than A nucleotides, such as U nucleotides (uridylate), G nucleotides (guanylate), or C nucleotides (cytidylate). In this context, “consists of” means that all nucleotides in the poly-A tail, i.e., 100% by number of nucleotides in the poly-A tail, are A nucleotides. The term “A nucleotide” or “A” refers to adenylate.

In some embodiments, a poly-A tail is attached during RNA transcription, e.g., during preparation of in vitro transcribed RNA, based on a DNA template comprising repeated dT nucleotides (deoxythymidylate) in the strand complementary to the coding strand. The DNA sequence encoding a poly-A tail (coding strand) is referred to as poly(A) cassette. In some embodiments, the poly(A) cassette present in the coding strand of DNA essentially consists of dA nucleotides, but is interrupted by a random sequence of the four nucleotides (dA, dC, dG, and dT). Such random sequence may be 5 to 50, 10 to 30, or 10 to 20 nucleotides in length. Such a cassette is disclosed in WO 2016/005324 A1, hereby incorporated by reference. Any poly(A) cassette disclosed in WO 2016/005324 A1 may be used in the present disclosure. A poly(A) cassette that essentially consists of dA nucleotides, but is interrupted by a random sequence having an equal distribution of the four nucleotides (dA, dC, dG, dT) and having a length of e.g., 5 to 50 nucleotides shows, on DNA level, constant propagation of plasmid DNA in E. coli and is still associated, on RNA level, with the beneficial properties with respect to supporting RNA stability and translational efficiency is encompassed. Consequently, in some embodiments, the poly-A tail contained in an RNA (in particular, mRNA) molecule described herein essentially consists of A nucleotides, but is interrupted by a random sequence of the four nucleotides (A, C, G, U). Such random sequence may be 5 to 50, 10 to 30, or 10 to 20 nucleotides in length.

In some embodiments, the poly(A) tail comprises 30 adenine nucleotides followed by 70 adenine nucleotides, wherein the 30 adenine nucleotides and 70 adenine nucleotides are separated by a linker sequence of 10 nucleotides.

In some embodiments, no nucleotides other than A nucleotides flank a poly-A tail at its 3′-end, i.e., the poly-A tail is not masked or followed at its 3′-end by a nucleotide other than A.

In some embodiments, a poly-A tail may comprise at least 20, at least 30, at least 40, at least 80, or at least 100 and up to 500, up to 400, up to 300, up to 200, or up to 150 nucleotides. In some embodiments, the poly-A tail may essentially consist of at least 20, at least 30, at least 40, at least 80, or at least 100 and up to 500, up to 400, up to 300, up to 200, or up to 150 nucleotides. In some embodiments, the poly-A tail may consist of at least 20, at least 30, at least 40, at least 80, or at least 100 and up to 500, up to 400, up to 300, up to 200, or up to 150 nucleotides. In some embodiments, the poly-A tail comprises the poly-A tail shown in SEQ ID NO: 41. In some embodiments, the poly-A tail comprises at least 100 nucleotides. In some embodiments, the poly-A tail comprises about 150 nucleotides. In some embodiments, the poly-A tail comprises about 120 nucleotides.

### Untranslated Regions (UTR)

In some embodiments, RNA (in particular, mRNA) described in present disclosure comprises a 5′-UTR and/or a 3′-UTR. The term “untranslated region” or “UTR” relates to a region in a DNA molecule which is transcribed but is not translated into an amino acid sequence, or to the corresponding region in an RNA molecule, such as an mRNA molecule. An untranslated region (UTR) can be present 5′ (upstream) of an open reading frame (5′-UTR) and/or 3′ (downstream) of an open reading frame (3′-UTR). A 5′-UTR, if present, is located at the 5′-end, upstream of the start codon of a protein-encoding region. A 5′-UTR is downstream of the 5′-cap (if present), e.g., directly adjacent to the 5′-cap. A 3′-UTR, if present, is located at the 3′-end, downstream of the termination codon of a protein-encoding region, but the term “3′-UTR” does generally not include the poly-A sequence. Thus, the 3′-UTR is upstream of the poly-A sequence (if present), e.g., directly adjacent to the poly-A sequence. Incorporation of a 3′-UTR into the 3′-non translated region of an RNA (preferably mRNA) molecule can result in an enhancement in translation efficiency. A synergistic effect may be achieved by incorporating two or more of such 3′-UTRs (which are preferably arranged in a head-to-tail orientation; cf., e.g., Holtkamp et al., Blood 108, 4009-4017 (2006)). The 3′-UTRs may be autologous or heterologous to the RNA (e.g., mRNA) into which they are introduced. In certain embodiments, the 3′-UTR is derived from a globin gene or mRNA, such as a gene or mRNA of alpha2-globin, alpha1-globin, or beta-globin, e.g., beta-globin, e.g., human beta-globin. For example, the RNA (e.g., mRNA) may be modified by the replacement of the existing 3′-UTR with or the insertion of one or more, e.g., two copies of a 3′-UTR derived from a globin gene, such as alpha2-globin, alpha1-globin, beta-globin, e.g., beta-globin, e.g., human beta-globin.

In some embodiments, a 5′-UTR is or comprises a modified human alpha-globin 5′-UTR. A particularly preferred 5′-UTR comprises the nucleotide sequence of SEQ ID NO: 39. In some embodiments, a 3′-UTR comprises a first sequence from the amino terminal enhancer of split (AES) messenger RNA and a second sequence from the mitochondrial encoded 12S ribosomal RNA. A particularly preferred 3′-UTR comprises the nucleotide sequence of SEQ ID NO: 40.

In some embodiments, RNA comprises a 5′-UTR comprising the nucleotide sequence of SEQ ID NO: 39, or a nucleotide sequence having at least 99%, 98%, 97%, 96%, 95%, 90%, 85%, or 80% identity to the nucleotide sequence of SEQ ID NO: 39.

In some embodiments, RNA comprises a 3′-UTR comprising the nucleotide sequence of SEQ ID NO: 40, or a nucleotide sequence having at least 99%, 98%, 97%, 96%, 95%, 90%, 85%, or 80% identity to the nucleotide sequence of SEQ ID NO: 40.

TABLE 1
Exemplary untranslated RNA sequences

| Sequence | Sequence (Amino Acid) |
|---|---|
| 5′-UTR | aactagtatt cttctggtcc ccacagactc agagagaacc cgccacc [SEQ ID NO: 39] |
| 3′-UTR | ctggtactgc atgcacgcaa tgctagctgc ccctttcccg tcctgggtac cccgagtctc |
| | ccccgacctc gggtoccagg tatgctccca cctccacctg ccccactcac cacctctgct |
| | agttccagac acctcccaag cacgcagcaa tgcagctcaa aacgcttagc ctagccacac |
| | ccccacggga aacagcagtg attaaccttt agcaataaac gaaagtttaa ctaagctata |
| | ctaaccccag ggttggtcaa tttcgtgcca gccacacc [SEQ ID NO: 40] |
| polyA | aaaaaaaaaa aaaaaaaaaa aaaaaaaaaa gcatatgact aaaaaaaaaa aaaaaaaaaa |
| | aaaaaaaaaa aaaaaaaaaa aaaaaaaaaa aaaaaaaaaa aaaaaaaaaa [SEQ ID NO: 41] |

### Chemical Modification

The RNA (in particular, mRNA) described herein may have modified ribonucleotides in order to increase its stability and/or decrease immunogenicity and/or decrease cytotoxicity. For example, in some embodiments, uridine in the RNA (in particular, mRNA) described herein is replaced (partially or completely, preferably completely) by a modified nucleoside. In some embodiments, the modified nucleoside is a modified uridine.

In some embodiments, the modified uridine replacing uridine is selected from the group consisting of pseudouridine (ψ), N1-methyl-pseudouridine (m1ψ), 5-methyl-uridine (m5U), and combinations thereof.

In some embodiments, the modified nucleoside replacing (partially or completely, preferably completely) uridine in the RNA may be any one or more of 3-methyl-uridine (m3U), 5-methoxy-uridine (mo5U), 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridineor 5-bromo-uridine), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 1-ethyl-pseudouridine, 5-methylaminomethyl-2-thio-uridine (mnm5s2U), 5-methylaminomethyl-2-seleno-uridine (mnm5se2U), 5-carbamoylmethyl-uridine (ncm5U), 5-carboxymethylaminomethyl-uridine (cmnm5U), 5-carboxymethylaminomethyl-2-thio-uridine (cmnm5s2U), 5-propynyl-uridine, 1-propynyl-pseudouridine, 5-taurinomethyl-uridine (Tm5U), 1-taurinomethyl-pseudouridine, 5-taurinomethyl-2-thio-uridine (Tm5s2U), 1-taurinomethyl-4-thio-pseudouridine), 5-methyl-2-thio-uridine (m5s2U), 1-methyl-4-thio-pseudouridine (m1s4ψ), 4-thio-1-methyl-pseudouridine, 3-methyl-pseudouridine (m3ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl) uridine (acp3U), 1-methyl-3-(3-amino-3-carboxypropyl) pseudouridine (acp3 ψ), 5-(isopentenylaminomethyl) uridine (inm5U), 5-(isopentenylaminomethyl)-2-thio-uridine (inm5s2U), α-thio-uridine, 2′-O-methyl-uridine (Um), 5,2′-O-dimethyl-uridine (m5Um), 2′-O-methyl-pseudouridine (ψm), 2-thio-2′-O-methyl-uridine (s2Um), 5-methoxycarbonylmethyl-2′-O-methyl-uridine (mcm5Um), 5-carbamoylmethyl-2′-O-methyl-uridine (ncm5Um), 5-carboxymethylaminomethyl-2′-O-methyl-uridine (cmnm5Um), 3,2′-O-dimethyl-uridine (m3Um), 5-(isopentenylaminomethyl)-2′-O-methyl-uridine (inm5Um), 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino) uridine, or any other modified uridine known in the art.

An RNA (preferably mRNA) which is modified by pseudouridine (replacing partially or completely, preferably completely, uridine) is referred to herein as “ψ-modified”, whereas the term “m1ψ-modified” means that the RNA (preferably mRNA) contains N(1)-methylpseudouridine (replacing partially or completely, preferably completely, uridine). Furthermore, the term “m5U-modified” means that the RNA (preferably mRNA) contains 5-methyluridine (replacing partially or completely, preferably completely, uridine). Such ψ- or m1ψ- or m5U-modified RNAs usually exhibit decreased immunogenicity compared to their unmodified forms and, thus, are preferred in applications where the induction of an immune response is to be avoided or minimized. In some embodiments, the RNA (preferably mRNA) contains N(1)-methylpseudouridine replacing completely uridine.

### Codon Optimization and GC Enrichment

The codons of the RNA (in particular, mRNA) described in the present disclosure may further be optimized, e.g., to increase the GC content of the RNA and/or to replace codons which are rare in the cell (or subject) in which the peptide or polypeptide of interest is to be expressed by codons which are synonymous frequent codons in said cell (or subject). In some embodiments, the amino acid sequence encoded by the RNA (in particular, mRNA) described in the present disclosure is encoded by a coding sequence which is codon-optimized and/or the G/C content of which is increased compared to wild type coding sequence. This also includes embodiments, wherein one or more sequence regions of the coding sequence are codon-optimized and/or increased in the G/C content compared to the corresponding sequence regions of the wild type coding sequence. In some embodiments, the codon-optimization and/or the increase in the G/C content preferably does not change the sequence of the encoded amino acid sequence.

The term “codon-optimized” refers to the alteration of codons in the coding region of a nucleic acid molecule to reflect the typical codon usage of a host organism without preferably altering the amino acid sequence encoded by the nucleic acid molecule. Within the context of the present disclosure, coding regions may be codon-optimized for optimal expression in a subject to be treated using the RNA (in particular, mRNA) described herein. Codon-optimization is based on the finding that the translation efficiency is also determined by a different frequency in the occurrence of tRNAs in cells. Thus, the sequence of RNA (in particular, mRNA) may be modified such that codons for which frequently occurring tRNAs are available are inserted in place of “rare codons”.

In some embodiments, the guanosine/cytosine (G/C) content of the coding region of the RNA (in particular, mRNA) described herein is increased compared to the G/C content of the corresponding coding sequence of the wild type RNA, wherein the amino acid sequence encoded by the RNA is preferably not modified compared to the amino acid sequence encoded by the wild type RNA. This modification of the RNA sequence is based on the fact that the sequence of any RNA region to be translated is important for efficient translation of that RNA. Sequences having an increased G (guanosine)/C (cytosine) content are more stable than sequences having an increased A (adenosine)/U (uracil) content. In respect to the fact that several codons code for one and the same amino acid (so-called degeneration of the genetic code), the most favorable codons for the stability can be determined (so-called alternative codon usage). Depending on the amino acid to be encoded by the RNA, there are various possibilities for modification of the RNA sequence, compared to its wild type sequence. In particular, codons which contain A and/or U nucleotides can be modified by substituting these codons by other codons, which code for the same amino acids but contain no A and/or U or contain a lower content of A and/or U nucleotides.

In various embodiments, the G/C content of the coding region of the RNA (in particular, mRNA) described herein is increased by at least 10%, at least 20%, at least 30%, at least 40%, at least 50%, at least 55%, or even more compared to the G/C content of the coding region of the wild type RNA.

### Non-Immunogenic RNA

The term “non-immunogenic RNA” (such as “non-immunogenic mRNA”) as used herein refers to RNA that does not induce a response by the immune system upon administration, e.g., to a mammal, or induces a weaker response than would have been induced by the same RNA that differs only in that it has not been subjected to the modifications and treatments that render the non-immunogenic RNA non-immunogenic, i.e., than would have been induced by standard RNA (stdRNA). In certain embodiments, non-immunogenic RNA is rendered non-immunogenic by incorporating modified nucleosides suppressing RNA-mediated activation of innate immune receptors into the RNA and/or limiting the amount of double-stranded RNA (dsRNA), e.g., by limiting the formation of double-stranded RNA (dsRNA), e.g., during in vitro transcription, and/or by removing double-stranded RNA (dsRNA), e.g., following in vitro transcription. In certain embodiments, non-immunogenic RNA is rendered non-immunogenic by incorporating modified nucleosides suppressing RNA-mediated activation of innate immune receptors into the RNA and/or by removing double-stranded RNA (dsRNA), e.g., following in vitro transcription.

For rendering the non-immunogenic RNA (especially mRNA) non-immunogenic by the incorporation of modified nucleosides, any modified nucleoside may be used as long as it lowers or suppresses immunogenicity of the RNA. Particularly preferred are modified nucleosides that suppress RNA-mediated activation of innate immune receptors. In some embodiments, the modified nucleosides comprise a replacement of one or more uridines with a nucleoside comprising a modified nucleobase. In some embodiments, the modified nucleobase is a modified uracil. In some embodiments, the nucleoside comprising a modified nucleobase is selected from the group consisting of 3-methyl-uridine (m³U), 5-methoxy-uridine (mo⁵U), 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho5U), 5-aminoallyl-uridine, 5-halo-uridine (e.g., 5-iodo-uridine or 5-bromo-uridine), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 1-ethyl-pseudouridine, 5-methylaminomethyl-2-thio-uridine (mnm⁵s2U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (Tm⁵U), 1-taurinomethyl-pseudouridine, 5-taurinomethyl-2-thio-uridine (Tm⁵s²U), 1-taurinomethyl-4-thio-pseudouridine), 5-methyl-2-thio-uridine (m5s2U), 1-methyl-4-thio-pseudouridine (m1s4ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl) uridine (acp³U), 1-methyl-3-(3-amino-3-carboxypropyl) pseudouridine (acp³ ψ), 5-(isopentenylaminomethyl) uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (m⁵Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino) uridine. In certain embodiments, the nucleoside comprising a modified nucleobase is pseudouridine (ψ), N¹-methyl-pseudouridine (m1ψ) or 5-methyl-uridine (m5U), in particular N¹-methyl-pseudouridine.

In some embodiments, the replacement of one or more uridines with a nucleoside comprising a modified nucleobase comprises a replacement of at least 1%, at least 2%, at least 3%, at least 4%, at least 5%, at least 10%, at least 25%, at least 50%, at least 75%, at least 90%, at least 95%, at least 96%, at least 97%, at least 98%, at least 99% or 100% of the uridines.

During synthesis of mRNA by in vitro transcription (IVT) using T7 RNA polymerase significant amounts of aberrant products, including double-stranded RNA (dsRNA) are produced due to unconventional activity of the enzyme. dsRNA induces inflammatory cytokines and activates effector enzymes leading to protein synthesis inhibition. Formation of dsRNA can be limited during synthesis of mRNA by in vitro transcription (IVT), for example, by limiting the amount of uridine triphosphate (UTP) during synthesis. Optionally, UTP may be added once or several times during synthesis of mRNA. Also, dsRNA can be removed from RNA such as IVT RNA, for example, by ion-pair reversed phase HPLC using a non-porous or porous C-18 polystyrene-divinylbenzene (PS-DVB) matrix. Alternatively, an enzymatic based method using E. coli RNaseIII that specifically hydrolyzes dsRNA but not ssRNA, thereby eliminating dsRNA contaminants from IVT RNA preparations can be used. Furthermore, dsRNA can be separated from ssRNA by using a cellulose material. In some embodiments, an RNA preparation is contacted with a cellulose material and the ssRNA is separated from the cellulose material under conditions which allow binding of dsRNA to the cellulose material and do not allow binding of ssRNA to the cellulose material. Suitable methods for providing ssRNA are disclosed, for example, in WO 2017/182524. As the term is used herein, “remove” or “removal” refers to the characteristic of a population of first substances, such as non-immunogenic RNA, being separated from the proximity of a population of second substances, such as dsRNA, wherein the population of first substances is not necessarily devoid of the second substance, and the population of second substances is not necessarily devoid of the first substance. However, a population of first substances characterized by the removal of a population of second substances has a measurably lower content of second substances as compared to the non-separated mixture of first and second substances.

In some embodiments, the amount of double-stranded RNA (dsRNA) is limited, e.g., dsRNA (especially dsmRNA) is removed from non-immunogenic RNA, such that less than 10%, less than 5%, less than 4%, less than 3%, less than 2%, less than 1%, less than 0.5%, less than 0.3%, less than 0.1%, less than 0.05%, less than 0.03%, less than 0.01%, less than 0.005%, less than 0.004%, less than 0.003%, less than 0.002%, less than 0.001%, or less than 0.0005% of the RNA in the non-immunogenic RNA composition is dsRNA. In some embodiments, the non-immunogenic RNA (especially mRNA) is free or essentially free of dsRNA. In some embodiments, the non-immunogenic RNA (especially mRNA) composition comprises a purified preparation of single-stranded nucleoside modified RNA. In some embodiments, the non-immunogenic RNA (especially mRNA) composition comprises single-stranded nucleoside modified RNA (especially mRNA) and is substantially free of double stranded RNA (dsRNA). In some embodiments, the non-immunogenic RNA (especially mRNA) composition comprises at least 90%, at least 91%, at least 92%, at least 93%, at least 94%, at least 95%, at least 96%, at least 97%, at least 98%, at least 99%, at least 99.5%, at least 99.9%, at least 99.99%, at least 99.991%, at least 99.992%, at least 99.993%, at least 99.994%, at least 99.995%, at least 99.996%, at least 99.997%, or at least 99.998% single stranded nucleoside modified RNA, relative to all other nucleic acid molecules (DNA, dsRNA, etc.).

Various methods can be used to determine the amount of dsRNA. For example, a sample may be contacted with dsRNA-specific antibody and the amount of antibody binding to RNA may be taken as a measure for the amount of dsRNA in the sample. A sample containing a known amount of dsRNA may be used as a reference.

For example, RNA may be spotted onto a membrane, e.g., nylon blotting membrane. The membrane may be blocked, e.g., in TBS-T buffer (20 mM TRIS pH 7.4, 137 mM NaCl, 0.1% (v/v) TWEEN-20) containing 5% (w/v) skim milk powder. For detection of dsRNA, the membrane may be incubated with dsRNA-specific antibody, e.g., dsRNA-specific mouse mAb (English & Scientific Consulting, Szirák, Hungary). After washing, e.g., with TBS-T, the membrane may be incubated with a secondary antibody, e.g., HRP-conjugated donkey anti-mouse IgG (Jackson ImmunoResearch, Cat #715-035-150), and the signal provided by the secondary antibody may be detected.

In some embodiments, the non-immunogenic RNA (especially mRNA) is translated in a cell more efficiently than standard RNA with the same sequence. In some embodiments, translation is enhanced by a factor of 2-fold relative to its unmodified counterpart. In some embodiments, translation is enhanced by a 3-fold factor. In some embodiments, translation is enhanced by a 4-fold factor. In some embodiments, translation is enhanced by a 5-fold factor. In some embodiments, translation is enhanced by a 6-fold factor. In some embodiments, translation is enhanced by a 7-fold factor. In some embodiments, translation is enhanced by an 8-fold factor. In some embodiments, translation is enhanced by a 9-fold factor. In some embodiments, translation is enhanced by a 10-fold factor. In some embodiments, translation is enhanced by a 15-fold factor. In some embodiments, translation is enhanced by a 20-fold factor. In some embodiments, translation is enhanced by a 50-fold factor. In some embodiments, translation is enhanced by a 100-fold factor. In some embodiments, translation is enhanced by a 200-fold factor. In some embodiments, translation is enhanced by a 500-fold factor. In some embodiments, translation is enhanced by a 1000-fold factor. In some embodiments, translation is enhanced by a 2000-fold factor. In some embodiments, the factor is 10-1000-fold. In some embodiments, the factor is 10-100-fold. In some embodiments, the factor is 10-200-fold. In some embodiments, the factor is 10-300-fold. In some embodiments, the factor is 10-500-fold. In some embodiments, the factor is 20-1000-fold. In some embodiments, the factor is 30-1000-fold. In some embodiments, the factor is 50-1000-fold. In some embodiments, the factor is 100-1000-fold. In some embodiments, the factor is 200-1000-fold. In some embodiments, translation is enhanced by any other significant amount or range of amounts.

In some embodiments, the non-immunogenic RNA (especially mRNA) exhibits significantly less innate immunogenicity than standard RNA with the same sequence. In some embodiments, the non-immunogenic RNA (especially mRNA) exhibits an innate immune response that is 2-fold less than its unmodified counterpart. In some embodiments, innate immunogenicity is reduced by a 3-fold factor. In some embodiments, innate immunogenicity is reduced by a 4-fold factor. In some embodiments, innate immunogenicity is reduced by a 5-fold factor. In some embodiments, innate immunogenicity is reduced by a 6-fold factor. In some embodiments, innate immunogenicity is reduced by a 7-fold factor. In some embodiments, innate immunogenicity is reduced by an 8-fold factor. In some embodiments, innate immunogenicity is reduced by a 9-fold factor. In some embodiments, innate immunogenicity is reduced by a 10-fold factor. In some embodiments, innate immunogenicity is reduced by a 15-fold factor. In some embodiments, innate immunogenicity is reduced by a 20-fold factor. In some embodiments, innate immunogenicity is reduced by a 50-fold factor. In some embodiments, innate immunogenicity is reduced by a 100-fold factor. In some embodiments, innate immunogenicity is reduced by a 200-fold factor. In some embodiments, innate immunogenicity is reduced by a 500-fold factor. In some embodiments, innate immunogenicity is reduced by a 1000-fold factor. In some embodiments, innate immunogenicity is reduced by a 2000-fold factor.

The term “exhibits significantly less innate immunogenicity” refers to a detectable decrease in innate immunogenicity. In some embodiments, the term refers to a decrease such that an effective amount of the non-immunogenic RNA (especially mRNA) can be administered without triggering a detectable innate immune response. In some embodiments, the term refers to a decrease such that the non-immunogenic RNA (especially mRNA) can be repeatedly administered without eliciting an innate immune response sufficient to detectably reduce production of the protein encoded by the non-immunogenic RNA. In some embodiments, the decrease is such that the non-immunogenic RNA (especially mRNA) can be repeatedly administered without eliciting an innate immune response sufficient to eliminate detectable production of the protein encoded by the non-immunogenic RNA.

“Immunogenicity” is the ability of a foreign substance, such as RNA, to provoke an immune response in the body of a human or other animal. The innate immune system is the component of the immune system that is relatively unspecific and immediate. It is one of two main components of the vertebrate immune system, along with the adaptive immune system.

### Antigen-Coding RNA and Use Thereof for Inducing an Immune Response

Generally, RNA (in particular, mRNA) described in the present disclosure comprises a nucleic acid sequence encoding a peptide or polypeptide comprising one or more Mycobacterium tuberculosis antigens, immunogenic variants or fragments thereof, for inducing an immune response against Mycobacterium tuberculosis in a subject. The peptide or polypeptide for inducing an immune response is also designated herein as “vaccine antigen” or simply “antigen”.

In some embodiments, the RNA (in particular, mRNA) is translated into the respective protein upon entering cells of a subject being administered the RNA, e.g., muscle cells or antigen-presenting cells (APCs).

In some embodiments, the RNA encoding the vaccine antigen is expressed in cells of the subject to provide the vaccine antigen. In some embodiments, the RNA encoding the vaccine antigen is transiently expressed in cells of the subject. In some embodiments, the vaccine antigen is presented in the context of MHC. In some embodiments, the vaccine antigen is secreted by cells of the subject.

In some embodiments, the RNA encoding the vaccine antigen is administered intramuscularly.

In some embodiments, the RNA encoding the vaccine antigen is administered systemically, e.g., intravenously. In some embodiments, after systemic administration of the RNA encoding the vaccine antigen, expression of the RNA encoding the vaccine antigen in spleen occurs. In some embodiments, after systemic administration of the RNA encoding the vaccine antigen, expression of the RNA encoding the vaccine antigen in antigen presenting cells, preferably professional antigen presenting cells occurs. In some embodiments, the antigen presenting cells are selected from the group consisting of dendritic cells, macrophages and B cells. In some embodiments, after systemic administration of the RNA encoding the vaccine antigen, no or essentially no expression of the RNA encoding the vaccine antigen in lung and/or liver occurs. In some embodiments, after systemic administration of the RNA encoding the vaccine antigen, expression of the RNA encoding the vaccine antigen in spleen is at least 5-fold the amount of expression in lung.

A vaccine antigen comprises an epitope for inducing an immune response against a disease-associated antigen, e.g., a protein of an infectious agent (e.g., Mtb antigen), in a subject. Accordingly, the vaccine antigen comprises an antigenic sequence for inducing an immune response against a disease-associated antigen in a subject. Such antigenic sequence may correspond to a target antigen or disease-associated antigen, an immunogenic variant thereof, or an immunogenic fragment of the target antigen or disease-associated antigen or the immunogenic variant thereof. Thus, the antigenic sequence may comprise at least an epitope of a target antigen or disease-associated antigen or an immunogenic variant thereof.

The antigenic sequences, e.g., epitopes, suitable for use according to the disclosure typically may be derived from a target antigen, i.e. the antigen against which an immune response is to be elicited. For example, the antigenic sequences contained within the vaccine antigen may be a target antigen or a fragment or variant of a target antigen. The antigenic sequence or a procession product thereof, e.g., a fragment thereof, may bind to an antigen receptor such as TCR carried by immune effector cells. In some embodiments, the antigenic sequence is selected from the group consisting of the antigen expressed by a target cell to which the immune effector cells are targeted or a fragment thereof, or a variant of the antigenic sequence or the fragment.

A vaccine antigen which may be provided to a subject according to the present disclosure by administering RNA encoding the vaccine antigen, preferably results in the induction of an immune response, e.g., in the stimulation, priming and/or expansion of immune effector cells, in the subject being provided the vaccine antigen. Said immune response, e.g., stimulated, primed and/or expanded immune effector cells, is preferably directed against a target antigen, in particular a target antigen expressed in diseased cells, tissues and/or organs, i.e., a disease-associated antigen. Thus, a vaccine antigen may comprise the disease-associated antigen, or a fragment or variant thereof. In some embodiments, such fragment or variant is immunologically equivalent to the disease-associated antigen.

The term “immunologically equivalent” means that the immunologically equivalent molecule such as the immunologically equivalent amino acid sequence exhibits the same or essentially the same immunological properties and/or exerts the same or essentially the same immunological effects, e.g., with respect to the type of the immunological effect. In the context of the present disclosure, the term “immunologically equivalent” is preferably used with respect to the immunological effects or properties of antigens or antigen variants used for immunization. For example, an amino acid sequence is immunologically equivalent to a reference amino acid sequence if said amino acid sequence when exposed to the immune system of a subject induces an immune reaction having a specificity of reacting with the reference amino acid sequence. Thus, in some embodiments, a molecule which is immunologically equivalent to an antigen exhibits the same or essentially the same properties and/or exerts the same or essentially the same effects regarding the stimulation, priming and/or expansion of T cells as the antigen to which the T cells are targeted. In the context of the present disclosure, the term “fragment of an antigen” or “variant of an antigen” means an agent which results in the induction of an immune response, e.g., in the stimulation, priming and/or expansion of immune effector cells, which immune response, e.g., stimulated, primed and/or expanded immune effector cells, targets the antigen, i.e. a disease-associated antigen, in particular when presented by diseased cells, tissues and/or organs. Thus, the vaccine antigen may correspond to or may comprise the disease-associated antigen, may correspond to or may comprise a fragment of the disease-associated antigen or may correspond to or may comprise an antigen which is homologous to the disease-associated antigen or a fragment thereof. If the vaccine antigen comprises a fragment of the disease-associated antigen or an amino acid sequence which is homologous to a fragment of the disease-associated antigen said fragment or amino acid sequence may comprise an epitope of the disease-associated antigen to which the antigen receptor of the immune effector cells is targeted or a sequence which is homologous to an epitope of the disease-associated antigen. Thus, according to the disclosure, a vaccine antigen may comprise an immunogenic fragment of a disease-associated antigen or an amino acid sequence being homologous to an immunogenic fragment of a disease-associated antigen. An “immunogenic fragment of an antigen” according to the disclosure preferably relates to a fragment of an antigen which is capable of inducing an immune response against, e.g., stimulating, priming and/or expanding immune effector cells carrying an antigen receptor binding to, the antigen or cells expressing the antigen. It is preferred that the vaccine antigen (similar to the disease-associated antigen) provides the relevant epitope for binding by the antigen receptor present on the immune effector cells. In some embodiments, the vaccine antigen or a fragment thereof (similar to the disease-associated antigen) is expressed on the surface of a cell such as an antigen-presenting cell (optionally in the context of MHC) so as to provide the relevant epitope for binding by immune effector cells. The vaccine antigen may be a recombinant antigen.

In some embodiments of all aspects described herein, the RNA encoding the vaccine antigen is expressed in cells of a subject to provide the antigen or a procession product thereof for binding by the antigen receptor expressed by immune effector cells, said binding resulting in stimulation, priming and/or expansion of the immune effector cells.

An “antigen” according to the present disclosure covers any substance that will elicit an immune response and/or any substance against which an immune response or an immune mechanism such as a cellular response and/or humoral response is directed. This also includes situations wherein the antigen is processed into antigen peptides and an immune response or an immune mechanism is directed against one or more antigen peptides, in particular if presented in the context of MHC molecules. In particular, an “antigen” relates to any substance, such as a peptide or polypeptide, that reacts specifically with antibodies or T-lymphocytes (T-cells). The term “antigen” may comprise a molecule that comprises at least one epitope, such as a T cell epitope. In some embodiments, an antigen is a molecule which, optionally after processing, induces an immune reaction, which may be specific for the antigen (including cells expressing the antigen). In some embodiments, an antigen is a disease-associated antigen, such as an Mtb antigen. In some embodiments, an antigen is presented or present on the surface of cells of the immune system such as antigen presenting cells like dendritic cells or macrophages. An antigen or a procession product thereof such as a T cell epitope is in some embodiments bound by an antigen receptor. Accordingly, an antigen or a procession product thereof may react specifically with immune effector cells such as T-lymphocytes (T cells).

According to the present disclosure, an antigen or a combination of antigens described herein may induce an immune response, wherein the immune response may comprise a humoral or cellular immune response, or both. In the context of some embodiments of the present disclosure, the antigen is presented by a cell, such as by an antigen presenting cell, in the context of MHC molecules, which results in an immune response against the antigen. An antigen may be a product which corresponds to or is derived from a naturally occurring antigen. According to the present disclosure, an antigen may correspond to a naturally occurring product.

The term “disease-associated antigen” is used in its broadest sense to refer to any antigen associated with a disease. In some embodiments, a disease-associated antigen is a molecule which contains epitopes that will stimulate a host's immune system to make a cellular antigen-specific immune response and/or a humoral antibody response against the disease. Disease-associated antigens include pathogen-associated antigens, i.e., antigens which are associated with infection by microbes, typically microbial antigens (such as bacterial or viral antigens, e.g., Mtb antigens), or antigens associated with cancer, typically tumors, such as tumor antigens.

The term “bacterial antigen” refers to any bacterial component having antigenic properties, i.e. being able to provoke an immune response in an individual. The bacterial antigen may be derived from the cell wall or cytoplasm membrane of the bacterium. The term “bacterial antigen” includes Mtb antigens, e.g., Mtb antigens as described herein.

The term “epitope” refers to an antigenic determinant in a molecule such as an antigen, i.e., to a part in or fragment of the molecule that is recognized by the immune system, for example, that is recognized by antibodies, T cells or B cells, in particular when presented in the context of MHC molecules. An epitope of a protein may comprises a continuous or discontinuous portion of said protein and, e.g., may be between about 5 and about 100, between about 5 and about 50, between about 8 and about 30, or about 10 and about 25 amino acids in length, for example, the epitope may be preferably 9, 10, 11, 12, 13, 14, 15, 16, 17, 18, 19, 20, 21, 22, 23, 24, or 25 amino acids in length. In some embodiments, the epitope in the context of the present disclosure is a T cell epitope.

Terms such as “epitope”, “fragment of an antigen”, “immunogenic peptide” and “antigen peptide” are used interchangeably herein and, e.g., may relate to an incomplete representation of an antigen which is, e.g., capable of eliciting an immune response against the antigen or a cell expressing or comprising and presenting the antigen. In some embodiments, the terms relate to an immunogenic portion of an antigen. In some embodiments, it is a portion of an antigen that is recognized (i.e., specifically bound) by a T cell receptor, in particular if presented in the context of MHC molecules. Certain preferred immunogenic portions bind to an MHC class I or class II molecule. The term “epitope” refers to a part or fragment of a molecule such as an antigen that is recognized by the immune system. For example, the epitope may be recognized by T cells, B cells or antibodies. An epitope of an antigen may include a continuous or discontinuous portion of the antigen and may be between about 5 and about 100, such as between about 5 and about 50, between about 8 and about 30, or between about 8 and about 25 amino acids in length, for example, the epitope may be 9, 10, 11, 12, 13, 14, 15, 16, 17, 18, 19, 20, 21, 22, 23, 24, or 25 amino acids in length. In some embodiments, an epitope is between about 10 and about 25 amino acids in length. The term “epitope” includes T cell epitopes.

The term “T cell epitope” refers to a part or fragment of a protein that is recognized by a T cell when presented in the context of MHC molecules, including epitopes predicted by bioinformatic means. The term “major histocompatibility complex” and the abbreviation “MHC” includes MHC class I and MHC class II molecules and relates to a complex of genes which is present in all vertebrates. MHC proteins or molecules are important for signaling between lymphocytes and antigen presenting cells or diseased cells in immune reactions, wherein the MHC proteins or molecules bind peptide epitopes and present them for recognition by T cell receptors on T cells. The proteins encoded by the MHC are expressed on the surface of cells, and display both self-antigens (peptide fragments from the cell itself) and non-self-antigens (e.g., fragments of invading microorganisms) to a T cell. In the case of class I MHC/peptide complexes, the binding peptides are typically about 8 to about 10 amino acids long although longer or shorter peptides may be effective. In the case of class II MHC/peptide complexes, the binding peptides are typically about 10 to about 25 amino acids long and are in particular about 13 to about 18 amino acids long, whereas longer and shorter peptides may be effective.

The peptide and polypeptide antigen can be 2 to 100 amino acids, including for example, 5 amino acids, 10 amino acids, 15 amino acids, 20 amino acids, 25 amino acids, 30 amino acids, 35 amino acids, 40 amino acids, 45 amino acids, or 50 amino acids in length. In some embodiments, a peptide can be greater than 50 amino acids. In some embodiments, the peptide can be greater than 100 amino acids.

The peptide or polypeptide antigen can be any peptide or polypeptide that can induce or increase the ability of the immune system to develop antibodies and T cell responses to the peptide or polypeptide.

In some embodiments, vaccine antigen, i.e., an antigen whose inoculation into a subject induces an immune response, is recognized by an immune effector cell. In some embodiments, the vaccine antigen if recognized by an immune effector cell is able to induce in the presence of appropriate co-stimulatory signals, stimulation, priming and/or expansion of the immune effector cell carrying an antigen receptor recognizing the vaccine antigen. In the context of the embodiments of the present disclosure, the vaccine antigen may be, e.g., presented or present on the surface of a cell, such as an antigen presenting cell.

In some embodiments, an antigen is expressed in a diseased cell (such as an infected cell).

In some embodiments, an antigen is presented by a diseased cell (such as an infected cell). In some embodiments, an antigen receptor is a TCR which binds to an epitope of an antigen presented in the context of MHC. In some embodiments, binding of a TCR when expressed by T cells and/or present on T cells to an antigen presented by cells such as antigen presenting cells results in stimulation, priming and/or expansion of said T cells. In some embodiments, binding of a TCR when expressed by T cells and/or present on T cells to an antigen presented on diseased cells results in cytolysis and/or apoptosis of the diseased cells, wherein said T cells release cytotoxic factors, e.g., perforins and granzymes.

In some embodiments, an antigen receptor is an antibody or B cell receptor which binds to an epitope in an antigen. In some embodiments, an antibody or B cell receptor binds to native epitopes of an antigen.

The terms “T cell” and “T lymphocyte” are used interchangeably herein and include T helper cells (CD4+ T cells) and cytotoxic T cells (CTLs, CD8+ T cells) which comprise cytolytic T cells. The term “antigen-specific T cell” or similar terms relate to a T cell which recognizes the antigen to which the T cell is targeted, in particular when presented on the surface of antigen presenting cells or diseased cells in the context of MHC molecules and preferably exerts effector functions of T cells. T cells are considered to be specific for antigen if the cells kill target cells expressing an antigen. T cell specificity may be evaluated using any of a variety of standard techniques, for example, within a chromium release assay or proliferation assay. Alternatively, synthesis of lymphokines (such as interferon-γ) can be measured. In some embodiments, the term “target” shall mean an agent such as a cell or tissue which is a target for an immune response such as a cellular immune response. Targets include cells that present an antigen or an antigen epitope, i.e., a peptide fragment derived from an antigen. In some embodiments, the target cell is a cell expressing an antigen and presenting said antigen with class I MHC.

“Antigen processing” refers to the degradation of an antigen into processing products which are fragments of said antigen (e.g., the degradation of a polypeptide into peptides) and the association of one or more of these fragments (e.g., via binding) with MHC molecules for presentation by cells, such as antigen-presenting cells to specific T-cells. Antigen-presenting cells can be distinguished in professional antigen presenting cells and non-professional antigen presenting cells.

The term “professional antigen presenting cells” relates to antigen presenting cells which constitutively express the Major Histocompatibility Complex class II (MHC class II) molecules required for interaction with naive T cells. If a T cell interacts with the MHC class II molecule complex on the membrane of the antigen presenting cell, the antigen presenting cell produces a co-stimulatory molecule inducing activation of the T cell. Professional antigen presenting cells comprise dendritic cells and macrophages.

The term “non-professional antigen presenting cells” relates to antigen presenting cells which do not constitutively express MHC class II molecules, but upon stimulation by certain cytokines such as interferon-gamma. Exemplary, non-professional antigen presenting cells include fibroblasts, thymic epithelial cells, thyroid epithelial cells, glial cells, pancreatic beta cells or vascular endothelial cells.

The term “dendritic cell” (DC) refers to a subtype of phagocytic cells belonging to the class of antigen presenting cells. In some embodiments, dendritic cells are derived from hematopoietic bone marrow progenitor cells. These progenitor cells initially transform into immature dendritic cells. These immature cells are characterized by high phagocytic activity and low T cell activation potential. Immature dendritic cells constantly sample the surrounding environment for pathogens such as viruses and bacteria. Once they have come into contact with a presentable antigen, they become activated into mature dendritic cells and begin to migrate to the spleen or to the lymph node. Immature dendritic cells phagocytose pathogens and degrade their proteins into small pieces and upon maturation present those fragments at their cell surface using MHC molecules. Simultaneously, they upregulate cell-surface receptors that act as co-receptors in T cell activation such as CD80, CD86, and CD40 greatly enhancing their ability to activate T cells. They also upregulate CCR7, a chemotactic receptor that induces the dendritic cell to travel through the blood stream to the spleen or through the lymphatic system to a lymph node. Here they act as antigen-presenting cells and activate helper T cells and killer T cells as well as B cells by presenting them antigens, alongside non-antigen specific co-stimulatory signals. Thus, dendritic cells can actively induce a T cell- or B cell-related immune response. In some embodiments, the dendritic cells are splenic dendritic cells.

The term “macrophage” refers to a subgroup of phagocytic cells produced by the differentiation of monocytes. Macrophages which are activated by inflammation, immune cytokines or microbial products nonspecifically engulf and kill foreign pathogens within the macrophage by hydrolytic and oxidative attack resulting in degradation of the pathogen. Peptides from degraded proteins are displayed on the macrophage cell surface where they can be recognized by T cells, and they can directly interact with antibodies on the B cell surface, resulting in T and B cell activation and further stimulation of the immune response. Macrophages belong to the class of antigen presenting cells. In some embodiments, the macrophages are splenic macrophages.

By “antigen-responsive CTL” is meant a CD8⁺ T-cell that is responsive to an antigen or a peptide derived from said antigen, which is presented with class I MHC on the surface of antigen presenting cells.

According to the disclosure, CTL responsiveness may include sustained calcium flux, cell division, production of cytokines such as IFN-γ and TNF-α, up-regulation of activation markers such as CD44 and CD69, and specific cytolytic killing of tumor antigen expressing target cells. CTL responsiveness may also be determined using an artificial reporter that accurately indicates CTL responsiveness.

“Activation” or “stimulation”, as used herein, refers to the state of a cell that has been sufficiently stimulated to induce detectable cellular proliferation, such as an immune effector cell such as T cell. Activation can also be associated with initiation of signaling pathways, induced cytokine production, and detectable effector functions. The term “activated immune effector cells” refers to, among other things, immune effector cells that are undergoing cell division.

The term “priming” refers to a process wherein an immune effector cell such as a T cell has its first contact with its specific antigen and causes differentiation into effector cells such as effector T cells.

The term “expansion” refers to a process wherein a specific entity is multiplied. In some embodiments, the term is used in the context of an immunological response in which immune effector cells are stimulated by an antigen, proliferate, and the specific immune effector cell recognizing said antigen is amplified. In some embodiments, expansion leads to differentiation of the immune effector cells.

The terms “immune response” and “immune reaction” are used herein interchangeably in their conventional meaning and refer to an integrated bodily response to an antigen and may refer to a cellular immune response, a humoral immune response, or both. According to the disclosure, the term “immune response to” or “immune response against” with respect to an agent such as an antigen, cell or tissue, relates to an immune response such as a cellular response directed against the agent. An immune response may comprise one or more reactions selected from the group consisting of developing antibodies against one or more antigens and expansion of antigen-specific T-lymphocytes, such as CD4+ and CD8+T-lymphocytes, e.g. CD8+T-lymphocytes, which may be detected in various proliferation or cytokine production tests in vitro.

The terms “inducing an immune response” and “eliciting an immune response” and similar terms in the context of the present disclosure refer to the induction of an immune response, such as the induction of a cellular immune response, a humoral immune response, or both. The immune response may be protective/preventive/prophylactic and/or therapeutic. The immune response may be directed against any immunogen or antigen or antigen peptide, such as against a pathogen-associated antigen (e.g., an antigen of Mtb). “Inducing” in this context may mean that there was no immune response against a particular antigen or pathogen before induction, but it may also mean that there was a certain level of immune response against a particular antigen or pathogen before induction and after induction said immune response is enhanced. Thus, “inducing the immune response” in this context also includes “enhancing the immune response”. In some embodiments, after inducing an immune response in an individual, said individual is protected from developing a disease such as an infectious disease or the disease condition is ameliorated by inducing an immune response.

The terms “cellular immune response”, “cellular response”, “cell-mediated immunity” or similar terms are meant to include a cellular response directed to cells characterized by expression of an antigen and/or presentation of an antigen with class I or class II MHC. The cellular response relates to cells called T cells or T lymphocytes which act as either “helpers” or “killers”. The helper T cells (also termed CD4+ T cells) play a central role by regulating the immune response and the killer cells (also termed cytotoxic T cells, cytolytic T cells, CD8+ T cells or CTLs) kill cells such as diseased cells.

The term “humoral immune response” refers to a process in living organisms wherein antibodies are produced in response to agents and organisms, which they ultimately neutralize and/or eliminate. The specificity of the antibody response is mediated by T and/or B cells through membrane-associated receptors that bind antigen of a single specificity. Following binding of an appropriate antigen and receipt of various other activating signals, B lymphocytes divide, which produces memory B cells as well as antibody secreting plasma cell clones, each producing antibodies that recognize the identical antigenic epitope as was recognized by its antigen receptor. Memory B lymphocytes remain dormant until they are subsequently activated by their specific antigen. These lymphocytes provide the cellular basis of memory and the resulting escalation in antibody response when re-exposed to a specific antigen.

The term “antibody” as used herein, refers to an immunoglobulin molecule, which is able to specifically bind to an epitope on an antigen. In particular, the term “antibody” refers to a glycoprotein comprising at least two heavy (H) chains and two light (L) chains inter-connected by disulfide bonds. The term “antibody” includes monoclonal antibodies, recombinant antibodies, human antibodies, humanized antibodies, chimeric antibodies and combinations of any of the foregoing. Each heavy chain is comprised of a heavy chain variable region (VH) and a heavy chain constant region (CH). Each light chain is comprised of a light chain variable region (VL) and a light chain constant region (CL). The variable regions and constant regions are also referred to herein as variable domains and constant domains, respectively. The VH and VL regions can be further subdivided into regions of hypervariability, termed complementarity determining regions (CDRs), interspersed with regions that are more conserved, termed framework regions (FRs). Each VH and VL is composed of three CDRs and four FRs, arranged from amino-terminus to carboxy-terminus in the following order: FR1, CDR1, FR2, CDR2, FR3, CDR3, FR4. The CDRs of a VH are termed HCDR1, HCDR2 and HCDR3, the CDRs of a VL are termed LCDR1, LCDR2 and LCDR3. The variable regions of the heavy and light chains contain a binding domain that interacts with an antigen. The constant regions of an antibody comprise the heavy chain constant region (CH) and the light chain constant region (CL), wherein CH can be further subdivided into constant domain CH1, a hinge region, and constant domains CH2 and CH3 (arranged from amino-terminus to carboxy-terminus in the following order: CH1, CH2, CH3). The constant regions of the antibodies may mediate the binding of the immunoglobulin to host tissues or factors, including various cells of the immune system (e.g., effector cells) and the first component (C1q) of the classical complement system. Antibodies can be intact immunoglobulins derived from natural sources or from recombinant sources and can be immunoactive portions of intact immunoglobulins. Antibodies are typically tetramers of immunoglobulin molecules. Antibodies may exist in a variety of forms including, for example, polyclonal antibodies, monoclonal antibodies, Fv, Fab and F(ab)₂, as well as single chain antibodies and humanized antibodies.

The term “immunoglobulin” relates to proteins of the immunoglobulin superfamily, such as to antigen receptors such as antibodies or the B cell receptor (BCR). The immunoglobulins are characterized by a structural domain, i.e., the immunoglobulin domain, having a characteristic immunoglobulin (Ig) fold. The term encompasses membrane bound immunoglobulins as well as soluble immunoglobulins. Membrane bound immunoglobulins are also termed surface immunoglobulins or membrane immunoglobulins, which are generally part of the BCR. Soluble immunoglobulins are generally termed antibodies. Immunoglobulins generally comprise several chains, typically two identical heavy chains and two identical light chains which are linked via disulfide bonds. These chains are primarily composed of immunoglobulin domains, such as the VL (variable light chain) domain, C_{L} (constant light chain) domain, VH (variable heavy chain) domain, and the CH (constant heavy chain) domains C_{H}1, C_{H}2, C_{H}3, and C_{H}4. There are five types of mammalian immunoglobulin heavy chains, i.e., α, δ, ε, γ, and μ which account for the different classes of antibodies, i.e., IgA, IgD, IgE, IgG, and IgM. As opposed to the heavy chains of soluble immunoglobulins, the heavy chains of membrane or surface immunoglobulins comprise a transmembrane domain and a short cytoplasmic domain at their carboxy-terminus. In mammals there are two types of light chains, i.e., lambda and kappa. The immunoglobulin chains comprise a variable region and a constant region. The constant region is essentially conserved within the different isotypes of the immunoglobulins, wherein the variable part is highly divers and accounts for antigen recognition.

The terms “vaccination” and “immunization” describe the process of treating an individual for therapeutic or prophylactic reasons and relate to the procedure of administering one or more immunogen(s) or antigen(s) or derivatives thereof, in particular in the form of RNA (especially mRNA) coding therefor, as described herein to an individual and stimulating an immune response against said one or more immunogen(s) or antigen(s) or cells characterized by presentation of said one or more immunogen(s) or antigen(s).

By “cell characterized by presentation of an antigen” or “cell presenting an antigen” or “MHC molecules which present an antigen on the surface of an antigen presenting cell” or similar expressions is meant a cell such as a diseased cell, in particular an infected cell, or an antigen presenting cell presenting the antigen or an antigen peptide, either directly or following processing, in the context of MHC molecules, such as MHC class I and/or MHC class II molecules. In some embodiments, the MHC molecules are MHC class I molecules.

### Embodiments of Antigen-Coding RNA

Generally, at least four formats useful for RNA pharmaceutical compositions may be used herein, namely non-modified uridine containing mRNA (uRNA), nucleoside modified mRNA (modRNA), self-amplifying RNA (saRNA), and trans-amplifying RNAs.

Features of modified uridine (e.g., pseudouridine) platform may include reduced adjuvant effect, blunted immune innate immune sensor activating capacity and thus augmented polypeptide (e.g., protein) expression.

Features of self-amplifying platform may include, for example, long duration of polypeptide (e.g., protein) expression, good tolerability and safety, higher likelihood for efficacy with very low RNA dose.

In some embodiments, a self-amplifying platform (e.g., RNA) comprises two nucleic acid molecules, wherein one nucleic acid molecule encodes a replicase (e.g., a viral replicase) and the other nucleic acid molecule is capable of being replicated (e.g., a replicon) by said replicase in trans (trans-replication system). In some embodiments, a self-amplifying platform (e.g., RNA) comprises a plurality of nucleic acid molecules, wherein said nucleic acids encode a plurality of replicases and/or replicons.

In some embodiments, a trans-replication system comprises the presence of both nucleic acid molecules in a single host cell.

In some such embodiments, a nucleic acid encoding a replicase (e.g., a viral replicase) is not capable of self-replication in a target cell and/or target organism. In some such embodiments, a nucleic acid encoding a replicase (e.g., a viral replicase) lacks at least one conserved sequence element important for (−) strand synthesis based on a (+) strand template and/or for (+) strand synthesis based on a (−) strand template.

In some embodiments, a self-amplifying RNA comprises a 3′ untranslated region (UTR), a 5′ UTR, a cap structure, a poly adenine (polyA) tail, and any combinations thereof.

In some embodiments, a self-amplifying platform does not require propagation of virus particles (e.g., is not associated with undesired virus-particle formation). In some embodiments, a self-amplifying platform is not capable of forming virus particles.

In some embodiments, RNA (e.g., a single stranded RNA) described herein has a length of at least 500 ribonucleotides (such as, e.g., at least 600 ribonucleotides, at least 700 ribonucleotides, at least 800 ribonucleotides, at least 900 ribonucleotides, at least 1000 ribonucleotides, at least 1250 ribonucleotides, at least 1500 ribonucleotides, at least 1750 ribonucleotides, at least 2000 ribonucleotides, at least 2500 ribonucleotides, at least 3000 ribonucleotides, at least 3500 ribonucleotides, at least 4000 ribonucleotides, at least 4500 ribonucleotides, at least 5000 ribonucleotides, or longer). In some embodiments, RNA described herein is single-stranded RNA having a length of about 800 ribonucleotides to 5000 ribonucleotides.

In some embodiments, a relevant RNA includes a polypeptide-encoding portion or a plurality of polypeptide-encoding portions. In some particular embodiments, such a portion or portions encode one or more polypeptides which are not endogenous (i.e., it is foreign) to the subject treated.

In some embodiments, the RNA described herein (e.g., contained in the compositions/formulations of the present disclosure and/or used in the methods of the present disclosure) is single-stranded RNA (in particular, mRNA) that may be translated into the respective protein upon entering cells, e.g., cells of a recipient, e.g., muscle cells or antigen-presenting cells (APCs). In addition to wild-type or codon-optimized sequences encoding an antigen sequence, the RNA may contain one or more structural elements optimized for maximal efficacy of the RNA with respect to stability and translational efficiency (5′ cap, 5′ UTR, 3′ UTR, poly(A)-tail). In some embodiments, the RNA contains all of these elements. In some embodiments, beta-S-ARCA (D1) (m₂^{7,2′-O}GppSpG) or m₂^{7,3′-O}Gppp (m₁^{2-O})ApG may be utilized as specific capping structure at the 5′-end of the RNA. As 5′-UTR sequence, the 5′-UTR sequence of the human alpha-globin mRNA, optionally with an optimized ‘Kozak sequence’ to increase translational efficiency may be used. As 3′-UTR sequence, a combination of two sequence elements (FI element) derived from the “amino terminal enhancer of split” (AES) mRNA (called F) and the mitochondrial encoded 12S ribosomal RNA (called I) placed between the coding sequence and the poly(A)-tail to assure higher maximum protein levels and prolonged persistence of the mRNA may be used (see WO 2017/060314, herein incorporated by reference). Furthermore, a poly(A)-tail measuring 110 nucleotides in length, consisting of a stretch of 30 adenosine residues, followed by a 10 nucleotide linker sequence (of random nucleotides) and another 70 adenosine residues may be used. In some embodiments, the 5′-UTR comprises the nucleotide sequence of SEQ ID NO: 39, or a nucleotide sequence having at least 99%, 98%, 97%, 96%, 95%, 90%, 85%, or 80% identity to the nucleotide sequence of SEQ ID NO: 39. In some embodiments, the 3′-UTR comprises the nucleotide sequence of SEQ ID NO: 40, or a nucleotide sequence having at least 99%, 98%, 97%, 96%, 95%, 90%, 85%, or 80% identity to the nucleotide sequence of SEQ ID NO: 40. In some embodiments, the poly(A) sequence comprises the nucleotide sequence of SEQ ID NO: 41, or a nucleotide sequence having at least 99%, 98%, 97%, 96%, 95%, 90%, 85%, or 80% identity to the nucleotide sequence of SEQ ID NO: 41.

In some embodiments, the RNA described herein is not chemically modified, i.e. it solely contains naturally occurring nucleosides, and preferably has the composition of naturally occurring RNA.

In some embodiments, the RNA described herein is modified for optimized efficacy of the RNA (e.g., increased translation efficacy, decreased immunogenicity, and/or decreased cytotoxicity) (e.g., by replacing (partially or completely, preferably completely) naturally occurring nucleosides (in particular uridine) with synthetic nucleosides (e.g., modified nucleosides, e.g., selected from the group consisting of pseudouridine (ψ), N1-methyl-pseudouridine (m1ψ), and 5-methyl-uridine); and/or codon-optimization). In some embodiments, the RNA comprises a modified nucleoside in place of uridine. In some embodiments, the modified nucleoside replacing (partially or completely, preferably completely) uridine is selected from the group consisting of pseudouridine (ψ), N1-methyl-pseudouridine (m1ψ), and 5-methyl-uridine. In some embodiments, the RNA encoding the vaccine antigen has a coding sequence (a) which is codon-optimized, (b) the G/C content of which is increased compared to the wild type coding sequence, or (c) both (a) and (b).

In some embodiments, the RNA described herein comprises a 5′ cap, a 5′ UTR, a 3′ UTR, and a poly(A) sequence (e.g., as described above); is modified by replacing (partially or completely, preferably completely) uridine with modified nucleosides, e.g., selected from the group consisting of pseudouridine (ψ), N1-methyl-pseudouridine (m1ψ), and 5-methyl-uridine; and has a coding sequence which is codon-optimized, and the G/C content of which is increased compared to the wild type coding sequence.

In some embodiments, if the present disclosure provides for a mixture of different RNA molecules, a composition comprising different RNA molecules or an administration of different RNA molecules, these different RNA molecules are present in approximately the same amount. Such different RNA molecules may be formulated in individual particulate formulations, mixed particulate formulations, or combined particulate formulations as described herein.

The present disclosure provides RNA (in particular, mRNA) comprising a nucleic acid sequence encoding an Mtb antigen, an immunogenic variant thereof, or an immunogenic fragment of the Mtb antigen or the immunogenic variant thereof. In some embodiments, RNA (in particular, mRNA) described in the present disclosure comprises a nucleic acid sequence encoding an Mtb antigen, an immunogenic variant thereof, or an immunogenic fragment of the Mtb antigen or the immunogenic variant thereof, and is capable of expressing said Mtb antigen, immunogenic variant, or immunogenic fragment, in particular if transferred into a cell or subject, preferably a human cell or subject. Thus, in some embodiments, the RNA (in particular, mRNA) described in the present disclosure contains a coding region (open reading frame (ORF)) encoding an Mtb antigen, an immunogenic variant thereof, or an immunogenic fragment of the Mtb antigen or the immunogenic variant thereof.

In some embodiments, RNA comprises a nucleic acid sequence encoding more than one Mtb antigen, immunogenic variant thereof, or immunogenic fragment of the Mtb antigen or the immunogenic variant thereof, e.g., two, three, four or more Mtb antigens, immunogenic variants thereof, or immunogenic fragments of the Mtb antigens or the immunogenic variants thereof. In some embodiments, two or more of such Mtb antigens, immunogenic variants thereof, or immunogenic fragments of the Mtb antigens or the immunogenic variants thereof are present as a fusion protein.

In some embodiments, the one or more Mtb antigens, immunogenic variants thereof, or immunogenic fragments of the Mtb antigens or the immunogenic variants thereof encoded by the RNA may comprise or consist of naturally occurring sequences, may comprise or consist of variants of naturally occurring sequences, or may comprise or consist of sequences which are not naturally occurring, e.g., recombinant sequences. In some embodiments, the peptide or polypeptide encoded by the RNA described herein may consist of the one or more Mtb antigens, immunogenic variants thereof, or immunogenic fragments of the Mtb antigens or the immunogenic variants thereof, or may comprise the one or more Mtb antigens, immunogenic variants thereof, or immunogenic fragments of the Mtb antigens or the immunogenic variants thereof and may comprise additional sequences such as secretion signals, extended-PK groups, tags and any other sequences. In some embodiments, the additional sequences are fused to the one or more Mtb antigens, immunogenic variants thereof, or immunogenic fragments of the Mtb antigens or the immunogenic variants thereof, in some embodiments, separated by a linker. In these embodiments, the one or more Mtb antigens, immunogenic variants thereof, or immunogenic fragments of the Mtb antigens or the immunogenic variants thereof may be considered the pharmaceutically active peptide or polypeptide even if additional sequences support the function or effect of the one or more Mtb antigens, immunogenic variants thereof, or immunogenic fragments of the Mtb antigens or the immunogenic variants thereof.

According to the present disclosure, the term “pharmaceutically active peptide or polypeptide” means a peptide or polypeptide that can be used in the treatment of an individual where the expression of the peptide or polypeptide would be of benefit, e.g., in ameliorating the symptoms of a disease. Preferably, a pharmaceutically active peptide or polypeptide has curative or palliative properties and may be administered to ameliorate, relieve, alleviate, reverse, delay onset of or lessen the severity of one or more symptoms of a disease. In some embodiments, a pharmaceutically active peptide or polypeptide has a positive or advantageous effect on the condition or disease state of an individual when administered to the individual in a therapeutically effective amount. A pharmaceutically active peptide or polypeptide may have prophylactic properties and may be used to delay the onset of a disease or to lessen the severity of such disease. The term “pharmaceutically active peptide or polypeptide” includes entire peptides or polypeptides, and can also refer to pharmaceutically active fragments thereof. It can also include pharmaceutically active variants and/or analogs of a peptide or polypeptide.

Specific examples of pharmaceutically active peptides and polypeptides include, but are not limited to, antigens for vaccination such as Mtb antigens, immunogenic variants thereof, or immunogenic fragments of the Mtb antigens or the immunogenic variants thereof.

Mtb antigens, immunogenic variants thereof, or immunogenic fragments of the Mtb antigens or the immunogenic variants thereof described herein can be prepared as fusion or chimeric polypeptides that include a portion which corresponds to one or more Mtb antigens, immunogenic variants thereof, or immunogenic fragments of the Mtb antigens or the immunogenic variants thereof and a heterologous polypeptide (i.e., a polypeptide that is not an Mtb antigen, immunogenic variant thereof, or immunogenic fragment of the Mtb antigen or the immunogenic variant thereof).

According to certain embodiments, a “signal peptide” (or signal sequence) is fused, either directly or through a linker, to the N-terminus of a chimeric protein described herein.

In some embodiments, an open reading frame of the RNA described herein encodes a polypeptide that includes a signal sequence, e.g., that is functional in mammalian cells.

In some embodiments, a utilized signal sequence is “intrinsic” in that it is, in nature, associated with (e.g., linked to) the full-length antigen or antigen fragment at the N-terminus of the chimeric protein.

In some embodiments, a utilized signal sequence is non-native to the encoded polypeptide—e.g., is not naturally part of a full-length antigen or antigen fragment whose sequences are included in the encoded chimeric protein. In some embodiments, signal peptides are sequences, which are typically characterized by a length of about 15 to 30 amino acids.

In many embodiments, signal peptides are positioned at the N-terminus of an encoded chimeric protein as described herein, without being limited thereto. In some embodiments, signal peptides preferably allow the transport of the polypeptide encoded by RNAs of the present disclosure with which they are associated into a defined cellular compartment, preferably the cell surface, the endoplasmic reticulum (ER) or the endosomal-lysosomal compartment. In some embodiments, an RNA sequence encodes a peptidoglycan hydrolase, e.g., an endolysin, that may comprise or otherwise be linked to a signal sequence (e.g., secretory sequence), such as those listed in Table 2 and 3, or a sequence having 1, 2, 3, 4, or 5 amino acid differences relative thereto. In some embodiments, a signal sequence such as MRVMAPRTLILLLSGALALTETWAGS [SEQ ID NO: 3], or a sequence having 1, 2, 3, 4, or at the most 5 amino acid differences relative thereto is utilized.

In some embodiments, a signal peptide is selected from those included in the Table 2 below and/or those encoded by the sequences in Table 3 below or a sequence having 1, 2, 3, 4, or 5 amino acid differences relative thereto:

TABLE 2
Exemplary signal sequences

| Signal | Sequence (Amino Acid) |
|---|---|
| HSV-1 gD SP | MGGAAARLGAVILFVVIVGLHGVRSKY [SEQ ID NO: 4] |
| HSV-1 gD SP | MGGAAARLGAVILFVVIVGLHGVRGKY [SEQ ID NO: 5] |
| HSV-2 gD SP | MGRLTSGVGTAALLVVAVGLRVVCA [SEQ ID NO: 6] |
| HSV-2 | MGRLTSGVGTAALLVVAVGLRVVCAKYA [SEQ ID NO: 7] |
| SARS-CoV-2-S | MFVFLVLLPLVSSQCVNLT [SEQ ID NO: 8] |
| human Ig heavy | MDWIWRILFLVGAATGAHSQM [SEQ ID NO: 9] |
| chain signal | |
| peptide (huSec) | |
| HuIgGk signal | METPAQLLFLLLLWLPDTTG [SEQ ID NO: 10] |
| peptide | |
| IgE heavy chain | MDWTWILFLVAAATRVHS [SEQ ID NO: 11] |
| epsilon-1signal | |
| peptide | |
| Japanese | MLGSNSGQRVVFTILLLLVAPAYS [SEQ ID NO: 12] |
| encephalitis PRM | |
| signal sequence | |
| VSVg protein | MKCLLYLAFLFIGVNCA [SEQ ID NO: 13] |
| signal sequence | |
| human Ig heavy | MELGLSWIFLLAILKGVQC [SEQ ID NO: 14] |
| chain signal | |
| peptide | |
| human Ig heavy | MELGLRWVFLVAILEGVQC [SEQ ID NO: 15] |
| chain signal | |
| peptide | |
| human Ig heavy | MKHLWFFLLLVAAPRWVLS [SEQ ID NO: 16] |
| chain signal | |
| peptide | |
| human Ig heavy | MDWTWRILFLVAAATGAHS [SEQ ID NO: 17] |
| chain signal | |
| peptide | |
| human Ig heavy | MDWTWRFLFVVAAATGVQS [SEQ ID NO: 18] |
| chain signal | |
| peptide | |
| human Ig heavy | MEFGLSWLFLVAILKGVQC [SEQ ID NO: 19] |
| chain signal | |
| peptide | |
| human Ig heavy | MEFGLSWVFLVALFRGVQC [SEQ ID NO: 20] |
| chain signal | |
| peptide | |
| human Ig heavy | MDLLHKNMKHLWFFLLLVAAPRWVLS [SEQ ID NO: 21] |
| chain signal | |
| peptide | |
| human Ig kappa | MDMRVPAQLLGLLLLWLSGARC [SEQ ID NO: 22] |
| chain signal | |
| peptide | |
| human Ig kappa | MKYLLPTAAAGLLLLAAQPAMA [SEQ ID NO: 23] |
| chain signal | |
| peptide | |

TABLE 3
Exemplary nucleotide sequences encoding signal sequences

| Signal | Sequence (Nucleotide) |
|---|---|
| HSV-1 gD SP | ATGGGGGGGGCTGCCGCCAGGTTGGGGGCCGTGATTTTGTTTGTCGTCATAGTGGG |
| wild-type | CCTCCATGGGGTCCGCAGCAAATAT [SEQ ID NO: 24] |
| HSV-1 gD SP | ATGggaggagccGCCGCCagactgggaGCCGTGatcctgttcgtggtgatcGTGgg |
| optimized | actgCATggagtgagaAGCaagtac [SEQ ID NO: 25] |
| nucleotide | |
| sequence | |
| SARS-CoV-2-S | ATGTTTGTGTTTCTTGTGCTGCTGCCTCTTGTGTCTTCTCAGTGTGTGAATTTGACA |
| | [SEQ ID NO: 26] |
| human Ig heavy | ATGGATTGGATTTGGAGAATCCTGTTCCTCGTGGGAGCCGCTACAGGAGCCCACTCC |
| chain signal | CAGATG [SEQ ID NO: 27] |
| peptide (huSec) | |
| human Ig heavy | ATGGAGTTGGGACTGAGCTGGATTTTCCTTTTGGCTATTTTAAAAGGTGTCCAGTGT |
| chain signal | [SEQ ID NO: 28] |
| peptide | |
| human Ig heavy | ATGGAACTGGGGCTCCGCTGGGTTTTCCTTGTTGCTATTTTAGAAGGTGTCCAGTGT |
| chain signal | [SEQ ID NO: 29] |
| peptide | |
| human Ig heavy | ATGAAACACCTGTGGTTCTTCCTCCTGCTGGTGGCAGCTCCCAGATGGGTCCTGTCC |
| chain signal | [SEQ ID NO: 30] |
| peptide | |
| human Ig heavy | ATGGACTGGACCTGGAGGATCCTCTTCTTGGTGGCAGCAGCAACAGGTGCCCACTC |
| chain signal | G [SEQ ID NO: 31] |
| peptide | |
| human Ig heavy | ATGGACTGGACCTGGAGGTTCCTCTTTGTGGTGGCAGCAGCTACAGGTGTCCAGTC |
| chain signal | C [SEQ ID NO: 32] |
| peptide | |
| human Ig heavy | ATGGAGTTTGGGCTGAGCTGGCTTTTTCTTGTGGCGATTCTAAAAGGTGTCCAGTG |
| chain signal | T [SEQ ID NO: 33] |
| peptide | |
| human Ig heavy | ATGGAGTTTGGGCTGAGCTGGGTTTTCCTCGTTGCTCTTTTTAGAGGTGTCCAGTGT |
| chain signal | [SEQ ID NO: 34] |
| peptide | |
| human Ig heavy | ATGGACCTCCTGCACAAGAACATGAAACACCTGTGGTTCTTCCTCCTCCTGGTGGCA |
| chain signal | GCTCCCAGATGGGTGCTGTCC [SEQ ID NO: 35] |
| peptide | |
| human Ig kappa | ATGGACATGAGGGTCCCTGCTCAGCTCCTGGGGCTCCTGCTGCTCTGGCTCTCAGG |
| chain signal | TGCCAGATGT [SEQ ID NO: 36] |
| peptide | |
| human Ig kappa | ATGAAATACCTATTGCCTACGGCAGCCGCTGGATTGTTATTACTCGCGGCCCAGCCG |
| chain signal | GCCATGGCC [SEQ ID NO: 37] |
| peptide | |

According to some embodiments, an amino acid sequence enhancing antigen processing and/or presentation is fused, either directly or through a linker, to an antigenic peptide or polypeptide (antigenic sequence), e.g., one or more Mtb antigens, immunogenic variants thereof, or immunogenic fragments of the Mtb antigens or the immunogenic variants thereof. Accordingly, in some embodiments, the RNA described herein comprises at least one coding region encoding an antigenic peptide or polypeptide and an amino acid sequence enhancing antigen processing and/or presentation. Such amino acid sequences enhancing antigen processing and/or presentation are preferably located at the C-terminus of the antigenic peptide or polypeptide, without being limited thereto. Amino acid sequences enhancing antigen processing and/or presentation as defined herein preferably improve antigen processing and presentation. In some embodiments, the amino acid sequence enhancing antigen processing and/or presentation as defined herein includes, without being limited thereto, sequences derived from the human MHC class I complex (HLA-B51, haplotype A2, B27/B51, Cw2/Cw3), in particular a sequence comprising the amino acid sequence of SEQ ID NO: 38 or a functional variant thereof. Such sequence is designated herein as MITD.

In some embodiments, an amino acid sequence enhancing antigen processing and/or presentation comprises the amino acid sequence of SEQ ID NO: 38, an amino acid sequence having at least 99%, 98%, 97%, 96%, 95%, 90%, 85%, or 80% identity to the amino acid sequence of SEQ ID NO: 38, or a functional fragment of the amino acid sequence of SEQ ID NO: 38, or the amino acid sequence having at least 99%, 98%, 97%, 96%, 95%, 90%, 85%, or 80% identity to the amino acid sequence of SEQ ID NO: 38. In some embodiments, an amino acid sequence enhancing antigen processing and/or presentation comprises the amino acid sequence of SEQ ID NO: 38.

Accordingly, in some embodiments, the RNA described herein comprises at least one coding region encoding an antigenic peptide or polypeptide and an amino acid sequence enhancing antigen processing and/or presentation, said amino acid sequence enhancing antigen processing and/or presentation preferably being fused to the antigenic peptide or polypeptide, more preferably to the C-terminus of the antigenic peptide or polypeptide as described herein. Furthermore, a secretory sequence, e.g., a sequence comprising the amino acid sequence selected from SEQ ID NOS: 3 to 23 or encoded by the nucleotide sequence selected from SEQ ID NOs: 24 to 37, may be fused to the N-terminus of the antigenic peptide or polypeptide.

Mtb antigens, immunogenic variants thereof, or immunogenic fragments of the Mtb antigens or the immunogenic variants thereof may be fused to an extended-PK group, which increases circulation half-life. Non-limiting examples of extended-PK groups are described herein. It should be understood that other PK groups that increase the circulation half-life of peptides or polypeptides such as Mtb antigens, immunogenic variants thereof, or immunogenic fragments of the Mtb antigens or the immunogenic variants thereof are also applicable to the present disclosure. In certain embodiments, the extended-PK group is a serum albumin domain (e.g., mouse serum albumin, human serum albumin, or recombinant serum albumin).

As used herein, the term “PK” is an acronym for “pharmacokinetic” and encompasses properties of a compound including, by way of example, absorption, distribution, metabolism, and elimination by a subject. As used herein, an “extended-PK group” refers to a protein, peptide, or moiety that increases the circulation half-life of a biologically active molecule when fused to or administered together with the biologically active molecule. Examples of an extended-PK group include serum albumin (e.g., HSA), Immunoglobulin Fc or Fc fragments and variants thereof, transferrin and variants thereof, and human serum albumin (HSA) binders (as disclosed in U.S. Publication Nos. 2005/0287153 and 2007/0003549). Other exemplary extended-PK groups are disclosed in Kontermann, Expert Opin Biol Ther, 2016 July; 16 (7): 903-15 which is herein incorporated by reference in its entirety. As used herein, an “extended-PK” polypeptide refers to a polypeptide moiety such as an Mtb antigen, immunogenic variant thereof, or immunogenic fragment of the Mtb antigen or the immunogenic variant thereof in combination with an extended-PK group. In some embodiments, the extended-PK polypeptide is a fusion protein in which a polypeptide moiety is linked or fused to an extended-PK group.

In certain embodiments, the serum half-life of an extended-PK polypeptide is increased relative to the polypeptide alone (i.e., the polypeptide not fused to an extended-PK group). In certain embodiments, the serum half-life of the extended-PK polypeptide is at least 20%, at least 40%, at least 60%, at least 80%, at least 100%, at least 120%, at least 150%, at least 180%, at least 200%, at least 400%, at least 600%, at least 800%, or at least 1000% longer relative to the serum half-life of the polypeptide alone. In certain embodiments, the serum half-life of the extended-PK polypeptide is at least 1.5-fold, 2-fold, 2.5-fold, 3-fold, 3.5-fold, 4-fold, 4.5-fold, 5-fold, 6-fold, 7-fold, 8-fold, 10-fold, 12-fold, 13-fold, 15-fold, 17-fold, 20-fold, 22-fold, 25-fold, 27-fold, 30-fold, 35-fold, 40-fold, or 50-fold greater than the serum half-life of the polypeptide alone. In certain embodiments, the serum half-life of the extended-PK polypeptide is at least 10 hours, 15 hours, 20 hours, 25 hours, 30 hours, 35 hours, 40 hours, 50 hours, 60 hours, 70 hours, 80 hours, 90 hours, 100 hours, 110 hours, 120 hours, 130 hours, 135 hours, 140 hours, 150 hours, 160 hours, or 200 hours.

As used herein, “half-life” refers to the time taken for the serum or plasma concentration of a compound such as a peptide or polypeptide to reduce by 50%, in vivo, for example due to degradation and/or clearance or sequestration by natural mechanisms. An extended-PK polypeptide suitable for use herein is stabilized in vivo and its half-life increased by, e.g., fusion to serum albumin (e.g., human serum albumin (HSA) or mouse serum albumin (MSA)), which resist degradation and/or clearance or sequestration. The half-life can be determined in any manner known per se, such as by pharmacokinetic analysis. Suitable techniques will be clear to the person skilled in the art, and may for example generally involve the steps of suitably administering a suitable dose of the amino acid sequence or compound to a subject; collecting blood samples or other samples from said subject at regular intervals; determining the level or concentration of the amino acid sequence or compound in said blood sample; and calculating, from (a plot of) the data thus obtained, the time until the level or concentration of the amino acid sequence or compound has been reduced by 50% compared to the initial level upon dosing. Further details are provided in, e.g., standard handbooks, such as Kenneth, A. et al., Chemical Stability of Pharmaceuticals: A Handbook for Pharmacists and in Peters et al., Pharmacokinetic Analysis: A Practical Approach (1996). Reference is also made to Gibaldi, M. et al., Pharmacokinetics, 2nd Rev. Edition, Marcel Dekker (1982).

In certain embodiments, the extended-PK group includes serum albumin, or fragments thereof or variants of the serum albumin or fragments thereof (all of which for the purpose of the present disclosure are comprised by the term “albumin”). Polypeptides described herein may be fused to albumin (or a fragment or variant thereof) to form albumin fusion proteins. Such albumin fusion proteins are described in U.S. Publication No. 20070048282.

As used herein, “albumin fusion protein” refers to a protein formed by the fusion of at least one molecule of albumin (or a fragment or variant thereof) to at least one molecule of a protein such as a therapeutic protein, in particular an Mtb antigen, immunogenic variant thereof, or immunogenic fragment of the Mtb antigen or the immunogenic variant thereof. The albumin fusion protein may be generated by translation of a nucleic acid in which a polynucleotide encoding a therapeutic protein is joined in-frame with a polynucleotide encoding an albumin. The therapeutic protein and albumin, once part of the albumin fusion protein, may each be referred to as a “portion”, “region” or “moiety” of the albumin fusion protein (e.g., a “therapeutic protein portion” or an “albumin protein portion”). In a highly preferred embodiment, an albumin fusion protein comprises at least one molecule of a therapeutic protein (including, but not limited to a mature form of the therapeutic protein) and at least one molecule of albumin (including but not limited to a mature form of albumin). In some embodiments, an albumin fusion protein is processed by a host cell such as a cell of the target organ for administered RNA, e.g. a liver cell, and secreted into the circulation. Processing of the nascent albumin fusion protein that occurs in the secretory pathways of the host cell used for expression of the RNA may include, but is not limited to signal peptide cleavage; formation of disulfide bonds; proper folding; addition and processing of carbohydrates (such as for example, N- and O-linked glycosylation); specific proteolytic cleavages; and/or assembly into multimeric proteins. An albumin fusion protein is preferably encoded by RNA in a non-processed form which in particular has a signal peptide at its N-terminus and following secretion by a cell is preferably present in the processed form wherein in particular the signal peptide has been cleaved off. In a most preferred embodiment, the “processed form of an albumin fusion protein” refers to an albumin fusion protein product which has undergone N-terminal signal peptide cleavage, herein also referred to as a “mature albumin fusion protein”.

In preferred embodiments, albumin fusion proteins comprising a therapeutic protein have a higher plasma stability compared to the plasma stability of the same therapeutic protein when not fused to albumin. Plasma stability typically refers to the time period between when the therapeutic protein is administered in vivo and carried into the bloodstream and when the therapeutic protein is degraded and cleared from the bloodstream, into an organ, such as the kidney or liver, that ultimately clears the therapeutic protein from the body. Plasma stability is calculated in terms of the half-life of the therapeutic protein in the bloodstream. The half-life of the therapeutic protein in the bloodstream can be readily determined by common assays known in the art.

As used herein, “albumin” refers collectively to albumin protein or amino acid sequence, or an albumin fragment or variant, having one or more functional activities (e.g., biological activities) of albumin. In particular, “albumin” refers to human albumin or fragments or variants thereof especially the mature form of human albumin, or albumin from other vertebrates or fragments thereof, or variants of these molecules. The albumin may be derived from any vertebrate, especially any mammal, for example human, cow, sheep, or pig. Non-mammalian albumins include, but are not limited to, hen and salmon. The albumin portion of the albumin fusion protein may be from a different animal than the therapeutic protein portion.

In certain embodiments, the albumin is human serum albumin (HSA), or fragments or variants thereof, such as those disclosed in U.S. Pat. No. 5,876,969, WO 2011/124718, WO 2013/075066, and WO 2011/0514789.

The terms, human serum albumin (HSA) and human albumin (HA) are used interchangeably herein. The terms, “albumin and “serum albumin” are broader, and encompass human serum albumin (and fragments and variants thereof) as well as albumin from other species (and fragments and variants thereof).

As used herein, a fragment of albumin sufficient to prolong the therapeutic activity or plasma stability of the therapeutic protein refers to a fragment of albumin sufficient in length or structure to stabilize or prolong the therapeutic activity or plasma stability of the protein so that the plasma stability of the therapeutic protein portion of the albumin fusion protein is prolonged or extended compared to the plasma stability in the non-fusion state.

The albumin portion of the albumin fusion proteins may comprise the full length of the albumin sequence, or may include one or more fragments thereof that are capable of stabilizing or prolonging the therapeutic activity or plasma stability. Such fragments may be of 10 or more amino acids in length or may include about 15, 20, 25, 30, 50, or more contiguous amino acids from the albumin sequence or may include part or all of specific domains of albumin. For instance, one or more fragments of HSA spanning the first two immunoglobulin-like domains may be used. In a preferred embodiment, the HSA fragment is the mature form of HSA.

Generally speaking, an albumin fragment or variant will be at least 100 amino acids long, preferably at least 150 amino acids long.

According to the disclosure, albumin may be naturally occurring albumin or a fragment or variant thereof. Albumin may be human albumin and may be derived from any vertebrate, especially any mammal.

Preferably, the albumin fusion protein comprises albumin as the N-terminal portion, and a therapeutic protein as the C-terminal portion. Alternatively, an albumin fusion protein comprising albumin as the C-terminal portion, and a therapeutic protein as the N-terminal portion may also be used. In other embodiments, the albumin fusion protein has a therapeutic protein fused to both the N-terminus and the C-terminus of albumin. In a preferred embodiment, the therapeutic proteins fused at the N- and C-termini are the same therapeutic proteins. In another preferred embodiment, the therapeutic proteins fused at the N- and C-termini are different therapeutic proteins. In some embodiments, the different therapeutic proteins are both Mtb antigens, immunogenic variants thereof, or immunogenic fragments of the Mtb antigens or the immunogenic variants thereof.

In some embodiments, the therapeutic protein(s) is (are) joined to the albumin through (a) peptide linker(s). A peptide linker between the fused portions may provide greater physical separation between the moieties and thus maximize the accessibility of the therapeutic protein portion, for instance, for binding to its cognate receptor. The peptide linker may consist of amino acids such that it is flexible or more rigid. The linker sequence may be cleavable by a protease or chemically.

As used herein, the term “Fc region” refers to the portion of a native immunoglobulin formed by the respective Fc domains (or Fc moieties) of its two heavy chains. As used herein, the term “Fc domain” refers to a portion or fragment of a single immunoglobulin (Ig) heavy chain wherein the Fc domain does not comprise an Fv domain. In certain embodiments, an Fc domain begins in the hinge region just upstream of the papain cleavage site and ends at the C-terminus of the antibody. Accordingly, a complete Fc domain comprises at least a hinge domain, a CH2 domain, and a CH3 domain. In certain embodiments, an Fc domain comprises at least one of: a hinge (e.g., upper, middle, and/or lower hinge region) domain, a CH2 domain, a CH3 domain, a CH4 domain, or a variant, portion, or fragment thereof. In certain embodiments, an Fc domain comprises a complete Fc domain (i.e., a hinge domain, a CH2 domain, and a CH3 domain). In certain embodiments, an Fc domain comprises a hinge domain (or portion thereof) fused to a CH3 domain (or portion thereof). In certain embodiments, an Fc domain comprises a CH2 domain (or portion thereof) fused to a CH3 domain (or portion thereof). In certain embodiments, an Fc domain consists of a CH3 domain or portion thereof. In certain embodiments, an Fc domain consists of a hinge domain (or portion thereof) and a CH3 domain (or portion thereof). In certain embodiments, an Fc domain consists of a CH2 domain (or portion thereof) and a CH3 domain. In certain embodiments, an Fc domain consists of a hinge domain (or portion thereof) and a CH2 domain (or portion thereof). In certain embodiments, an Fc domain lacks at least a portion of a CH2 domain (e.g., all or part of a CH2 domain). An Fc domain herein generally refers to a polypeptide comprising all or part of the Fc domain of an immunoglobulin heavy-chain. This includes, but is not limited to, polypeptides comprising the entire CH1, hinge, CH2, and/or CH3 domains as well as fragments of such peptides comprising only, e.g., the hinge, CH2, and CH3 domain. The Fc domain may be derived from an immunoglobulin of any species and/or any subtype, including, but not limited to, a human IgG1, IgG2, IgG3, IgG4, IgD, IgA, IgE, or IgM antibody. The Fc domain encompasses native Fc and Fc variant molecules. As set forth herein, it will be understood by one of ordinary skill in the art that any Fc domain may be modified such that it varies in amino acid sequence from the native Fc domain of a naturally occurring immunoglobulin molecule. In certain embodiments, the Fc domain has reduced effector function (e.g., FcγR binding). The Fc domains of a polypeptide described herein may be derived from different immunoglobulin molecules. For example, an Fc domain of a polypeptide may comprise a CH2 and/or CH3 domain derived from an IgG1 molecule and a hinge region derived from an IgG3 molecule. In another example, an Fc domain can comprise a chimeric hinge region derived, in part, from an IgG1 molecule and, in part, from an IgG3 molecule. In another example, an Fc domain can comprise a chimeric hinge derived, in part, from an IgG1 molecule and, in part, from an IgG4 molecule. In certain embodiments, an extended-PK group includes an Fc domain or fragments thereof or variants of the Fc domain or fragments thereof (all of which for the purpose of the present disclosure are comprised by the term “Fc domain”). The Fc domain does not contain a variable region that binds to antigen. Fc domains suitable for use in the present disclosure may be obtained from a number of different sources. In certain embodiments, an Fc domain is derived from a human immunoglobulin. In certain embodiments, the Fc domain is from a human IgG1 constant region. It is understood, however, that the Fc domain may be derived from an immunoglobulin of another mammalian species, including for example, a rodent (e.g. a mouse, rat, rabbit, guinea pig) or non-human primate (e.g. chimpanzee, macaque) species.

Moreover, the Fc domain (or a fragment or variant thereof) may be derived from any immunoglobulin class, including IgM, IgG, IgD, IgA, and IgE, and any immunoglobulin isotype, including IgG1, IgG2, IgG3, and IgG4.

A variety of Fc domain gene sequences (e.g., mouse and human constant region gene sequences) are available in the form of publicly accessible deposits. Constant region domains comprising an Fc domain sequence can be selected lacking a particular effector function and/or with a particular modification to reduce immunogenicity. Many sequences of antibodies and antibody-encoding genes have been published and suitable Fc domain sequences (e.g. hinge, CH2, and/or CH3 sequences, or fragments or variants thereof) can be derived from these sequences using art recognized techniques.

In certain embodiments, the extended-PK group is a serum albumin binding protein such as those described in US2005/0287153, US2007/0003549, US2007/0178082, US2007/0269422, US2010/0113339, WO2009/083804, and WO2009/133208, which are herein incorporated by reference in their entirety. In certain embodiments, the extended-PK group is transferrin, as disclosed in U.S. Pat. Nos. 7,176,278 and 8,158,579, which are herein incorporated by reference in their entirety. In certain embodiments, the extended-PK group is a serum immunoglobulin binding protein such as those disclosed in US2007/0178082, US2014/0220017, and US2017/0145062, which are herein incorporated by reference in their entirety. In certain embodiments, the extended-PK group is a fibronectin (Fn)-based scaffold domain protein that binds to serum albumin, such as those disclosed in US2012/0094909, which is herein incorporated by reference in its entirety. Methods of making fibronectin-based scaffold domain proteins are also disclosed in US2012/0094909. A non-limiting example of a Fn3-based extended-PK group is Fn3 (HSA), i.e., a Fn3 protein that binds to human serum albumin.

In certain aspects, the extended-PK polypeptide, suitable for use according to the disclosure, can employ one or more peptide linkers. As used herein, the term “peptide linker” refers to a peptide or polypeptide sequence which connects two or more domains (e.g., the extended-PK moiety and a polypeptide moiety, e.g., an Mtb antigen, immunogenic variant thereof, or immunogenic fragment of the Mtb antigen or the immunogenic variant thereof) in a linear amino acid sequence of a polypeptide chain. For example, peptide linkers may be used to connect an Mtb antigen, immunogenic variant thereof, or immunogenic fragment of the Mtb antigen or the immunogenic variant thereof to a HSA domain.

In the following, embodiments of vaccine RNAs are described, wherein certain terms used when describing elements thereof have the following meanings:
cap: 5′-cap structure, e.g., selected from the group consisting of m₂^{7,2′O}G(5′)ppSp(5′)G (in particular its D1 diastereomer), m₂^{7,3′O}G(5′)ppp(5′)G, and m₂^{7,3-O}Gppp (m₁^{2′-O})ApG.
hAg-Kozak: 5′-UTR sequence of the human alpha-globin mRNA with an optimized ‘Kozak sequence’ to increase translational efficiency.
sec/MITD: Fusion-protein tags derived from the sequence encoding the human MHC class I complex (HLA-B51, haplotype A2, B27/B51, Cw2/Cw3), which have been shown to improve antigen processing and presentation. Sec corresponds to the 78 bp fragment coding for the secretory signal peptide, which guides translocation of the nascent polypeptide chain into the endoplasmatic reticulum. MITD corresponds to the transmembrane and cytoplasmic domain of the MHC class I molecule, also called MHC class I trafficking domain.

Antigen: Sequences encoding the respective vaccine antigen(s)/epitope(s), i.e., one or more Mtb antigens, immunogenic variants thereof, or immunogenic fragments of the Mtb antigens or the immunogenic variants thereof.

Glycine-serine linker (GS): Sequences coding for short peptide linkers predominantly consisting of the amino acids glycine (G) and serine(S), as commonly used for fusion proteins.

FI element: The 3′-UTR is a combination of two sequence elements derived from the “amino terminal enhancer of split” (AES) mRNA (called F) and the mitochondrial encoded 12S ribosomal RNA (called I). These were identified by an ex vivo selection process for sequences that confer RNA stability and augment total protein expression.

A30L70: A poly(A)-tail measuring 110 nucleotides in length, consisting of a stretch of 30 adenosine residues, followed by a 10 nucleotide linker sequence and another 70 adenosine residues designed to enhance RNA stability and translational efficiency in dendritic cells.

In some embodiments, vaccine RNA described herein has one of the following structures:

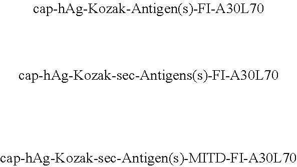

In some embodiments, vaccine antigen described herein has the structure:

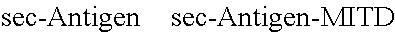

In some embodiments, hAg-Kozak comprises the nucleotide sequence of SEQ ID NO: 39. In some embodiments, sec of the encoded vaccine antigen/epitope comprises an amino acid sequence selected from SEQ ID NOs: 3 to 23 or encoded by the nucleotide sequence selected from SEQ ID NOs: 24 to 37. In some embodiments, MITD of the encoded vaccine antigen/epitope comprises the amino acid sequence of SEQ ID NO: 38. In some embodiments, FI comprises the nucleotide sequence of SEQ ID NO: 40. In some embodiments, A30L70 comprises the nucleotide sequence of SEQ ID NO: 41.

In some embodiments, the sequence encoding the vaccine antigen/epitope comprises a modified nucleoside replacing (partially or completely, preferably completely) uridine, wherein the modified nucleoside is selected from the group consisting of pseudouridine (ψ), N1-methyl-pseudouridine (m1ψ), and 5-methyl-uridine.

In some embodiments, the sequence encoding the vaccine antigen/epitope is codon-optimized.

In some embodiments, the G/C content of the sequence encoding the vaccine antigen/epitope is increased compared to the wild type coding sequence.

In some embodiments, the RNA (in particular, mRNA) described herein comprises:
a 5′ UTR comprising the nucleotide sequence of SEQ ID NO: 39, or a nucleotide sequence having at least 99%, 98%, 97%, 96%, 95%, 90%, 85%, or 80% identity to the nucleotide sequence of SEQ ID NO: 39;
a 3′ UTR comprising the nucleotide sequence of SEQ ID NO: 40, or a nucleotide sequence having at least 99%, 98%, 97%, 96%, 95%, 90%, 85%, or 80% identity to the nucleotide sequence of SEQ ID NO: 40; and
a poly-A sequence comprising the nucleotide sequence of SEQ ID NO: 41.

In some embodiments, the RNA (in particular, mRNA) described herein comprises:
m₂^{7,3-O}Gppp(m₁^{2′-O})ApG as capping structure at the 5′-end of the mRNA;
a 5′ UTR comprising the nucleotide sequence of SEQ ID NO: 39, or a nucleotide sequence having at least 99%, 98%, 97%, 96%, 95%, 90%, 85%, or 80% identity to the nucleotide sequence of SEQ ID NO: 39;
a 3′ UTR comprising the nucleotide sequence of SEQ ID NO: 40, or a nucleotide sequence having at least 99%, 98%, 97%, 96%, 95%, 90%, 85%, or 80% identity to the nucleotide sequence of SEQ ID NO: 40; and
a poly-A sequence comprising the nucleotide sequence of SEQ ID NO: 41.

In some embodiments, the RNA is unmodified. In some embodiments, the RNA is modified. In some embodiments, the RNA comprises N1-methyl-pseudouridine (m1ψ) in place of at least one uridine (e.g., in place of each uridine).

In some embodiments, the RNA (in particular, mRNA) described herein comprises:
m₂^{7,3-O}Gppp(m₁^{2′-O})ApG as capping structure at the 5′-end of the mRNA;
a 5′ UTR comprising the nucleotide sequence of SEQ ID NO: 39, or a nucleotide sequence having at least 99%, 98%, 97%, 96%, 95%, 90%, 85%, or 80% identity to the nucleotide sequence of SEQ ID NO: 39;
a 3′ UTR comprising the nucleotide sequence of SEQ ID NO: 40, or a nucleotide sequence having at least 99%, 98%, 97%, 96%, 95%, 90%, 85%, or 80% identity to the nucleotide sequence of SEQ ID NO: 40; and
a poly-A sequence comprising the nucleotide sequence of SEQ ID NO: 41; and
N1-methyl-pseudouridine (m1ψ) in place of at least one uridine (e.g., in place of each uridine).

In some embodiments, a vaccine antigen or epitope described herein is derived from Mycobacterium tuberculosis.

In some embodiments, a vaccine antigen or epitope described herein is derived from a Mycobacterium tuberculosis protein, an immunogenic variant thereof, or an immunogenic fragment of the Mycobacterium tuberculosis protein or the immunogenic variant thereof. Thus, in some embodiments, the RNA, e.g., mRNA, used in the present disclosure encodes an amino acid sequence comprising an Mtb protein, an immunogenic variant thereof, or an immunogenic fragment of the Mtb protein or the immunogenic variant thereof.

In some embodiments, a vaccine antigen or epitope described herein is derived from an Mtb protein from the acute phase of the Mtb life cycle, an immunogenic variant thereof, or an immunogenic fragment of the Mtb protein from the acute phase of the Mtb life cycle or the immunogenic variant thereof.

In some embodiments, a vaccine antigen or epitope described herein is derived from an Mtb protein from the latent phase of the Mtb life cycle, an immunogenic variant thereof, or an immunogenic fragment of the Mtb protein from the latent phase of the Mtb life cycle or the immunogenic variant thereof.

In some embodiments, a vaccine antigen or epitope described herein is derived from an Mtb protein from the resuscitation phase of the Mtb life cycle, an immunogenic variant thereof, or an immunogenic fragment of the Mtb protein from the resuscitation phase of the Mtb life cycle or the immunogenic variant thereof.

In some embodiments, RNA (in particular, mRNA) described herein (e.g., contained in the compositions/formulations of the present disclosure and/or used in the methods of the present disclosure) may be presented as a product containing the vaccine RNA as active substance and other ingredients comprising: ALC-0315 ((4-hydroxybutyl) azanediyl)bis(hexane-6,1-diyl)bis(2-hexyldecanoate), ALC-0159 (2-[(polyethylene glycol)-2000]-N,N-ditetradecylacetamide), 1,2-Distearoyl-sn-glycero-3-phosphocholine (DSPC), and cholesterol.

In some embodiments, the RNA (in particular, mRNA) described herein is formulated or is to be formulated as a liquid, a solid, or a combination thereof.

In some embodiments, the RNA (in particular, mRNA) described herein is formulated or is to be formulated for injection. In some embodiments, the RNA (in particular, mRNA) described herein is formulated or is to be formulated for intramuscular administration.

In some embodiments, the RNA (in particular, mRNA) described herein is formulated or is to be formulated as a composition, e.g., a pharmaceutical composition.

In some embodiments, the composition comprises a cationically ionizable lipid.

In some embodiments, the composition comprises a cationically ionizable lipid and one or more additional lipids. In some embodiments, the one or more additional lipids are selected from polymer-conjugated lipids, neutral lipids, and combinations thereof. In some embodiments, the neutral lipids include phospholipids, steroid lipids, and combinations thereof. In some embodiments, the one or more additional lipids are a combination of a polymer-conjugated lipid, a phospholipid, and a steroid lipid.

In some embodiments, the composition comprises a cationically ionizable lipid; a polymer-conjugated lipid which is a PEG-conjugated lipid; cholesterol; and a phospholipid. In some embodiments, the phospholipid is DSPC. In some embodiments, the phospholipid is DOPE.

In some embodiments, the composition comprises a cationically ionizable lipid; a polymer-conjugated lipid which is 2-[(polyethylene glycol)-2000]-N,N-ditetradecylacetamide; cholesterol; and a phospholipid. In some embodiments, the phospholipid is DSPC. In some embodiments, the phospholipid is DOPE.

In some embodiments, the composition comprises a cationically ionizable lipid which is ((4-hydroxybutyl) azanediyl)bis(hexane-6,1-diyl)bis(2-hexyldecanoate); a polymer-conjugated lipid which is 2-[(polyethylene glycol)-2000]-N,N-ditetradecylacetamide; cholesterol; and a phospholipid. In some embodiments, the phospholipid is DSPC. In some embodiments, the phospholipid is DOPE. In some embodiments, at least a portion of (i) the RNA, (ii) the cationically ionizable lipid, and if present, (iii) the one or more additional lipids is present in particles. In some embodiments, the particles are nanoparticles, such as lipid nanoparticles (LNPs).

In some embodiments, the composition, in particular the pharmaceutical composition, is a vaccine.

In some embodiments, the composition, in particular the pharmaceutical composition, further comprises one or more pharmaceutically acceptable carriers, diluents and/or excipients.

In some embodiments, the RNA and/or the composition, in particular the pharmaceutical composition, is/are a component of a kit.

In some embodiments, the kit further comprises instructions for use of the RNA for inducing an immune response against Mycobacterium tuberculosis in a subject.

In some embodiments, the kit further comprises instructions for use of the RNA for therapeutically or prophylactically treating a Mycobacterium tuberculosis infection in a subject.

In some embodiments, the subject is a human.

In some embodiments, the RNA (in particular, mRNA), e.g., RNA encoding vaccine antigen, described in the present disclosure is non-immunogenic. RNA encoding an immunostimulant may be administered according to the present disclosure to provide an adjuvant effect. The RNA encoding an immunostimulant may be standard RNA or non-immunogenic RNA.

Embodiments of Mycobacterium tuberculosis (Mtb) Antigens

The present disclosure describes Mtb antigens, immunogenic variants thereof, and immunogenic fragments of the Mtb antigens or the immunogenic variants thereof (referred to as “Mtb antigens” herein) and RNA encoding these antigens.

Mtb antigens described herein include LpqH and PstS1.

### LpqH

In some embodiments, the Mtb antigen LpqH comprises the amino acid sequence according to any one of SEQ ID NOs: 1, 43 or 45. A full-length antigen representing the antigen LpqH is characterized in that it comprises the full-length amino acid sequence according to SEQ ID NO: 1, 43 or 45, whereas an antigen fragment representing the antigen LpqH is characterized in that it comprises an amino acid sequence which is only a part of SEQ ID NO: 1, 43 or 45 but which still is able to induce an immune reaction to LpqH, when delivered to a subject.

An immunogenic variant of the Mtb antigen LpqH comprises an amino acid sequence which is “immunologically equivalent” to LpqH and thus, is able to induce an immune reaction to LpqH, when delivered to a subject. In some embodiments, an immunogenic variant of the Mtb antigen LpqH comprises an amino acid sequence differing from SEQ ID NO: 1, 43 or 45 by one or more deletions, insertions or substitutions in the amino acid sequence while having at least 99%, 98%, 97%, 96%, 95%, 90%, 85%, or 80% identity to SEQ ID NO: 1, 43 or 45 (e.g. determined by sequence alignment using known sequence alignment algorithms such as BLASTP). A full-length antigen representing an immunogenic variant of the antigen LpqH is characterized in that it comprises the full-length amino acid sequence of the immunogenic variant, whereas an antigen fragment representing an immunogenic variant of the antigen LpqH is characterized in that it comprises an amino acid sequence which is only a part of the full-length amino acid sequence of the immunogenic variant but which still is able to induce an immune reaction to LpqH, when delivered to a subject.

In some embodiments, the Mtb antigen LpqH is encoded by a nucleotide sequence according to any one of SEQ IDs NO: 47 to 52.

In some embodiments, an immunogenic variant of the Mtb antigen LpqH is encoded by a nucleotide sequence having at least 99%, 98%, 97%, 96%, 95%, 90%, 85%, or 80% identity to any one of SEQ ID NOs: 47 to 52 (e.g. determined by sequence alignment using known sequence alignment algorithms such as BLASTN).

### PstS1

In some embodiments, the Mtb antigen PstS1 comprises the amino acid sequence according to any one of SEQ ID NO: 2, 44 or 46. A full-length antigen representing the antigen PstS1 is characterized in that it comprises the full-length amino acid sequence according to SEQ ID NO: 2, 44 or 46, whereas an antigen fragment representing the antigen PstS1 is characterized in that it comprises an amino acid sequence which is only a part of SEQ ID NO: 2, 44 or 46 but which still is able to induce an immune reaction to PstS1, when delivered to a subject.

An immunogenic variant of the Mtb antigen PstS1 comprises an amino acid sequence which is “immunologically equivalent” to PstS1 and thus, is able to induce an immune reaction to PstS1, when delivered to a subject. In some embodiments, an immunogenic variant of the Mtb antigen PstS1 comprises an amino acid sequence differing from SEQ ID NO: 2, 44 or 46 by one or more deletions, insertions or substitutions in the amino acid sequence while having at least 99%, 98%, 97%, 96%, 95%, 90%, 85%, or 80% identity to SEQ ID NO: 2, 44 or 46 (e.g. determined by sequence alignment using known sequence alignment algorithms such as BLASTP). A full-length antigen representing an immunogenic variant of the antigen PstS1 is characterized in that it comprises the full-length amino acid sequence of the immunogenic variant, whereas an antigen fragment representing an immunogenic variant of the antigen PstS1 is characterized in that it comprises an amino acid sequence which is only a part of the full-length amino acid sequence of the immunogenic variant but which still is able to induce an immune reaction to PstS1, when delivered to a subject. In some embodiments, the Mtb antigen PstS1 is encoded by a nucleotide sequence according to any one of SEQ ID NOs: 53 to 58.

In some embodiments, an immunogenic variant of the Mtb antigen PstS1 is encoded by a nucleotide sequence having at least 99%, 98%, 97%, 96%, 95%, 90%, 85%, or 80% identity to any one of SEQ ID NOs: 53 to 58 (e.g. determined by sequence alignment using known sequence alignment algorithms such as BLASTN).

TABLE 4
Native amino acid sequences of Mycobacterium tuberculosis
antigens used of Mycobacterium tuberculosis antigens used

| Antigen | Amino acid sequence |
|---|---|
| LpqH | MKRGLTVAVAGAAILVAGLSGCSSNKSTTGSGETTTAAGTTASPGAASG |
| | PKWVIDGKDQNVTGSVVCTTAAGNVNIAIGGAATGIAAVLTDGNPPEVK |
| | SVGLGNVNGVTLGYTSGTGQGNASATKDGSHYKITGTATGVDMANPMSP |
| | VNKSFEIEVTCS |
| | [SEQ ID NO: 1] |
| PstS1 | MKIRLHTLLAVLTAAPLLLAAAGCGSKPPSGSPETGAGAGTVATTPASS |
| | PVTLAETGSTLLYPLFNLWGPAFHERYPNVTITAQGTGSGAGIAQAAAG |
| | TVNIGASDAYLSEGDMAAHKGLMNIALAISAQQVNYNLPGVSEHLKLNG |
| | KVLAAMYQGTIKTWDDPQIAALNPGVNLPGTAVVPLHRSDGSGDTFLFT |
| | QYLSKQDPEGWGKSPGFGTTVDFPAVPGALGENGNGGMVTGCAETPGCV |
| | AYIGISFLDQASQRGLGEAQLGNSSGNFLLPDAQSIQAAAAGFASKTPA |
| | NQAISMIDGPAPDGYPIINYEYAIVNNRQKDAATAQTLQAFLHWAITDG |
| | NKASFLDQVHFQPLPPAVVKLSDALIATISS |
| | [SEQ ID NO: 2] |
| LpqH | MKRGLTVAVAGAAILVAGLSGCSSQKSTTGSGETTTAAGTTASPGAASG |
| (N-glyc N to | PKVVIDGKDQQVTGSVVCTTAAGNVNIAIGGAATGIAAVLTDGNPPEVK |
| Q sequence) | SVGLGNVNGVTLGYTSGTGQGQASATKDGSHYKITGTATGVDMANPMSP |
| | VNKSFEIEVTCS [SEQ ID NO: 43] |
| PstS1 (N-glyc | MKIRLHTLLAVLTAAPLLLAAAGCGSKPPSGSPETGAGAGTVATTPASS |
| N to Q | PVTLAETGSTLLYPLFNLWGPAFHERYPQVTITAQGTGSGAGIAQAAAG |
| sequence) | TVNIGASDAYLSEGDMAAHKGLMNIALAISAQQVNYNLPGVSEHLKLNG |
| | KVLAAMYQGTIKTWDDPQIAALNPGVNLPGTAVVPLHRSDGSGDTFLFT |
| | QYLSKQDPEGWGKSPGFGTTVDFPAVPGALGENGNGGMVTGCAETPGCV |
| | AYIGISFLDQASQRGLGEAQLGQSSGNFLLPDAQSIQAAAAGFASKTPA |
| | NQAISMIDGPAPDGYPIINYEYAIVNNRQKDAATAQTLQAFLHWAITDG |
| | NKASFLDQVHFQPLPPAVVKLSDALIATISS [SEQ ID NO: 44] |
| LpqH (d1-25) | KSTTGSGETTTAAGTTASPGAASGPKVVIDGKDQNVTGSVVCTTAAGNV |
| | NIAIGGAATGIAAVLTDGNPPEVKSVGLGNVNGVTLGYTSGTGQGNASA |
| | TKDGSHYKITGTATGVDMANPMSPVNKSFEIEVTCS |
| | [SEQ ID NO: 45] |
| PstS1 (d1-26) | KPPSGSPETGAGAGTVATTPASSPVTLAETGSTLLYPLFNLWGPAFHER |
| | YPNVTITAQGTGSGAGIAQAAAGTVNIGASDAYLSEGDMAAHKGLMNIA |
| | LAISAQQVNYNLPGVSEHLKLNGKVLAAMYQGTIKTWDDPQIAALNPGV |
| | NLPGTAVVPLHRSDGSGDTFLFTQYLSKQDPEGWGKSPGFGTTVDFPAV |
| | PGALGENGNGGMVTGCAETPGCVAYIGISFLDQASQRGLGEAQLGNSSG |
| | NFLLPDAQSIQAAAAGFASKTPANQAISMIDGPAPDGYPIINYEYAIVN |
| | NRQKDAATAQTLQAFLHWAITDGNKASFLDQVHFQPLPPAVVKLSDALI |
| | ATISS [SEQ ID NO: 46] |

TABLE 5
Nucleotide sequence of Mycobacterium tuberculosis antigens used

| Antigen | Nucleotide sequence |
|---|---|
| LpqH (s) | GTGAAAAGGGGCCTGACAGTTGCTGTTGCCGGCGCTGCTATTCTGGTGGCTGGAC |
| | TGTCTGGCTGCAGCAGCAACAAGAGCACAACAGGCAGCGGCGAGACAACAACAGC |
| | CGCCGGAACAACAGCTTCTCCTGGCGCTGCTTCTGGCCCCAAGGTTGTGATCGAT |
| | GGCAAGGACCAGAACGTGACCGGCAGCGTCGTGTGTACAACCGCCGCTGGCAATG |
| | TGAATATCGCCATTGGCGGAGCCGCCACAGGCATTGCTGCTGTGCTGACAGATGG |
| | CAACCCTCCTGAAGTGAAGTCTGTCGGCCTGGGCAATGTCAACGGCGTGACACTG |
| | GGCTACACAAGCGGAACAGGACAGGGCAATGCCAGCGCCACAAAGGATGGCAGCC |
| | ACTACAAGATCACCGGCACAGCTACAGGCGTGGACATGGCCAATCCTATGAGCCC |
| | CGTGAACAAGTCCTTCGAGATCGAAGTGACCTGCAGC [SEQ ID NO: 47] |
| LpqH (l) | ATGAGAGTGATGGCCCCCAGAACCCTGATCCTGCTGCTGTCTGGCGCCCTGGCCC |
| | TGACAGAGACATGGGCCGGAAGCGACTACAAAGACGATGACGACAAGGTGAAAAG |
| | GGGCCTGACAGTTGCTGTTGCCGGCGCTGCTATTCTGGTGGCTGGACTGTCTGGC |
| | TGCAGCAGCAACAAGAGCACAACAGGCAGCGGCGAGACAACAACAGCCGCCGGAA |
| | CAACAGCTTCTCCTGGCGCTGCTTCTGGCCCCAAGGTTGTGATCGATGGCAAGGA |
| | CCAGAACGTGACCGGCAGCGTCGTGTGTACAACCGCCGCTGGCAATGTGAATATC |
| | GCCATTGGCGGAGCCGCCACAGGCATTGCTGCTGTGCTGACAGATGGCAACCCTC |
| | CTGAAGTGAAGTCTGTCGGCCTGGGCAATGTCAACGGCGTGACACTGGGCTACAC |
| | AAGCGGAACAGGACAGGGCAATGCCAGCGCCACAAAGGATGGCAGCCACTACAAG |
| | ATCACCGGCACAGCTACAGGCGTGGACATGGCCAATCCTATGAGCCCCGTGAACA |
| | AGTCCTTCGAGATCGAAGTGACCTGCAGCTGATGA [SEQ ID NO: 48] |
| LpqH (N-glyc N to | ATGAAAAGGGGCCTGACAGTTGCTGTTGCCGGCGCTGCTATTCTGGTGGCTGGAC |
| Q sequence) | TGTCTGGCTGCAGCAGCCAGAAAAGCACAACAGGCAGCGGCGAGACAACAACAGC |
| | CGCCGGAACAACAGCTTCTCCTGGCGCTGCTTCTGGCCCCAAGGTTGTGATCGAT |
| | GGCAAGGACCAGCAGGTGACCGGCAGCGTCGTGTGTACAACCGCCGCTGGCAATG |
| | TGAATATCGCCATTGGCGGAGCCGCCACAGGCATTGCTGCTGTGCTGACAGATGG |
| | CAACCCTCCTGAAGTGAAGTCTGTCGGCCTGGGCAATGTCAACGGCGTGACACTG |
| | GGCTACACAAGCGGAACAGGACAGGGCCAGGCCAGCGCCACAAAGGATGGCAGCC |
| | ACTACAAGATCACCGGCACAGCTACAGGCGTGGACATGGCCAATCCTATGAGCCC |
| | CGTGAACAAGTCCTTCGAGATCGAAGTGACCTGCAGC [SEQ ID NO: 49] |
| LpqH (d1-25) (s) | AAGTCCACAACAGGCAGCGGCGAGACAACAACAGCCGCCGGAACAACAGCTTCTC |
| | CTGGCGCTGCTTCTGGCCCCAAGGTTGTGATCGATGGCAAGGACCAGAACGTGAC |
| | CGGCAGCGTCGTGTGTACAACCGCCGCTGGCAATGTGAATATCGCCATTGGCGGA |
| | GCCGCCACAGGCATTGCTGCTGTGCTGACAGATGGCAACCCTCCTGAAGTGAAGT |
| | CTGTCGGCCTGGGCAATGTCAACGGCGTGACACTGGGCTACACAAGCGGAACAGG |
| | ACAGGGCAATGCCAGCGCCACAAAGGATGGCAGCCACTACAAGATCACCGGCACA |
| | GCTACAGGCGTGGACATGGCCAATCCTATGAGCCCCGTGAACAAGTCCTTCGAGA |
| | TCGAAGTGACCTGCAGC [SEQ ID NO: 50] |
| LpqH (d1-25) (l) | ATGAGAGTGATGGCCCCCAGAACCCTGATCCTGCTGCTGTCTGGCGCCCTGGCCC |
| | TGACAGAGACATGGGCCGGAAGCGACTACAAAGACGATGACGACAAGAAGTCCAC |
| | AACAGGCAGCGGCGAGACAACAACAGCCGCCGGAACAACAGCTTCTCCTGGCGCT |
| | GCTTCTGGCCCCAAGGTTGTGATCGATGGCAAGGACCAGAACGTGACCGGCAGCG |
| | TCGTGTGTACAACCGCCGCTGGCAATGTGAATATCGCCATTGGCGGAGCCGCCAC |
| | AGGCATTGCTGCTGTGCTGACAGATGGCAACCCTCCTGAAGTGAAGTCTGTCGGC |
| | CTGGGCAATGTCAACGGCGTGACACTGGGCTACACAAGCGGAACAGGACAGGGCA |
| | ATGCCAGCGCCACAAAGGATGGCAGCCACTACAAGATCACCGGCACAGCTACAGG |
| | CGTGGACATGGCCAATCCTATGAGCCCCGTGAACAAGTCCTTCGAGATCGAAGTG |
| | ACCTGCAGCTGATGA [SEQ ID NO: 51] |
| LpqH (wt) | gtgaagcgtggactgacggtcgcggtagccggagccgccattctggtcgcaggtc |
| | tttccggatgttcaagcaacaagtcgactacaggaagcggtgagaccacgaccgc |
| | ggcaggcacgacggcaagccccggcgccgcctccgggccgaaggtcgtcatcgac |
| | ggtaaggaccagaacgtcaccggctccgtggtgtgcacaaccgcggccggcaatg |
| | tcaacatcgcgatcggcggggcggcgaccggcattgccgccgtgctcaccgacgg |
| | caaccctccggaggtgaagtccgttgggctcggtaacgtcaacggcgtcacgctg |
| | ggatacacgtcgggcaccggacagggtaacgcctcggcaaccaaggacggcagcc |
| | actacaagatcactgggaccgctaccggggtcgacatggccaacccgatgtcacc |
| | ggtgaacaagtcgttcgaaatcgaggtgacctgttcctaa [SEQ ID NO: 52] |
| PstS1 (s) | GTGAAGATCAGACTGCACACACTGCTGGCCGTGCTGACAGCTGCTCCTCTGCTTC |
| | TTGCTGCCGCCGGATGTGGATCTAAGCCTCCTTCTGGCTCTCCTGAAACAGGCGC |
| | TGGTGCTGGCACAGTGGCTACAACACCAGCTTCTAGCCCTGTGACACTGGCCGAG |
| | ACAGGCTCTACACTGCTGTACCCTCTGTTCAACCTGTGGGGCCCTGCCTTCCACG |
| | AGAGATACCCCAACGTGACCATCACAGCCCAAGGCACAGGATCTGGCGCCGGAAT |
| | TGCTCAAGCTGCCGCTGGCACCGTGAATATCGGAGCCTCTGATGCCTACCTGAGC |
| | GAGGGCGATATGGCCGCTCACAAGGGCCTGATGAATATTGCCCTGGCCATCAGCG |
| | CCCAGCAAGTGAACTATAACCTGCCTGGCGTGTCCGAGCACCTGAAGCTGAATGG |
| | AAAAGTGCTGGCCGCCATGTACCAGGGCACCATCAAGACCTGGGACGACCCTCAG |
| | ATCGCCGCTCTGAATCCGGGTGTTAATCTGCCTGGAACCGCCGTGGTGCCTCTGC |
| | ACAGATCTGATGGAAGCGGCGACACCTTCCTGTTCACCCAGTACCTGAGCAAGCA |
| | GGACCCTGAAGGCTGGGGCAAGTCTCCTGGCTTTGGCACCACCGTGGATTTCCCT |
| | GCTGTTCCTGGTGCTCTGGGCGAGAATGGCAATGGCGGCATGGTTACCGGATGCG |
| | CCGAAACACCTGGCTGCGTGGCCTATATCGGCATCAGCTTTCTGGACCAGGCCAG |
| | CCAGAGAGGACTGGGAGAAGCTCAGCTGGGCAACAGCAGCGGCAATTTCCTGCTG |
| | CCTGACGCTCAGTCTATCCAGGCTGCAGCTGCCGGCTTTGCCTCTAAGACACCAG |
| | CCAATCAGGCCATCTCCATGATCGACGGCCCTGCTCCTGATGGCTACCCCATCAT |
| | CAACTACGAGTACGCCATCGTGAACAACCGGCAGAAGGATGCCGCCACAGCTCAG |
| | ACACTGCAGGCCTTTCTGCACTGGGCCATCACCGATGGAAACAAGGCCAGCTTCC |
| | TGGATCAGGTGCACTTCCAGCCACTGCCTCCAGCCGTGGTCAAACTGTCTGATGC |
| | CCTGATCGCCACCATCAGCAGC [SEQ ID NO: 53] |
| PstS1 (l) | ATGAGAGTGATGGCCCCCAGAACCCTGATCCTGCTGCTGTCTGGCGCCCTGGCCC |
| | TGACAGAGACATGGGCCGGAAGCGACTACAAAGACGATGACGACAAGGTGAAGAT |
| | CAGACTGCACACACTGCTGGCCGTGCTGACAGCTGCTCCTCTGCTTCTTGCTGCC |
| | GCCGGATGTGGATCTAAGCCTCCTTCTGGCTCTCCTGAAACAGGCGCTGGTGCTG |
| | GCACAGTGGCTACAACACCAGCTTCTAGCCCTGTGACACTGGCCGAGACAGGCTC |
| | TACACTGCTGTACCCTCTGTTCAACCTGTGGGGCCCTGCCTTCCACGAGAGATAC |
| | CCCAACGTGACCATCACAGCCCAAGGCACAGGATCTGGCGCCGGAATTGCTCAAG |
| | CTGCCGCTGGCACCGTGAATATCGGAGCCTCTGATGCCTACCTGAGCGAGGGCGA |
| | TATGGCCGCTCACAAGGGCCTGATGAATATTGCCCTGGCCATCAGCGCCCAGCAA |
| | GTGAACTATAACCTGCCTGGCGTGTCCGAGCACCTGAAGCTGAATGGAAAAGTGC |
| | TGGCCGCCATGTACCAGGGCACCATCAAGACCTGGGACGACCCTCAGATCGCCGC |
| | TCTGAATCCGGGTGTTAATCTGCCTGGAACCGCCGTGGTGCCTCTGCACAGATCT |
| | GATGGAAGCGGCGACACCTTCCTGTTCACCCAGTACCTGAGCAAGCAGGACCCTG |
| | AAGGCTGGGGCAAGTCTCCTGGCTTTGGCACCACCGTGGATTTCCCTGCTGTTCC |
| | TGGTGCTCTGGGCGAGAATGGCAATGGCGGCATGGTTACCGGATGCGCCGAAACA |
| | CCTGGCTGCGTGGCCTATATCGGCATCAGCTTTCTGGACCAGGCCAGCCAGAGAG |
| | GACTGGGAGAAGCTCAGCTGGGCAACAGCAGCGGCAATTTCCTGCTGCCTGACGC |
| | TCAGTCTATCCAGGCTGCAGCTGCCGGCTTTGCCTCTAAGACACCAGCCAATCAG |
| | GCCATCTCCATGATCGACGGCCCTGCTCCTGATGGCTACCCCATCATCAACTACG |
| | AGTACGCCATCGTGAACAACCGGCAGAAGGATGCCGCCACAGCTCAGACACTGCA |
| | GGCCTTTCTGCACTGGGCCATCACCGATGGAAACAAGGCCAGCTTCCTGGATCAG |
| | GTGCACTTCCAGCCACTGCCTCCAGCCGTGGTCAAACTGTCTGATGCCCTGATCG |
| | CCACCATCAGCAGCTGATGA [SEQ ID NO: 54] |
| PstS1 (N-glyc N to | ATGAAGATCAGACTGCACACACTGCTGGCCGTGCTGACAGCTGCTCCTCTGCTTC |
| Q sequence) | TTGCTGCCGCCGGATGTGGATCTAAGCCTCCTTCTGGCTCTCCTGAAACAGGCGC |
| | TGGTGCTGGCACAGTGGCTACAACACCAGCTTCTAGCCCTGTGACACTGGCCGAG |
| | ACAGGCTCTACACTGCTGTACCCTCTGTTCAACCTGTGGGGCCCTGCCTTCCACG |
| | AGAGATACCCCCAGGTGACCATCACAGCCCAAGGCACAGGATCTGGCGCCGGAAT |
| | TGCTCAAGCTGCCGCTGGCACCGTGAATATCGGAGCCTCTGATGCCTACCTGAGC |
| | GAGGGCGATATGGCCGCTCACAAGGGCCTGATGAATATTGCCCTGGCCATCAGCG |
| | CCCAGCAAGTGAACTATAACCTGCCTGGCGTGTCCGAGCACCTGAAGCTGAATGG |
| | AAAAGTGCTGGCCGCCATGTACCAGGGCACCATCAAGACCTGGGACGACCCTCAG |
| | ATCGCCGCTCTGAATCCGGGTGTTAATCTGCCTGGAACCGCCGTGGTGCCTCTGC |
| | ACAGATCTGATGGAAGCGGCGACACCTTCCTGTTCACCCAGTACCTGAGCAAGCA |
| | GGACCCTGAAGGCTGGGGCAAGTCTCCTGGCTTTGGCACCACCGTGGATTTCCCT |
| | GCTGTTCCTGGTGCTCTGGGCGAGAATGGCAATGGCGGCATGGTTACCGGATGCG |
| | CCGAAACACCTGGCTGCGTGGCCTATATCGGCATCAGCTTTCTGGACCAGGCCAG |
| | CCAGAGAGGACTGGGAGAAGCTCAGCTGGGCCAGAGCAGCGGCAATTTCCTGCTG |
| | CCTGACGCTCAGTCTATCCAGGCTGCAGCTGCCGGCTTTGCCTCTAAGACACCAG |
| | CCAATCAGGCCATCTCCATGATCGACGGCCCTGCTCCTGATGGCTACCCCATCAT |
| | CAACTACGAGTACGCCATCGTGAACAACCGGCAGAAGGATGCCGCCACAGCTCAG |
| | ACACTGCAGGCCTTTCTGCACTGGGCCATCACCGATGGAAACAAGGCCAGCTTCC |
| | TGGATCAGGTGCACTTCCAGCCACTGCCTCCAGCCGTGGTCAAACTGTCTGATGC |
| | CCTGATCGCCACCATCAGCAGC [SEQ ID NO: 55] |
| PstS1 (d1-26) (s) | AAGCCTCCTAGCGGCTCTCCTGAAACAGGTGCTGGTGCTGGCACAGTGGCCACCA |
| | CACCAGCATCTTCTCCAGTGACACTGGCCGAGACAGGCTCTACCCTGCTGTACCC |
| | TCTGTTCAACCTGTGGGGCCCTGCCTTCCACGAGAGATACCCCAACGTGACCATC |
| | ACAGCCCAAGGCACAGGATCTGGCGCCGGAATTGCTCAAGCTGCCGCCGGAACAG |
| | TGAATATCGGAGCCTCTGATGCCTACCTGAGCGAGGGCGATATGGCCGCTCACAA |
| | GGGCCTGATGAATATTGCCCTGGCCATCAGCGCCCAGCAAGTGAACTATAACCTG |
| | CCTGGCGTGTCCGAGCACCTGAAGCTGAATGGAAAAGTGCTGGCCGCCATGTACC |
| | AGGGCACCATCAAGACCTGGGACGACCCTCAGATCGCCGCTCTGAATCCGGGTGT |
| | TAATCTGCCTGGAACCGCCGTGGTGCCTCTGCACAGATCTGATGGAAGCGGCGAC |
| | ACCTTCCTGTTCACCCAGTACCTGAGCAAGCAGGACCCTGAAGGCTGGGGCAAGT |
| | CTCCTGGCTTTGGCACCACCGTGGATTTCCCTGCTGTTCCTGGTGCTCTGGGCGA |
| | GAATGGCAATGGCGGCATGGTTACCGGATGCGCCGAAACACCTGGCTGCGTGGCC |
| | TATATCGGCATCAGCTTTCTGGACCAGGCCAGCCAGAGAGGACTGGGAGAAGCTC |
| | AGCTGGGCAACAGCAGCGGCAATTTCCTGCTGCCTGACGCTCAGTCTATCCAGGC |
| | TGCTGCTGCCGGCTTTGCCTCTAAGACACCAGCCAATCAGGCCATCTCCATGATC |
| | GACGGCCCTGCTCCTGATGGCTACCCCATCATCAACTACGAGTACGCCATCGTGA |
| | ACAACCGGCAGAAGGATGCCGCCACAGCTCAGACACTGCAGGCCTTTCTGCACTG |
| | GGCCATCACCGATGGAAACAAGGCCAGCTTCCTGGATCAGGTGCACTTCCAGCCA |
| | CTGCCTCCAGCCGTGGTCAAACTGTCTGATGCCCTGATCGCCACCATCAGCAGC |
| | [SEQ ID NO: 56] |
| PstS1 (d1-26) (l) | ATGAGAGTGATGGCCCCCAGAACCCTGATCCTGCTGCTGTCTGGCGCCCTGGCCC |
| | TGACAGAGACATGGGCCGGAAGCGACTACAAAGACGATGACGACAAGAAGCCTCC |
| | TAGCGGCTCTCCTGAAACAGGTGCTGGTGCTGGCACAGTGGCCACCACACCAGCA |
| | TCTTCTCCAGTGACACTGGCCGAGACAGGCTCTACCCTGCTGTACCCTCTGTTCA |
| | ACCTGTGGGGCCCTGCCTTCCACGAGAGATACCCCAACGTGACCATCACAGCCCA |
| | AGGCACAGGATCTGGCGCCGGAATTGCTCAAGCTGCCGCCGGAACAGTGAATATC |
| | GGAGCCTCTGATGCCTACCTGAGCGAGGGCGATATGGCCGCTCACAAGGGCCTGA |
| | TGAATATTGCCCTGGCCATCAGCGCCCAGCAAGTGAACTATAACCTGCCTGGCGT |
| | GTCCGAGCACCTGAAGCTGAATGGAAAAGTGCTGGCCGCCATGTACCAGGGCACC |
| | ATCAAGACCTGGGACGACCCTCAGATCGCCGCTCTGAATCCGGGTGTTAATCTGC |
| | CTGGAACCGCCGTGGTGCCTCTGCACAGATCTGATGGAAGCGGCGACACCTTCCT |
| | GTTCACCCAGTACCTGAGCAAGCAGGACCCTGAAGGCTGGGGCAAGTCTCCTGGC |
| | TTTGGCACCACCGTGGATTTCCCTGCTGTTCCTGGTGCTCTGGGCGAGAATGGCA |
| | ATGGCGGCATGGTTACCGGATGCGCCGAAACACCTGGCTGCGTGGCCTATATCGG |
| | CATCAGCTTTCTGGACCAGGCCAGCCAGAGAGGACTGGGAGAAGCTCAGCTGGGC |
| | AACAGCAGCGGCAATTTCCTGCTGCCTGACGCTCAGTCTATCCAGGCTGCTGCTG |
| | CCGGCTTTGCCTCTAAGACACCAGCCAATCAGGCCATCTCCATGATCGACGGCCC |
| | TGCTCCTGATGGCTACCCCATCATCAACTACGAGTACGCCATCGTGAACAACCGG |
| | CAGAAGGATGCCGCCACAGCTCAGACACTGCAGGCCTTTCTGCACTGGGCCATCA |
| | CCGATGGAAACAAGGCCAGCTTCCTGGATCAGGTGCACTTCCAGCCACTGCCTCC |
| | AGCCGTGGTCAAACTGTCTGATGCCCTGATCGCCACCATCAGCAGCTGATGA |
| | [SEQ ID NO: 57] |
| PstS1 (wt) | Gtgaaaattcgtttgcatacgctgttggccgtgttgaccgctgcgccgctgctgc |
| | tagcagcggcgggctgtggctcgaaaccaccgagcggttcgcctgaaacgggcgc |
| | cggcgccggtactgtcgcgactacccccgcgtcgtcgccggtgacgttggcggag |
| | accggtagcacgctgctctacccgctgttcaacctgtggggtccggcctttcacg |
| | agaggtatccgaacgtcacgatcaccgctcagggcaccggttctggtgccgggat |
| | cgcgcaggccgccgccgggacggtcaacattggggcctccgacgcctatctgtcg |
| | gaaggtgatatggccgcgcacaaggggctgatgaacatcgcgctagccatctccg |
| | ctcagcaggtcaactacaacctgcccggagtgagcgagcacctcaagctgaacgg |
| | aaaagtcctggcggccatgtaccagggcaccatcaaaacctgggacgacccgcag |
| | atcgctgcgctcaaccccggcgtgaacctgcccggcaccgcggtagttccgctgc |
| | accgctccgacgggtccggtgacaccttcttgttcacccagtacctgtccaagca |
| | agatcccgagggctggggcaagtcgcccggcttcggcaccaccgtcgacttcccg |
| | gcggtgccgggtgcgctgggtgagaacggcaacggcggcatggtgaccggttgcg |
| | ccgagacaccgggctgcgtggcctatatcggcatcagcttcctcgaccaggccag |
| | tcaacggggactcggcgaggcccaactaggcaatagctctggcaatttcttgttg |
| | cccgacgcgcaaagcattcaggccgcggcggctggcttcgcatcgaaaaccccgg |
| | cgaaccaggcgatttcgatgatcgacgggcccgccccggacggctacccgatcat |
| | caactacgagtacgccatogtcaacaaccggcaaaaggacgccgccaccgcgcag |
| | accttgcaggcatttctgcactgggcgatcaccgacggcaacaaggcctcgttcc |
| | tcgaccaggttcatttccagccgctgccgcccgcggtggtgaagttgtctgacgc |
| | gttgatcgcgacgatttccagctag |
| | [SEQ ID NO: 58] |

### RNA Delivery

RNA described herein may be delivered for therapeutic applications described herein using any appropriate methods known in the art, including, e.g., delivery as naked RNA, or delivery mediated by delivery vehicles.

Some aspects of the disclosure involve the targeted delivery of the RNA disclosed herein to certain cells or tissues. In some embodiments, after administration of the RNA (in particular, mRNA) compositions/formulations described herein, at least a portion of the RNA is delivered to a target cell or target organ. In some embodiments, at least a portion of the RNA is delivered to the cytosol of the target cell. In some embodiments, the RNA (in particular, mRNA) is translated by the target cell to produce the encoded peptide or polypeptide. In some embodiments, the target cell is a muscle cell. In some embodiments, the target cell is a cell in the liver. In some embodiments, the target cell is a cell in the lung. In some embodiments, the disclosure involves targeting the lymphatic system, in particular secondary lymphoid organs, more specifically spleen. In some embodiments, the target cell is a cell in the lymph nodes. In some embodiments, the target cell is a spleen cell. In some embodiments, the target cell is an antigen presenting cell such as a professional antigen presenting cell in the spleen. In some embodiments, the target cell is a dendritic cell in the spleen. Thus, RNA (in particular, mRNA) compositions/formulations described herein may be used for delivering RNA to such target cell. The “lymphatic system” is part of the circulatory system and an important part of the immune system, comprising a network of lymphatic vessels that carry lymph. The lymphatic system consists of lymphatic organs, a conducting network of lymphatic vessels, and the circulating lymph. The primary or central lymphoid organs generate lymphocytes from immature progenitor cells. The thymus and the bone marrow constitute the primary lymphoid organs. Secondary or peripheral lymphoid organs, which include lymph nodes and the spleen, maintain mature naive lymphocytes and initiate an adaptive immune response. Lipid-based RNA delivery systems have an inherent preference to the liver, where, depending on the composition of the RNA delivery systems used, RNA expression in the liver can be obtained. Liver accumulation is caused by the discontinuous nature of the hepatic vasculature or the lipid metabolism (liposomes and lipid or cholesterol conjugates). In some embodiments, the target organ for RNA expression is liver and the target tissue is liver tissue. The delivery to such target tissue is preferred, in particular, if presence of RNA or of the encoded peptide or polypeptide in this organ or tissue is desired and/or if it is desired to express large amounts of the encoded peptide or polypeptide and/or if systemic presence of the encoded peptide or polypeptide, in particular in significant amounts, is desired or required.

### Delivery Vehicles

To overcome the barriers to safe and effective RNA delivery, RNA may be administered with one or more delivery vehicles that protect the RNA from degradation, maximize delivery to on-target cells and minimize exposure to off-target cells. Such RNA delivery vehicles may complex or encapsulate RNA and include a range of materials, including polymers and lipids. In some embodiments, such RNA delivery vehicles may form particles with RNA.

RNA, in particular mRNA, described herein may be present in particles comprising (i) the RNA, and (ii) at least one cationic or cationically ionizable compound such as a polymer or lipid complexing the RNA. Electrostatic interactions between positively charged molecules such as polymers and lipids and negatively charged RNA are involved in particle formation. This results in complexation and spontaneous formation of RNA particles.

Different types of RNA containing particles have been described previously to be suitable for delivery of RNA in particulate form (cf., e.g., Kaczmarek, J. C. et al., 2017, Genome Medicine 9, 60). For non-viral RNA delivery vehicles, nanoparticle encapsulation of RNA physically protects RNA from degradation and, depending on the specific chemistry, can aid in cellular uptake and endosomal escape.

In the context of the present disclosure, the term “particle” relates to a structured entity formed by molecules or molecule complexes, in particular particle forming compounds. In some embodiments, the particle contains an envelope (e.g., one or more layers or lamellas) made of one or more types of amphiphilic substances (e.g., amphiphilic lipids). In this context, the expression “amphiphilic substance” means that the substance possesses both hydrophilic and lipophilic properties. The envelope may also comprise additional substances (e.g., additional lipids) which do not have to be amphiphilic. Thus, the particle may be a monolamellar or multilamellar structure, wherein the substances constituting the one or more layers or lamellas comprise one or more types of amphiphilic substances (in particular selected from the group consisting of amphiphilic lipids) optionally in combination with additional substances (e.g., additional lipids) which do not have to be amphiphilic. In some embodiments, the term “particle” relates to a micro- or nano-sized structure, such as a micro- or nano-sized compact structure. According to the present disclosure, the term “particle” includes nanoparticles.

An “RNA particle” can be used to deliver RNA to a target site of interest (e.g., cell, tissue, organ, and the like). An RNA particle may be formed from lipids comprising at least one cationic or cationically ionizable lipid. Without intending to be bound by any theory, it is believed that the cationic or cationically ionizable lipid combines together with the RNA to form aggregates, and this aggregation results in colloidally stable particles.

RNA particles described herein include lipid nanoparticle (LNP)-based and lipoplex (LPX)-based formulations.

A lipoplex (LPX) described herein is obtainable from mixing two aqueous phases, namely a phase comprising RNA and a phase comprising a dispersion of lipids. In some embodiments, the lipid phase comprises liposomes.

In some embodiments, liposomes are self-closed unilamellar or multilamellar vesicular particles wherein the lamellae comprise lipid bilayers and the encapsulated lumen comprises an aqueous phase. A prerequisite for using liposomes for nanoparticle formation is that the lipids in the mixture as required are able to form lamellar (bilayer) phases in the applied aqueous environment.

In some embodiments, liposomes comprise unilamellar or multilamellar phospholipid bilayers enclosing an aqueous core (also referred to herein as an aqueous lumen). They may be prepared from materials possessing polar head (hydrophilic) groups and nonpolar tail (hydrophobic) groups. In some embodiments, cationic lipids employed in formulating liposomes designed for the delivery of RNA are amphiphilic in nature and consist of a positively charged (cationic) amine head group linked to a hydrocarbon chain or cholesterol derivative via glycerol.

In some embodiments, lipoplexes are multilamellar liposome-based formulations that form upon electrostatic interaction of cationic liposomes with RNAs. In some embodiments, formed lipoplexes possess distinct internal arrangements of molecules that arise due to the transformation from liposomal structure into compact RNA-lipoplexes.

In some embodiments, an LPX particle comprises an amphiphilic lipid, in particular cationic or cationically ionizable amphiphilic lipid, and RNA (especially mRNA) as described herein. In some embodiments, electrostatic interactions between positively charged liposomes (made from one or more amphiphilic lipids, in particular cationic or cationically ionizable amphiphilic lipids) and negatively charged RNA (especially mRNA) results in complexation and spontaneous formation of RNA lipoplex particles. Positively charged liposomes may be generally synthesized using a cationic or cationically ionizable amphiphilic lipid, such as DOTMA and/or DODMA, and optionally additional lipids, such as DOPE or DSPC. In some embodiments, an RNA (especially mRNA) lipoplex particle is a nanoparticle.

In general, a lipid nanoparticle (LNP) is obtainable from direct mixing of RNA in an aqueous phase with lipids in a phase comprising an organic solvent, such as ethanol. In that case, lipids or lipid mixtures can be used for particle formation, which do not form lamellar (bilayer) phases in water.

In some embodiments, LNPs comprise or consist of a cationic/cationically ionizable lipid and helper lipids such as phospholipids, cholesterol, and/or polymer-conjugated lipids (e.g., polyethylene glycol (PEG) lipids). In some embodiments, in the RNA LNPs described herein the RNA (in particular, mRNA) is bound by cationically ionizable lipid that occupies the central core of the LNP. In some embodiments, polymer-conjugated lipid forms the surface of the LNP, along with phospholipids. In some embodiments, the surface comprises a bilayer. In some embodiments, cholesterol and cationically ionizable lipid in charged and uncharged forms can be distributed throughout the LNP.

In some embodiments, RNA (e.g., mRNA) described herein may be noncovalently associated with a particle as described herein. In embodiments, the RNA (especially mRNA) may be adhered to the outer surface of the particle (surface RNA (especially surface mRNA)) and/or may be contained in the particle (encapsulated RNA (especially encapsulated mRNA)).

In some embodiments, the particles (e.g., LNPs and LPXs) described herein have a size (such as a diameter) in the range of about 10 to about 2000 nm, such as at least about 15 nm (e.g., at least about 20 nm, at least about 25 nm, at least about 30 nm, at least about 35 nm, at least about 40 nm, at least about 45 nm, at least about 50 nm, at least about 55 nm, at least about 60 nm, at least about 65 nm, at least about 70 nm, at least about 75 nm, at least about 80 nm, at least about 85 nm, at least about 90 nm, at least about 95 nm, or at least about 100 nm) and/or at most about 1900 nm (e.g., at most about 1800 nm, at most about 1700 nm, at most about 1600 nm, at most about 1500 nm, at most about 1400 nm, at most about 1300 nm, at most about 1200 nm, at most about 1100 nm, at most about 1000 nm, at most about 950 nm, at most about 900 nm, at most about 850 nm, at most about 800 nm, at most about 750 nm, at most about 700 nm, at most about 650 nm, at most about 600 nm, at most about 550 nm, or at most about 500 nm), such as in the range of about 20 to about 1500 nm, such as about 30 to about 1200 nm, about 40 to about 1100 nm, about 50 to about 1000 nm, about 60 to about 900 nm, about 70 to about 800 nm, about 80 to about 700 nm, about 90 to about 600 nm, or about 50 to about 500 nm or about 100 to about 500 nm, such as in the range of 10 to 1000 nm, 15 to 500 nm, 20 to 450 nm, 25 to 400 nm, 30 to 350 nm, 40 to 300 nm, 50 to 250 nm, 60 to 200 nm, 70 to 150 nm, or 80 to 150 nm. In some embodiments, the particles (e.g., LNPs and LPXs) described herein have a size (such as a diameter) in the range of from about 40 nm to about 200 nm, such as from about 50 nm to about 180 nm, from about 60 nm to about 160 nm, from about 80 nm to about 150 nm or from about 80 nm to about 120 nm.

In some embodiments, the particles (e.g., LNPs and LPXs) described herein have an average diameter that in some embodiments ranges from about 50 nm to about 1000 nm, from about 50 nm to about 800 nm, from about 50 nm to about 700 nm, from about 50 nm to about 600 nm, from about 50 nm to about 500 nm, from about 50 nm to about 450 nm, from about 50 nm to about 400 nm, from about 50 nm to about 350 nm, from about 50 nm to about 300 nm, from about 50 nm to about 250 nm, from about 50 nm to about 200 nm, from about 100 nm to about 1000 nm, from about 100 nm to about 800 nm, from about 100 nm to about 700 nm, from about 100 nm to about 600 nm, from about 100 nm to about 500 nm, from about 100 nm to about 450 nm, from about 100 nm to about 400 nm, from about 100 nm to about 350 nm, from about 100 nm to about 300 nm, from about 100 nm to about 250 nm, from about 100 nm to about 200 nm, from about 150 nm to about 1000 nm, from about 150 nm to about 800 nm, from about 150 nm to about 700 nm, from about 150 nm to about 600 nm, from about 150 nm to about 500 nm, from about 150 nm to about 450 nm, from about 150 nm to about 400 nm, from about 150 nm to about 350 nm, from about 150 nm to about 300 nm, from about 150 nm to about 250 nm, from about 150 nm to about 200 nm, from about 200 nm to about 1000 nm, from about 200 nm to about 800 nm, from about 200 nm to about 700 nm, from about 200 nm to about 600 nm, from about 200 nm to about 500 nm, from about 200 nm to about 450 nm, from about 200 nm to about 400 nm, from about 200 nm to about 350 nm, from about 200 nm to about 300 nm, from about 200 nm to about 250 nm, or from about 80 to about 150 nm. In some embodiments, the particles (e.g., LNPs and LPXs) described herein have an average diameter that in some embodiments ranges from about 40 nm to about 200 nm, such as from about 50 nm to about 180 nm, from about 60 nm to about 160 nm, from about 80 nm to about 150 nm or from about 80 nm to about 120 nm.

In some embodiments, the particles described herein are nanoparticles. The term “nanoparticle” relates to a nano-sized particle comprising nucleic acid (especially mRNA) as described herein and at least one cationic or cationically ionizable lipid, wherein all three external dimensions of the particle are in the nanoscale, i.e., at least about 1 nm and below about 1000 nm. Preferably, the size of a particle is its diameter.

RNA particles (especially mRNA particles) described herein may exhibit a polydispersity index (PDI) less than about 0.5, less than about 0.4, less than about 0.3, less than about 0.2, less than about 0.1, or less than about 0.05. By way of example, the RNA particles can exhibit a polydispersity index in a range of about 0.01 to about 0.4 or about 0.1 to about 0.3.

The N/P ratio gives the ratio of the nitrogen groups in the lipid to the number of phosphate groups in the RNA. It is correlated to the charge ratio, as the nitrogen atoms (depending on the pH) are usually positively charged and the phosphate groups are negatively charged. The N/P ratio, where a charge equilibrium exists, depends on the pH. Lipid formulations are frequently formed at N/P ratios larger than four up to twelve, because positively charged nanoparticles are considered favorable for transfection. In that case, RNA is considered to be completely bound to nanoparticles.

RNA particles (especially mRNA particles) described herein can be prepared using a wide range of methods that may involve obtaining a colloid from at least one cationic or cationically ionizable lipid and mixing the colloid with RNA to obtain RNA particles.

The term “colloid” as used herein relates to a type of homogeneous mixture in which dispersed particles do not settle out. The insoluble particles in the mixture are microscopic, with particle sizes between 1 and 1000 nanometers. The mixture may be termed a colloid or a colloidal suspension. Sometimes the term “colloid” only refers to the particles in the mixture and not the entire suspension.

For the preparation of colloids comprising at least one cationic or cationically ionizable lipid methods are applicable herein that are conventionally used for preparing liposomal vesicles and are appropriately adapted. The most commonly used methods for preparing liposomal vesicles share the following fundamental stages: (i) lipids dissolution in organic solvents, (ii) drying of the resultant solution, and (iii) hydration of dried lipid (using various aqueous media).

In the film hydration method, lipids are firstly dissolved in a suitable organic solvent, and dried down to yield a thin film at the bottom of the flask. The obtained lipid film is hydrated using an appropriate aqueous medium to produce a liposomal dispersion. Furthermore, an additional downsizing step may be included.

Reverse phase evaporation is an alternative method to the film hydration for preparing liposomal vesicles that involves formation of a water-in-oil emulsion between an aqueous phase and an organic phase containing lipids. A brief sonication of this mixture is required for system homogenization. The removal of the organic phase under reduced pressure yields a milky gel that turns subsequently into a liposomal suspension.

The term “ethanol injection technique” refers to a process, in which an ethanol solution comprising lipids is rapidly injected into an aqueous solution through a needle. This action disperses the lipids throughout the solution and promotes lipid structure formation, for example lipid vesicle formation such as liposome formation. Generally, the RNA (especially mRNA) lipoplex particles described herein are obtainable by adding RNA (especially mRNA) to a colloidal liposome dispersion. Using the ethanol injection technique, such colloidal liposome dispersion is, in some embodiments, formed as follows: an ethanol solution comprising lipids, such as cationic or cationically ionizable lipids (like DOTMA and/or DODMA) and additional lipids, is injected into an aqueous solution under stirring. In some embodiments, the RNA (especially mRNA) lipoplex particles described herein are obtainable without a step of extrusion.

The term “extruding” or “extrusion” refers to the creation of particles having a fixed, cross-sectional profile. In particular, it refers to the downsizing of a particle, whereby the particle is forced through filters with defined pores. Other methods having organic solvent free characteristics may also be used according to the present disclosure for preparing a colloid.

In some embodiments, LNPs comprise four components: cationically ionizable lipids, neutral lipids such as phospholipids, a steroid such as cholesterol, and a polymer-conjugated lipid. In some embodiments, LNPs may be prepared by mixing lipids dissolved in ethanol rapidly with RNA in an aqueous buffer. While RNA particles described herein may comprise polymer-conjugated lipids such as PEG lipids, provided herein are also RNA particles which do not comprise PEG lipids, or do not comprise any polymer-conjugated lipids.

In some embodiments, the LNPs comprising RNA and at least one cationic or cationically ionizable lipid described herein are prepared by (a) preparing an RNA solution containing water and a buffering system; (b) preparing an ethanolic solution comprising the cationic or cationically ionizable lipid and, if present, one or more additional lipids; and (c) mixing the RNA solution prepared under (a) with the ethanolic solution prepared under (b), thereby preparing the formulation comprising LNPs. After step (c) one or more steps selected from diluting and filtrating, such as tangential flow filtrating, can follow.

In some embodiments, the LNPs comprising RNA and at least one cationic or cationically ionizable lipid described herein are prepared by (a′) preparing liposomes or a colloidal preparation of the cationic or cationically ionizable lipid and, if present, one or more additional lipids in an aqueous phase; and (b′) preparing an RNA solution containing water and a buffering system; and (c′) mixing the liposomes or colloidal preparation prepared under (a′) with the RNA solution prepared under (b′). After step (c′) one or more steps selected from diluting and filtrating, such as tangential flow filtrating, can follow.

The present disclosure describes compositions comprising RNA (especially mRNA) and at least one cationic or cationically ionizable lipid which associates with the RNA to form RNA particles and formulations comprising such particles. The RNA particles may comprise RNA which is complexed in different forms by non-covalent interactions to the particle. The particles described herein are not viral particles, in particular infectious viral particles, i.e., they are not able to virally infect cells.

Suitable cationic or cationically ionizable lipids are those that form RNA particles and are included by the term “particle forming components” or “particle forming agents”. The term “particle forming components” or “particle forming agents” relates to any components which associate with RNA to form RNA particles. Such components include any component which can be part of RNA particles.

In some embodiments, RNA particles (especially mRNA particles) comprise more than one type of RNA molecules, where the molecular parameters of the RNA molecules may be similar or different from each other, like with respect to molar mass or fundamental structural elements such as molecular architecture, capping, coding regions or other features,

In particulate formulation, it is possible that each RNA species is separately formulated as an individual particulate formulation. In that case, each individual particulate formulation will comprise one RNA species. The individual particulate formulations may be present as separate entities, e.g. in separate containers. Such formulations are obtainable by providing each RNA species separately (typically each in the form of an RNA-containing solution) together with a particle-forming agent, thereby allowing the formation of particles. Respective particles will contain exclusively the specific RNA species that is being provided when the particles are formed (individual particulate formulations). In some embodiments, a composition such as a pharmaceutical composition comprises more than one individual particle formulation. Respective pharmaceutical compositions are referred to as mixed particulate formulations. Mixed particulate formulations according to the present disclosure are obtainable by forming, separately, individual particulate formulations, followed by a step of mixing of the individual particulate formulations. By the step of mixing, a formulation comprising a mixed population of RNA-containing particles is obtainable. Individual particulate populations may be together in one container, comprising a mixed population of individual particulate formulations. Alternatively, it is possible that all RNA species of the pharmaceutical composition are formulated together as a combined particulate formulation. Such formulations are obtainable by providing a combined formulation (typically combined solution) of all RNA species together with a particle-forming agent, thereby allowing the formation of particles. As opposed to a mixed particulate formulation, a combined particulate formulation will typically comprise particles which comprise more than one RNA species. In a combined particulate composition different RNA species are typically present together in a single particle.

### Polymers

Given their high degree of chemical flexibility, polymers are commonly used materials for nanoparticle-based delivery. Typically, cationic polymers are used to electrostatically condense the negatively charged RNA into nanoparticles. These positively charged groups often consist of amines that change their state of protonation in the pH range between 5.5 and 7.5, thought to lead to an ion imbalance that results in endosomal rupture. Polymers such as poly-L-lysine, polyamidoamine, protamine and polyethyleneimine, as well as naturally occurring polymers such as chitosan have all been applied to nucleic acid delivery and are suitable as cationic polymers herein. In addition, some investigators have synthesized polymers specifically for nucleic acid delivery. Poly(β-amino esters), in particular, have gained widespread use in nucleic acid delivery owing to their ease of synthesis and biodegradability. Such synthetic polymers are also suitable as cationic polymers herein.

A “polymer,” as used herein, is given its ordinary meaning, i.e., a molecular structure comprising one or more repeat units (monomers), connected by covalent bonds. The repeat units can all be identical, or in some cases, there can be more than one type of repeat unit present within the polymer. In some cases, the polymer is biologically derived, i.e., a biopolymer such as a protein. In some cases, additional moieties can also be present in the polymer, for example targeting moieties.

If more than one type of repeat unit is present within the polymer, then the polymer is said to be a “copolymer.” It is to be understood that the polymer being employed herein can be a copolymer. The repeat units forming the copolymer can be arranged in any fashion. For example, the repeat units can be arranged in a random order, in an alternating order, or as a “block” copolymer, i.e., comprising one or more regions each comprising a first repeat unit (e.g., a first block), and one or more regions each comprising a second repeat unit (e.g., a second block), etc. Block copolymers can have two (a diblock copolymer), three (a triblock copolymer), or more numbers of distinct blocks.

In certain embodiments, the polymer is biocompatible. Biocompatible polymers are polymers that typically do not result in significant cell death at moderate concentrations. In certain embodiments, the biocompatible polymer is biodegradable, i.e., the polymer is able to degrade, chemically and/or biologically, within a physiological environment, such as within the body.

In certain embodiments, polymer may be protamine or polyalkyleneimine.

The term “protamine” refers to any of various strongly basic proteins of relatively low molecular weight that are rich in arginine and are found associated especially with DNA in place of somatic histones in the sperm cells of various animals (as fish). In particular, the term “protamine” refers to proteins found in fish sperm that are strongly basic, are soluble in water, are not coagulated by heat, and yield chiefly arginine upon hydrolysis. In purified form, they are used in a long-acting formulation of insulin and to neutralize the anticoagulant effects of heparin.

According to the disclosure, the term “protamine” as used herein is meant to comprise any protamine amino acid sequence obtained or derived from natural or biological sources including fragments thereof and multimeric forms of said amino acid sequence or fragment thereof as well as (synthesized) polypeptides which are artificial and specifically designed for specific purposes and cannot be isolated from native or biological sources.

In one embodiment, the polyalkyleneimine comprises polyethylenimine and/or polypropylenimine, preferably polyethyleneimine. A preferred polyalkyleneimine is polyethyleneimine (PEI). The average molecular weight of PEI is preferably 0.75·102 to 107 Da, preferably 1000 to 105 Da, more preferably 10000 to 40000 Da, more preferably 15000 to 30000 Da, even more preferably 20000 to 25000 Da.

Preferred according to the disclosure is linear polyalkyleneimine such as linear polyethyleneimine (PEI).

Cationic polymers (including polycationic polymers) contemplated for use herein include any cationic polymers which are able to electrostatically bind nucleic acid. In one embodiment, cationic polymers contemplated for use herein include any cationic polymers with which nucleic acid can be associated, e.g. by forming complexes with the nucleic acid or forming vesicles in which the nucleic acid is enclosed or encapsulated.

Particles described herein may also comprise polymers other than cationic polymers, i.e., non-cationic polymers and/or anionic polymers. Collectively, anionic and neutral polymers are referred to herein as non-cationic polymers.

### Lipids

The terms “lipid” and “lipid-like material” are broadly defined herein as molecules which comprise one or more hydrophobic moieties or groups and optionally also one or more hydrophilic moieties or groups. Molecules comprising hydrophobic moieties and hydrophilic moieties are also frequently denoted as amphiphiles. Lipids are usually insoluble or poorly soluble in water, but soluble in many organic solvents. In an aqueous environment, the amphiphilic nature allows the molecules to self-assemble into organized structures and different phases. One of those phases consists of lipid bilayers, as they are present in vesicles, multilamellar/unilamellar liposomes, or membranes in an aqueous environment. Hydrophobicity can be conferred by the inclusion of apolar groups that include, but are not limited to, long-chain saturated and unsaturated aliphatic hydrocarbon groups and such groups substituted by one or more aromatic, cycloaliphatic, or heterocyclic group(s). The hydrophilic groups may comprise polar and/or charged groups and include carbohydrates, phosphate, carboxylic, sulfate, amino, sulfhydryl, nitro, hydroxyl, and other like groups.

As used herein, the term “hydrophobic” refers to any a molecule, moiety or group which is substantially immiscible or insoluble in aqueous solution. The term hydrophobic group includes hydrocarbons having at least 6 carbon atoms. The monovalent radical of a hydrocarbon is referred to as hydrocarbyl herein. The hydrophobic group can have functional groups (e.g., ether, ester, halide, etc.) and atoms other than carbon and hydrogen as long as the group satisfies the condition of being substantially immiscible or insoluble in aqueous solution.

The term “hydrocarbon” includes non-cyclic, e.g., linear (straight) or branched, hydrocarbyl groups, such as alkyl, alkenyl, or alkynyl as defined herein. It should be appreciated that one or more of the hydrogen atoms in alkyl, alkenyl, or alkynyl may be substituted with other atoms, e.g., halogen, oxygen or sulfur. Unless stated otherwise, hydrocarbon groups can also include a cyclic (alkyl, alkenyl or alkynyl) group or an aryl group, provided that the overall polarity of the hydrocarbon remains relatively nonpolar.

The term “alkyl” refers to a saturated linear or branched monovalent hydrocarbon moiety which may have one to thirty, typically one to twenty, often six to eighteen carbon atoms. Exemplary nonpolar alkyl groups include, but are not limited to, methyl, ethyl, propyl, isopropyl, hexyl, decyl, dodecyl, tetradecyl, hexadecyl, octadecyl, and the like.

The term “alkenyl” refers to a linear or branched monovalent hydrocarbon moiety having at least one carbon-carbon double bond in which the total carbon atoms may be six to thirty, typically six to twenty often six to eighteen. Generally, the maximal number of carbon-carbon double bonds in the alkenyl group can be equal to the integer which is calculated by dividing the number of carbon atoms in the alkenyl group by 2 and, if the number of carbon atoms in the alkenyl group is uneven, rounding the result of the division down to the next integer. For example, for an alkenyl group having 9 carbon atoms, the maximum number of carbon-carbon double bonds is 4. Preferably, the alkenyl group has 1 to 6 (such as 1 to 4), i.e., 1, 2, 3, 4, 5, or 6, carbon-carbon double bonds.

The term “alkynyl” refers to a linear or branched monovalent hydrocarbon moiety having at least one carbon-carbon triple bond in which the total carbon atoms may be six to thirty, typically six to twenty, often six to eighteen. Alkynyl groups can optionally have one or more carbon-carbon double bonds. Generally, the maximal number of carbon-carbon triple bonds in the alkynyl group can be equal to the integer which is calculated by dividing the number of carbon atoms in the alkynyl group by 2 and, if the number of carbon atoms in the alkynyl group is uneven, rounding the result of the division down to the next integer. For example, for an alkynyl group having 9 carbon atoms, the maximum number of carbon-carbon triple bonds is 4. Preferably, the alkynyl group has 1 to 6 (such as 1 to 4), i.e., 1, 2, 3, 4, 5, or 6, more preferably 1 or 2 carbon-carbon triple bonds.

The term “alkylene” refers to a saturated linear or branched divalent hydrocarbon moiety which may have one to thirty, typically two to twenty, often four to twelve carbon atoms. Exemplary nonpolar alkylene groups include, but are not limited to, methylene, ethylene, trimethylene, hexamethylene, decamethylene, dodecamethylene, tetradecamethylene, hexadecamethylene, octadecmethylene, and the like.

The term “alkenylene” refers to a linear or branched divalent hydrocarbon moiety having at least one carbon-carbon double bond in which the total carbon atoms may be two to thirty, typically two to twenty, often four to twelve. Generally, the maximal number of carbon-carbon double bonds in the alkenylene group can be equal to the integer which is calculated by dividing the number of carbon atoms in the alkenylene group by 2 and, if the number of carbon atoms in the alkenylene group is uneven, rounding the result of the division down to the next integer. For example, for an alkenylene group having 9 carbon atoms, the maximum number of carbon-carbon double bonds is 4. Preferably, the alkenylene group has 1 to 6 (such as 1 to 4), i.e., 1, 2, 3, 4, 5, or 6, carbon-carbon double bonds.

The term “cycloalkyl” represents cyclic non-aromatic versions of “alkyl” and “alkenyl” with preferably 3 to 14 carbon atoms, such as 3 to 12 or 3 to 10 carbon atoms, i.e., 3, 4, 5, 6, 7, 8, 9, 10, 11, 12, 13, or 14 carbon atoms (such as 3, 4, 5, 6, 7, 8, 9, or 10 carbon atoms), more preferably 3 to 7 carbon atoms. Exemplary cycloalkyl groups include cyclopropyl, cyclopropenyl, cyclobutyl, cyclobutenyl, cyclopentyl, cyclopentenyl, cyclohexyl, cyclohexenyl, cycloheptyl, cycloheptenyl, cyclooctyl, cyclooctenyl, cyclononyl, cyclononenyl, cylcodecyl, cylcodecenyl, and adamantyl. The cycloalkyl group may consist of one ring (monocyclic), two rings (bicyclic), or more than two rings (polycyclic).

The term “aryl” refers to a monoradical of an aromatic cyclic hydrocarbon. Preferably, the aryl group contains 3 to 14 (e.g., 5, 6, 7, 8, 9, or 10, such as 5, 6, or 10) carbon atoms which can be arranged in one ring (e.g., phenyl) or two or more condensed rings (e.g., naphthyl). Exemplary aryl groups include cyclopropenylium, cyclopentadienyl, phenyl, indenyl, naphthyl, azulenyl, fluorenyl, anthryl, and phenanthryl. Preferably, “aryl” refers to a monocyclic ring containing 6 carbon atoms or an aromatic bicyclic ring system containing 10 carbon atoms. Preferred examples are phenyl and naphthyl. Aryl does not encompass fullerenes.

The term “aromatic” as used in the context of hydrocarbons means that the whole molecule has to be aromatic. For example, if a monocyclic aryl is hydrogenated (either partially or completely) the resulting hydrogenated cyclic structure is classified as cycloalkyl for the purposes of the present disclosure. Likewise, if a bi- or polycyclic aryl (such as naphthyl) is hydrogenated the resulting hydrogenated bi- or polycyclic structure (such as 1,2-dihydronaphthyl) is classified as cycloalkyl for the purposes of the present disclosure (even if one ring, such as in 1,2-dihydronaphthyl, is still aromatic).

As used herein, the term “amphiphilic” refers to a molecule having both a polar portion and a non-polar portion. Often, an amphiphilic compound has a polar head attached to a long hydrophobic tail. In some embodiments, the polar portion is soluble in water, while the non-polar portion is insoluble in water. In addition, the polar portion may have either a formal positive charge, or a formal negative charge. Alternatively, the polar portion may have both a formal positive and a negative charge, and be a zwitterion or inner salt. For purposes of the disclosure, the amphiphilic compound can be, but is not limited to, one or a plurality of natural or non-natural lipids and lipid-like compounds.

The term “lipid-like material”, “lipid-like compound” or “lipid-like molecule” relates to substances, in particular amphiphilic substances, that structurally and/or functionally relate to lipids but may not be considered as lipids in a strict sense. For example, the term includes compounds that are able to form amphiphilic layers as they are present in vesicles, multilamellar/unilamellar liposomes, or membranes in an aqueous environment and includes surfactants, or synthesized compounds with both hydrophilic and hydrophobic moieties. Generally speaking, the term includes molecules, which comprise hydrophilic and hydrophobic moieties with different structural organization, which may or may not be similar to that of lipids. Examples of lipid-like compounds capable of spontaneous integration into cell membranes include functional lipid constructs such as synthetic function-spacer-lipid constructs (FSL), synthetic function-spacer-sterol constructs (FSS) as well as artificial amphipathic molecules. Lipids comprising two long alkyl chains and a polar head group are generally cylindrical. The area occupied by the two alkyl chains is similar to the area occupied by the polar head group. Such lipids have low solubility as monomers and tend to aggregate into planar bilayers that are water insoluble. Traditional surfactant monomers comprising only one linear alkyl chain and a hydrophilic head group are generally cone shaped. The hydrophilic head group tends to occupy more molecular space than the linear alkyl chain. In some embodiments, surfactants tend to aggregate into spherical or elliptoid micelles that are water soluble. While lipids also have the same general structure as surfactants—a polar hydrophilic head group and a nonpolar hydrophobic tail—lipids differ from surfactants in the shape of the monomers, in the type of aggregates formed in solution, and in the concentration range required for aggregation. As used herein, the term “lipid” is to be construed to cover both lipids and lipid-like materials unless otherwise indicated herein or clearly contradicted by context.

Generally, lipids may be divided into eight categories: fatty acids, glycerolipids, glycerophospholipids, sphingolipids, saccharolipids, polyketides (derived from condensation of ketoacyl subunits), sterol lipids and prenol lipids (derived from condensation of isoprene subunits). Although the term “lipid” is sometimes used as a synonym for fats, fats are a subgroup of lipids called triglycerides. Lipids also encompass molecules such as fatty acids and their derivatives (including tri-, di-, monoglycerides, and phospholipids), as well as steroids, i.e., sterol-containing metabolites such as cholesterol or a derivative thereof. Examples of cholesterol derivatives include, but are not limited to, cholestanol, cholestanone, cholestenone, coprostanol, cholesteryl-2′-hydroxyethyl ether, cholesteryl-4′-hydroxybutyl ether, tocopherol and derivatives thereof, and mixtures thereof.

Fatty acids, or fatty acid residues are a diverse group of molecules made of a hydrocarbon chain that terminates with a carboxylic acid group; this arrangement confers the molecule with a polar, hydrophilic end, and a nonpolar, hydrophobic end that is insoluble in water. The carbon chain, typically between four and 24 carbons long, may be saturated or unsaturated, and may be attached to functional groups containing oxygen, halogens, nitrogen, and sulfur. If a fatty acid contains a double bond, there is the possibility of either a cis or trans geometric isomerism, which significantly affects the molecule's configuration. Cis-double bonds cause the fatty acid chain to bend, an effect that is compounded with more cis double bonds in the chain. Other major lipid classes in the fatty acid category are the fatty esters and fatty amides.

Glycerolipids are composed of mono-, di-, and tri-substituted glycerols, the best-known being the fatty acid triesters of glycerol, called triglycerides. The word “triacylglycerol” is sometimes used synonymously with “triglyceride”. In these compounds, the three hydroxyl groups of glycerol are each esterified, typically by different fatty acids. Additional subclasses of glycerolipids are represented by glycosylglycerols, which are characterized by the presence of one or more sugar residues attached to glycerol via a glycosidic linkage.

The glycerophospholipids are amphipathic molecules (containing both hydrophobic and hydrophilic regions) that contain a glycerol core linked to two fatty acid-derived “tails” by ester linkages and to one “head” group by a phosphate ester linkage. Examples of glycerophospholipids, usually referred to as phospholipids (though sphingomyelins are also classified as phospholipids) are phosphatidylcholine (also known as PC, GPCho or lecithin), phosphatidylethanolamine (PE or GPEtn) and phosphatidylserine (PS or GPSer).

Sphingolipids are a complex family of compounds that share a common structural feature, a sphingoid base backbone. The major sphingoid base in mammals is commonly referred to as sphingosine. Ceramides (N-acyl-sphingoid bases) are a major subclass of sphingoid base derivatives with an amide-linked fatty acid. The fatty acids are typically saturated or mono-unsaturated with chain lengths from 16 to 26 carbon atoms. The major phosphosphingolipids of mammals are sphingomyelins (ceramide phosphocholines), whereas insects contain mainly ceramide phosphoethanolamines and fungi have phytoceramide phosphoinositols and mannose-containing headgroups. The glycosphingolipids are a diverse family of molecules composed of one or more sugar residues linked via a glycosidic bond to the sphingoid base. Examples of these are the simple and complex glycosphingolipids such as cerebrosides and gangliosides.

Sterol lipids, such as cholesterol and its derivatives, or tocopherol and its derivatives, are an important component of membrane lipids, along with the glycerophospholipids and sphingomyelins.

Saccharolipids describe compounds in which fatty acids are linked directly to a sugar backbone, forming structures that are compatible with membrane bilayers. In the saccharolipids, a monosaccharide substitutes for the glycerol backbone present in glycerolipids and glycerophospholipids. The most familiar saccharolipids are the acylated glucosamine precursors of the Lipid A component of the lipopolysaccharides in Gram-negative bacteria. Typical lipid A molecules are disaccharides of glucosamine, which are derivatized with as many as seven fatty-acyl chains. The minimal lipopolysaccharide required for growth in E. coli is Kdo2-Lipid A, a hexa-acylated disaccharide of glucosamine that is glycosylated with two 3-deoxy-D-manno-octulosonic acid (Kdo) residues.

Polyketides are synthesized by polymerization of acetyl and propionyl subunits by classic enzymes as well as iterative and multimodular enzymes that share mechanistic features with the fatty acid synthases. They comprise a large number of secondary metabolites and natural products from animal, plant, bacterial, fungal and marine sources, and have great structural diversity. Many polyketides are cyclic molecules whose backbones are often further modified by glycosylation, methylation, hydroxylation, oxidation, or other processes.

According to the disclosure, lipids and lipid-like materials may be cationic, anionic or neutral. Neutral lipids or lipid-like materials exist in an uncharged or neutral zwitterionic form at a selected pH.

### Cationic/Cationically Ionizable Lipids

In some embodiments, the RNA compositions and formulations and RNA particles described herein comprise at least one cationic or cationically ionizable lipid as particle forming agent. Cationic or cationically ionizable lipids contemplated for use herein include any cationic or cationically ionizable lipids (including lipid-like materials) which are able to electrostatically bind nucleic acid. In some embodiments, cationic or cationically ionizable lipids contemplated for use herein can be associated with nucleic acid, e.g. by forming complexes with the nucleic acid or forming vesicles in which the nucleic acid is enclosed or encapsulated.

As used herein, a “cationic lipid” refers to a lipid or lipid-like material having a net positive charge. Cationic lipids bind negatively charged nucleic acid by electrostatic interaction. Generally, cationic lipids possess a lipophilic moiety, such as a sterol, an acyl chain, a diacyl or more acyl chains, and the head group of the lipid typically carries the positive charge.

In some embodiments, a cationic lipid has a net positive charge only at certain pH, in particular acidic pH, while it has preferably no net positive charge, preferably has no charge, i.e., it is neutral, at a different, preferably higher pH such as physiological pH. This ionizable behavior is thought to enhance efficacy through helping with endosomal escape and reducing toxicity as compared with particles that remain cationic at physiological pH.

As used herein, a “cationically ionizable lipid” refers to a lipid or lipid-like material which has a net positive charge or is neutral, i.e., which is not permanently cationic. Thus, depending on the pH of the composition in which the cationically ionizable lipid is solved, the cationically ionizable lipid is either positively charged or neutral. For purposes of the present disclosure, cationically ionizable lipids are covered by the term “cationic lipid” unless contradicted by the circumstances.

In some embodiments, the cationic or cationically ionizable lipid comprises a head group which includes at least one nitrogen atom (N) which is positive charged or capable of being protonated, e.g., under physiological conditions.

Examples of cationic or cationically ionizable lipids include, but are not limited to N,N-dimethyl-2,3-dioleyloxypropylamine (DODMA), 1,2-dioleoyl-3-trimethylammonium propane (DOTAP); 1,2-di-O-octadecenyl-3-trimethylammonium propane (DOTMA), 3-(N—(N′,N′-dimethylaminoethane)-carbamoyl) cholesterol (DC-Chol), dimethyldioctadecylammonium (DDAB); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-distearyloxy-N,N-dimethyl-3-aminopropane (DSDMA), 2,3-di(tetradecoxy) propyl-(2-hydroxyethyl)-dimethylazanium (DMRIE), 1,2-dimyristoyl-sn-glycero-3-ethylphosphocholine (DMEPC), 1,2-dimyristoyl-3-trimethylammonium propane (DMTAP), 1,2-dioleyloxypropyl-3-dimethyl-hydroxyethyl ammonium bromide (DORIE), and 2,3-dioleoyloxy-N-[2 (spermine carboxamide)ethyl]-N,N-dimethyl-I-propanamium trifluoroacetate (DOSPA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-1-(cis,cis-9,12-oc-tadecadienoxy) propane (CLinDMA), 2-[5′-(cholest-5-en-3-beta-oxy)-3′-oxapentoxy)-3-dimethyl-1-(cis,cis-9′,12′-octadecadienoxy) propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino)butanoate (DLin-MC3-DMA), N-(2-Hydroxyethyl)-N,N-dimethyl-2,3-bis(tetradecyloxy)-1-propanaminium bromide (DMRIE), (±)-N-(3-aminopropyl)-N,N-dimethyl-2,3-bis(cis-9-tetradecenyloxy)-1-propanaminium (GAP-DMORIE), (±)-N-(3-aminopropyl)-N,N-dimethyl-2,3-bis(dodecyloxy)-1-propanaminium bromide bromide (GAP-DLRIE), (±)-N-(3-aminopropyl)-N,N-dimethyl-2,3-bis(tetradecyloxy)-1-propanaminium bromide (GAP-DMRIE), N-(2-Aminoethyl)-N,N-dimethyl-2,3-bis(tetradecyloxy)-1-propanaminium bromide (BAE-DMRIE), N-(4-carboxybenzyl)-N,N-dimethyl-2,3-bis(oleoyloxy) propan-1-aminium (DOBAQ), 2-({8-[(3B)-cholest-5-en-3-yloxy]octyl}oxy)-N,N-dimethyl-3-[(9Z,12Z)-octadeca-9,12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), N¹-[2-((1S)-1-[(3-aminopropyl)amino]-4-[di(3-amino-propyl)amino]butylcarboxamido)ethyl]-3,4-di [oleyloxy]-benzamide (MVL5), 1,2-dioleoyl-sn-glycero-3-ethylphosphocholine (DOEPC), 2,3-bis(dodecyloxy)-N-(2-hydroxyethyl)-N,N-dimethylpropan-1-ammonium bromide (DLRIE), N-(2-aminoethyl)-N,N-dimethyl-2,3-bis(tetradecyloxy) propan-1-aminium bromide (DMORIE), di((Z)-non-2-en-1-yl) 8,8′-((((2 (dimethylamino)ethyl)thio) carbonyl) azanediyl)dioctanoate (ATX), N,N-dimethyl-2,3-bis(dodecyloxy) propan-1-amine (DLDMA), N,N-dimethyl-2,3-bis(tetradecyloxy) propan-1-amine (DMDMA), Di((Z)-non-2-en-1-yl)-9-((4-(dimethylaminobutanoyl)oxy) heptadecanedioate (L319), N-Dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethylamino) propionamide (lipidoid 98N₁₂-5), 1-[2-[bis(2-hydroxydodecyl)amino]ethyl-[2-[4-[2-[bis(2 hydroxydodecyl)amino]ethyl]piperazin-1-yl]ethyl]amino]dodecan-2-ol (lipidoid C12-200).

In some embodiments, the cationic or cationically ionizable lipid is DOTMA. In some embodiments, the cationic or cationically ionizable lipid is DODMA.

DOTMA is a cationic lipid with a quaternary amine headgroup. The structure of DOTMA may be represented as follows:

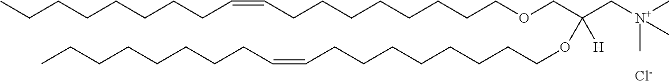 [SMILES: CCCCCCCC/C=C\CCCCCCCCOCC(C[N+](C)(C)C)OCCCCCCCC/C=C\CCCCCCCC ; [Cl-]]

DODMA is an ionizable cationic lipid with a tertiary amine headgroup. The structure of DODMA may be represented as follows:

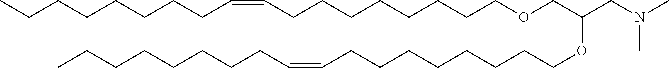 [SMILES: CCCCCCCC/C=C\CCCCCCCCOCC(CN(C)C)OCCCCCCCC/C=C\CCCCCCCC]

In some embodiments, the cationic or cationically ionizable lipid may comprise from about 10 mol % to about 95 mol %, from about 20 mol % to about 95 mol %, from about 20 mol % to about 90 mol %, from about 30 mol % to about 90 mol %, from about 40 mol % to about 90 mol %, or from about 40 mol % to about 80 mol % of the total lipid present in the particle.

### Additional Lipids

The RNA compositions and formulations and RNA particles described herein may also comprise lipids (including lipid-like materials) other than cationic or cationically ionizable lipids (also collectively referred to herein as cationic lipids), i.e., non-cationic lipids (including non-cationic or non-cationically ionizable lipids or lipid-like materials). Collectively, anionic and neutral lipids or lipid-like materials are referred to herein as non-cationic lipids. Optimizing the formulation of RNA particles by addition of other hydrophobic moieties, such as cholesterol and lipids, in addition to a cationic or cationically ionizable lipid may enhance particle stability and efficacy of RNA delivery.

One or more additional lipids may or may not affect the overall charge of the RNA particles. In some embodiments, the one or more additional lipids are a non-cationic lipid or lipid-like material. The non-cationic lipid may comprise, e.g., one or more anionic lipids and/or neutral lipids. As used herein, an “anionic lipid” refers to any lipid that is negatively charged at a selected pH. As used herein, a “neutral lipid” refers to any of a number of lipid species that exist either in an uncharged or neutral zwitterionic form at a selected pH.

In some embodiments, the RNA compositions and formulations and RNA particles described herein comprise a cationic or cationically ionizable lipid and one or more additional lipids.

Without wishing to be bound by theory, the amount of the cationic or cationically ionizable lipid compared to the amount of the one or more additional lipids may affect important RNA particle characteristics, such as charge, particle size, stability, tissue selectivity, and bioactivity of the RNA. Accordingly, in some embodiments, the molar ratio of the cationic or cationically ionizable lipid to the one or more additional lipids is from about 10:0 to about 1:9, about 4:1 to about 1:2, about 4:1 to about 1:1, about 3:1 to about 1:1, or about 3:1 to about 2:1.

In some embodiments, the one or more additional lipids comprised in the RNA compositions and formulations and RNA particles described herein comprise one or more of the following: neutral lipids, steroids, and combinations thereof.

In some embodiments, the one or more additional lipids comprise a neutral lipid which is a phospholipid. In some embodiments, the phospholipid is selected from the group consisting of phosphatidylcholines, phosphatidylethanolamines, phosphatidylglycerols, phosphatidic acids, phosphatidylserines and sphingomyelins. Specific phospholipids that can be used include, but are not limited to, phosphatidylcholines, phosphatidylethanolamines, phosphatidylglycerols, phosphatidic acids, phosphatidylserines or sphingomyelin. Such phospholipids include in particular diacylphosphatidylcholines, such as distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC) and phosphatidylethanolamines, in particular diacylphosphatidylethanolamines, such as dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), diphytanoyl-phosphatidylethanolamine (DPyPE), 1,2-di-(9Z-octadecenoyl)-sn-glycero-3-phosphocholine (DOPG), 1,2-dipalmitoyl-sn-glycero-3-phospho-(1′-rac-glycerol) (DPPG), 1-palmitoyl-2-oleoyl-sn-glycero-3-phosphoethanolamine (POPE), N-palmitoyl-D-erythro-sphingosylphosphorylcholine (SM), and further phosphatidylethanolamine lipids with different hydrophobic chains. In some embodiments, the neutral lipid is selected from the group consisting of DSPC, DOPC, DMPC, DPPC, POPC, DOPE, DOPG, DPPG, POPE, DPPE, DMPE, DSPE, and SM. In some embodiments, the neutral lipid is selected from the group consisting of DSPC, DPPC, DMPC, DOPC, POPC, DOPE and SM. In some embodiments, the neutral lipid is DSPC. In some embodiments, the neutral lipid is DOPE.

In some embodiments, the additional lipid comprises one of the following: (1) a phospholipid, (2) cholesterol or a derivative thereof; or (3) a mixture of a phospholipid and cholesterol or a derivative thereof. Examples of cholesterol derivatives include, but are not limited to, cholestanol, cholestanone, cholestenone, coprostanol, cholesteryl-2′-hydroxyethyl ether, cholesteryl-4′-hydroxybutyl ether, tocopherol and derivatives thereof, and mixtures thereof. Thus, in some embodiments, the RNA compositions and formulations and RNA particles described herein comprise (1) a cationic or cationically ionizable lipid, and a phospholipid such as DSPC or DOPE or (2) a cationic or cationically ionizable lipid and a phospholipid such as DSPC or DOPE and cholesterol.

In some embodiments, the RNA particles (especially the particles comprising mRNA) described herein comprise (1) DOTMA and DOPE, (2) DOTMA, DOPE and cholesterol, (3) DODMA and DOPE or (4) DODMA, DOPE and cholesterol.

DSPC is a neutral phospholipid. The structure of DSPC may be represented as follows:

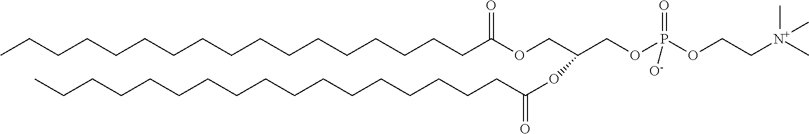 [SMILES: CCCCCCCCCCCCCCCCCC(=O)OC[C@H](COP(=O)([O-])OCC[N+](C)(C)C)OC(=O)CCCCCCCCCCCCCCCCC]

DOPE is a neutral phospholipid. The structure of DOPE may be represented as follows:

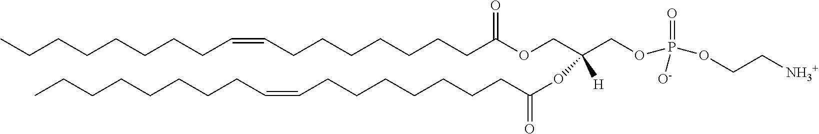 [SMILES: CCCCCCCC/C=C\CCCCCCCC(=O)OC[C@H](COP(=O)([O-])OCC[NH3+])OC(=O)CCCCCCC/C=C\CCCCCCCC]

The structure of cholesterol may be represented as follows:

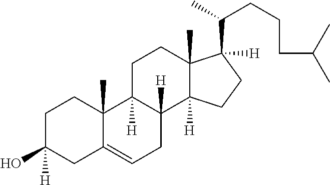

In some embodiments, RNA compositions and formulations and RNA particles described herein do not include a polymer conjugated lipid such as a pegylated lipid. The term “pegylated lipid” refers to a molecule comprising both a lipid portion and a polyethylene glycol portion. Pegylated lipids are known in the art.

In some embodiments, the additional lipid (e.g., one or more phospholipids and/or cholesterol) may comprise from about 0 mol % to about 90 mol %, from about 0 mol % to about 80 mol %, from about 2 mol % to about 80 mol %, from about 5 mol % to about 80 mol %, from about 5 mol % to about 60 mol %, from about 5 mol % to about 50 mol %, from about 7.5 mol % to about 50 mol %, or from about 10 mol % to about 40 mol % of the total lipid present in the particle. In some embodiments, the additional lipid (e.g., one or more phospholipids and/or cholesterol) comprises about 10 mol %, about 15 mol %, or about 20 mol % of the total lipid present in the particle.

In some embodiments, the additional lipid comprises a mixture of: (i) a phospholipid such as DOPE; and (ii) cholesterol or a derivative thereof. In some embodiments, the molar ratio of the phospholipid such as DOPE to the cholesterol or a derivative thereof is from about 9:0 to about 1:10, about 2:1 to about 1:4, about 1:1 to about 1:4, or about 1:1 to about 1:3.

### Polymer-Conjugated Lipids

In some embodiments, RNA compositions and formulations and RNA particles described herein may comprise at least one polymer-conjugated lipid. A polymer-conjugated lipid is typically a molecule comprising a lipid portion and a polymer portion conjugated thereto. In some embodiments, a polymer-conjugated lipid is a PEG-conjugated lipid, also referred to herein as pegylated lipid or PEG-lipid. The term “pegylated lipid” refers to a molecule comprising both a lipid portion and a polyethylene glycol portion. Pegylated lipids are known in the art. In some embodiments, a polymer-conjugated lipid is a polysarcosine-conjugated lipid, also referred to herein as sarcosinylated lipid or pSar-lipid. The term “sarcosinylated lipid” refers to a molecule comprising both a lipid portion and a polysarcosine portion.

In some embodiments, a polymer-conjugated lipid is designed to sterically stabilize a lipid particle by forming a protective hydrophilic layer that shields the hydrophobic lipid layer. In some embodiments, a polymer-conjugated lipid can reduce its association with serum proteins and/or the resulting uptake by the reticuloendothelial system when such lipid particles are administered in vivo.

### Polyethyleneglycol (PEG)-Conjugated Lipids

In some embodiments, RNA compositions/formulations and RNA particles described herein comprise a PEG-conjugated lipid.

In some embodiments, the PEG-conjugated lipid (pegylated lipid) is a lipid having the structure of the following general formula:

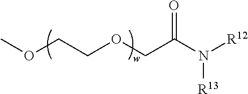

or a pharmaceutically acceptable salt, tautomer or stereoisomer thereof, wherein:
each of R¹² and R¹³ is each independently a straight or branched, alkyl or alkenyl chain containing from 10 to 30 carbon atoms, wherein the alkyl/alkenyl chain is optionally interrupted by one or more ester bonds; and w has a mean value ranging from 30 to 60.

In some embodiments of this formula, each of R¹² and R¹³ is independently a straight alkyl chain containing from 10 to 18 carbon atoms, preferably from 12 to 16 carbon atoms.

In some embodiments of this formula, R¹² and R¹³ are identical. In some embodiments, each of R¹² and R¹³ is a straight alkyl chain containing 12 carbon atoms. In some embodiments, each of R¹² and R¹³ is a straight alkyl chain containing 14 carbon atoms. In some embodiments, each of R¹² and R¹³ is a straight alkyl chain containing 16 carbon atoms.

In some embodiments of this formula, R¹² and R¹³ are different. In some embodiments, one of R¹² and R¹³ is a straight alkyl chain containing 12 carbon atoms and the other of R¹² and R¹³ is a straight alkyl chain containing 14 carbon atoms.

In some embodiments of this formula, w has a mean value ranging from 40 to 50, such as a mean value of 45.

In some embodiments of this formula, w is within a range such that the PEG portion of the pegylated lipid has an average molecular weight of from about 400 to about 6000 g/mol, such as from about 1000 to about 5000 g/mol, from about 1500 to about 4000 g/mol, or from about 2000 to about 3000 g/mol. In some embodiments, each of R¹² and R¹³ is a straight alkyl chain containing 14 carbon atoms and w has a mean value of 45.

Various PEG-conjugated lipids are known in the art and include, but are not limited to pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy) propyl-1-O-(ω-methoxy (polyethoxy)ethyl) butanedioate (PEG-S-DMG), a pegylated ceramide (PEG-cer), or a PEG dialkoxypropylcarbamate such as ω-methoxy (polyethoxy)ethyl-N-(2,3-di(tetradecanoxy) propyl) carbamate or 2,3-di(tetradecanoxy) propyl-N-(ωmethoxy (polyethoxy)ethyl) carbamate, and the like.

In some embodiments, the PEG-conjugated lipid (pegylated lipid) is or comprises 2-[(polyethylene glycol)-2000]-N,N-ditetradecylacetamide. In some embodiments, the pegylated lipid has the following structure:

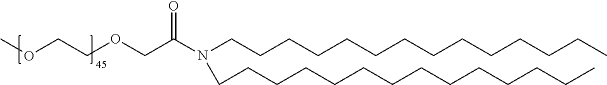

In some embodiments, the PEG-conjugated lipid (pegylated lipid) is DMG-PEG 2000, e.g., having the following structure:

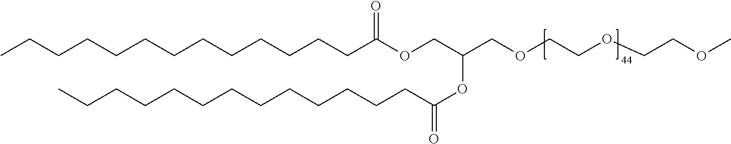

In some embodiments, the PEG-conjugated lipid (pegylated lipid) has the following structure:
wherein n has a mean value ranging from 30 to 60, such as about 50. In one embodiment, the PEG-conjugated

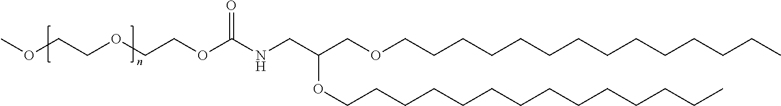

lipid (pegylated lipid) is PEG₂₀₀₀-C-DMA which preferably refers to 3-N-[(ω-methoxy poly(ethylene glycol) 2000) carbamoyl]-1,2-dimyristyloxy-propylamine (MPEG-(2 kDa)-C-DMA) or methoxy-polyethylene glycol-2,3-bis(tetradecyloxy) propylcarbamate (2000).

In some embodiments, RNA compositions/formulations described herein may comprise one or more PEG-conjugated lipids or pegylated lipids as described in WO 2017/075531 and WO 2018/081480, the entire contents of each of which are incorporated herein by reference for the purposes described herein.

In some embodiments, the pegylated lipid comprises from about 1 mol % to about 10 mol %, preferably from about 1 mol % to about 5 mol %, more preferably from about 1 mol % to about 2.5 mol % of the total lipid present in the RNA compositions/formulations and RNA particles described herein.

### Embodiments of Lipoplex Particles

In some embodiments of the present disclosure, the RNA described herein may be present in RNA lipoplex particles. Lipoplexes (LPX) are electrostatic complexes which are generally formed by mixing preformed cationic lipid liposomes with anionic RNA. Formed lipoplexes possess distinct internal arrangements of molecules that arise due to the transformation from liposomal structure into compact RNA-lipoplexes.

In certain embodiments, the RNA lipoplex particles include both a cationic lipid and an additional lipid. In an exemplary embodiment, the cationic lipid is DOTMA and the additional lipid is DOPE.

In some embodiments, the molar ratio of the at least one cationic lipid to the at least one additional lipid is from about 10:0 to about 1:9, about 4:1 to about 1:2, or about 3:1 to about 1:1. In specific embodiments, the molar ratio may be about 3:1, about 2.75:1, about 2.5:1, about 2.25:1, about 2:1, about 1.75:1, about 1.5:1, about 1.25:1, or about 1:1. In an exemplary embodiment, the molar ratio of the at least one cationic lipid to the at least one additional lipid is about 2:1.

RNA lipoplex particles described herein have an average diameter that in some embodiments ranges from about 200 nm to about 1000 nm, from about 200 nm to about 800 nm, from about 250 to about 700 nm, from about 400 to about 600 nm, from about 300 nm to about 500 nm, or from about 350 nm to about 400 nm. In specific embodiments, the RNA lipoplex particles have an average diameter of about 200 nm, about 225 nm, about 250 nm, about 275 nm, about 300 nm, about 325 nm, about 350 nm, about 375 nm, about 400 nm, about 425 nm, about 450 nm, about 475 nm, about 500 nm, about 525 nm, about 550 nm, about 575 nm, about 600 nm, about 625 nm, about 650 nm, about 675 nm, about 700 nm, about 725 nm, about 750 nm, about 775 nm, about 800 nm, about 825 nm, about 850 nm, about 875 nm, about 900 nm, about 925 nm, about 950 nm, about 975 nm, or about 1000 nm. In some embodiments, the RNA lipoplex particles have an average diameter that ranges from about 250 nm to about 700 nm. In some embodiments, the RNA lipoplex particles have an average diameter that ranges from about 300 nm to about 500 nm. In an exemplary embodiment, the RNA lipoplex particles have an average diameter of about 400 nm.

The RNA lipoplex particles and compositions comprising RNA lipoplex particles described herein are useful for delivery of RNA to a target tissue after parenteral administration, in particular after intravenous administration. Spleen targeting RNA lipoplex particles are described in WO 2013/143683, herein incorporated by reference. It has been found that RNA lipoplex particles having a net negative charge may be used to preferentially target spleen tissue or spleen cells such as antigen-presenting cells, in particular dendritic cells. Accordingly, following administration of the RNA lipoplex particles, RNA accumulation and/or RNA expression in the spleen occurs. Thus, RNA lipoplex particles of the disclosure may be used for expressing RNA in the spleen. In an embodiment, after administration of the RNA lipoplex particles, no or essentially no RNA accumulation and/or RNA expression in the lung and/or liver occurs. In some embodiments, after administration of the RNA lipoplex particles, RNA accumulation and/or RNA expression in antigen presenting cells, such as professional antigen presenting cells in the spleen occurs. Thus, RNA lipoplex particles of the disclosure may be used for targeting RNA, e.g., RNA encoding an antigen or at least one epitope, to the lymphatic system, in particular secondary lymphoid organs, more specifically spleen. Targeting the lymphatic system, in particular secondary lymphoid organs, more specifically spleen is in particular preferred if the RNA administered is RNA encoding vaccine antigen. In some embodiments, the target cell is a spleen cell. In some embodiments, the target cell is an antigen presenting cell such as a professional antigen presenting cell in the spleen. In some embodiments, the target cell is a dendritic cell in the spleen.

The electric charge of the RNA lipoplex particles of the present disclosure is the sum of the electric charges present in the at least one cationic lipid and the electric charges present in the RNA. The charge ratio is the ratio of the positive charges present in the at least one cationic lipid to the negative charges present in the RNA. The charge ratio of the positive charges present in the at least one cationic lipid to the negative charges present in the RNA is calculated by the following equation: charge ratio=[(cationic lipid concentration (mol))*(the total number of positive charges in the cationic lipid)]/[(RNA concentration (mol))*(the total number of negative charges in RNA)]. The concentration of RNA and the at least one cationic lipid amount can be determined using routine methods by one skilled in the art.

In some embodiments, at physiological pH the charge ratio of positive charges to negative charges in the RNA lipoplex particles is from about 1.6:2 to about 1:2, or about 1.6:2 to about 1.1:2. In specific embodiments, the charge ratio of positive charges to negative charges in the RNA lipoplex particles at physiological pH is about 1.6:2.0, about 1.5:2.0, about 1.4:2.0, about 1.3:2.0, about 1.2:2.0, about 1.1:2.0, or about 1:2.0.

### Embodiments of Lipid Nanoparticles (LNPs)

In some embodiments, RNA described herein is present in the form of lipid nanoparticles (LNPs). The LNP may comprise any lipid capable of forming a particle to which the one or more RNA molecules are attached, or in which the one or more RNA molecules are encapsulated.

LNPs typically comprise four components: cationically ionizable lipid, neutral lipids such as phospholipids, a steroid such as cholesterol, and a polymer-conjugated lipid such as PEG-lipid. LNPs may be prepared by mixing lipids dissolved in ethanol with RNA in an aqueous buffer.

In some embodiments, in the RNA LNPs described herein the RNA is bound by cationically ionizable lipid that occupies the central core of the LNP. Polymer-conjugated lipid forms the surface of the LNP, along with phospholipids. In some embodiments, the surface comprises a bilayer. In some embodiments, cholesterol and cationically ionizable lipid in charged and uncharged forms can be distributed throughout the LNP.

In some embodiments, the LNP comprises one or more cationically ionizable lipids, and one or more stabilizing lipids. Stabilizing lipids include neutral lipids and polymer-conjugated lipids.

In some embodiments, the LNP comprises a cationically ionizable lipid, a neutral lipid, a steroid, a polymer-conjugated lipid; and the RNA, encapsulated within or associated with the lipid nanoparticle.

In some embodiments, the LNP comprises from 40 to 60 mol percent, 40 to 55 mol percent, from 45 to 55 mol percent, or from 45 to 50 mol percent of the cationically ionizable lipid.

In some embodiments, the neutral lipid is present in a concentration ranging from 5 to 15 mol percent, from 7 to 13 mol percent, or from 9 to 11 mol percent.

In some embodiments, the steroid is present in a concentration ranging from 30 to 50 mol percent, from 30 to 45 mol percent, from 35 to 45 mol percent or from 35 to 43 mol percent.

In some embodiments, the LNP comprises from 1 to 10 mol percent, from 1 to 5 mol percent, or from 1 to 2.5 mol percent of the polymer-conjugated lipid.

In some embodiments, the LNP comprises from 45 to 55 mol percent of a cationically ionizable lipid; from 5 to 15 mol percent of a neutral lipid; from 30 to 45 mol percent of a steroid; from 1 to 5 mol percent of a polymer-conjugated lipid; and the RNA, encapsulated within or associated with the lipid nanoparticle.

In some embodiments, the mol percent is determined based on total mol of lipid present in the lipid nanoparticle. In some embodiments, the mol percent is determined based on total mol of cationically ionizable lipid, neutral lipid, steroid and polymer-conjugated lipid present in the lipid nanoparticle.

In some embodiments, the neutral lipid is selected from the group consisting of DSPC, DPPC, DMPC, DOPC, POPC, DOPE, DOPG, DPPG, POPE, DPPE, DMPE, DSPE, and SM. In some embodiments, the neutral lipid is selected from the group consisting of DSPC, DPPC, DMPC, DOPC, POPC, DOPE and SM. In some embodiments, the neutral lipid is DSPC.

In some embodiments, the steroid is cholesterol.

In some embodiments, the polymer conjugated lipid is a pegylated lipid, e.g., a pegylated lipid as described above.

In some embodiments, the cationically ionizable lipid component of the LNPs has the structure of Formula (III):

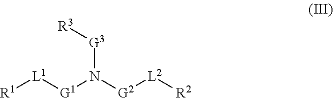

or a pharmaceutically acceptable salt, tautomer, prodrug or stereoisomer thereof, wherein:
one of L¹ or L² is-O(C═O)—, —(C═O)O—, —C(═O)—, —O—, —S(O)ₓ—, —S—S—, —C(═O)S—, SC(═O)—, —NR^{a}C(═O)—, —C(═O) NR^{a}—, NR^{a}C(═O) NR^{a}—, —OC(═O)NR^{a}— or —NR^{a}C(═O)O—, and the other of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O) NR^{a}—, NR^{a}C(═O)NR^{a}—, —OC(═O)NR^{a}— or —NR^{a}C(═O)O— or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C6-C₂₄ alkenyl;
R³ is H, OR⁵, CN, —C(═O)OR⁴, —OC(═O)R⁴ or —NR⁵C(═O) R⁴;
R⁴ is C₁-C₁₂ alkyl;
R⁵ is H or C₁-C₆ alkyl; and
x is 0, 1 or 2.

In some of the foregoing embodiments of Formula (III), the lipid has one of the following structures (IIIA) or (IIIB):

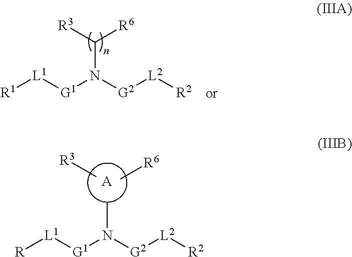

wherein:
A is a 3 to 8-membered cycloalkyl or cycloalkylene ring;
R⁶ is, at each occurrence, independently H, OH or C₁-C₂₄ alkyl;
n is an integer ranging from 1 to 15.

In some of the foregoing embodiments of Formula (III), the lipid has structure (IIIA), and in other embodiments, the lipid has structure (IIIB).

In other embodiments of Formula (III), the lipid has one of the following structures (IIIC) or (IIID):

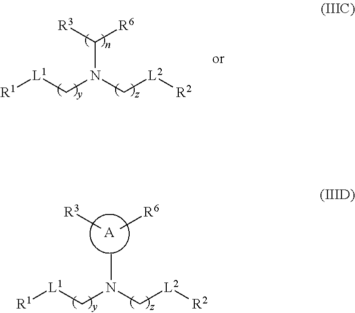

wherein y and z are each independently integers ranging from 1 to 12.

In any of the foregoing embodiments of Formula (III), one of L¹ or L² is —O(C═O)—. For example, in some embodiments each of L¹ and L² are —O(C═O)—. In some different embodiments of any of the foregoing, L¹ and L² are each independently —(C═O)O— or —O(C═O)—. For example, in some embodiments each of L¹ and L² is —(C═O)O—. In some different embodiments of Formula (III), the lipid has one of the following structures (IIIE) or (IIIF):

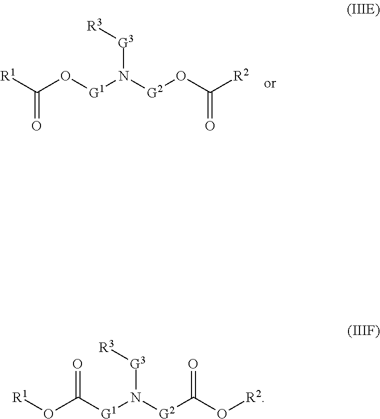

In some of the foregoing embodiments of Formula (III), the lipid has one of the following structures (IIIG), (IIIH), (IIII), or (IIIJ):

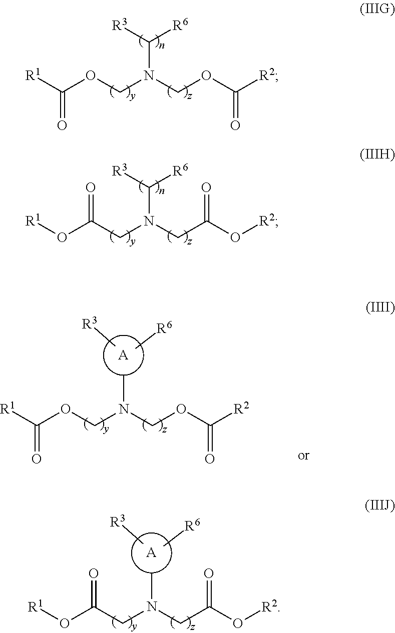

In some of the foregoing embodiments of Formula (III), n is an integer ranging from 2 to 12, for example from 2 to 8 or from 2 to 4. For example, in some embodiments, n is 3, 4, 5 or 6. In some embodiments, n is 3. In some embodiments, n is 4. In some embodiments, n is 5. In some embodiments, n is 6.

In some other of the foregoing embodiments of Formula (III), y and z are each independently an integer ranging from 2 to 10. For example, in some embodiments, y and z are each independently an integer ranging from 4 to 9 or from 4 to 6.

In some of the foregoing embodiments of Formula (III), R⁶ is H. In other of the foregoing embodiments, R⁶ is C₁-C₂₄ alkyl. In other embodiments, R⁶ is OH.

In some embodiments of Formula (III), G³ is unsubstituted. In other embodiments, G³ is substituted. In various different embodiments, G³ is linear C₁-C₂₄ alkylene or linear C₁-C₂₄ alkenylene.

In some other foregoing embodiments of Formula (III), R¹ or R², or both, is C₆-C₂₄ alkenyl. For example, in some embodiments, R¹ and R² each, independently have the following structure:

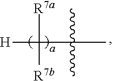

wherein:
R^{7a} and R^{7b} are, at each occurrence, independently H or C₁-C₁₂ alkyl; and
a is an integer from 2 to 12,
wherein R^{7a}, R^{7b} and a are each selected such that R¹ and R² each independently comprise from 6 to 20 carbon atoms. For example, in some embodiments a is an integer ranging from 5 to 9 or from 8 to 12.

In some of the foregoing embodiments of Formula (III), at least one occurrence of R^{7a} is H. For example, in some embodiments, R^{7b} is H at each occurrence. In other different embodiments of the foregoing, at least one occurrence of R^{7b} is C₁-C₈ alkyl. For example, in some embodiments, C₁-C₈ alkyl is methyl, ethyl, n-propyl, iso-propyl, n-butyl, iso-butyl, tert-butyl, n-hexyl or n-octyl.

In different embodiments of Formula (III), R¹ or R², or both, has one of the following structures:

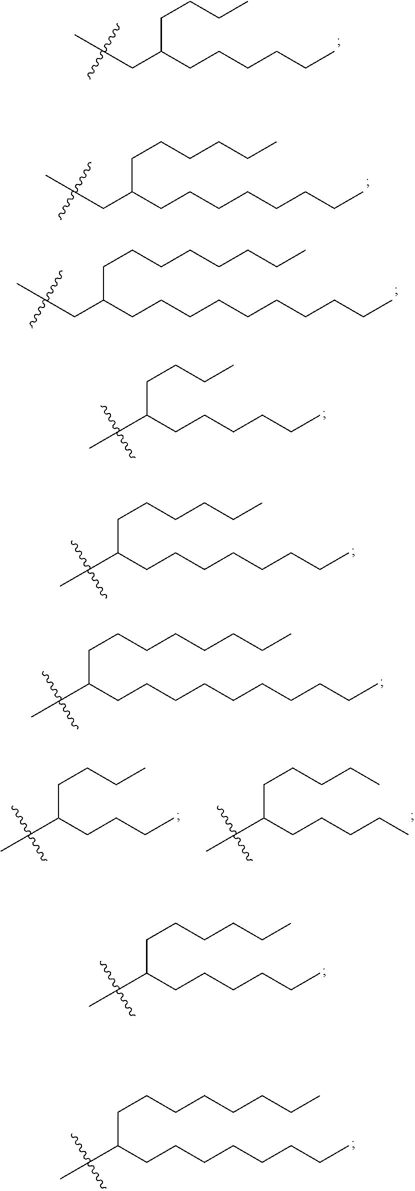

In some of the foregoing embodiments of Formula (III), R³ is OH, CN, —C(═O)OR⁴, —OC(═O)R⁴ or —NHC(═O)R⁴. In some embodiments, R⁴ is methyl or ethyl.

In various different embodiments, the cationic lipid of Formula (III) has one of the structures set forth in the table below.

TABLE 6
Representative Compounds of Formula (III).

| No. | Structure |
|---|---|
| III-1 | 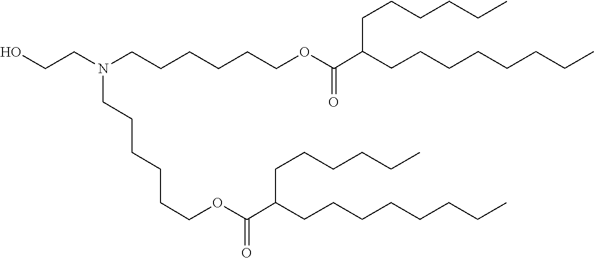 [SMILES: CCCCCCCCC(CCCCCC)C(=O)OCCCCCCN(CCO)CCCCCCOC(=O)C(CCCCCC)CCCCCCCC] |
| III-2 | 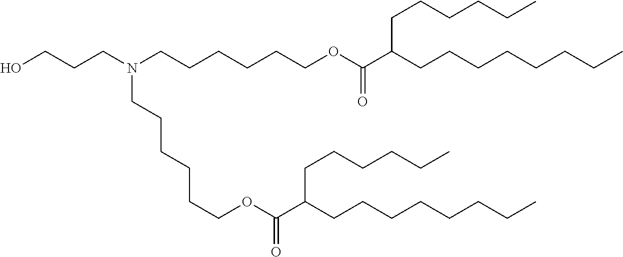 [SMILES: CCCCCCCCC(CCCCCC)C(=O)OCCCCCCN(CCCO)CCCCCCOC(=O)C(CCCCCC)CCCCCCCC] |
| III-3 | 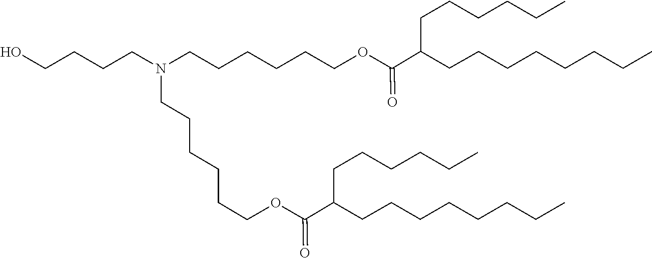 [SMILES: CCCCCCCCC(CCCCCC)C(=O)OCCCCCCN(CCCCO)CCCCCCOC(=O)C(CCCCCC)CCCCCCCC] |
| III-4 | 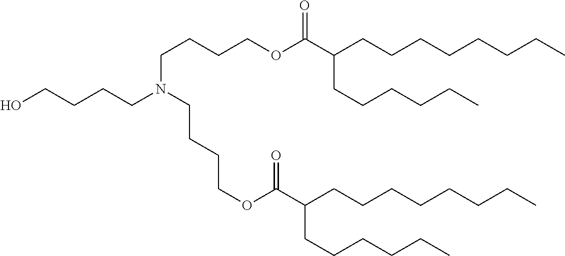 [SMILES: CCCCCCCCC(CCCCCC)C(=O)OCCCCN(CCCCO)CCCCOC(=O)C(CCCCCC)CCCCCCCC] |
| III-5 | 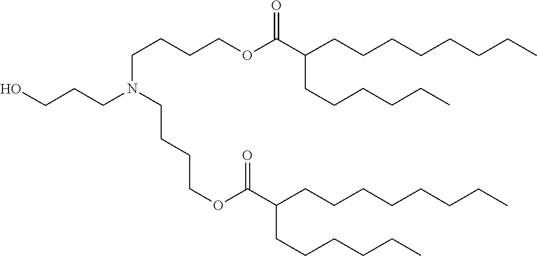 [SMILES: CCCCCCCCC(CCCCCC)C(=O)OCCCCN(CCCO)CCCCOC(=O)C(CCCCCC)CCCCCCCC] |
| III-6 | 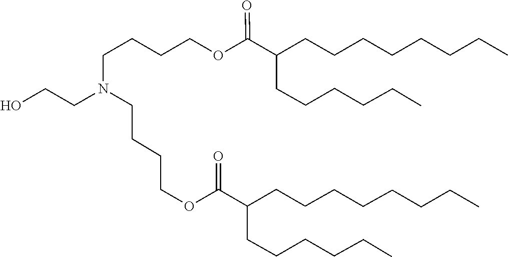 [SMILES: CCCCCCCCC(CCCCCC)C(=O)OCCCCN(CCO)CCCCOC(=O)C(CCCCCC)CCCCCCCC] |
| III-7 | 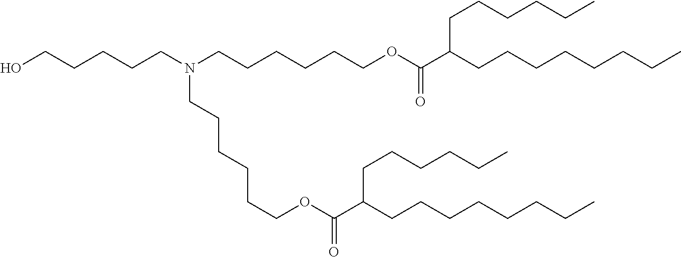 [SMILES: CCCCCCCCC(CCCCCC)C(=O)OCCCCCCN(CCCCCO)CCCCCCOC(=O)C(CCCCCC)CCCCCCCC] |
| III-8 | 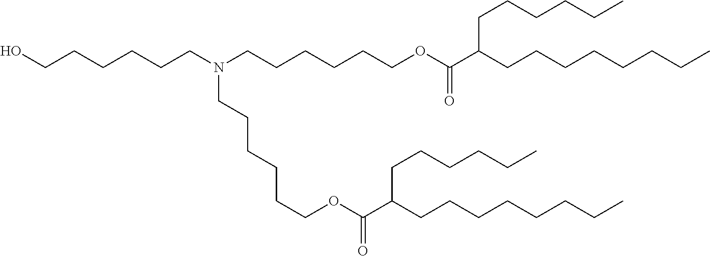 [SMILES: CCCCCCCCC(CCCCCC)C(=O)OCCCCCCN(CCCCCCO)CCCCCCOC(=O)C(CCCCCC)CCCCCCCC] |
| III-9 | 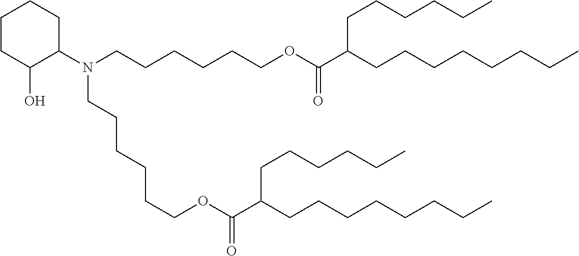 [SMILES: CCCCCCCCC(CCCCCC)C(=O)OCCCCCCN(CCCCCCOC(=O)C(CCCCCC)CCCCCCCC)C1CCCCC1O] |
| III-10 | 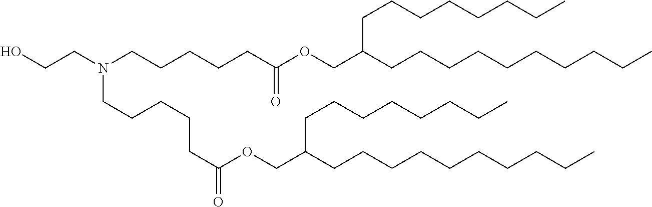 [SMILES: CCCCCCCCCCC(CCCCCCCC)COC(=O)CCCCCN(CCO)CCCCCC(=O)OCC(CCCCCCCC)CCCCCCCCCC] |
| III-11 | 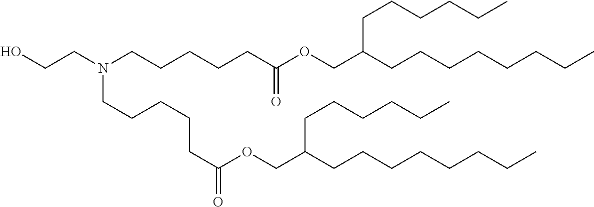 [SMILES: CCCCCCCCC(CCCCCC)COC(=O)CCCCCN(CCO)CCCCCC(=O)OCC(CCCCCC)CCCCCCCC] |
| III-12 | 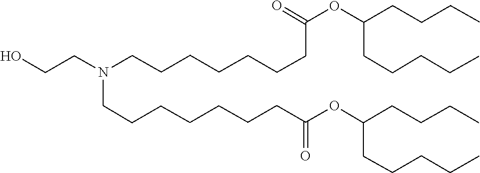 [SMILES: CCCCCC(CCCC)OC(=O)CCCCCCCN(CCO)CCCCCCCC(=O)OC(CCCC)CCCCC] |
| III-13 | 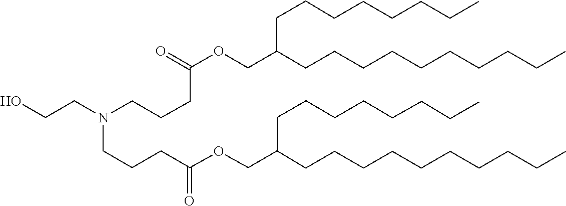 [SMILES: CCCCCCCCCCC(CCCCCCCC)COC(=O)CCCN(CCO)CCCC(=O)OCC(CCCCCCCC)CCCCCCCCCC] |
| III-14 | 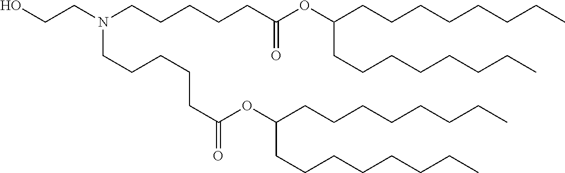 [SMILES: CCCCCCCCC(CCCCCCCC)OC(=O)CCCCCN(CCO)CCCCCC(=O)OC(CCCCCCCC)CCCCCCCC] |
| III-15 | 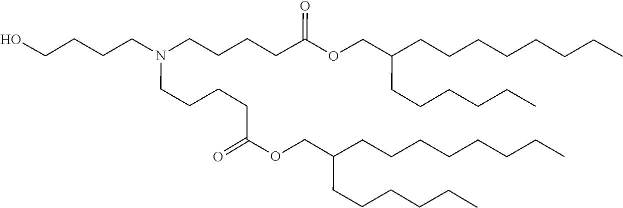 [SMILES: CCCCCCCCC(CCCCCC)COC(=O)CCCCN(CCCCO)CCCCC(=O)OCC(CCCCCC)CCCCCCCC] |
| III-16 | 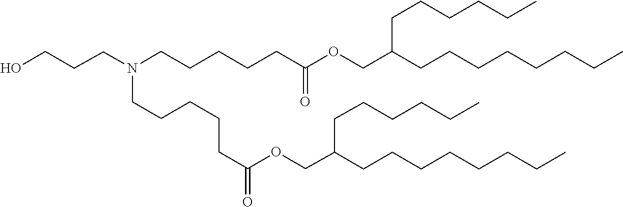 [SMILES: CCCCCCCCC(CCCCCC)COC(=O)CCCCCN(CCCO)CCCCCC(=O)OCC(CCCCCC)CCCCCCCC] |
| III-17 | 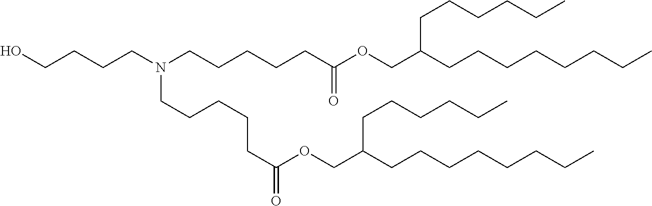 [SMILES: CCCCCCCCC(CCCCCC)COC(=O)CCCCCN(CCCCO)CCCCCC(=O)OCC(CCCCCC)CCCCCCCC] |
| III-18 | 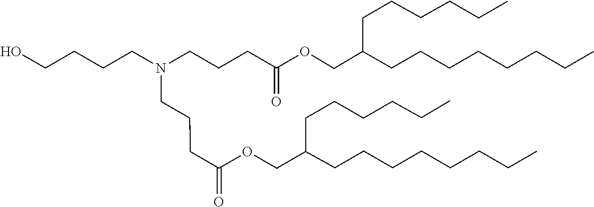 [SMILES: CCCCCCCCC(CCCCCC)COC(=O)CCCN(CCCCO)CCCC(=O)OCC(CCCCCC)CCCCCCCC] |
| III-19 | 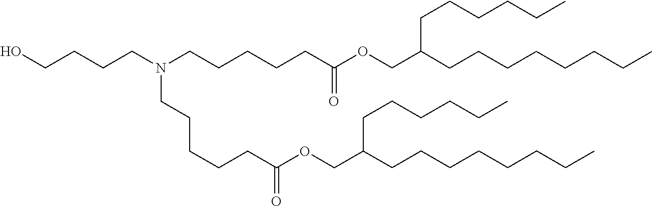 [SMILES: CCCCCCCCC(CCCCCC)COC(=O)CCCCCN(CCCCO)CCCCCC(=O)OCC(CCCCCC)CCCCCCCC] |
| III-20 | 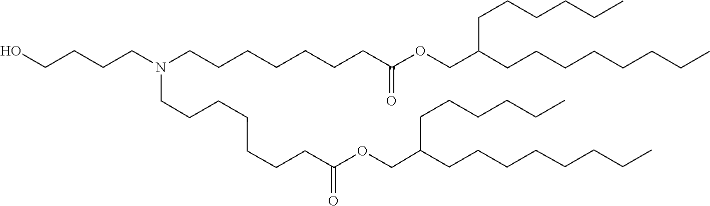 [SMILES: CCCCCCCCC(CCCCCC)COC(=O)CCCCCCCN(CCCCO)CCCCCCCC(=O)OCC(CCCCCC)CCCCCCCC] |
| III-21 | 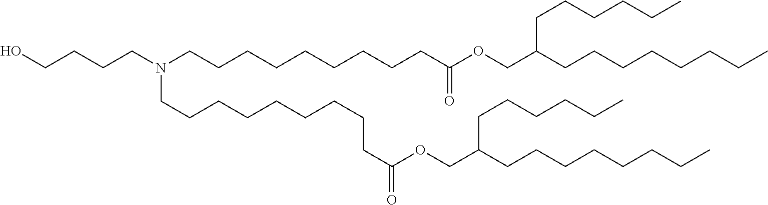 [SMILES: CCCCCCCCC(CCCCCC)COC(=O)CCCCCCCCCN(CCCCO)CCCCCCCCCC(=O)OCC(CCCCCC)CCCCCCCC] |
| III-22 | 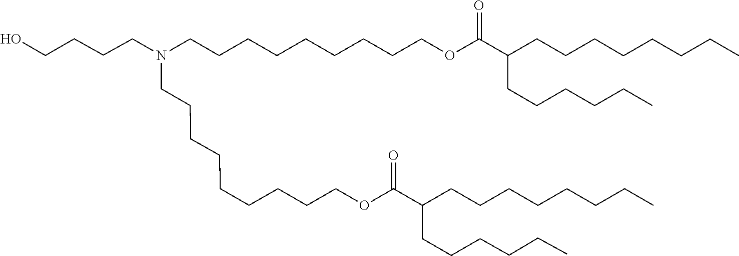 [SMILES: CCCCCCCCC(CCCCCC)C(=O)OCCCCCCCCCN(CCCCO)CCCCCCCCCOC(=O)C(CCCCCC)CCCCCCCC] |
| III-23 | 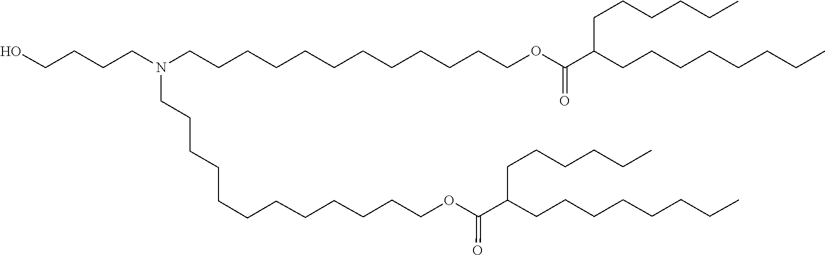 [SMILES: CCCCCCCCC(CCCCCC)C(=O)OCCCCCCCCCCCCN(CCCCO)CCCCCCCCCCCCOC(=O)C(CCCCCC)CCCCCCCC] |
| III-24 | 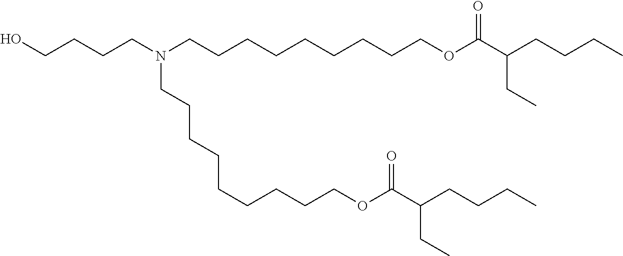 [SMILES: CCCCC(CC)C(=O)OCCCCCCCCCN(CCCCO)CCCCCCCCCOC(=O)C(CC)CCCC] |
| III-25 | 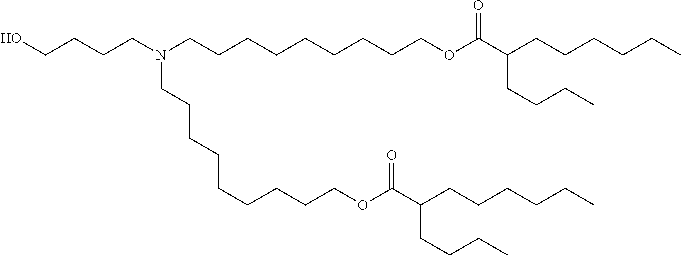 [SMILES: CCCCCCC(CCCC)C(=O)OCCCCCCCCCN(CCCCO)CCCCCCCCCOC(=O)C(CCCC)CCCCCC] |
| III-26 | 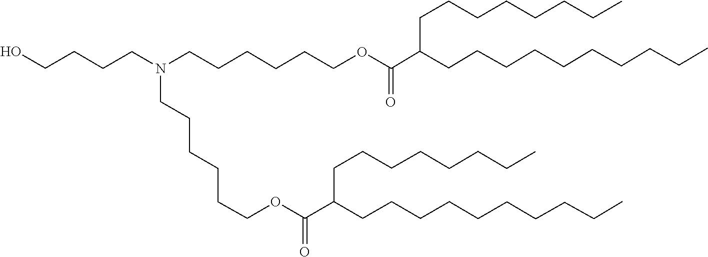 [SMILES: CCCCCCCCCCC(CCCCCCCC)C(=O)OCCCCCCN(CCCCO)CCCCCCOC(=O)C(CCCCCCCC)CCCCCCCCCC] |
| III-27 | 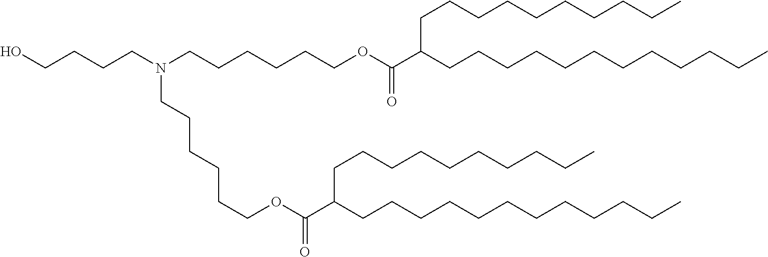 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] |
| III-28 | 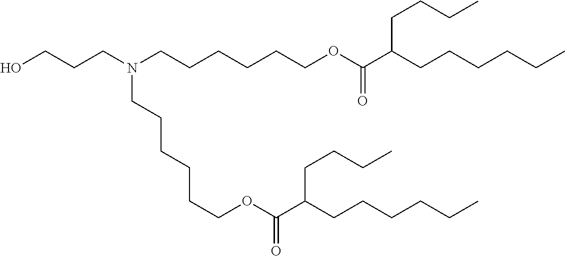 [SMILES: CCCCCCC(CCCC)C(=O)OCCCCCCN(CCCO)CCCCCCOC(=O)C(CCCC)CCCCCC] |
| III-29 | 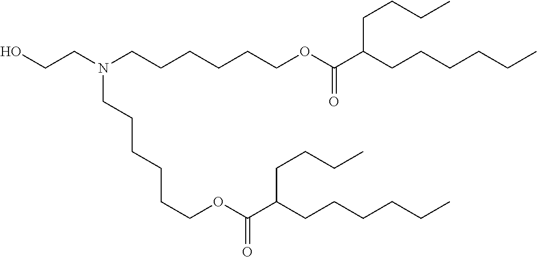 [SMILES: CCCCCCC(CCCC)C(=O)OCCCCCCN(CCO)CCCCCCOC(=O)C(CCCC)CCCCCC] |
| III-30 | 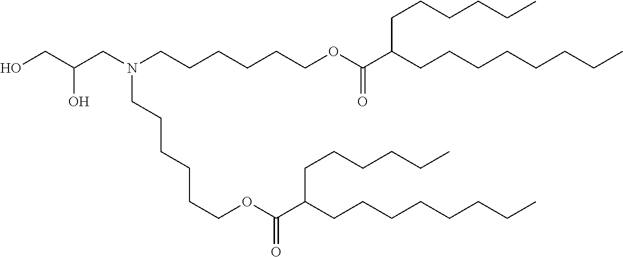 [SMILES: CCCCCCCCC(CCCCCC)C(=O)OCCCCCCN(CCCCCCOC(=O)C(CCCCCC)CCCCCCCC)CC(O)CO] |
| III-31 | 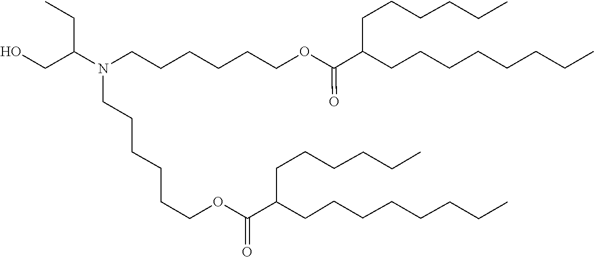 [SMILES: CCCCCCCCC(CCCCCC)C(=O)OCCCCCCN(CCCCCCOC(=O)C(CCCCCC)CCCCCCCC)C(CC)CO] |
| III-32 | 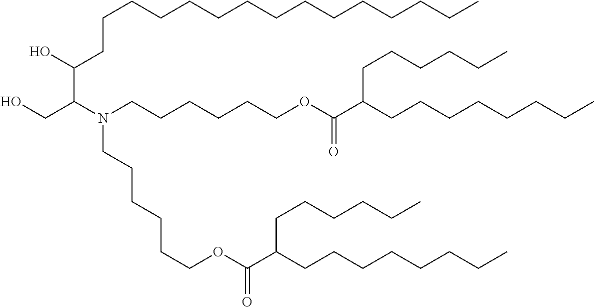 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] |
| III-33 | 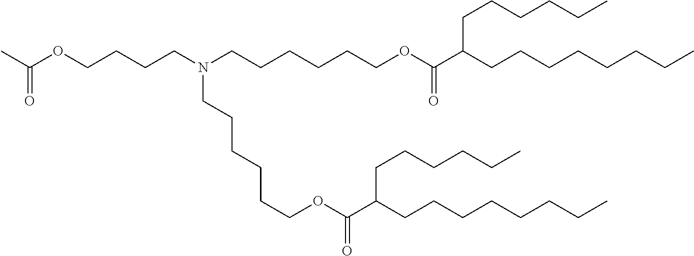 [SMILES: CCCCCCCCC(CCCCCC)C(=O)OCCCCCCN(CCCCCCOC(=O)C(CCCCCC)CCCCCCCC)CCCCOC(C)=O] |
| III-34 | 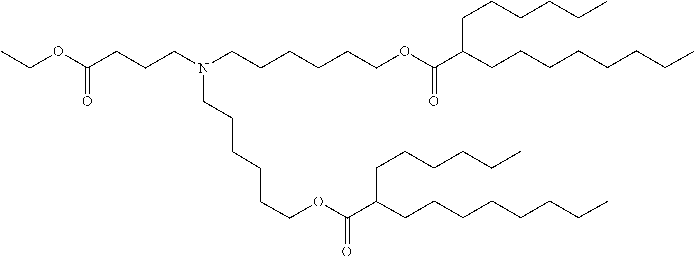 [SMILES: CCCCCCCCC(CCCCCC)C(=O)OCCCCCCN(CCCCCCOC(=O)C(CCCCCC)CCCCCCCC)CCCC(=O)OCC] |
| III-35 | 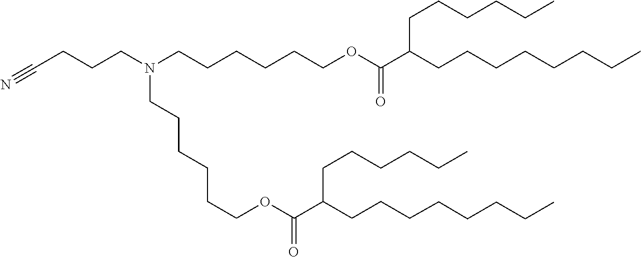 [SMILES: CCCCCCCCC(CCCCCC)C(=O)OCCCCCCN(CCCC#N)CCCCCCOC(=O)C(CCCCCC)CCCCCCCC] |
| III-36 | 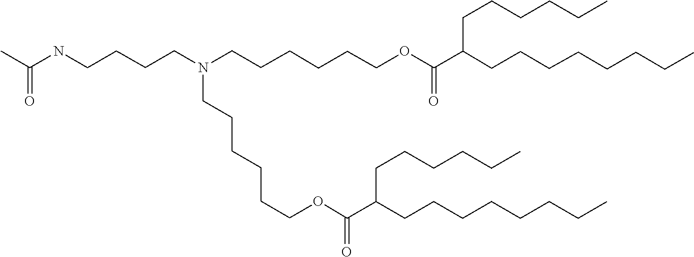 |
| III-37 | 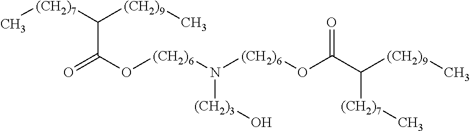 |
| III-38 | 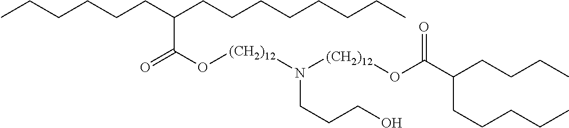 |
| III-39 | 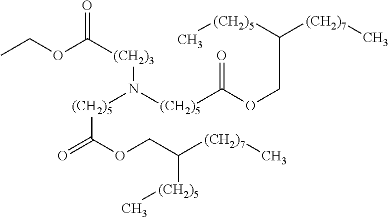 |
| III-40 | 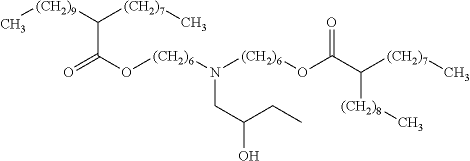 |
| III-41 | 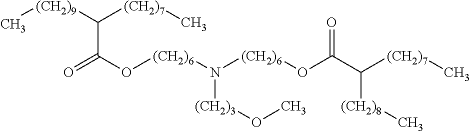 |
| III-42 | 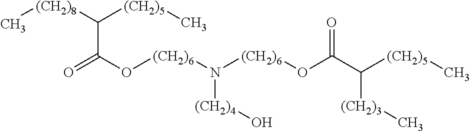 |
| III-43 | 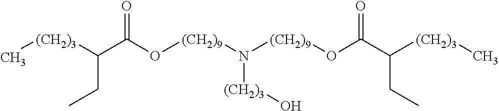 |
| III-44 | 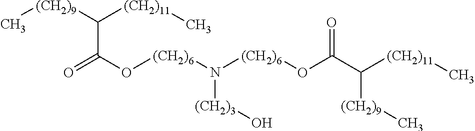 |
| III-45 | 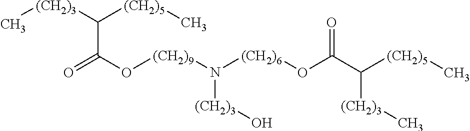 |
| III-46 | 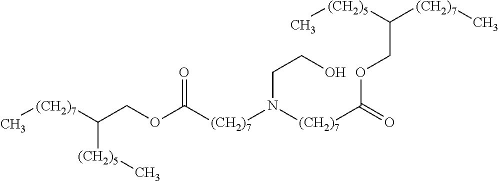 |
| III-47 | 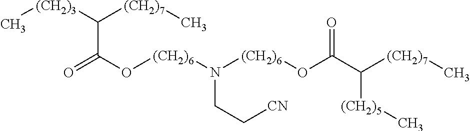 |
| III-48 | 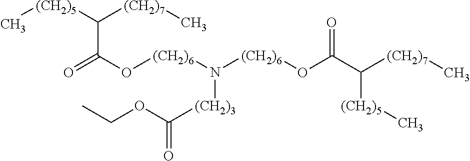 |
| III-49 | 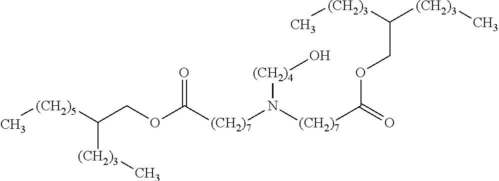 |

TABLE 7
Further representative cationically ionizable lipids

| No. | Structure |
|---|---|
| A | 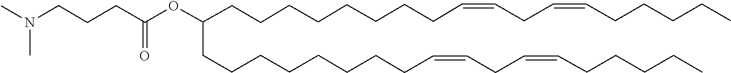 [SMILES: CCCCC/C=C\C/C=C\CCCCCCCCC(CCCCCCCC/C=C\C/C=C\CCCCC)OC(=O)CCCN(C)C] |
| B | 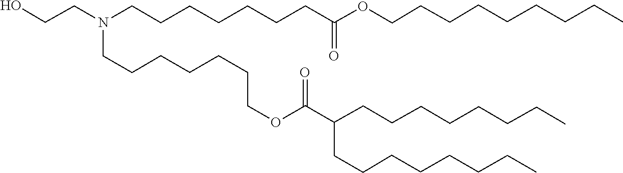 [SMILES: CCCCCCCCCOC(=O)CCCCCCCN(CCO)CCCCCCCOC(=O)C(CCCCCCCC)CCCCCCCC] |
| C | 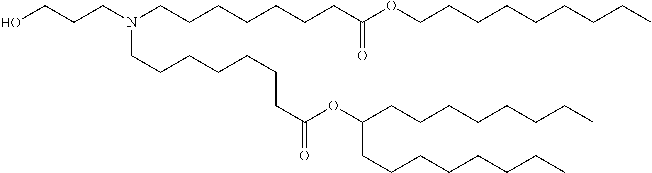 [SMILES: CCCCCCCCCOC(=O)CCCCCCCN(CCCO)CCCCCCCC(=O)OC(CCCCCCCC)CCCCCCCC] |
| D | 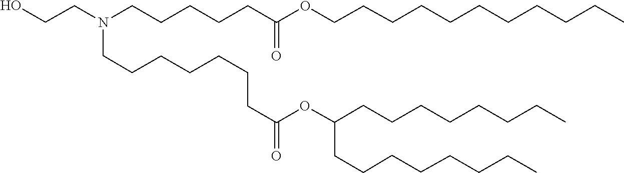 [SMILES: CCCCCCCCCCCOC(=O)CCCCCN(CCO)CCCCCCCC(=O)OC(CCCCCCCC)CCCCCCCC] |
| E | 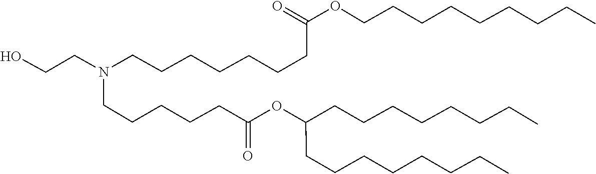 [SMILES: CCCCCCCCCOC(=O)CCCCCCCN(CCO)CCCCCC(=O)OC(CCCCCCCC)CCCCCCCC] |
| F | 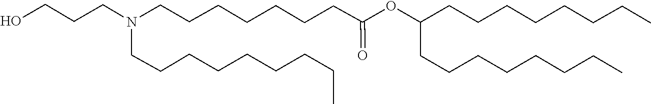 [SMILES: CCCCCCCCCN(CCCO)CCCCCCCC(=O)OC(CCCCCCCC)CCCCCCCC] |

In some embodiments, RNA described herein is formulated in an LNP composition comprising a cationically ionizable lipid, e.g., a cationically ionizable lipid as shown above, a neutral lipid, a steroid, and a polymer conjugated lipid. In some embodiments, RNA described herein is formulated in an LNP composition comprising a cationically ionizable lipid of Formula III, a neutral lipid, a steroid, and a polymer conjugated lipid.

In some embodiments, RNA described herein is formulated in an LNP composition comprising a cationically ionizable lipid shown in the above tables, a neutral lipid, a steroid, and a polymer conjugated lipid.

In some embodiments, RNA described herein is formulated in an LNP composition comprising 3D-P-DMA, a neutral lipid, a steroid, and a polymer conjugated lipid.

In some embodiments, RNA described herein is formulated in an LNP composition comprising ALC-0366, a neutral lipid, a steroid, and a polymer conjugated lipid.

In some embodiments, RNA described herein is formulated in an LNP composition comprising ALC-0315, a neutral lipid, a steroid, and a polymer conjugated lipid.

In some embodiments, the neutral lipid is DSPC. In some embodiments, the steroid is cholesterol. In some embodiments, the polymer conjugated lipid is a pegylated lipid, e.g., DMG-PEG 2000, PEG₂₀₀₀-C-DMA, or ALC-0159. In some embodiments, RNA described herein is formulated in an LNP composition comprising a cationically ionizable lipid, e.g., a cationically ionizable lipid as shown above, a neutral lipid, a steroid, and a pegylated lipid.

In some embodiments, RNA described herein is formulated in an LNP composition comprising a cationically ionizable lipid of Formula III, a neutral lipid, a steroid, and a pegylated lipid.

In some embodiments, RNA described herein is formulated in an LNP composition comprising a cationically ionizable lipid shown in the above tables, a neutral lipid, a steroid, and a pegylated lipid.

In some embodiments, RNA described herein is formulated in an LNP composition comprising 3D-P-DMA, a neutral lipid, a steroid, and a pegylated lipid.

In some embodiments, RNA described herein is formulated in an LNP composition comprising ALC-0366, a neutral lipid, a steroid, and a pegylated lipid.

In some embodiments, RNA described herein is formulated in an LNP composition comprising ALC-0315, a neutral lipid, a steroid, and a pegylated lipid.

In some embodiments, the neutral lipid is DSPC. In some embodiments, the steroid is cholesterol. In some embodiments, the pegylated lipid is DMG-PEG 2000, PEG₂₀₀₀-C-DMA, or ALC-0159.

In some embodiments, RNA described herein is formulated in an LNP composition comprising a cationically ionizable lipid, e.g., a cationically ionizable lipid as shown above, DSPC, cholesterol, and a pegylated lipid. In some embodiments, RNA described herein is formulated in an LNP composition comprising a cationically ionizable lipid of Formula III, DSPC, cholesterol, and a pegylated lipid.

In some embodiments, RNA described herein is formulated in an LNP composition comprising a cationically ionizable lipid shown in the above tables, DSPC, cholesterol, and a pegylated lipid.

In some embodiments, RNA described herein is formulated in an LNP composition comprising 3D-P-DMA, DSPC, cholesterol, and a pegylated lipid.

In some embodiments, RNA described herein is formulated in an LNP composition comprising ALC-0366, DSPC, cholesterol, and a pegylated lipid.

In some embodiments, RNA described herein is formulated in an LNP composition comprising ALC-0315, DSPC, cholesterol, and a pegylated lipid.

In some embodiments, the pegylated lipid is DMG-PEG 2000, PEG₂₀₀₀-C-DMA, or ALC-0159.

In some embodiments, RNA described herein is formulated in an LNP composition comprising a cationically ionizable lipid, e.g., a cationically ionizable lipid as shown above, DSPC, cholesterol, and DMG-PEG 2000.

In some embodiments, RNA described herein is formulated in an LNP composition comprising a cationically ionizable lipid of Formula III, DSPC, cholesterol, and DMG-PEG 2000.

In some embodiments, RNA described herein is formulated in an LNP composition comprising a cationically ionizable lipid shown in the above tables, DSPC, cholesterol, and DMG-PEG 2000.

In some embodiments, RNA described herein is formulated in an LNP composition comprising 3D-P-DMA, DSPC, cholesterol, and DMG-PEG 2000.

In some embodiments, RNA described herein is formulated in an LNP composition comprising ALC-0366, DSPC, cholesterol, and DMG-PEG 2000.

In some embodiments, RNA described herein is formulated in an LNP composition comprising ALC-0315, DSPC, cholesterol, and DMG-PEG 2000.

In some embodiments, RNA described herein is formulated in an LNP composition comprising a cationically ionizable lipid, e.g., a cationically ionizable lipid as shown above, DSPC, cholesterol, and PEG₂₀₀₀-C-DMA.

In some embodiments, RNA described herein is formulated in an LNP composition comprising a cationically ionizable lipid of Formula III, DSPC, cholesterol, and PEG₂₀₀₀-C-DMA.

In some embodiments, RNA described herein is formulated in an LNP composition comprising a cationically ionizable lipid shown in the above tables, DSPC, cholesterol, and PEG₂₀₀₀-C-DMA.

In some embodiments, RNA described herein is formulated in an LNP composition comprising 3D-P-DMA, DSPC, cholesterol, and PEG₂₀₀₀-C-DMA.

In some embodiments, RNA described herein is formulated in an LNP composition comprising ALC-0366, DSPC, cholesterol, and PEG₂₀₀₀-C-DMA.

In some embodiments, RNA described herein is formulated in an LNP composition comprising ALC-0315, DSPC, cholesterol, and PEG₂₀₀₀-C-DMA.

In some embodiments, RNA described herein is formulated in an LNP composition comprising a cationically ionizable lipid, e.g., a cationically ionizable lipid as shown above, DSPC, cholesterol, and ALC-0159.

In some embodiments, RNA described herein is formulated in an LNP composition comprising a cationically ionizable lipid of Formula III, DSPC, cholesterol, and ALC-0159.

In some embodiments, RNA described herein is formulated in an LNP composition comprising a cationically ionizable lipid shown in the above tables, DSPC, cholesterol, and ALC-0159.

In some embodiments, RNA described herein is formulated in an LNP composition comprising 3D-P-DMA, DSPC, cholesterol, and ALC-0159.

In some embodiments, RNA described herein is formulated in an LNP composition comprising ALC-0366, DSPC, cholesterol, and ALC-0159.

In some embodiments, RNA described herein is formulated in an LNP composition comprising ALC-0315, DSPC, cholesterol, and ALC-0159.

### 3D-P-DMA: (6Z,16Z)-12-((Z)-dec-4-en-1-yl) docosa-6,16-dien-11-yl 5-(dimethylamino) pentanoate

### ALC-0366: ((3-hydroxypropyl)azanediyl)bis(nonane-9,1-diyl)bis(2-butyloctanoate)

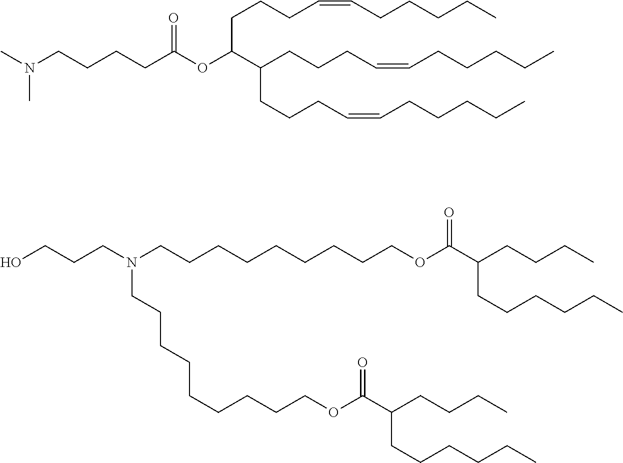 [SMILES: CCCCC/C=C\CCCC(CCC/C=C\CCCCC)C(CCC/C=C\CCCCC)OC(=O)CCCCN(C)C ; CCCCCCC(CCCC)C(=O)OCCCCCCCCCN(CCCO)CCCCCCCCCOC(=O)C(CCCC)CCCCCC]

### ALC-0315: ((4-hydroxybutyl)azanediyl)bis(hexane-6,1-diyl)bis(2-hexyldecanoate)/N-6-(2-hexyldecanoyloxy)hexyl-N-(4-hydroxybutyl)amino]hexyl 2-hexyldecanoate/6-[

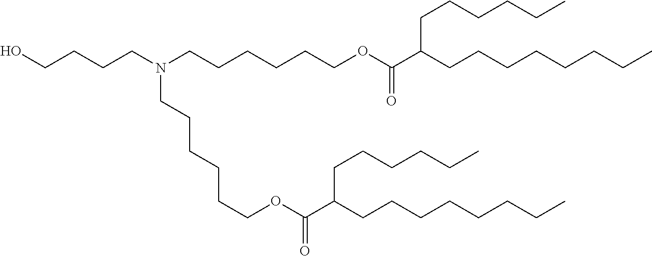 [SMILES: CCCCCCCCC(CCCCCC)C(=O)OCCCCCCN(CCCCO)CCCCCCOC(=O)C(CCCCCC)CCCCCCCC]

### DMG-PEG 2000:

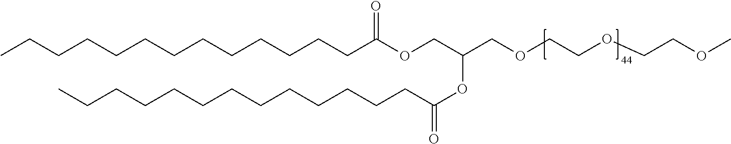

### PEG₂₀₀₀-C-DMA: 3-N-[(ω-Methoxy poly(ethylene glycol) 2000) carbamoyl]-1,2-dimyristyloxy-propylamine (MPEG-(2 kDa)-C-DMA or Methoxy-polyethylene glycol-2,3-bis(tetradecyloxy) propylcarbamate (2000))

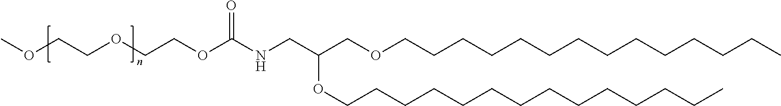

wherein n has a mean value ranging from 30 to 60, such as about 50.

### ALC-0159:2-[(polyethylene glycol)-2000]-N,N-ditetradecylacetamide/2-[2-(ω-methoxy (polyethyleneglycol2000) ethoxy]-N,N-ditetradecylacetamide

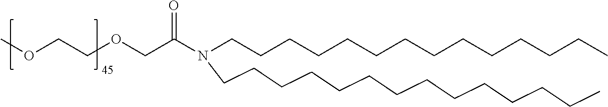

DSPC: 1,2-Distearoyl-sn-glycero-3-phosphocholine

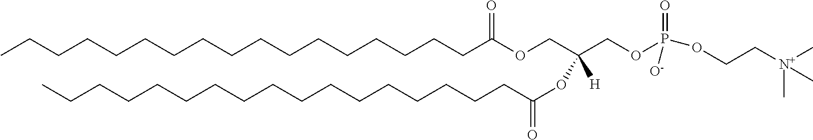 [SMILES: CCCCCCCCCCCCCCCCCC(=O)OC[C@H](COP(=O)([O-])OCC[N+](C)(C)C)OC(=O)CCCCCCCCCCCCCCCCC]

### Cholesterol:

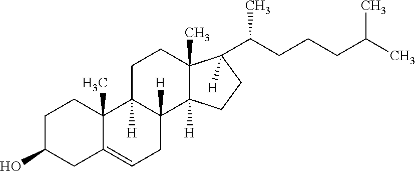

The N/P value is preferably at least about 4. In some embodiments, the N/P value ranges from 4 to 20, 4 to 12, 4 to 10, 4 to 8, or 5 to 7. In some embodiments, the N/P value is about 6.

### Doses

The term “dose” as used herein refers in general to a “dose amount” which relates to the amount of RNA administered per administration, i.e., per dosing.

In some embodiments, administration of RNA of the present disclosure may be performed by single administration or boosted by multiple administrations.

In some embodiments, an amount the RNA described herein from 0.1 μg to 300 μg, 0.5 μg to 200 μg, or 1 μg to 100 μg, such as about 1 μg, about 3 μg, about 10 μg, about 30 μg, about 50 μg, or about 100 μg may be administered per dose.

In some embodiments, a regimen described herein includes at least one dose. In some embodiments, a regimen includes a first dose and at least one subsequent dose. In some embodiments, a regimen includes a first dose and two subsequent doses. In some embodiments, the first dose is the same amount as at least one subsequent dose. In some embodiments, the first dose is the same amount as all subsequent doses. In some embodiments, the first dose is a different amount as at least one subsequent dose. In some embodiments, the first dose is a different amount than all subsequent doses. In some embodiments, a regimen comprises two doses. In some embodiments, a regimen consists of two doses. In some embodiments, a regimen comprises three doses. In some embodiments, a regimen consists of three doses.

In one embodiment, the disclosure envisions administration of a single dose. In one embodiment, the disclosure envisions administration of a priming dose followed by one or more booster doses. The booster dose or the first booster dose may be administered 7 to 90 days, 14 to 60 days, or 30 to 60 days following administration of the priming dose. For example, the booster dose or the first booster dose may be administered about 56 days following administration of the priming dose. The second booster dose may be administered 120 to 270 days, or 150 to 210 days following administration of the priming dose. For example, the second booster dose may be administered about 180 days following administration of the priming dose.

In some embodiments, an amount of the RNA described herein of 60 μg or lower, 50 μg or lower, or 40 μg or lower may be administered per dose.

In some embodiments, an amount of the RNA described herein of at least 0.25 μg, at least 0.5 μg, at least 1 μg, at least 2 μg, at least 3 μg, at least 4 μg, at least 5 μg, at least 10 μg, at least 20 μg, at least 30 μg, or at least 40 μg may be administered per dose.

In some embodiments, an amount of the RNA described herein of 0.25 μg to 60 μg, 0.5 μg to 55 μg, 1 μg to 50 ug, 5 μg to 40 μg, or 10 μg to 30 μg may be administered per dose.

In some embodiments, a regimen administered to a subject may comprise a plurality of doses (e.g., at least two doses, at least three doses, or more). In some embodiments, a regimen administered to a subject may comprise a first dose and at least one further dose (e.g., a second or a second and a third dose), which doses are given at least 2 weeks apart, at least 3 weeks apart, at least 4 weeks apart, or more. In some embodiments, such doses may be at least 1 month, at least 2 months, at least 3 months, at least 4 months, at least 5 months, at least 6 months, at least 7 months, at least 8 months, at least 9 months, at least 10 months, at least 11 months, at least 12 months, or more apart. In some embodiments, doses may be administered days apart, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more days apart. In some embodiments, doses may be administered about 1 to about 3 weeks apart, or about 1 to about 4 weeks apart, or about 1 to about 5 weeks apart, or about 1 to about 6 weeks apart, or about 1 to more than 6 weeks apart. In some embodiments, a minimum number of days between doses may be about 1, 2, 3, 4, 5, 6, 7, 8, 9, 10, 11, 12, 13, 14, 15, 16, 17, 18, 19, 20, 21 or more.

In some embodiments, a first dose and a second dose (and/or other subsequent doses) may be administered by intramuscular injection. In some embodiments, a first dose and a second dose (and/or other subsequent doses) may be administered in the deltoid muscle. In some embodiments, a first dose and a second dose (and/or other subsequent doses) may be administered in the same arm. In some embodiments, an mRNA composition described herein is administered (e.g., by intramuscular injection) as a series of three doses. In some embodiments, each dose is about 30 μg. In some embodiments, each dose is about 10 μg. In some embodiments, each dose is about 3 μg. In some embodiments, each dose is about 1 μg.

In some such embodiments, an mRNA composition described herein is administered to subjects of age 12 or older. In some such embodiments, an mRNA composition described herein is administered to subjects of age 5 to 11. In some such embodiments, an mRNA composition described herein is administered to subjects of age 2 to less than 5.

In some such embodiments, an mRNA composition described herein is administered to subjects of age 12 or older and each dose is about 30 μg. In some such embodiments, an mRNA composition described herein is administered to subjects of age 5 to 11 and each dose is about 10 μg. In some such embodiments, an mRNA composition described herein is administered to subjects of age 2 to less than 5 and each dose is about 3 μg.

### Compositions Comprising Nucleic Acid

A composition comprising one or more RNAs described herein, e.g., in the form of RNA particles, may comprise salts, buffers, or other components as further described below.

In some embodiments, a salt for use in the compositions described herein comprises sodium chloride. Without wishing to be bound by theory, sodium chloride functions as an ionic osmolality agent for preconditioning RNA prior to mixing with lipids. In some embodiments, the compositions described herein may comprise alternative organic or inorganic salts. Alternative salts include, without limitation, potassium chloride, dipotassium phosphate, monopotassium phosphate, potassium acetate, potassium bicarbonate, potassium sulfate, disodium phosphate, monosodium phosphate, sodium acetate, sodium bicarbonate, sodium sulfate, lithium chloride, magnesium chloride, magnesium phosphate, calcium chloride, and sodium salts of ethylenediaminetetraacetic acid (EDTA). Generally, compositions for storing RNA particles such as for freezing RNA particles comprise low sodium chloride concentrations, or comprises a low ionic strength. In some embodiments, the sodium chloride is at a concentration from 0 mM to about 50 mM, from 0 mM to about 40 mM, or from about 10 mM to about 50 mM.

According to the present disclosure, the RNA particle compositions described herein have a pH suitable for the stability of the RNA particles and, in particular, for the stability of the RNA. Without wishing to be bound by theory, the use of a buffer system maintains the pH of the particle compositions described herein during manufacturing, storage and use of the compositions. In some embodiments of the present disclosure, the buffer system may comprise a solvent (in particular, water, such as deionized water, in particular water for injection) and a buffering substance. The buffering substance may be selected from 2-[4-(2-hydroxyethyl) piperazin-1-yl]ethanesulfonic acid (HEPES), 2-amino-2-(hydroxymethyl) propane-1,3-diol (Tris), acetate, and histidine. In some embodiments, the buffering substance is HEPES. In some embodiments, the buffering substance is Tris.

Compositions (in particular, RNA compositions/formulations) described herein may also comprise a cryoprotectant and/or a surfactant as stabilizer to avoid substantial loss of the product quality and, in particular, substantial loss of RNA activity during storage, freezing, and/or lyophilization, for example to reduce or prevent aggregation, particle collapse, RNA degradation and/or other types of damage.

In some embodiments, the cryoprotectant is a carbohydrate. The term “carbohydrate”, as used herein, refers to and encompasses monosaccharides, disaccharides, trisaccharides, oligosaccharides and polysaccharides. In some embodiments, the cryoprotectant is a monosaccharide. The term “monosaccharide”, as used herein refers to a single carbohydrate unit (e.g., a simple sugar) that cannot be hydrolyzed to simpler carbohydrate units. Exemplary monosaccharide cryoprotectants include glucose, fructose, galactose, xylose, ribose and the like.

In some embodiments, the cryoprotectant is a disaccharide. The term “disaccharide”, as used herein refers to a compound or a chemical moiety formed by 2 monosaccharide units that are bonded together through a glycosidic linkage, for example through 1-4 linkages or 1-6 linkages. A disaccharide may be hydrolyzed into two monosaccharides. Exemplary disaccharide cryoprotectants include sucrose, trehalose, lactose, maltose and the like. In some embodiments, the cryoprotectant is sucrose.

The term “trisaccharide” means three sugars linked together to form one molecule. Examples of a trisaccharides include raffinose and melezitose.

In some embodiments, the cryoprotectant is an oligosaccharide. The term “oligosaccharide”, as used herein refers to a compound or a chemical moiety formed by 3 to about 15, such as 3 to about 10 monosaccharide units that are bonded together through glycosidic linkages, for example through 1-4 linkages or 1-6 linkages, to form a linear, branched or cyclic structure. Exemplary oligosaccharide cryoprotectants include cyclodextrins, raffinose, melezitose, maltotriose, stachyose, acarbose, and the like. An oligosaccharide can be oxidized or reduced.

In an embodiment, the cryoprotectant is a cyclic oligosaccharide. The term “cyclic oligosaccharide”, as used herein refers to a compound or a chemical moiety formed by 3 to about 15, such as 6, 7, 8, 9, or 10 monosaccharide units that are bonded together through glycosidic linkages, for example through 1-4 linkages or 1-6 linkages, to form a cyclic structure. Exemplary cyclic oligosaccharide cryoprotectants include cyclic oligosaccharides that are discrete compounds, such as a cyclodextrin, β cyclodextrin, or γcyclodextrin.

Other exemplary cyclic oligosaccharide cryoprotectants include compounds which include a cyclodextrin moiety in a larger molecular structure, such as a polymer that contains a cyclic oligosaccharide moiety. A cyclic oligosaccharide can be oxidized or reduced, for example, oxidized to dicarbonyl forms. The term “cyclodextrin moiety”, as used herein refers to cyclodextrin (e.g., an α, β, or γcyclodextrin) radical that is incorporated into, or a part of, a larger molecular structure, such as a polymer. A cyclodextrin moiety can be bonded to one or more other moieties directly, or through an optional linker. A cyclodextrin moiety can be oxidized or reduced, for example, oxidized to dicarbonyl forms.

Carbohydrate cryoprotectants, e.g., cyclic oligosaccharide cryoprotectants, can be derivatized carbohydrates. For example, in an embodiment, the cryoprotectant is a derivatized cyclic oligosaccharide, e.g., a derivatized cyclodextrin, e.g., 2-hydroxypropyl-β-cyclodextrin, e.g., partially etherified cyclodextrins (e.g., partially etherified β cyclodextrins).

An exemplary cryoprotectant is a polysaccharide. The term “polysaccharide”, as used herein refers to a compound or a chemical moiety formed by at least 16 monosaccharide units that are bonded together through glycosidic linkages, for example through 1-4 linkages or 1-6 linkages, to form a linear, branched or cyclic structure, and includes polymers that comprise polysaccharides as part of their backbone structure. In backbones, the polysaccharide can be linear or cyclic. Exemplary polysaccharide cryoprotectants include glycogen, amylase, cellulose, dextran, maltodextrin and the like.

In some embodiments, RNA particle compositions may include sucrose. Without wishing to be bound by theory, sucrose functions to promote cryoprotection of the compositions, thereby preventing RNA (especially mRNA) particle aggregation and maintaining chemical and physical stability of the composition. In some embodiments, RNA particle compositions may include alternative cryoprotectants to sucrose. Alternative stabilizers include, without limitation, trehalose and glucose. In a specific embodiment, an alternative stabilizer to sucrose is trehalose or a mixture of sucrose and trehalose.

A preferred cryoprotectant is selected from the group consisting of sucrose, trehalose, glucose, and a combination thereof, such as a combination of sucrose and trehalose. In a preferred embodiment, the cryoprotectant is sucrose. Some embodiments of the present disclosure contemplate the use of a chelating agent in an RNA composition described herein. Chelating agents refer to chemical compounds that are capable of forming at least two coordinate covalent bonds with a metal ion, thereby generating a stable, water-soluble complex. Without wishing to be bound by theory, chelating agents reduce the concentration of free divalent ions, which may otherwise induce accelerated RNA degradation in the present disclosure. Examples of suitable chelating agents include, without limitation, ethylenediaminetetraacetic acid (EDTA), a salt of EDTA, desferrioxamine B, deferoxamine, dithiocarb sodium, penicillamine, pentetate calcium, a sodium salt of pentetic acid, succimer, trientine, nitrilotriacetic acid, trans-diaminocyclohexanetetraacetic acid diethylenetriaminepentaacetic acid (DCTA), (DTPA), and bis(aminoethyl)glycolether-N,N,N′,N′-tetraacetic acid. In some embodiments, the chelating agent is EDTA or a salt of EDTA. In some embodiments, the chelating agent is EDTA disodium dihydrate. In some embodiments, the EDTA is at a concentration from about 0.05 mM to about 5 mM, from about 0.1 mM to about 2.5 mM or from about 0.25 mM to about 1 mM.

In an alternative embodiment, the RNA particle compositions described herein do not comprise a chelating agent.

### Pharmaceutical Compositions

The agents described herein may be administered in pharmaceutical compositions or medicaments and may be administered in the form of any suitable pharmaceutical composition. In some embodiments, the pharmaceutical composition is for therapeutic or prophylactic treatments, e.g., for use in treating or preventing a disease involving an antigen, in particular tuberculosis.

The term “pharmaceutical composition” relates to a composition comprising a therapeutically effective agent, preferably together with pharmaceutically acceptable carriers, diluents and/or excipients. Said pharmaceutical composition is useful for treating, preventing, or reducing the severity of a disease by administration of said pharmaceutical composition to a subject.

The pharmaceutical compositions of the present disclosure may comprise one or more adjuvants or may be administered with one or more adjuvants. The term “adjuvant” relates to a compound which prolongs, enhances or accelerates an immune response. Adjuvants comprise a heterogeneous group of compounds such as oil emulsions (e.g., Freund's adjuvants), mineral compounds (such as alum), bacterial products (such as Bordetella pertussis toxin), or immune-stimulating complexes. Examples of adjuvants include, without limitation, LPS, GP96, CpG oligodeoxynucleotides, growth factors, and cytokines, such as monokines, lymphokines, interleukins, chemokines. The chemokines may be IL-1, IL-2, IL-3, IL-4, IL-5, IL-6, IL-7, IL-8, IL-9, IL-10, IL-12, INFa, INF-γ, GM-CSF, LT-a. Further known adjuvants are aluminum hydroxide, Freund's adjuvant or oil such as Montanide® ISA51. Other suitable adjuvants for use in the present disclosure include lipopeptides, such as Pam3Cys, as well as lipophilic components, such as saponins, trehalose-6,6-dibehenate (TDB), monophosphoryl lipid-A (MPL), monomycoloyl glycerol (MMG), or glucopyranosyl lipid adjuvant (GLA).

The pharmaceutical compositions of the present disclosure may be in a storable form (e.g., in a frozen or lyophilized/freeze-dried form) or in a “ready-to-use form” (i.e., in a form which can be immediately administered to a subject, e.g., without any processing such as diluting). Thus, prior to administration of a storable form of a pharmaceutical composition, this storable form has to be processed or transferred into a ready-to-use or administrable form. E.g., a frozen pharmaceutical composition has to be thawed, or a freeze-dried pharmaceutical composition has to be reconstituted, e.g. by using a suitable solvent (e.g., deionized water, such as water for injection) or liquid (e.g., an aqueous solution).

The pharmaceutical compositions according to the present disclosure are generally applied in a “pharmaceutically effective amount” and in “a pharmaceutically acceptable preparation”.

The term “pharmaceutically acceptable” refers to the non-toxicity of a material which does not interact with the action of the active component of the pharmaceutical composition.

The term “pharmaceutically effective amount” refers to the amount which achieves a desired reaction or a desired effect alone or together with further doses. In some embodiments relating to the treatment of a particular disease, the desired reaction may relate to inhibition of the course of the disease. This comprises slowing down the progress of the disease and, in some embodiments, interrupting or reversing the progress of the disease. The desired reaction in a treatment of a disease may also be delay of the onset or a prevention of the onset of said disease or said condition, or symptoms thereof. An effective amount of the pharmaceutical compositions described herein will depend on the condition to be treated, the severeness of the disease, the individual parameters of the patient, including age, physiological condition, size and weight, the duration of treatment, the type of an accompanying therapy (if present), the specific route of administration and similar factors. Accordingly, the doses administered of the pharmaceutical compositions described herein may depend on various of such parameters. In the case that a reaction in a patient is insufficient with an initial dose, higher doses (or effectively higher doses achieved by a different, more localized route of administration) may be used.

The pharmaceutical compositions of the present disclosure may contain buffers, preservatives, and optionally other therapeutic agents. In some embodiments, the pharmaceutical compositions of the present disclosure comprise one or more pharmaceutically acceptable carriers, diluents and/or excipients.

Suitable preservatives for use in the pharmaceutical compositions of the present disclosure include, without limitation, benzalkonium chloride, chlorobutanol, paraben and thimerosal.

The term “excipient” as used herein refers to a substance which may be present in a pharmaceutical composition of the present disclosure but is not an active ingredient. Examples of excipients, include without limitation, carriers, binders, diluents, lubricants, thickeners, surface active agents, preservatives, stabilizers, emulsifiers, buffers, flavoring agents, or colorants

The term “diluent” relates a diluting and/or thinning agent. Moreover, the term “diluent” includes any one or more of fluid, liquid or solid suspension and/or mixing media. Examples of suitable diluents include ethanol, glycerol and water.

The term “carrier” refers to a component which may be natural, synthetic, organic, inorganic in which the active component is combined in order to facilitate, enhance or enable administration of the pharmaceutical composition.

A carrier as used herein may be one or more compatible solid or liquid fillers, diluents or encapsulating substances, which are suitable for administration to subject. Suitable carriers include, without limitation, sterile water, Ringer, Ringer lactate, sterile sodium chloride solution, isotonic saline, polyalkylene glycols, hydrogenated naphthalenes and, in particular, biocompatible lactide polymers, lactide/glycolide copolymers or polyoxyethylene/polyoxy-propylene copolymers. In some embodiments, the pharmaceutical composition of the present disclosure includes isotonic saline.

Pharmaceutically acceptable carriers, excipients or diluents for therapeutic use are well known in the pharmaceutical art, and are described, for example, in Remington's Pharmaceutical Sciences, Mack Publishing Co. (A. R Gennaro edit. 1985).

Pharmaceutical carriers, excipients or diluents can be selected with regard to the intended route of administration and standard pharmaceutical practice.

### Routes of Administration of Pharmaceutical Compositions

In some embodiments, the pharmaceutical compositions described herein may be administered intravenously, intraarterially, subcutaneously, intradermally, dermally, intranodally, or intramuscularly. In some embodiments, the pharmaceutical compositions described herein may be administered intramuscularly. In some embodiments, the pharmaceutical composition is formulated for local administration or systemic administration. Systemic administration may include enteral administration, which involves absorption through the gastrointestinal tract, or parenteral administration. As used herein, “parenteral administration” refers to the administration in any manner other than through the gastrointestinal tract, such as by intravenous injection. In some embodiments, the pharmaceutical compositions are formulated for systemic administration. In some embodiments, the systemic administration is by intravenous administration. In some embodiments, the pharmaceutical compositions are formulated for intramuscular administration.

In some embodiments, intramuscular administration comprises administration into the upper arm, in particular into the Musculus deltoideus. If more than one dose, e.g., three doses, of a pharmaceutical composition described herein is administered, the different administrations may be into the same arm.

### Use of Compositions

Compositions described herein may be used in the therapeutic or prophylactic treatment of diseases wherein provision of one or more peptides or polypeptides, i.e., vaccine antigens, described herein to a subject results in a therapeutic or prophylactic effect. In some embodiments, the disease is infection with Mycobacterium tuberculosis. In some embodiments, the disease is tuberculosis.

The term “disease” (also referred to as “disorder” herein) refers to an abnormal condition that affects the body of an individual. A disease is often construed as a medical condition associated with specific symptoms and signs. A disease may be caused by factors originally from an external source, such as infectious disease, or it may be caused by internal dysfunctions, such as autoimmune diseases. In humans, “disease” is often used more broadly to refer to any condition that causes pain, dysfunction, distress, social problems, or death to the individual afflicted, or similar problems for those in contact with the individual. In this broader sense, it sometimes includes injuries, disabilities, disorders, syndromes, infections, isolated symptoms, deviant behaviors, and atypical variations of structure and function, while in other contexts and for other purposes these may be considered distinguishable categories. Diseases usually affect individuals not only physically, but also emotionally, as contracting and living with many diseases can alter one's perspective on life, and one's personality.

The term “disease involving an antigen” refers to any disease which implicates an antigen, e.g. a disease which is characterized by the presence of an antigen. The disease involving an antigen can be an infectious disease. The antigen may be a disease-associated antigen, such as a bacterial antigen. In some embodiments, a disease involving an antigen is a disease involving cells comprising and/or expressing an antigen, and preferably presenting the antigen on the cell surface, e.g., in the context of MHC.

The term “infectious disease” refers to any disease which can be transmitted from individual to individual or from organism to organism, and is caused by a microbial agent (e.g. common cold). Infectious diseases are known in the art and include, for example, a viral disease, a bacterial disease, or a parasitic disease, which diseases are caused by a virus, a bacterium, and a parasite, respectively. In this regard, the infectious disease can be, for example, hepatitis, sexually transmitted diseases (e.g. chlamydia or gonorrhea), tuberculosis, HIV/acquired immune deficiency syndrome (AIDS), diphtheria, hepatitis B, hepatitis C, cholera, severe acute respiratory syndrome (SARS), the bird flu, and influenza.

In the present context, the term “treatment”, “treating” or “therapeutic intervention” relates to the management and care of a subject for the purpose of combating a condition such as a disease. The term is intended to include the full spectrum of treatments for a given condition from which the subject is suffering, such as administration of the therapeutically effective compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of an individual for the purpose of combating the disease, condition or disorder and includes the administration of the active compounds to prevent the onset of the symptoms or complications.

The term “therapeutic treatment” relates to any treatment which improves the health status and/or prolongs (increases) the lifespan of an individual. Said treatment may eliminate the disease in an individual, arrest or slow the development of a disease in an individual, inhibit or slow the development of a disease in an individual, decrease the frequency or severity of symptoms in an individual, and/or decrease the recurrence in an individual who currently has or who previously has had a disease.

The terms “prophylactic treatment” or “preventive treatment” relate to any treatment that is intended to prevent a disease from occurring in an individual. The terms “prophylactic treatment” or “preventive treatment” are used herein interchangeably.

The terms “individual” and “subject” are used herein interchangeably. They refer to a human or another mammal (e.g., mouse, rat, rabbit, dog, cat, cattle, swine, sheep, horse or primate), or any other non-mammal-animal, including birds (chicken), fish or any other animal species that can be afflicted with or is susceptible to a disease (e.g., cancer, infectious diseases) but may or may not have the disease, or may have a need for prophylactic intervention such as vaccination, or may have a need for interventions such as by protein replacement. In many embodiments, the individual is a human being. Unless otherwise stated, the terms “individual” and “subject” do not denote a particular age, and thus encompass adults, elderlies, children, and newborns. In some embodiments of the present disclosure, the “individual” or “subject” is a “patient”. In some embodiments, the terms “individual” and “subject” relate to pregnant women and immunocompromised persons.

The term “patient” means an individual or subject for treatment, in particular a diseased individual or subject. RNA described herein may be administered to a subject for delivering the RNA to cells of the subject.

RNA described herein may be administered to a subject for delivering a therapeutic or prophylactic peptide or polypeptide (e.g., a pharmaceutically active peptide or polypeptide) to the subject, wherein the RNA encodes a therapeutic or prophylactic peptide or polypeptide.

RNA described herein may be administered to a subject for treating or preventing a disease in a subject, wherein delivering the RNA to cells of the subject is beneficial in treating or preventing the disease.

RNA described herein may be administered to a subject for treating or preventing a disease in a subject, wherein the RNA encodes a therapeutic or prophylactic peptide or polypeptide and wherein delivering the therapeutic or prophylactic peptide or polypeptide to the subject is beneficial in treating or preventing the disease.

In some embodiments of the disclosure, the aim is to induce an immune response by providing RNA described herein.

A person skilled in the art will know that one of the principles of immunotherapy and vaccination is based on the fact that an immunoprotective reaction to a disease is produced by immunizing a subject with an antigen or an epitope, which is immunologically relevant with respect to the disease to be treated. Accordingly, RNA described herein is applicable for inducing or enhancing an immune response. RNA described herein is thus useful in a prophylactic and/or therapeutic treatment of a disease involving an antigen or epitope.

In some embodiments of the disclosure, the aim is to provide an immune response against cells comprising an antigen, e.g., Mtb antigen.

In some embodiments of the disclosure, the aim is to prophylactically or therapeutically treat tuberculosis by vaccination.

Due to the high degree of sequence conservation of the disclosed antigens between different Mycobacterium species, including Mycobacterium tuberculosis, Mycobacterium bovis, Mycobacterium caprae, Mycobacterium orygis, Mycobacterium africanum, Mycobacterium microti, Mycobacterium canetti and Mycobacterium pinnipedii, exposure of a subject to Mycobacterium tuberculosis antigens will result in a high degree of cross-reactivity with antigens of other Mycobacterium species. Therefore, a vaccine based on or directed at Mycobacterium tuberculosis antigens will elicit a robust immune response against other Mycobacterium species, in particular Mycobacterium bovis, Mycobacterium caprae, Mycobacterium orygis, Mycobacterium africanum, Mycobacterium microti, Mycobacterium canetti and Mycobacterium pinnipedii as well.

In some embodiments of the disclosure, the aim is to provide an immune response against a Mycobacterium selected from Mycobacterium tuberculosis, Mycobacterium bovis, Mycobacterium caprae, Mycobacterium orygis, Mycobacterium africanum, Mycobacterium microti, Mycobacterium canetti and Mycobacterium pinnipedii and to prevent or treat tuberculosis.

In preferred embodiments of the disclosure, the aim is to provide an immune response against Mycobacterium tuberculosis.

In some embodiments of the disclosure, the aim is to treat an infection with Mycobacterium tuberculosis.

In some embodiments of the disclosure, the aim is to prevent or treat disease symptoms caused by an infection with Mycobacterium tuberculosis.

In some embodiments of the disclosure, the aim is to provide protection against an infection with Mycobacterium tuberculosis by vaccination.

In some embodiments of the disclosure, the aim is to provide protection against an outbreak of disease in a subject infected with Mycobacterium tuberculosis. In some embodiments of the disclosure, the aim is to provide protection against symptoms of tuberculosis in a subjected infected with Mycobacterium tuberculosis.

In some embodiments, the RNA is present in a composition as described herein.

In some embodiments, the RNA is administered in a pharmaceutically effective amount.

In some embodiments, the subject is a mammal. In some embodiments, the mammal is a human.

In some embodiments, the subject treated had been exposed to Mycobacterium tuberculosis. In some embodiments, the subject treated had not been exposed to Mycobacterium tuberculosis.

In some embodiments, the treatments described herein involve pre- or post-exposure vaccination against Mycobacterium tuberculosis, or a combination thereof.

Citation of documents and studies referenced herein is not intended as an admission that any of the foregoing is pertinent prior art. All statements as to the contents of these documents are based on the information available to the applicants and do not constitute any admission as to the correctness of the contents of these documents.

The description (including the following examples) is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

### EXAMPLES

### Example 1: Constructs and In Vitro Expression of Full-Length Antigens

All antigens were analyzed for the presence of a predicted human signal peptide or a predicted transmembrane domain. If needed constructs with (WT), or without (ΔSP) the signal peptide sequence were designed, since such modification have the potential to improve immunogenicity of encoded antigens. All constructs were modified with a human MHC-I derived signal peptide (husec) followed by an N-terminal FLAG (DYKDDDDK—SEQ ID NO: 42). The coding sequences were codon-optimized for expression in humans and were encoded on nucleoside modified RNA (ModRNA).

Cells from a human embryonic kidney cell line (HEK293T) were transfected with 1 μg of RNA encoding the antigens and modified as described above. Lysates of the transfected cell lines were generated 18 hours after transfection and were analyzed for expression of the flag-tag labeled antigens by SDS-PAGE followed by Western blot as well as by Flow cytometry.

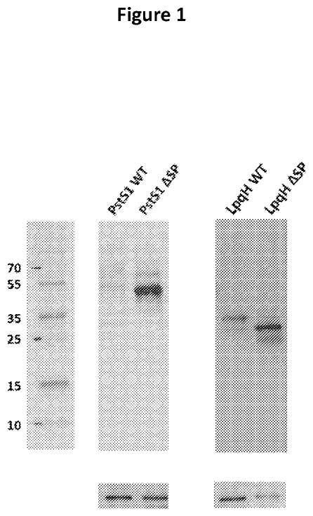

Depicted in FIG. 1 are Western blots stained with anti-FLAG (top) and anti-tubulin antibody as a loading control (bottom). The molecular weight (kDa) is depicted on the left of each blot.

LpqH and PstS1 expressed better in their modified version compared to the wild-type construct. Both proteins were detected at the expected molecular weight.

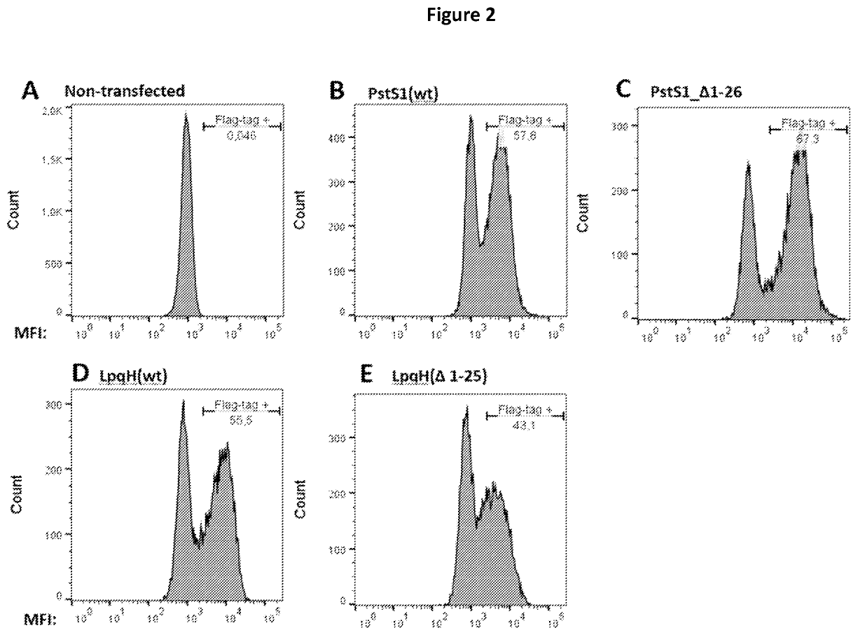

Depicted in FIG. 2 is the mean fluorescent intensity of HEK293T cell stained with an anti-FlagTag-BV421 conjugate antibody. The horizontal bar in the plots depicts the gate for Flag-Tag positive cells. For PstS1, cells transfected with the flag-tagged full-length construct (B) expressed at a slightly lower mean fluorescent intensity (MFI) compared to the PstS1_Δ1-26 (C). In contrast for LpqH the full-length construct expressed slightly higher compared to LpqH_Δ 1-25. In summary, both antigens can be well expressed in either the full-length form or the truncated form in human cells.

### Example 2: In Vivo Expression and Immunogenicity of Full-Length Antigens

C57BL/6 mice were vaccinated (Day 0) and boosted (Day 21) with modRNA encoding LpqH or PstS1-ΔSP modified with the human MHC-I derived signal peptide followed by an N-terminal FLAG (SEQ ID NO: 42). The RNA was formulated in an in-house lipid nanoparticle (LNP). Three weeks after the boost (Day 42), mice were sacrificed and splenocytes were isolated. Total splenocytes were analyzed for cytokine production per animal as described below. In addition, splenocytes were pooled per experimental group and separated into CD4+ and CD8+ cell fraction by antibody labeling followed by magnetic associated cell sorting.

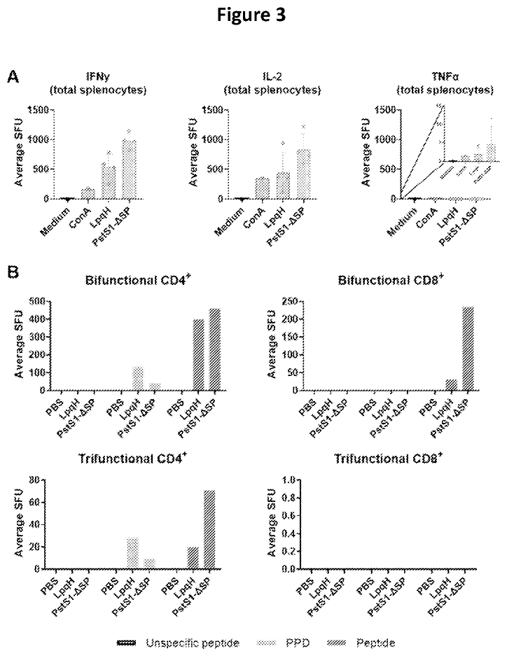

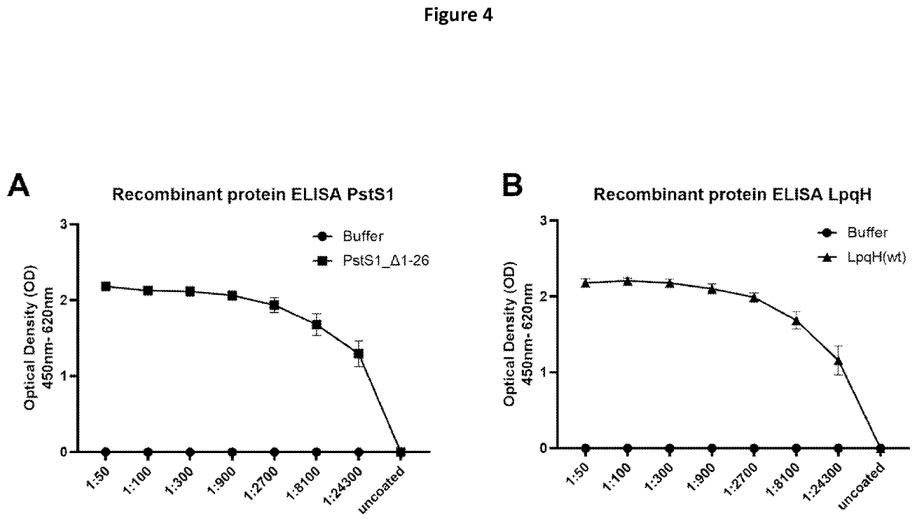

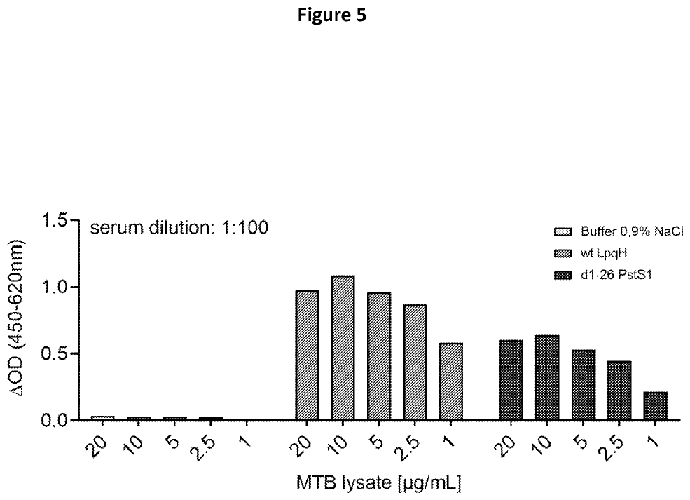

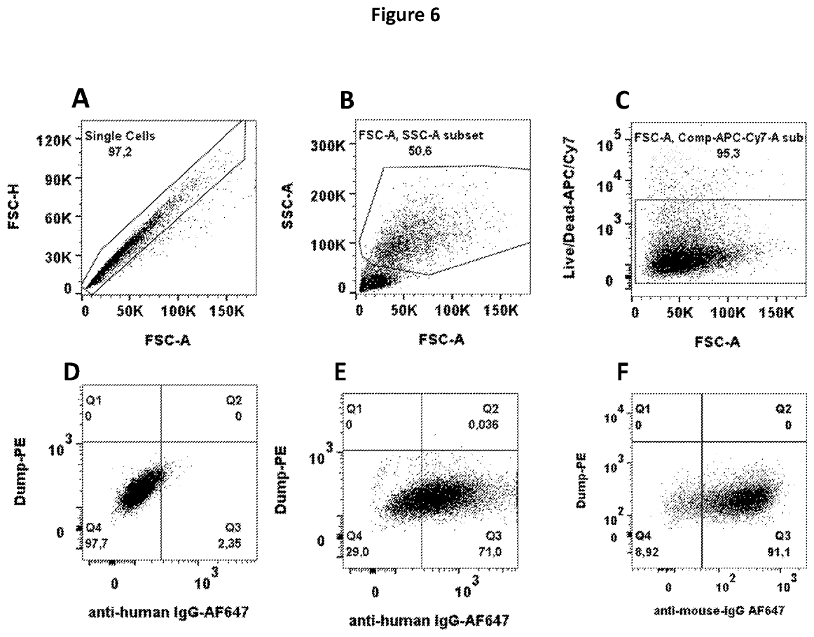

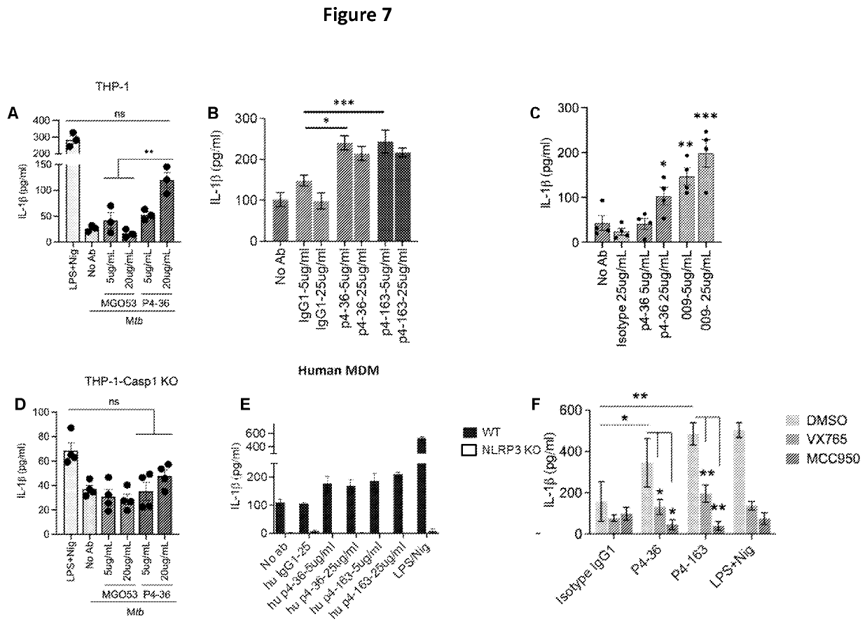

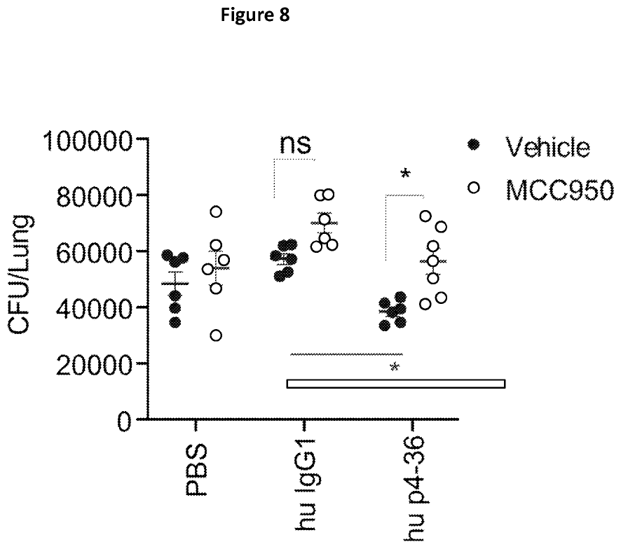

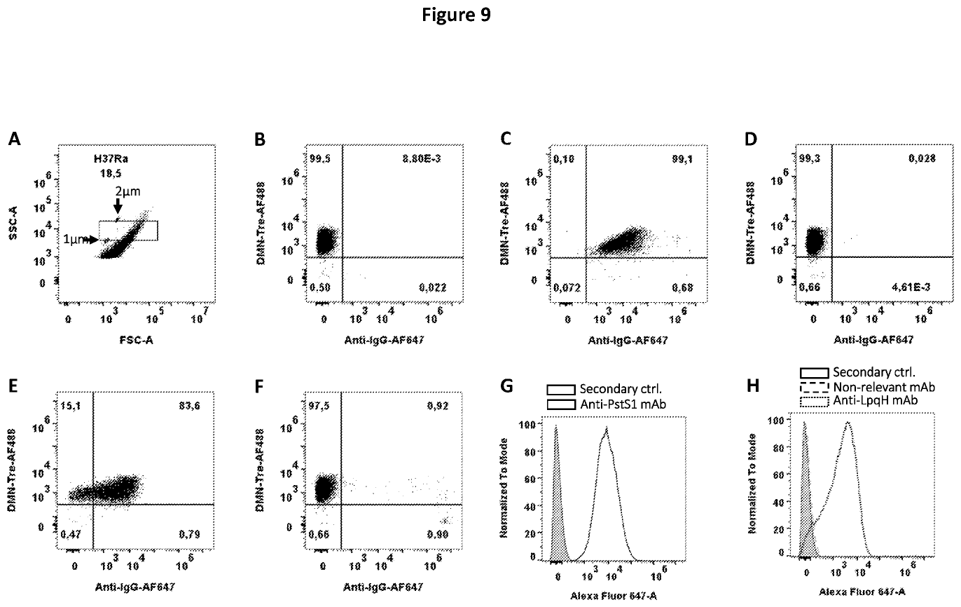

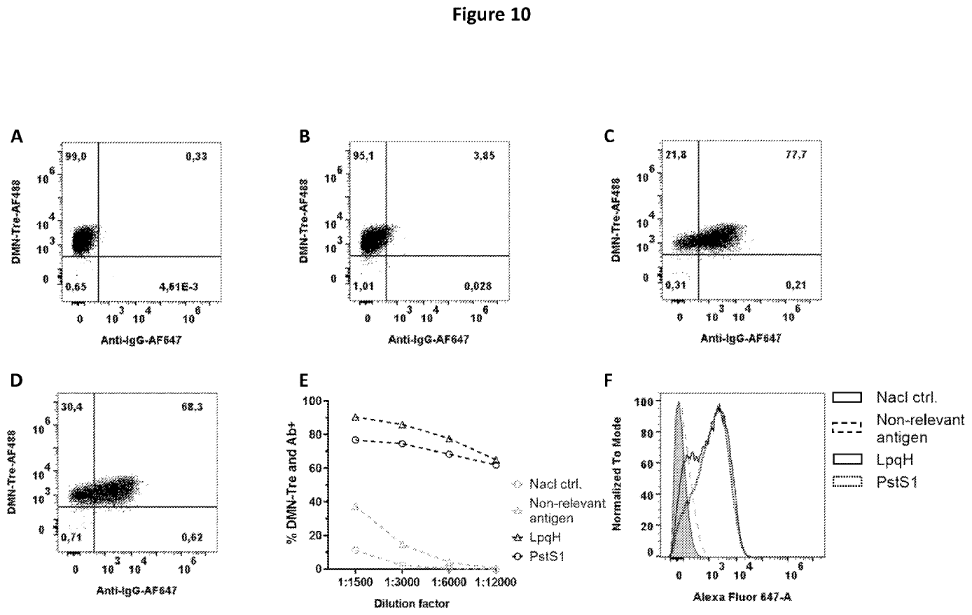

All splenocyte fractions (total, or CD4+ and CD8+ MACS-isolated) were added to pre-coated fluorospot plates and incubated with a library of overlapping 15-mer peptides with a 5 amino acid offset (i.e. peptide one covers amino acids 1-15 of the antigen, peptide 2 covers amino acid 6-20 and so forth), or indicated controls. Antigen-specific T-cell responses were determined by staining the plates with primary antibodies recognizing murine IFNγ, IL-2 and TNFα, followed by staining with fluorophore labeled secondary antibodies. After incubation with fluorescence enhancer and drying of the plates, fluorescent signals were measured by a fluorospot plate reader. Both antigens induced strong IFNγ and IL-2 production from total splenocytes, although PstS1-ΔSP slightly outperformed LpqH (FIG. 3A). Similarly, with separated CD4+ and CD8+ cells, all antigens induced robust CD4+ responses (Data not shown). Differences between constructs were most apparent in trifunctional and bifunctional T cell responses in the CD4+ and CD8+ cells respectively (FIG. 3B). PstS1-ΔSP induced the highest levels of trifunctional CD4+ and bifunctional CD8+ responses respectively (FIG. 3B). Of note, restimulation of the CD4+ splenocytes with purified protein derivate (PPD) from Mycobacterium tuberculosis also induced specific cytokine responses in mice immunized with both RNA candidates. This indicates that the T cells raised by our vaccine can be activated to produce relative cytokine responses when recognizing native Mycobacterium tuberculosis antigens.

### Example 3: MRNA Encoded LpqH and PstS1 Induce Strong Antibody Responses In Vivo

To evaluate whether the PstS1-A1-26 or LpqH-full length constructs induce antibodies specific for their respective targets, enzyme-linked immunosorbent assays (ELISA) were conducted. 96-well Maxisorp ELISA plates were coated with 100 μl of recombinant protein at a concentration of 1 μg/ml. The plates were covered and incubated at 4° C. overnight. After incubation, the plates were washed three times with 300 μL/well PBS-Tween and then blocked by adding 250 μL of 1× Blocking buffer (BB) per well and incubated for 1 hour at 37° C. on a shaker. The plates were then washed again three times with 300 μL/well PBS-Tween. The serum samples were serially diluted in 1×BB in 1:3 steps starting at a dilution of 1:100. 100 μL of the diluted samples or negative controls was added in each corresponding well. The plate was incubated for 1 hour at 37° C. on a shaker and then washed three times with 300 μL/well PBS-Tween. Afterwards, anti-mouse IgG-Horseradish peroxidase (HRP) secondary antibody was diluted 1:15000 in 1×BB, 100 μL antibody was added to each well, and the plate was incubated for 45 minutes at 37° C. on a shaker. The plate was washed three times with 300 μL/well PBS-Tween. 100 μl of 3,3′,5,5′-Tetramethylbenzidin (TMB)-substrate was added per well, and the plate was incubated for 8 minutes at room temperature. The reaction was stopped by adding 100 μl of 25% sulphuric acid per well. The absorbance was read on a plate reader at 450 nm and 620 nm (reference).

The ELISA experiments revealed that polyclonal antibodies induced by the PstS1-41-26 or LpqH-full length constructs were reactive for the recombinant PstS1 (FIG. 4A) and LpqH (FIG. 4B), even at very high dilutions of 1:24300 indicating the potential of the vaccine formulation to induce high antibody titers against the specific proteins.

### Example 4: MRNA Encoded LpgH and PstS1 Induce Specific Antibodies that Bind to Mycobacterium tuberculosis Lysate

The same serum as analyzed in example 3 was also tested in an ELISA against H37Rv lysate at a concentration range of 20 μg/ml-1 μg/ml. A specific signal was detected at different lysate concentrations only when serum from mice immunized with LpqH or PstS1 encoding mRNA was used (FIG. 5). Importantly, the reactivity of serum-derived antibodies to recognize Mtb lysate is indicative of major implications. Firstly, it means that antibodies induced by mRNA vaccine candidates are able to recognize these antigens in their native conformations as produced by Mycobacterium tuberculosis and may therefore be relevant to combat human infection. Secondly, it means that the titers of these antibodies and their binding avidity are sufficient to induce a specific signal in the context of many other bacterial proteins and other components present in the lysate, increasing the likelihood that such antibodies are protective in the context of an infection.

### Example 5: MRNA Encoded PstS1 is Translated and Recognized by a Monoclonal Antibody that Protects Against Tuberculosis at Least in Part Through Potentiating Inflammasome Activation

A human embryonic kidney cell line HEK293T was transfected with 0.5 μg of mRNA-encoded PstS1 or left untransfected. Transfected cells were collected after overnight incubation and stained with monoclonal antibody p4-163 (Watson et al 2021), followed by an alexa-fluor-647 labeled secondary anti-human IgG antibody. After PFA-fixation, stained cells were acquired by flow assorted cell sorting (FACS) and gated for single cells (Gating strategy depicted in FIG. 6, top panels). Cells transfected with RNA-encoded PstS1 were efficiently stained with the p4-163 antibody (FIG. 6, bottom right, 71% positivity in gate Q3) and this staining was specific, because untransfected cells were not labeled with the antibody (FIG. 6, bottom left, 2.35% positivity in gate Q3). These data show that mRNA encoded PstS1 is translated into a protein that can be recognized by a protective antibody isolated from an Mtb infected patient. These data show that antibodies raised by immunization with this RNA have the potential to induce antibodies with protective function against the same or other epitopes. Since the p4-163 antibody recognizes a conformational epitope, these data furthermore suggest that the encoded antigen has correct folding compared to the antigen produced by Mtb despite being produced in a eukaryotic human expression system.

To investigate how mRNA vaccines against PstS1 and LpqH can mediate protection against tuberculosis not just through T-cell responses but also through antibody mediated responses specifically, experiments were performed with human monoclonal antibodies recognizing these antigens. First, a single-cell suspension of Mtb-HN878, grown detergent free, was used to infect macrophages at a multiplicity of infection (MOI) of 1:1-5:1, depending on the experiment. Differentiated macrophages were infected with preformed Mtb:Ab immune complexes. These infections were performed in multiple models. A first experiment was performed in the human monocyte-like cell-line THP-1 differentiated to macrophages with PMA (10 ng/ml) for 48 hrs then rested for an additional 24 hrs prior to infection (depicted in FIG. 7A). A second experiment was performed in primary murine bone-marrow-derived macrophages (BMDM), which were isolated from femurs of euthanized animals and differentiated by murine M-CSF (20 ng/ml) for 6 days prior to infection (depicted in FIG. 7B,C). Macrophages were infected for 4 hrs in serum-free medium prior to incubation with amikacin to kill extracellular bacteria. Supernatants were collected at 24 hrs post-infection for cytokine analysis. ELISAs against IL-1b were purchased from Biolegend and performed according to manufacturer's instruction. These data show that antibodies against PstS1 (FIG. 7A-C) and LpqH (FIG. 7C) increase protective immune responses coordinated by the IL-1β cytokine axis. Genetic knock-out in THP1 macrophages of the gene encoding Caspase 1 resulted in loss of the immune activating effect (FIG. 7D). BMDM's from mice lacking a functional NLRP3 inflammasome were similarly unable to mount this protective response (FIG. 7E). Both these findings were potentiated by chemical inhibition of either caspase 1 by the caspase 1 inhibitor (VX765-50 μM) or the NLRP3 inhibitor (MCC950-10 μM).

An in vivo study confirmed, that antibody-mediated potentiation of IL-1B immune responses is indeed a protective mechanism against tuberculosis. C57/BL6 WT mice were injected once intraperitoneally with IgG1 isotype or anti-PstS1 antibody (p4-36) (0.1 mg). The next day mice were infected with a low dose of Mtb-HN878 (10-20 CFU). Mice were treated daily with either the specific NLRP3 inhibitor MCC950 or vehicle, both by intraperitoneal injection starting on d-3. Mice were euthanized 2 weeks post infection, and lung bacterial burden was measured (FIG. 8). Indeed, a protective effect of specific PstS1 antibodies was seen by a significant (p<0.05) reduction in recovered CFU at the end of the experiment compared to a control human IgG antibody. Strikingly, this protection was fully abrogated in the context of the NLRP3 inflammasome inhibitor MCC950.

Together, these data show protection against tuberculosis specifically by human antibodies against PstS1 and LpqH. Since the data presented here show that mRNA transfection with constructs encoding PstS1 or LpqH was able to trigger binding of such antibodies, it is likely that human vaccination with these constructs would induce antibody responses that protect against tuberculosis, by a previously undescribed mechanism.

### Example 6: Removal of N-Glycosylation Sites to Improve Antigen Expression and Folding

Eukaryotic cells modify proteins by a system called N-linked glycosylation, which does not occur in Mycobacterium tuberculosis. This post-translational modification can have pronounced effects on a protein's folding in their correct structure or the ability of antibodies to bind to an antigen. Therefore, especially when designing a vaccine against a bacterial pathogen like Mycobacterium tuberculosis, it can be essential or advantageous to mutate N-linked glycosylation sites. In order to do so, asparagine (N) residues in the identified putative glycosylations sites are mutated to glutamine (Q). For PstS1 this is relevant for asaparagine residues 78 and 268 of the wild-type sequence (SEQ ID NO: 2). N to Q mutation of these residues results in the sequence of SEQ ID NO: 44. For LpqH this concerns residues 25, 60 and 120 from the wildtype sequence (SEQ ID NO: 1), resulting in the sequence of SEQ ID NO: 43, after the N to Q mutations.

### Example 7: Antibodies Induced by mRNA-Encoded LpqH and PstS1 Bind to the Surface of Live Mycobacterium tuberculosis

To investigate the capability of LpqH and PstS1 encoding mRNAs in inducing antibodies specific to their respective targets on live bacteria, a H37Ra binding assay was established. To visualize mycobacteria for flow cytometric analysis, the bacteria were stained with 4-N,N-Dimethylamino-1,8-naphthalimide conjugated to trehalose. The green-fluorescent probe is actively incorporated into the membrane of metabolically active bacteria. The probe was added at a concentration of 1 mM to the culture at a density of 0.5 OD600 and incubated overnight at 37° C. while shaking. The following morning, the bacteria were washed and stained with either a murine anti-LpqH, human anti-PstS1 p4-163, a non-relevant monoclonal control antibody, or with serum from mice repeatedly immunized with LpqH-, or PstS1-encoding mRNAs. The bacteria were incubated for one hour at room temperature in the presence of the primary antibody. Subsequently, the H37Ra culture was washed, and the respective secondary antibody was added (coupled to Alexa Fluor 647). As negative controls, samples were stained with only the respective secondary antibodies, a non-related primary antibody, serum from NaCl control animals, or serum from mice immunized with a non-related mRNA-encoded antigen. To ensure gating on events of correct size and to maintain reproducibility of the data, beads of 1 and 2 μm size were included and used to define the borders of gates (FIG. 9A). The level of DMN-Tre staining and antibody surface staining was assessed through flow cytometry by gating on Alexa Fluor 488 (DMN-Tre) or Alexa Fluor 647 (surface IgG) double positive H37Ra. Both, the monoclonal anti-PstS1 p4-163 and anti-LpqH antibodies (FIGS. 9C and E) led to high frequencies of surface-labeled bacteria. In contrast, the frequencies remained low if the bacteria were stained with the respective secondary antibody only (FIGS. 9B and D) or with the non-related primary monoclonal murine antibody (FIG. 9F). The data for H37Ra surface staining using monoclonal antibodies against PstS1 and LpqH are summarized in FIGS. 9G and H as histograms.

The H37Ra bacteria were also subjected to staining with polyclonal serum raised by two doses, 21 days apart, of mRNA-encoded LpqH, PstS1 or controls in C57BL/6 mice (FIGS. 10A and B). Bacteria stained with polyclonal serum from NaCl-injected negative control mice (FIG. 10A), or from mice immunized with a non-related antigen (FIG. 10B) resulted in consistently low surface binding, reaching baseline levels at a dilution of 1:6000 (FIG. 10E). In contrast, H37Ra stained with sera from PstS1 and LpqH immunized animals showed consistently high surface labeling also at these dilutions (FIGS. 10C and D). The titration curve in FIG. 10E reveals that the specific signal remained high even at dilutions of 1:6000 and 1:12000. The data obtained from H37Ra surface staining using serum samples from PstS1 and LpqH immunized animals are summarized in the histograms presented in FIG. 10F.

1. RNA molecule comprising a sequence encoding at least one full-length antigen or antigen fragment representing at least one Mycobacterium tuberculosis antigen or immunogenic variant thereof, wherein the Mycobacterium tuberculosis antigen is selected from the group of LpqH and PstS1.

2. The RNA molecule of claim 1, wherein the sequence of one or more of the at least one full-length antigens or antigen fragments is altered by removal of a predicted bacterial signal peptide.

3. The RNA molecule of claim 1 or 2, wherein the sequence of one or more of the at least one full-length antigens or antigen fragments is altered by addition of a non-native human, bacterial or viral signal peptide to its N-terminus.

4. The RNA molecule of claim 3, wherein the non-native signal peptide is selected from the group comprising a HSV-1 glycoprotein D signal peptide, a HSV-2 glycoprotein D signal peptide, a human Ig heavy chain signal peptide, a HuIgGk signal peptide, an IgE heavy chain epsilon-1 signal peptide, a Japanese encephalitis PRM signal sequence or a VSVg protein signal sequence.

5. The RNA molecule of claim 4, wherein the non-native signal peptide is a viral signal peptide, preferably, wherein the non-native signal peptide is a HSV-1 glycoprotein D signal peptide.

6. The RNA molecule of any one of claims 1 to 5, wherein the amino acid sequence of one or more of the at least one full-length antigens or antigen fragments is altered by replacing at least one transmembrane domain with a disrupted transmembrane domain.

7. The RNA molecule of any one of claims 1 to 5, wherein the amino acid sequence of one or more of the at least one full-length antigens or antigen fragments is altered by addition of a non-native trafficking domain and/or non-native transmembrane domain to its C-terminus.

8. The RNA molecule of claim 7, wherein the non-native trafficking domain is an MHC class I trafficking domain and/or wherein the non-native transmembrane domain is a human, bacterial or viral transmembrane domain.

9. The RNA molecule of any one of claims 1 to 8, wherein:
a) the LpqH antigen comprises the amino acid sequence of any one of SEQ ID NOs: 1, 43 or 45 and an immunogenic variant thereof comprises an amino acid sequence having at least 99%, 98%, 97%, 96%, 95%, 90%, 85%, or 80% identity to the amino acid sequence of SEQ ID NO: 1, 43 or 45;
b) the PstS1 antigen comprises the amino acid sequence of any one of SEQ ID NOs: 2, 44 or 46 and an immunogenic variant thereof comprises an amino acid sequence having at least 99%, 98%, 97%, 96%, 95%, 90%, 85%, or 80% identity to the amino acid sequence of SEQ ID NO: 2, 44 or 46;
c) the non-native signal peptide comprises an amino acid sequence selected from the group of SEQ ID NOs: 3 to 23, amino acid sequences having at least 96%, 92%, 88%, 84% or 80% identity to SEQ ID NOs: 3 to 23, amino acid sequences encoded by a nucleotide sequence selected from the group of SEQ ID NOs: 24 to 37 and amino acid sequences having at least 96%, 92%, 88%, 84% or 80% identity to amino acid sequences encoded by a nucleotide sequence selected from the group of SEQ ID NOs: 24 to 37; and/or
d) the non-native trafficking signal comprises the amino acid sequence SEQ ID NO: 38 or an amino acid sequence having at least 98%, 96%, 94%, 92%, 90%, 85% or 80% identity to the amino acid sequence of SEQ ID NO: 38.

10. The RNA molecule of any one of claims 1 to 9, wherein the RNA molecule comprises a 5′ cap.

11. The RNA molecule of claim 10, wherein the 5′ cap comprises a cap1 structure.

12. The RNA molecule of claim 10, wherein the 5′-cap comprises m₂^{7,3′-O}Gppp (m₁^{2′-O})ApG.

13. The RNA molecule of any one of claims 1 to 12, wherein the RNA molecule comprises a 5′-UTR.

14. The RNA molecule of claim 13, wherein the 5′-UTR comprises a modified human alpha-globin 5′-UTR.

15. The RNA molecule of claim 13 or 14, wherein the 5′-UTR comprises the nucleotide sequence of SEQ ID NO: 39, or a nucleotide sequence having at least 98%, 96%, 94%, or 80% identity to the nucleotide sequence of SEQ ID NO: 39.

16. The RNA molecule of any one of claims 1 to 15, wherein the RNA comprises a 3′-UTR.

17. The RNA molecule of claim 16, wherein the 3′-UTR comprises a first sequence from the amino terminal enhancer of split (AES) messenger RNA and a second sequence from the mitochondrial encoded 12S ribosomal RNA.

18. The RNA molecule of claim 16 or 17, wherein the 3′-UTR comprises the nucleotide sequence of SEQ ID NO: 40, or a nucleotide sequence having at least 99%, 98%, 97%, 96%, 95%, 90%, 85%, or 80% identity to the nucleotide sequence of SEQ ID NO: 40.

19. The RNA molecule of any one of claims 1 to 18, wherein the RNA molecule comprises a polyA sequence.

20. The RNA molecule of claim 19, wherein the polyA sequence is an interrupted sequence of A nucleotides.

21. The RNA molecule of claim 19 or 20, wherein the polyA sequence comprises 30 adenine nucleotides followed by 70 adenine nucleotides, wherein the 30 adenine nucleotides and 70 adenine nucleotides are separated by a nucleotide linker sequence of 10 nucleotides.

22. The RNA molecule of any one of claims 19 to 21, wherein the polyA sequence is or comprises the nucleotide sequence of SEQ ID NO: 41, or a nucleotide sequence having at least 99%, 98%, 97%, 96%, 95%, 90%, 85%, or 80% identity to the nucleotide sequence of SEQ ID NO: 41.

23. The RNA molecule of any one of claims 19 to 22, wherein the RNA molecule comprises a 5′-cap, a 5′-UTR, a 3′-UTR, and a polyA sequence.

24. The RNA molecule of any one of claims 1 to 23, wherein the RNA molecule comprises modified nucleotides, nucleosides or nucleobases.

25. The RNA molecule of claim 24, wherein the RNA molecule comprises modified uridines.

26. The RNA molecule of claim 25, wherein the RNA molecule comprises modified uridines in place of all uridines.

27. The RNA molecule of claim 25 or 26, wherein the modified uridines are N1-methyl-pseudouridine.

28. The RNA molecule of any one of claims 1 to 27, wherein the coding sequence of the RNA molecule is codon-optimized and/or is characterized in that its G/C content is increased compared to the parental sequence.

29. Protein encoded by the RNA molecule of any one of claims 1 to 28.

30. DNA molecule encoding the RNA molecule of any one of claims 1 to 28.

31. Pharmaceutical composition comprising one or more RNA molecules of any one of claims 1 to 28.

32. The pharmaceutical composition of claim 31, wherein the one or more RNA molecule is formulated in a lipid formulation, such as in lipid nanoparticles or liposomes.

33. The pharmaceutical composition of claim 32, wherein the lipid formulation comprises each of:
a) a cationically ionizable lipid;
b) a steroid;
c) a neutral lipid; and
d) a polymer-conjugated lipid.

34. The pharmaceutical composition of claim 33, wherein the cationically ionizable lipid is present in a concentration ranging from about 40 to about 60 mol percent of the total lipids.

35. The pharmaceutical composition of claim 33 or 34, wherein the steroid is present in a concentration ranging from about 30 to about 50 mol percent of the total lipids.

36. The pharmaceutical composition of any one of claims 33 to 35, wherein the neutral lipid is present in a concentration ranging from about 5 to about 15 mol percent of the total lipids.

37. The pharmaceutical composition of any one of claims 33 to 36, wherein the polymer-conjugated lipid is present in a concentration ranging from about 1 to about 10 mol percent of the total lipids.

38. The pharmaceutical composition of any one of claims 33 to 37, wherein the cationically ionizable lipid is within a range of about 40 to about 60 mole percent, the steroid is within a range of about 30 to about 50 mole percent, the neutral lipid is within a range of about 5 to about 15 mole percent, and the polymer-conjugated lipid is within a range of about 1 to about 10 mole percent.

39. The pharmaceutical composition of any one of claims 33 to 38, wherein the steroid comprises cholesterol.

40. The pharmaceutical composition of any one of claims 33 to 39, wherein the neutral lipid comprises a phospholipid.

41. The pharmaceutical composition of claim 40, wherein the phospholipid comprises distearoylphosphatidylcholine (DSPC).

42. The pharmaceutical composition of any one of claims 33 to 41, wherein the polymer-conjugated lipid comprises a polyethylene glycol (PEG)-lipid.

43. The pharmaceutical composition of any one of claims 31 to 42, wherein the pharmaceutical composition further comprises one or more pharmaceutically acceptable carriers, diluents, excipients and/or adjuvants.

44. The pharmaceutical composition of claim 43, wherein the adjuvants comprise an RNA encoding one or more immunomodulating molecules, such as cytokines.

45. The pharmaceutical composition of claim 43 or 44, wherein the adjuvants comprise one or more immunity inducing or immune-modulating moieties.

46. The pharmaceutical composition of claim 45, wherein the one or more immunity inducing or immune-modulating moieties comprise a peptidoglycan moiety.

47. The pharmaceutical composition of any one of claims 31 to 46, wherein the one or more RNA molecules are in a liquid formulation.

48. The pharmaceutical composition of any one of claims 31 to 46, wherein the one or more RNA molecules are in a frozen formulation.

49. The pharmaceutical composition of any one of claims 31 to 46, wherein the one or more RNA molecules are in a lyophilized formulation.

50. The pharmaceutical composition of any one of claims 31 to 49, wherein the one or more RNA molecules are formulated for injection.

51. The pharmaceutical composition of claim 50, wherein the one or more RNA molecules are formulated for intramuscular administration.

52. The pharmaceutical composition of any one of claims 31 to 51, wherein the pharmaceutical composition is formulated for administration in human.

53. Kit comprising one or more pharmaceutical composition of any one of claims 31 to 52.

54. The kit of claim 53, comprising two or more pharmaceutical compositions which comprise the same or different RNA molecules of any one of claims 1 to 28 in separate vials.

55. The kit of claim 53 or 54, further comprising instructions for use of the one or more pharmaceutical composition for treating or preventing tuberculosis.

56. RNA molecule of any one of claims 1 to 28, protein of claim 29, DNA molecule of claim 30, pharmaceutical composition of any one of claims 31 to 52 or kit of any one of claims 53 to 55 for use as a medicament.

57. The RNA molecule, protein, DNA molecule, pharmaceutical composition or kit for use of claim 56, wherein the use comprises a therapeutic or prophylactic treatment of a disease or disorder in a subject.

58. The RNA molecule, protein, DNA molecule, pharmaceutical composition or kit for use of claim 56 or 57, wherein the use comprises the use as a vaccine against a disease or disorder in a subject.

59. The RNA molecule, protein, DNA molecule, pharmaceutical composition or kit for use of claim 57 or 58, wherein the subject is a human infected with the disease or disorder or in danger of contracting the disease or disorder.

60. RNA molecule of any one of claims 1 to 28, protein of claim 29, DNA molecule of claim 30, pharmaceutical composition of any one of claims 31 to 52 or the kit of any one of claims 53 to 55 for use in treating or preventing tuberculosis in a subject.

61. The RNA molecule, protein, DNA molecule, pharmaceutical composition or kit for use of claim 60, wherein the subject is a human infected with tuberculosis or in danger of contracting tuberculosis.

62. The RNA molecule, protein, DNA molecule, pharmaceutical composition or kit for use of claim 60 or 61, wherein the use is as a vaccine for preventing tuberculosis.

63. Use of the RNA molecule of any one of claims 1 to 28, the protein of claim 29, the DNA molecule of claim 30, the pharmaceutical composition of any one of claims 31 to 52 or the kit of any one of claims 53 to 55 for the manufacture of a medicament for treating or preventing tuberculosis.

64. Method of vaccinating a subject comprising administering the RNA molecule of any one of claims 1 to 28, the protein of claim 29, the DNA molecule of claim 30, the pharmaceutical composition of any one of claims 31 to 52 or the kit of any one of claims 53 to 55 to the subject.

65. The method of claim 64, wherein the vaccination is for preventing tuberculosis.

66. The method of claim 64 or 65, wherein administration is intramuscular administration.

67. The method of any one of claims 64 to 66, comprising administering to the subject at least one dose of the RNA molecule, protein, DNA molecule, pharmaceutical composition or kit.

68. The method of any one of claims 64 to 67, comprising administering to the subject at least two doses of the RNA molecule, protein, DNA molecule, pharmaceutical composition or kit.

69. The method of any one of claims 64 to 68, wherein an amount of the RNA molecule of at least 10 μg per dose is administered.

70. The method of any one of claims 64 to 69, wherein the subject is a human.

71. The RNA molecule, chimeric protein, pharmaceutical composition or kit for use of any one of claims 60 to 62, the use of claim 63 or the method of any one of claims 65 to 70, wherein the tuberculosis is caused by an infection with a Mycobacterium.

72. The RNA molecule, chimeric protein, pharmaceutical composition or kit for use, the use or the method of claim 71, wherein the Mycobacterium is selected from the group of Mycobacterium tuberculosis, Mycobacterium bovis, Mycobacterium caprae, Mycobacterium orygis, Mycobacterium africanum, Mycobacterium microti, Mycobacterium canetti and Mycobacterium pinnipedii.

73. The RNA molecule, chimeric protein, pharmaceutical composition or kit for use, the use or the method of claim 72, wherein the Mycobacterium is Mycobacterium tuberculosis.
